SEQUENCE LISTING (739 sequences)

SEQ ID NO: 1 - protein, 7 aa, synthetic construct
GGHSPGG

SEQ ID NO: 2 - DNA, 24 bp, synthetic construct
  source 1..24: mol_type = other DNA
tcatttgctaatagtagtagcatt

SEQ ID NO: 3 - DNA, 24 bp, synthetic construct
  source 1..24: mol_type = other DNA
accctcattcagggatagcaagcc

SEQ ID NO: 4 - DNA, 24 bp, synthetic construct
  source 1..24: mol_type = other DNA
agaatatcagacgacgacaataaa

SEQ ID NO: 5 - DNA, 24 bp, synthetic construct
  source 1..24: mol_type = other DNA
tcatatgcgttatacaaaggcgtt

SEQ ID NO: 6 - DNA, 24 bp, synthetic construct
  source 1..24: mol_type = other DNA
cgggagaatttaatggaaacagta

SEQ ID NO: 7 - DNA, 24 bp, synthetic construct
  source 1..24: mol_type = other DNA
gcgcgtactttcctcgttagaatc

SEQ ID NO: 8 - DNA, 30 bp, synthetic construct
  source 1..30: mol_type = other DNA
ttaagagggtccaatactgcggatagcgag

SEQ ID NO: 9 - DNA, 30 bp, synthetic construct
  source 1..30: mol_type = other DNA
aggcttttcaggtagaaagattcaattacc

SEQ ID NO: 10 - DNA, 30 bp, synthetic construct
  source 1..30: mol_type = other DNA
ttatgcgattgacaagaaccggaggtcaat

SEQ ID NO: 11 - DNA, 30 bp, synthetic construct
  source 1..30: mol_type = other DNA
cataagggacactaaaacactcacattaaa

SEQ ID NO: 12 - DNA, 30 bp, synthetic construct
  source 1..30: mol_type = other DNA
cgggtaaaattcggtcgctgaggaatgaca

SEQ ID NO: 13 - DNA, 30 bp, synthetic construct
  source 1..30: mol_type = other DNA
gtctctgacaccctcagagccacatcaaaa

SEQ ID NO: 14 - DNA, 30 bp, synthetic construct
  source 1..30: mol_type = other DNA
tcaccggaaacgtcaccaatgaattattca

SEQ ID NO: 15 - DNA, 30 bp, synthetic construct
  source 1..30: mol_type = other DNA
ttaaaggtacatataaaagaaacaaacgca

SEQ ID NO: 16 - DNA, 30 bp, synthetic construct
  source 1..30: mol_type = other DNA
ataataactcagagagataacccgaagcgc

SEQ ID NO: 17 - DNA, 30 bp, synthetic construct
  source 1..30: mol_type = other DNA
attagacggagcgtctttccagagctacaa

SEQ ID NO: 18 - DNA, 30 bp, synthetic construct
  source 1..30: mol_type = other DNA
tatataacgtaaatcgtcgctatatttgaa

SEQ ID NO: 19 - DNA, 30 bp, synthetic construct
  source 1..30: mol_type = other DNA
ttacctttacaataacggattcgcaaaatt

SEQ ID NO: 20 - DNA, 30 bp, synthetic construct
  source 1..30: mol_type = other DNA
atttgcaccattttgcggaacaaatttgag

SEQ ID NO: 21 - DNA, 30 bp, synthetic construct
  source 1..30: mol_type = other DNA
gatttagattgctgaacctcaaagtattaa

SEQ ID NO: 22 - DNA, 30 bp, synthetic construct
  source 1..30: mol_type = other DNA
caccgcctgaaagcgtaagaatacattctg

SEQ ID NO: 23 - DNA, 30 bp, synthetic construct
  source 1..30: mol_type = other DNA
tgagtgttcagctgattgcccttgcgcggg

SEQ ID NO: 24 - DNA, 30 bp, synthetic construct
  source 1..30: mol_type = other DNA
gagaggcgacaacatacgagccgctgcagg

SEQ ID NO: 25 - DNA, 30 bp, synthetic construct
  source 1..30: mol_type = other DNA
tcgactctgaagggcgatcggtgcggcctc

SEQ ID NO: 26 - DNA, 30 bp, synthetic construct
  source 1..30: mol_type = other DNA
aggaagatcattaaatgtgagcgtttttaa

SEQ ID NO: 27 - DNA, 30 bp, synthetic construct
  source 1..30: mol_type = other DNA
ccaataggaaactagcatgtcaaggagcaa

SEQ ID NO: 28 - DNA, 32 bp, synthetic construct
  source 1..32: mol_type = other DNA
aagcaaacctattatagtcagaagattgaatc

SEQ ID NO: 29 - DNA, 32 bp, synthetic construct
  source 1..32: mol_type = other DNA
aaatattccaaagcggattgcatcgagcttca

SEQ ID NO: 30 - DNA, 32 bp, synthetic construct
  source 1..32: mol_type = other DNA
cccctcaacctcgtttaccagacggaatacca

SEQ ID NO: 31 - DNA, 32 bp, synthetic construct
  source 1..32: mol_type = other DNA
agatttagacgataaaaaccaaaaatcgtcat

SEQ ID NO: 32 - DNA, 32 bp, synthetic construct
  source 1..32: mol_type = other DNA
agtcaggacataggctggctgacctttgaaag

SEQ ID NO: 33 - DNA, 32 bp, synthetic construct
  source 1..32: mol_type = other DNA
acccaaataactttaatcattgtgatcagttg

SEQ ID NO: 34 - DNA, 32 bp, synthetic construct
  source 1..32: mol_type = other DNA
caaagctgtccatgttacttagccattatacc

SEQ ID NO: 35 - DNA, 32 bp, synthetic construct
  source 1..32: mol_type = other DNA
ccccagcgggaacgaggcgcagactattcatt

SEQ ID NO: 36 - DNA, 32 bp, synthetic construct
  source 1..32: mol_type = other DNA
aagcgcgagaggactaaagactttggccgctt

SEQ ID NO: 37 - DNA, 32 bp, synthetic construct
  source 1..32: mol_type = other DNA
gagttaaattcatgaggaagtttctctttgac

SEQ ID NO: 38 - DNA, 32 bp, synthetic construct
  source 1..32: mol_type = other DNA
tttcacgtcgatagttgcgccgaccttgcagg

SEQ ID NO: 39 - DNA, 32 bp, synthetic construct
  source 1..32: mol_type = other DNA
gagccgccttaaagccagaatggagatgatac

SEQ ID NO: 40 - DNA, 32 bp, synthetic construct
  source 1..32: mol_type = other DNA
caccaccagcccccttattagcgtccgtaatc

SEQ ID NO: 41 - DNA, 32 bp, synthetic construct
  source 1..32: mol_type = other DNA
agtagcgaattgagggagggaaggataagttt

SEQ ID NO: 42 - DNA, 32 bp, synthetic construct
  source 1..32: mol_type = other DNA
gccatttgcaaacgtagaaaatacctggcatg

SEQ ID NO: 43 - DNA, 32 bp, synthetic construct
  source 1..32: mol_type = other DNA
attttgtcagccgaacaaagttacgcccaata

SEQ ID NO: 44 - DNA, 32 bp, synthetic construct
  source 1..32: mol_type = other DNA
ataagagcagcctttacagagagaacaaaata

SEQ ID NO: 45 - DNA, 32 bp, synthetic construct
  source 1..32: mol_type = other DNA
caaatcagtgctattttgcacccagcctaatt

SEQ ID NO: 46 - DNA, 32 bp, synthetic construct
  source 1..32: mol_type = other DNA
aaataagaactttttcaaatatatctgagaga

SEQ ID NO: 47 - DNA, 32 bp, synthetic construct
  source 1..32: mol_type = other DNA
ctacctttagaatccttgaaaacaagaaaaca

SEQ ID NO: 48 - DNA, 32 bp, synthetic construct
  source 1..32: mol_type = other DNA
tttcccttttaacctccggcttagcaaagaac

SEQ ID NO: 49 - DNA, 32 bp, synthetic construct
  source 1..32: mol_type = other DNA
aaattaataccaagttacaaaatcctgaataa

SEQ ID NO: 50 - DNA, 32 bp, synthetic construct
  source 1..32: mol_type = other DNA
ctttgaattacatttaacaatttctaattaat

SEQ ID NO: 51 - DNA, 32 bp, synthetic construct
  source 1..32: mol_type = other DNA
gtagatttgttattaattttaaaaaacaattc

SEQ ID NO: 52 - DNA, 32 bp, synthetic construct
  source 1..32: mol_type = other DNA
tggaagggagcggaattatcatcaactaatag

SEQ ID NO: 53 - DNA, 32 bp, synthetic construct
  source 1..32: mol_type = other DNA
aacattatgtaaaacagaaataaattttacat

SEQ ID NO: 54 - DNA, 32 bp, synthetic construct
  source 1..32: mol_type = other DNA
ccagaaggttagaacctaccatatcctgattg

SEQ ID NO: 55 - DNA, 32 bp, synthetic construct
  source 1..32: mol_type = other DNA
attagagcaatatctggtcagttgcagcagaa

SEQ ID NO: 56 - DNA, 32 bp, synthetic construct
  source 1..32: mol_type = other DNA
gcatcaccagtattagactttacagtttgagt

SEQ ID NO: 57 - DNA, 32 bp, synthetic construct
  source 1..32: mol_type = other DNA
cctcaatccgtcaatagataatacagaaacca

SEQ ID NO: 58 - DNA, 32 bp, synthetic construct
  source 1..32: mol_type = other DNA
gataaaactttttgaatggctattttcaccag

SEQ ID NO: 59 - DNA, 32 bp, synthetic construct
  source 1..32: mol_type = other DNA
agacaataagaggtgaggcggtcatatcaaac

SEQ ID NO: 60 - DNA, 32 bp, synthetic construct
  source 1..32: mol_type = other DNA
tcacacgatgcaacaggaaaaacggaagaact

SEQ ID NO: 61 - DNA, 32 bp, synthetic construct
  source 1..32: mol_type = other DNA
ccagccatccagtaataaaagggacgtggcac

SEQ ID NO: 62 - DNA, 32 bp, synthetic construct
  source 1..32: mol_type = other DNA
aaaaaccgaaatcccttataaatcgagagttg

SEQ ID NO: 63 - DNA, 32 bp, synthetic construct
  source 1..32: mol_type = other DNA
ggccctgaaaaagaatagcccgagcgtggact

SEQ ID NO: 64 - DNA, 32 bp, synthetic construct
  source 1..32: mol_type = other DNA
cagcaagcgtcgtgccagctgcatagcctggg

SEQ ID NO: 65 - DNA, 32 bp, synthetic construct
  source 1..32: mol_type = other DNA
aagtgtaataatgaatcggccaaccaccgcct

SEQ ID NO: 66 - DNA, 32 bp, synthetic construct
  source 1..32: mol_type = other DNA
gaattcgtgccattcgccattcagttccggca

SEQ ID NO: 67 - DNA, 32 bp, synthetic construct
  source 1..32: mol_type = other DNA
ttcgctattgccaagcttgcatgcgaagcata

SEQ ID NO: 68 - DNA, 32 bp, synthetic construct
  source 1..32: mol_type = other DNA
ctggcgaacatctgccagtttgagattctccg

SEQ ID NO: 69 - DNA, 32 bp, synthetic construct
  source 1..32: mol_type = other DNA
cccgtcgggggacgacgacagtatcgggcctc

SEQ ID NO: 70 - DNA, 32 bp, synthetic construct
  source 1..32: mol_type = other DNA
tgggaacaattcgcattaaatttttgataatc

SEQ ID NO: 71 - DNA, 32 bp, synthetic construct
  source 1..32: mol_type = other DNA
accccggttgttaaatcagctcatagtaacaa

SEQ ID NO: 72 - DNA, 32 bp, synthetic construct
  source 1..32: mol_type = other DNA
agacagtccattgcctgagagtcttcatatgt

SEQ ID NO: 73 - DNA, 40 bp, synthetic construct
  source 1..40: mol_type = other DNA
caactaaagtacggtgggatggcttagagcttcagaccgg

SEQ ID NO: 74 - DNA, 40 bp, synthetic construct
  source 1..40: mol_type = other DNA
actggctccggaacaacattattagcaaaagaagttttgc

SEQ ID NO: 75 - DNA, 40 bp, synthetic construct
  source 1..40: mol_type = other DNA
ttaggattagcggggtggaacctattattctgactggtaa

SEQ ID NO: 76 - DNA, 40 bp, synthetic construct
  source 1..40: mol_type = other DNA
accgtcaccattagcaaggccggaaccagagccaccaccg

SEQ ID NO: 77 - DNA, 40 bp, synthetic construct
  source 1..40: mol_type = other DNA
aaagccggcgaacgtgtgccgtaaagcactaaaaagggcg

SEQ ID NO: 78 - DNA, 40 bp, synthetic construct
  source 1..40: mol_type = other DNA
cccgggtaccgctcacaattccacgtttgcgtattgggcg

SEQ ID NO: 79 - DNA, 48 bp, synthetic construct
  source 1..48: mol_type = other DNA
ttgcgggattaaacagcttgatactgaaaatctccaaaaaagcggagt

SEQ ID NO: 80 - DNA, 48 bp, synthetic construct
  source 1..48: mol_type = other DNA
gacggaaaaccatcgatagcagcattgccatcttttcatacaccctca

SEQ ID NO: 81 - DNA, 48 bp, synthetic construct
  source 1..48: mol_type = other DNA
tgccagttataacataaaaacaggacaagaattgagttaacagaagga

SEQ ID NO: 82 - DNA, 48 bp, synthetic construct
  source 1..48: mol_type = other DNA
aacagccattaaatcaagattagtatatagaaggcttatccaagccgt

SEQ ID NO: 83 - DNA, 48 bp, synthetic construct
  source 1..48: mol_type = other DNA
agaaaagcacaaaggctatcaggtaaatcaccatcaatatcaatgcct

SEQ ID NO: 84 - DNA, 78 bp, synthetic construct
  source 1..78: mol_type = other DNA
tttggtggcatcaattctagggcgcgagctgaaaatttcgtttctttgcttgtgtcttgtcttcgtcgtgcttgttcc

SEQ ID NO: 85 - DNA, 78 bp, synthetic construct
  source 1..78: mol_type = other DNA
ttttcccaattctgcgaacccatataacagttgattttcgtttctttgcttgtgtcttgtcttcgtcgtgcttgttcc

SEQ ID NO: 86 - DNA, 78 bp, synthetic construct
  source 1..78: mol_type = other DNA
tttattgctccttttgatattagagagtacctttatttcgtttctttgcttgtgtcttgtcttcgtcgtgcttgttcc

SEQ ID NO: 87 - DNA, 78 bp, synthetic construct
  source 1..78: mol_type = other DNA
tttccataaatcaaaaatccagaaaacgagaatgatttcgtttctttgcttgtgtcttgtcttcgtcgtgcttgttcc

SEQ ID NO: 88 - DNA, 78 bp, synthetic construct
  source 1..78: mol_type = other DNA
tttcgaggcatagtaagagacgccaaaaggaattatttcgtttctttgcttgtgtcttgtcttcgtcgtgcttgttcc

SEQ ID NO: 89 - DNA, 78 bp, synthetic construct
  source 1..78: mol_type = other DNA
tttgaaacaccagaacgagaggcttgccctgacgatttcgtttctttgcttgtgtcttgtcttcgtcgtgcttgttcc

SEQ ID NO: 90 - DNA, 78 bp, synthetic construct
  source 1..78: mol_type = other DNA
tttctgataaattgtgtcgagatttgtatcatcgctttcgtttctttgcttgtgtcttgtcttcgtcgtgcttgttcc

SEQ ID NO: 91 - DNA, 78 bp, synthetic construct
  source 1..78: mol_type = other DNA
tttgaacgagggtagcaacgcgaaagacagcatcgtttcgtttctttgcttgtgtcttgtcttcgtcgtgcttgttcc

SEQ ID NO: 92 - DNA, 78 bp, synthetic construct
  source 1..78: mol_type = other DNA
tttggtttatcagcttgctagcctttaattgtatctttcgtttctttgcttgtgtcttgtcttcgtcgtgcttgttcc

SEQ ID NO: 93 - DNA, 78 bp, synthetic construct
  source 1..78: mol_type = other DNA
tttgggattttgctaaacaaatgaattttctgtattttcgtttctttgcttgtgtcttgtcttcgtcgtgcttgttcc

SEQ ID NO: 94 - DNA, 78 bp, synthetic construct
  source 1..78: mol_type = other DNA
tttacaaactacaacgcctgagtttcgtcaccagttttcgtttctttgcttgtgtcttgtcttcgtcgtgcttgttcc

SEQ ID NO: 95 - DNA, 78 bp, synthetic construct
  source 1..78: mol_type = other DNA
tttagccaccaccctcattgaaccgccaccctcagtttcgtttctttgcttgtgtcttgtcttcgtcgtgcttgttcc

SEQ ID NO: 96 - DNA, 78 bp, synthetic construct
  source 1..78: mol_type = other DNA
tttgagagggttgatataagcggataagtgccgtctttcgtttctttgcttgtgtcttgtcttcgtcgtgcttgttcc

SEQ ID NO: 97 - DNA, 78 bp, synthetic construct
  source 1..78: mol_type = other DNA
tttgtataaacagttaatgttgagtaacagtgccctttcgtttctttgcttgtgtcttgtcttcgtcgtgcttgttcc

SEQ ID NO: 98 - DNA, 78 bp, synthetic construct
  source 1..78: mol_type = other DNA
tttgcaggtcagacgattgttgacaggaggttgagtttcgtttctttgcttgtgtcttgtcttcgtcgtgcttgttcc

SEQ ID NO: 99 - DNA, 78 bp, synthetic construct
  source 1..78: mol_type = other DNA
ttttagcgcgttttcatcgctttagcgtcagactgtttcgtttctttgcttgtgtcttgtcttcgtcgtgcttgttcc

SEQ ID NO: 100 - DNA, 78 bp, synthetic construct
  source 1..78: mol_type = other DNA
tttgcgccaaagacaaaagttcatatggtttaccatttcgtttctttgcttgtgtcttgtcttcgtcgtgcttgttcc

SEQ ID NO: 101 - DNA, 78 bp, synthetic construct
  source 1..78: mol_type = other DNA
tttccgaagccctttttaaagcaatagctatcttatttcgtttctttgcttgtgtcttgtcttcgtcgtgcttgttcc

SEQ ID NO: 102 - DNA, 78 bp, synthetic construct
  source 1..78: mol_type = other DNA
tttttttttgtttaacgtctccaaataagaaacgatttcgtttctttgcttgtgtcttgtcttcgtcgtgcttgttcc

SEQ ID NO: 103 - DNA, 78 bp, synthetic construct
  source 1..78: mol_type = other DNA
tttaacctcccgacttgcggcgaggcgttttagcgtttcgtttctttgcttgtgtcttgtcttcgtcgtgcttgttcc

SEQ ID NO: 104 - DNA, 78 bp, synthetic construct
  source 1..78: mol_type = other DNA
ttttaaaccaagtaccgcattccaagaacgggtattttcgtttctttgcttgtgtcttgtcttcgtcgtgcttgttcc

SEQ ID NO: 105 - DNA, 78 bp, synthetic construct
  source 1..78: mol_type = other DNA
tttagataagtcctgaacacctgtttatcaacaattttcgtttctttgcttgtgtcttgtcttcgtcgtgcttgttcc

SEQ ID NO: 106 - DNA, 78 bp, synthetic construct
  source 1..78: mol_type = other DNA
tttgtaaagtaattctgtcaaagtaccgacaaaagtttcgtttctttgcttgtgtcttgtcttcgtcgtgcttgttcc

SEQ ID NO: 107 - DNA, 78 bp, synthetic construct
  source 1..78: mol_type = other DNA
tttagtagggcttaattgaaaagccaacgctcaactttcgtttctttgcttgtgtcttgtcttcgtcgtgcttgttcc

SEQ ID NO: 108 - DNA, 78 bp, synthetic construct
  source 1..78: mol_type = other DNA
tttaatggtttgaaatacccttctgacctaaattttttcgtttctttgcttgtgtcttgtcttcgtcgtgcttgttcc

SEQ ID NO: 109 - DNA, 78 bp, synthetic construct
  source 1..78: mol_type = other DNA
tttagtcaatagtgaatttttaagacgctgagaagtttcgtttctttgcttgtgtcttgtcttcgtcgtgcttgttcc

SEQ ID NO: 110 - DNA, 78 bp, synthetic construct
  source 1..78: mol_type = other DNA
ttttgagcaaaagaagatgattcatttcaattacctttcgtttctttgcttgtgtcttgtcttcgtcgtgcttgttcc

SEQ ID NO: 111 - DNA, 78 bp, synthetic construct
  source 1..78: mol_type = other DNA
tttcaatataatcctgattgatgatggcaattcattttcgtttctttgcttgtgtcttgtcttcgtcgtgcttgttcc

SEQ ID NO: 112 - DNA, 78 bp, synthetic construct
  source 1..78: mol_type = other DNA
tttgttatctaaaatatctaaaggaattgaggaagtttcgtttctttgcttgtgtcttgtcttcgtcgtgcttgttcc

SEQ ID NO: 113 - DNA, 78 bp, synthetic construct
  source 1..78: mol_type = other DNA
tttacatcgccattaaaaaaactgatagccctaaatttcgtttctttgcttgtgtcttgtcttcgtcgtgcttgttcc

SEQ ID NO: 114 - DNA, 78 bp, synthetic construct
  source 1..78: mol_type = other DNA
ttttcgtctgaaatggattacattttgacgctcaatttcgtttctttgcttgtgtcttgtcttcgtcgtgcttgttcc

SEQ ID NO: 115 - DNA, 78 bp, synthetic construct
  source 1..78: mol_type = other DNA
tttttgattagtaataacattgtagcaatacttcttttcgtttctttgcttgtgtcttgtcttcgtcgtgcttgttcc

SEQ ID NO: 116 - DNA, 78 bp, synthetic construct
  source 1..78: mol_type = other DNA
tttaggaacggtacgccagtaaagggattttagactttcgtttctttgcttgtgtcttgtcttcgtcgtgcttgttcc

SEQ ID NO: 117 - DNA, 78 bp, synthetic construct
  source 1..78: mol_type = other DNA
tttgagcacgtataacgtgctatggttgctttgactttcgtttctttgcttgtgtcttgtcttcgtcgtgcttgttcc

SEQ ID NO: 118 - DNA, 78 bp, synthetic construct
  source 1..78: mol_type = other DNA
tttcgggcgctagggcgctaagaaagcgaaaggagtttcgtttctttgcttgtgtcttgtcttcgtcgtgcttgttcc

SEQ ID NO: 119 - DNA, 78 bp, synthetic construct
  source 1..78: mol_type = other DNA
tttatcacccaaatcaagtgcccactacgtgaacctttcgtttctttgcttgtgtcttgtcttcgtcgtgcttgttcc

SEQ ID NO: 120 - DNA, 78 bp, synthetic construct
  source 1..78: mol_type = other DNA
tttatcctgtttgatggtggccccagcaggcgaaatttcgtttctttgcttgtgtcttgtcttcgtcgtgcttgttcc

SEQ ID NO: 121 - DNA, 78 bp, synthetic construct
  source 1..78: mol_type = other DNA
tttgctcactgcccgctttacattaattgcgttgctttcgtttctttgcttgtgtcttgtcttcgtcgtgcttgttcc

SEQ ID NO: 122 - DNA, 78 bp, synthetic construct
  source 1..78: mol_type = other DNA
tttgtaacgccagggttttaaggcgattaagttggtttcgtttctttgcttgtgtcttgtcttcgtcgtgcttgttcc

SEQ ID NO: 123 - DNA, 78 bp, synthetic construct
  source 1..78: mol_type = other DNA
tttcgttggtgtagatggggtaatgggataggtcatttcgtttctttgcttgtgtcttgtcttcgtcgtgcttgttcc

SEQ ID NO: 124 - DNA, 78 bp, synthetic construct
  source 1..78: mol_type = other DNA
ttttttaaattgtaaacgtattgtataagcaaatatttcgtttctttgcttgtgtcttgtcttcgtcgtgcttgttcc

SEQ ID NO: 125 - DNA, 78 bp, synthetic construct
  source 1..78: mol_type = other DNA
tttgccggagagggtagcttagctgataaattaattttcgtttctttgcttgtgtcttgtcttcgtcgtgcttgttcc

SEQ ID NO: 126 - DNA, 78 bp, synthetic construct
  source 1..78: mol_type = other DNA
tttaaatttttagaaccctttcaacgcaaggataatttcgtttctttgcttgtgtcttgtcttcgtcgtgcttgttcc

SEQ ID NO: 127 - DNA, 78 bp, synthetic construct
  source 1..78: mol_type = other DNA
ttttaagcaataaagcctcaaagaattagcaaaattttcgtttctttgcttgtgtcttgtcttcgtcgtgcttgttcc

SEQ ID NO: 128 - DNA, 25 bp, synthetic construct
  source 1..25: mol_type = other DNA
tgccactactttttttgccaccctc

SEQ ID NO: 129 - DNA, 25 bp, synthetic construct
  source 1..25: mol_type = other DNA
aactgaacattttttttgaataacc

SEQ ID NO: 130 - DNA, 25 bp, synthetic construct
  source 1..25: mol_type = other DNA
gccacgctgtttttttaccagtgag

SEQ ID NO: 131 - DNA, 25 bp, synthetic construct
  source 1..25: mol_type = other DNA
caaaaataattttttttgtttagac

SEQ ID NO: 132 - DNA, 29 bp, synthetic construct
  source 1..29: mol_type = other DNA
gatacatttcgcttttttgaccctgtaat

SEQ ID NO: 133 - DNA, 29 bp, synthetic construct
  source 1..29: mol_type = other DNA
accgtactcaggtttttgatctaaagttt

SEQ ID NO: 134 - DNA, 29 bp, synthetic construct
  source 1..29: mol_type = other DNA
aacatgtaattttttttgaaaccaatcaa

SEQ ID NO: 135 - DNA, 29 bp, synthetic construct
  source 1..29: mol_type = other DNA
gcgtaaccaccatttttgagtaaaagagt

SEQ ID NO: 136 - DNA, 31 bp, synthetic construct
  source 1..31: mol_type = other DNA
cagagggggttttgccttcctgtagccagct

SEQ ID NO: 137 - DNA, 31 bp, synthetic construct
  source 1..31: mol_type = other DNA
gaaccgcctctttacctaaaacgaaagaggc

SEQ ID NO: 138 - DNA, 31 bp, synthetic construct
  source 1..31: mol_type = other DNA
cataaatcaatttagtcagagggtaattgag

SEQ ID NO: 139 - DNA, 31 bp, synthetic construct
  source 1..31: mol_type = other DNA
ccagggtggttttgcaaatgaaaaatctaaa

SEQ ID NO: 140 - DNA, 34 bp, synthetic construct
  source 1..34: mol_type = other DNA
acaaccattttttcatacatggcttttaagcgca

SEQ ID NO: 141 - DNA, 34 bp, synthetic construct
  source 1..34: mol_type = other DNA
ttttatcttttttatccaatcgcaagagttgggt

SEQ ID NO: 142 - DNA, 34 bp, synthetic construct
  source 1..34: mol_type = other DNA
gccaacattttttccactattaaagaaatagggt

SEQ ID NO: 143 - DNA, 34 bp, synthetic construct
  source 1..34: mol_type = other DNA
acaagagtttttttcgcgttttaattcaaaaaga

SEQ ID NO: 144 - DNA, 36 bp, synthetic construct
  source 1..36: mol_type = other DNA
tggatagcaagcccgatttttaatcgtaaacgccat

SEQ ID NO: 145 - DNA, 36 bp, synthetic construct
  source 1..36: mol_type = other DNA
agaaccgcatttaccgttttaccgatatatacgtaa

SEQ ID NO: 146 - DNA, 36 bp, synthetic construct
  source 1..36: mol_type = other DNA
ttgcttcttatatgtattttacgctaacggagaatt

SEQ ID NO: 147 - DNA, 36 bp, synthetic construct
  source 1..36: mol_type = other DNA
acgggcaagttccagttttttctgacctgcaacagt

SEQ ID NO: 148 - DNA, 38 bp, synthetic construct
  source 1..38: mol_type = other DNA
gagaatagaaaggaacaactattttctcaagagaagga

SEQ ID NO: 149 - DNA, 38 bp, synthetic construct
  source 1..38: mol_type = other DNA
ttttattttcatcgtaggaatttttagcctgtttagta

SEQ ID NO: 150 - DNA, 38 bp, synthetic construct
  source 1..38: mol_type = other DNA
caaactatcggccttgctggtttttgagcttgacgggg

SEQ ID NO: 151 - DNA, 38 bp, synthetic construct
  source 1..38: mol_type = other DNA
gagtaatgtgtaggtaaagattttttgttttaaatatg

SEQ ID NO: 152 - DNA, 43 bp, synthetic construct
  source 1..43: mol_type = other DNA
aggacagatgattttttcaccagtagcaccattaccgacttga

SEQ ID NO: 153 - DNA, 43 bp, synthetic construct
  source 1..43: mol_type = other DNA
attaagactcctttttaatatacagtaacagtaccgaaattgc

SEQ ID NO: 154 - DNA, 43 bp, synthetic construct
  source 1..43: mol_type = other DNA
gacaactcgtattttttcctgtgtgaaattgttatccgagctc

SEQ ID NO: 155 - DNA, 43 bp, synthetic construct
  source 1..43: mol_type = other DNA
ccgcttctggttttttcgttaataaaacgaactaaattatacc

SEQ ID NO: 156 - DNA, 45 bp, synthetic construct
  source 1..45: mol_type = other DNA
tgtcgtctcagccctcatatttttttcgccaccctcaggtgtatc

SEQ ID NO: 157 - DNA, 45 bp, synthetic construct
  source 1..45: mol_type = other DNA
taatcggccatcctaattttttttttttttcgagccaacaacgcc

SEQ ID NO: 158 - DNA, 45 bp, synthetic construct
  source 1..45: mol_type = other DNA
ctgtccatttttataatcatttttttcttaatgcgcccacgctgc

SEQ ID NO: 159 - DNA, 45 bp, synthetic construct
  source 1..45: mol_type = other DNA
acttttgcatcggttgtactttttttaacctgtttaggaccatta

SEQ ID NO: 160 - DNA, 51 bp, synthetic construct
  source 1..51: mol_type = other DNA
aagcgaacaattgctgaatataatgctgtatttttttgtgagaaaggccgg

SEQ ID NO: 161 - DNA, 51 bp, synthetic construct
  source 1..51: mol_type = other DNA
aggagtgtaaacatgaaagtattaagaggctttttttgcgaataataattt

SEQ ID NO: 162 - DNA, 51 bp, synthetic construct
  source 1..51: mol_type = other DNA
gcgagaaaataaacaccggaatcataattatttttttcgcccaatagcaag

SEQ ID NO: 163 - DNA, 51 bp, synthetic construct
  source 1..51: mol_type = other DNA
ccaacgtcatcggaaccctaaagggagccctttttttgaacaatattaccg

SEQ ID NO: 164 - DNA, 60 bp, synthetic construct
  source 1..60: mol_type = other DNA
ggaattagagcttttttttcagaccaggcgcgttgggaagattttttttccaggcaaagc

SEQ ID NO: 165 - DNA, 60 bp, synthetic construct
  source 1..60: mol_type = other DNA
aatcatggtcatttttttttttgcccgaactcaggtttaacttttttttcagtatgttag

SEQ ID NO: 166 - DNA, 53 bp, synthetic construct
  source 1..53: mol_type = other DNA
gtcaggaagaggtcatttttgctctggaagtttacagttattagcccgcattt

SEQ ID NO: 167 - DNA, 53 bp, synthetic construct
  source 1..53: mol_type = other DNA
atacatacaacactatcataacatgctttatttacagttattagcccgcattt

SEQ ID NO: 168 - DNA, 53 bp, synthetic construct
  source 1..53: mol_type = other DNA
acaacggaaatccgcgacctgcctcattcatttacagttattagcccgcattt

SEQ ID NO: 169 - DNA, 53 bp, synthetic construct
  source 1..53: mol_type = other DNA
caaaaggttcgaggtgaatttctcgtcacctttacagttattagcccgcattt

SEQ ID NO: 170 - DNA, 53 bp, synthetic construct
  source 1..53: mol_type = other DNA
cagtgccccccctgcctatttctttgctcatttacagttattagcccgcattt

SEQ ID NO: 171 - DNA, 53 bp, synthetic construct
  source 1..53: mol_type = other DNA
agtttgcgcattttcggtcatagagccgcctttacagttattagcccgcattt

SEQ ID NO: 172 - DNA, 53 bp, synthetic construct
  source 1..53: mol_type = other DNA
atgaaatgaaaagtaagcagatacaatcaatttacagttattagcccgcattt

SEQ ID NO: 173 - DNA, 53 bp, synthetic construct
  source 1..53: mol_type = other DNA
taagaacggaggttttgaagcctattattttttacagttattagcccgcattt

SEQ ID NO: 174 - DNA, 53 bp, synthetic construct
  source 1..53: mol_type = other DNA
atttcatgaccgtgtgataaataattcttatttacagttattagcccgcattt

SEQ ID NO: 175 - DNA, 53 bp, synthetic construct
  source 1..53: mol_type = other DNA
gcgaattatgaaacaaacatcatagcgatatttacagttattagcccgcattt

SEQ ID NO: 176 - DNA, 53 bp, synthetic construct
  source 1..53: mol_type = other DNA
acagttgttaggagcactaacatattcctgtttacagttattagcccgcattt

SEQ ID NO: 177 - DNA, 53 bp, synthetic construct
  source 1..53: mol_type = other DNA
aatacctatttacattggcagaagtctttatttacagttattagcccgcattt

SEQ ID NO: 178 - DNA, 53 bp, synthetic construct
  source 1..53: mol_type = other DNA
ggcgatgtttttggggtcgagggcgagaaatttacagttattagcccgcattt

SEQ ID NO: 179 - DNA, 53 bp, synthetic construct
  source 1..53: mol_type = other DNA
ctaactcccagtcgggaaacctggtccacgtttacagttattagcccgcattt

SEQ ID NO: 180 - DNA, 53 bp, synthetic construct
  source 1..53: mol_type = other DNA
attgacccgcatcgtaaccgtgagggggattttacagttattagcccgcattt

SEQ ID NO: 181 - DNA, 53 bp, synthetic construct
  source 1..53: mol_type = other DNA
accgttcatttttgagagatctcccaaaaatttacagttattagcccgcattt

SEQ ID NO: 182 - DNA, 63 bp, synthetic construct
  source 1..63: mol_type = other DNA
ccatgtaccgtaacactgtagcattccacagattccagactttacagttattagcccgcattt

SEQ ID NO: 183 - DNA, 63 bp, synthetic construct
  source 1..63: mol_type = other DNA
agctaatgcagaacgcgagaaaaataatatcctgtctttctttacagttattagcccgcattt

SEQ ID NO: 184 - DNA, 63 bp, synthetic construct
  source 1..63: mol_type = other DNA
ctaaacaggaggccgataatcctgagaagtgtcacgcaaatttacagttattagcccgcattt

SEQ ID NO: 185 - DNA, 63 bp, synthetic construct
  source 1..63: mol_type = other DNA
aatcatacaggcaaggcagagcataaagctaagggagaagtttacagttattagcccgcattt

SEQ ID NO: 186 - DNA, 30 bp, synthetic construct
  source 1..30: mol_type = other DNA
tttcattgagtagatttagtttctatattt

SEQ ID NO: 187 - DNA, 30 bp, synthetic construct
  source 1..30: mol_type = other DNA
aacagttaggtctttaccctgatccaacag

SEQ ID NO: 188 - DNA, 30 bp, synthetic construct
  source 1..30: mol_type = other DNA
gtgaatatagtaaattgggctttaatgcag

SEQ ID NO: 189 - DNA, 30 bp, synthetic construct
  source 1..30: mol_type = other DNA
ctcagcaggctacagaggctttaacaaagt

SEQ ID NO: 190 - DNA, 30 bp, synthetic construct
  source 1..30: mol_type = other DNA
gttagtaactttcaacagtttcaaaggctc

SEQ ID NO: 191 - DNA, 30 bp, synthetic construct
  source 1..30: mol_type = other DNA
gtaccaggtatagcccggaatagaaccgcc

SEQ ID NO: 192 - DNA, 30 bp, synthetic construct
  source 1..30: mol_type = other DNA
gccagcagccttgatattcacaaacggggt

SEQ ID NO: 193 - DNA, 30 bp, synthetic construct
  source 1..30: mol_type = other DNA
tagaaaaggcgacattcaaccgcagaatca

SEQ ID NO: 194 - DNA, 30 bp, synthetic construct
  source 1..30: mol_type = other DNA
atcccaaaaaaatgaaaatagcaagaaaca

SEQ ID NO: 195 - DNA, 30 bp, synthetic construct
  source 1..30: mol_type = other DNA
cttatcactcatcgagaacaagcggtattc

SEQ ID NO: 196 - DNA, 30 bp, synthetic construct
  source 1..30: mol_type = other DNA
ccagtatgaatcgccatatttagtaataag

SEQ ID NO: 197 - DNA, 30 bp, synthetic construct
  source 1..30: mol_type = other DNA
gcttagaatcaaaatcataggttttagtta

SEQ ID NO: 198 - DNA, 30 bp, synthetic construct
  source 1..30: mol_type = other DNA
attatcagtttggattatacttgcgcagag

SEQ ID NO: 199 - DNA, 30 bp, synthetic construct
  source 1..30: mol_type = other DNA
atgcgcgtaccgaacgaaccacgcaaatca

SEQ ID NO: 200 - DNA, 30 bp, synthetic construct
  source 1..30: mol_type = other DNA
ttaaccgtcacttgcctgagtactcatgga

SEQ ID NO: 201 - DNA, 30 bp, synthetic construct
  source 1..30: mol_type = other DNA
ggaagggggcaagtgtagcggtgctacagg

SEQ ID NO: 202 - DNA, 30 bp, synthetic construct
  source 1..30: mol_type = other DNA
ctggtttgttccgaaatcggcatctatcag

SEQ ID NO: 203 - DNA, 30 bp, synthetic construct
  source 1..30: mol_type = other DNA
gtgctgccccagtcacgacgtttgagtgag

SEQ ID NO: 204 - DNA, 30 bp, synthetic construct
  source 1..30: mol_type = other DNA
caggaagtaatattttgttaaaaacggcgg

SEQ ID NO: 205 - DNA, 30 bp, synthetic construct
  source 1..30: mol_type = other DNA
cctttatcatatattttaaatggatattca

SEQ ID NO: 206 - DNA, 48 bp, synthetic construct
  source 1..48: mol_type = other DNA
aaaagaataaccgaactgaccaacttcatcaagagtaatcttttaaga

SEQ ID NO: 207 - DNA, 48 bp, synthetic construct
  source 1..48: mol_type = other DNA
cgctaataggaatacccaaaagaaatacataaaggtggcagaattatc

SEQ ID NO: 208 - DNA, 48 bp, synthetic construct
  source 1..48: mol_type = other DNA
ttcatcaacgcactccagccagctgctgcgcaactgttggagaggatc

SEQ ID NO: 209 - DNA, 59 bp, synthetic construct
  source 1..59: mol_type = other DNA
aaaagaataaccgaactgaccaacttcatcaagagtaatcttttaagatttatacatct

SEQ ID NO: 210 - DNA, 59 bp, synthetic construct
  source 1..59: mol_type = other DNA
cgctaataggaatacccaaaagaaatacataaaggtggcagaattatctttatacatct

SEQ ID NO: 211 - DNA, 59 bp, synthetic construct
  source 1..59: mol_type = other DNA
ttcatcaacgcactccagccagctgctgcgcaactgttggagaggatctttatacatct

SEQ ID NO: 212 - DNA, 61 bp, synthetic construct
  source 1..61: mol_type = other DNA
aaaagaataaccgaactgaccaacttcatcaagagtaatcttttaagatttatacatctag

SEQ ID NO: 213 - DNA, 61 bp, synthetic construct
  source 1..61: mol_type = other DNA
cgctaataggaatacccaaaagaaatacataaaggtggcagaattatctttatacatctag

SEQ ID NO: 214 - DNA, 61 bp, synthetic construct
  source 1..61: mol_type = other DNA
ttcatcaacgcactccagccagctgctgcgcaactgttggagaggatctttatacatctag

SEQ ID NO: 215 - DNA, 32 bp, synthetic construct
  source 1..32: mol_type = other DNA
cattcaacgagatggtttaatttccaacgtaa

SEQ ID NO: 216 - DNA, 32 bp, synthetic construct
  source 1..32: mol_type = other DNA
taagttttaacaaataaatcctcaaccagaac

SEQ ID NO: 217 - DNA, 32 bp, synthetic construct
  source 1..32: mol_type = other DNA
gtgcctaagtaaaacgacggccagtacgccag

SEQ ID NO: 218 - DNA, 43 bp, synthetic construct
  source 1..43: mol_type = other DNA
cattcaacgagatggtttaatttccaacgtaatttatacatct

SEQ ID NO: 219 - DNA, 43 bp, synthetic construct
  source 1..43: mol_type = other DNA
taagttttaacaaataaatcctcaaccagaactttatacatct

SEQ ID NO: 220 - DNA, 43 bp, synthetic construct
  source 1..43: mol_type = other DNA
gtgcctaagtaaaacgacggccagtacgccagtttatacatct

SEQ ID NO: 221 - DNA, 45 bp, synthetic construct
  source 1..45: mol_type = other DNA
cattcaacgagatggtttaatttccaacgtaatttatacatctag

SEQ ID NO: 222 - DNA, 45 bp, synthetic construct
  source 1..45: mol_type = other DNA
taagttttaacaaataaatcctcaaccagaactttatacatctag

SEQ ID NO: 223 - DNA, 45 bp, synthetic construct
  source 1..45: mol_type = other DNA
gtgcctaagtaaaacgacggccagtacgccagtttatacatctag

SEQ ID NO: 224 - DNA, 75 bp, synthetic construct
  source 1..75: mol_type = other DNA
aaccgagggcaaagacaccacggataaatatttttcactcacctccatctccactcctacccatccaactcccac

SEQ ID NO: 225 - DNA, 10 bp, synthetic construct
  source 1..10: mol_type = other DNA
atacatctag

SEQ ID NO: 226 - DNA, 10 bp, synthetic construct
  source 1..10: mol_type = other DNA
atctgtctta

SEQ ID NO: 227 - DNA, 24 bp, synthetic construct
  source 1..24: mol_type = other DNA
caactaaagtacggtgggatggct

SEQ ID NO: 228 - DNA, 24 bp, synthetic construct
  source 1..24: mol_type = other DNA
cattattagcaaaagaagttttgc

SEQ ID NO: 229 - DNA, 24 bp, synthetic construct
  source 1..24: mol_type = other DNA
ttaggattagcggggtggaaccta

SEQ ID NO: 230 - DNA, 24 bp, synthetic construct
  source 1..24: mol_type = other DNA
aggccggaaccagagccaccaccg

SEQ ID NO: 231 - DNA, 24 bp, synthetic construct
  source 1..24: mol_type = other DNA
aaagccggcgaacgtgtgccgtaa

SEQ ID NO: 232 - DNA, 24 bp, synthetic construct
  source 1..24: mol_type = other DNA
aattccacgtttgcgtattgggcg

SEQ ID NO: 233 - DNA, 32 bp, synthetic construct
  source 1..32: mol_type = other DNA
tagagcttcagaccggaagcaaacctattata

SEQ ID NO: 234 - DNA, 32 bp, synthetic construct
  source 1..32: mol_type = other DNA
gtcagaagattgaatccccctcaacctcgttt

SEQ ID NO: 235 - DNA, 32 bp, synthetic construct
  source 1..32: mol_type = other DNA
accagacggaataccacattcaacgagatggt

SEQ ID NO: 236 - DNA, 32 bp, synthetic construct
  source 1..32: mol_type = other DNA
ttaatttccaacgtaacaaagctgtccatgtt

SEQ ID NO: 237 - DNA, 32 bp, synthetic construct
  source 1..32: mol_type = other DNA
acttagccattataccaagcgcgagaggacta

SEQ ID NO: 238 - DNA, 32 bp, synthetic construct
  source 1..32: mol_type = other DNA
aaaagaataaccgaactgaccaacttcatcaa

SEQ ID NO: 239 - DNA, 32 bp, synthetic construct
  source 1..32: mol_type = other DNA
aagactttggccgcttttgcgggattaaacag

SEQ ID NO: 240 - DNA, 32 bp, synthetic construct
  source 1..32: mol_type = other DNA
cttgatactgaaaatctccaaaaaagcggagt

SEQ ID NO: 241 - DNA, 32 bp, synthetic construct
  source 1..32: mol_type = other DNA
ttattctgactggtaataagttttaacaaata

SEQ ID NO: 242 - DNA, 32 bp, synthetic construct
  source 1..32: mol_type = other DNA
aatcctcaaccagaaccaccaccagccccctt

SEQ ID NO: 243 - DNA, 32 bp, synthetic construct
  source 1..32: mol_type = other DNA
attagcgtccgtaatcagtagcgaattgaggg

SEQ ID NO: 244 - DNA, 32 bp, synthetic construct
  source 1..32: mol_type = other DNA
agggaaggataagtttattttgtcagccgaac

SEQ ID NO: 245 - DNA, 32 bp, synthetic construct
  source 1..32: mol_type = other DNA
accacggataaatattgacggaaaaccatcga

SEQ ID NO: 246 - DNA, 32 bp, synthetic construct
  source 1..32: mol_type = other DNA
aaagttacgcccaataataagagcagccttta

SEQ ID NO: 247 - DNA, 32 bp, synthetic construct
  source 1..32: mol_type = other DNA
cgctaataggaatacccaaaagaaatacataa

SEQ ID NO: 248 - DNA, 32 bp, synthetic construct
  source 1..32: mol_type = other DNA
tgagttaacagaaggaaaccgagggcaaagac

SEQ ID NO: 249 - DNA, 32 bp, synthetic construct
  source 1..32: mol_type = other DNA
cagagagaacaaaataaacagccattaaatca

SEQ ID NO: 250 - DNA, 32 bp, synthetic construct
  source 1..32: mol_type = other DNA
agattagtatatagaaggcttatccaagccgt

SEQ ID NO: 251 - DNA, 32 bp, synthetic construct
  source 1..32: mol_type = other DNA
agcactaaaaagggcgaaaaaccgaaatccct

SEQ ID NO: 252 - DNA, 32 bp, synthetic construct
  source 1..32: mol_type = other DNA
tataaatcgagagttgcagcaagcgtcgtgcc

SEQ ID NO: 253 - DNA, 32 bp, synthetic construct
  source 1..32: mol_type = other DNA
agctgcatagcctggggtgcctaagtaaaacg

SEQ ID NO: 254 - DNA, 32 bp, synthetic construct
  source 1..32: mol_type = other DNA
acggccagtacgccagctggcgaacatctgcc

SEQ ID NO: 255 - DNA, 32 bp, synthetic construct
  source 1..32: mol_type = other DNA
agtttgagattctccgtgggaacaattcgcat

SEQ ID NO: 256 - DNA, 32 bp, synthetic construct
  source 1..32: mol_type = other DNA
ttcatcaacgcactccagccagctgctgcgca

SEQ ID NO: 257 - DNA, 32 bp, synthetic construct
  source 1..32: mol_type = other DNA
taaatttttgataatcagaaaagcacaaaggc

SEQ ID NO: 258 - DNA, 32 bp, synthetic construct
  source 1..32: mol_type = other DNA
tatcaggtaaatcaccatcaatatcaatgcct

SEQ ID NO: 259 - DNA, 40 bp, synthetic construct
  source 1..40: mol_type = other DNA
agctaatgcagaacgcgagaaaaataatatcctgtctttc

SEQ ID NO: 260 - DNA, 40 bp, synthetic construct
  source 1..40: mol_type = other DNA
aatcatacaggcaaggcagagcataaagctaagggagaag

SEQ ID NO: 261 - DNA, 88 bp, synthetic construct
  source 1..88: mol_type = other DNA
agatttagacgataaaaaccaaaaatcgtcattttcactcacctccatctccactcctacccatccaactcccactttctagatgtat

SEQ ID NO: 262 - DNA, 88 bp, synthetic construct
  source 1..88: mol_type = other DNA
gagtaatcttttaagaactggctccggaacaatttcactcacctccatctccactcctacccatccaactcccactttctagatgtat

SEQ ID NO: 263 - DNA, 88 bp, synthetic construct
  source 1..88: mol_type = other DNA
gagttaaattcatgaggaagtttctctttgactttcactcacctccatctccactcctacccatccaactcccactttctagatgtat

SEQ ID NO: 264 - DNA, 88 bp, synthetic construct
  source 1..88: mol_type = other DNA
tagcagcattgccatcttttcatacaccctcatttcactcacctccatctccactcctacccatccaactcccactttctagatgtat

SEQ ID NO: 265 - DNA, 88 bp, synthetic construct
  source 1..88: mol_type = other DNA
aggtggcagaattatcaccgtcaccattagcatttcactcacctccatctccactcctacccatccaactcccactttctagatgtat

SEQ ID NO: 266 - DNA, 88 bp, synthetic construct
  source 1..88: mol_type = other DNA
tgccagttataacataaaaacaggacaagaattttcactcacctccatctccactcctacccatccaactcccactttctagatgtat

SEQ ID NO: 267 - DNA, 88 bp, synthetic construct
  source 1..88: mol_type = other DNA
ctttgaattacatttaacaatttctaattaattttcactcacctccatctccactcctacccatccaactcccactttctagatgtat

SEQ ID NO: 268 - DNA, 88 bp, synthetic construct
  source 1..88: mol_type = other DNA
aacattatgtaaaacagaaataaattttacattttcactcacctccatctccactcctacccatccaactcccactttctagatgtat

SEQ ID NO: 269 - DNA, 88 bp, synthetic construct
  source 1..88: mol_type = other DNA
agacaataagaggtgaggcggtcatatcaaactttcactcacctccatctccactcctacccatccaactcccactttctagatgtat

SEQ ID NO: 270 - DNA, 88 bp, synthetic construct
  source 1..88: mol_type = other DNA
aagtgtaataatgaatcggccaaccaccgccttttcactcacctccatctccactcctacccatccaactcccactttctagatgtat

SEQ ID NO: 271 - DNA, 88 bp, synthetic construct
  source 1..88: mol_type = other DNA
actgttggagaggatccccgggtaccgctcactttcactcacctccatctccactcctacccatccaactcccactttctagatgtat

SEQ ID NO: 272 - DNA, 88 bp, synthetic construct
  source 1..88: mol_type = other DNA
accccggttgttaaatcagctcatagtaacaatttcactcacctccatctccactcctacccatccaactcccactttctagatgtat

SEQ ID NO: 273 - DNA, 33 bp, synthetic construct
  source 1..33: mol_type = other DNA
  modified_base 33: mod_base = OTHER; note = 3' biotinylated modified nucleotide
gtcaggaagaggtcatttttgctctggaagttt

SEQ ID NO: 274 - DNA, 33 bp, synthetic construct
  source 1..33: mol_type = other DNA
  modified_base 33: mod_base = OTHER; note = 3' biotinylated modified nucleotide
acaacggaaatccgcgacctgcctcattcattt

SEQ ID NO: 275 - DNA, 33 bp, synthetic construct
  source 1..33: mol_type = other DNA
  modified_base 33: mod_base = OTHER; note = 3' biotinylated modified nucleotide
agtttgcgcattttcggtcatagagccgccttt

SEQ ID NO: 276 - DNA, 33 bp, synthetic construct
  source 1..33: mol_type = other DNA
  modified_base 33: mod_base = OTHER; note = 3' biotinylated modified nucleotide
taagaacggaggttttgaagcctattatttttt

SEQ ID NO: 277 - DNA, 33 bp, synthetic construct
  source 1..33: mol_type = other DNA
  modified_base 33: mod_base = OTHER; note = 3' biotinylated modified nucleotide
atttcatgaccgtgtgataaataattcttattt

SEQ ID NO: 278 - DNA, 33 bp, synthetic construct
  source 1..33: mol_type = other DNA
  modified_base 33: mod_base = OTHER; note = 3' biotinylated modified nucleotide
acagttgttaggagcactaacatattcctgttt

SEQ ID NO: 279 - DNA, 33 bp, synthetic construct
  source 1..33: mol_type = other DNA
  modified_base 33: mod_base = OTHER; note = 3' biotinylated modified nucleotide
ctaactcccagtcgggaaacctggtccacgttt

SEQ ID NO: 280 - DNA, 33 bp, synthetic construct
  source 1..33: mol_type = other DNA
  modified_base 33: mod_base = OTHER; note = 3' biotinylated modified nucleotide
accgttcatttttgagagatctcccaaaaattt

SEQ ID NO: 281 - DNA, 43 bp, synthetic construct
  source 1..43: mol_type = other DNA
  modified_base 43: mod_base = OTHER; note = 3' biotinylated modified nucleotide
ccatgtaccgtaacactgtagcattccacagattccagacttt

SEQ ID NO: 282 - DNA, 43 bp, synthetic construct
  source 1..43: mol_type = other DNA
  modified_base 43: mod_base = OTHER; note = 3' biotinylated modified nucleotide
ctaaacaggaggccgataatcctgagaagtgtcacgcaaattt

SEQ ID NO: 283 - DNA, 30 bp, synthetic construct
  source 1..30: mol_type = other DNA
atacatacaacactatcataacatgcttta

SEQ ID NO: 284 - DNA, 30 bp, synthetic construct
  source 1..30: mol_type = other DNA
caaaaggttcgaggtgaatttctcgtcacc

SEQ ID NO: 285 - DNA, 30 bp, synthetic construct
  source 1..30: mol_type = other DNA
cagtgccccccctgcctatttctttgctca

SEQ ID NO: 286 - DNA, 30 bp, synthetic construct
  source 1..30: mol_type = other DNA
atgaaatgaaaagtaagcagatacaatcaa

SEQ ID NO: 287 - DNA, 30 bp, synthetic construct
  source 1..30: mol_type = other DNA
gcgaattatgaaacaaacatcatagcgata

SEQ ID NO: 288 - DNA, 30 bp, synthetic construct
  source 1..30: mol_type = other DNA
aatacctatttacattggcagaagtcttta

SEQ ID NO: 289 - DNA, 30 bp, synthetic construct
  source 1..30: mol_type = other DNA
ggcgatgtttttggggtcgagggcgagaaa

SEQ ID NO: 290 - DNA, 30 bp, synthetic construct
  source 1..30: mol_type = other DNA
attgacccgcatcgtaaccgtgagggggat

SEQ ID NO: 291 - DNA, 10 bp, synthetic construct
  source 1..10: mol_type = other DNA
ctagatgtat

SEQ ID NO: 292 - DNA, 10 bp, synthetic construct
  source 1..10: mol_type = other DNA
taagacagat

SEQ ID NO: 293 - DNA, 33 bp, synthetic construct
  source 1..33: mol_type = other DNA
gtggatgggtaggagtggagatggaggtgagtg

SEQ ID NO: 294 - DNA, 34 bp, synthetic construct
  source 1..34: mol_type = other DNA
cactcacctccatctccactcctacccatccaac

SEQ ID NO: 295 - DNA, 32 bp, synthetic construct
  source 1..32: mol_type = other DNA
gagtaatcttttaagaactggctccggaacaa

SEQ ID NO: 296 - DNA, 32 bp, synthetic construct
  source 1..32: mol_type = other DNA
tagcagcattgccatcttttcatacaccctca

SEQ ID NO: 297 - DNA, 32 bp, synthetic construct
  source 1..32: mol_type = other DNA
aggtggcagaattatcaccgtcaccattagca

SEQ ID NO: 298 - DNA, 32 bp, synthetic construct
  source 1..32: mol_type = other DNA
tgccagttataacataaaaacaggacaagaat

SEQ ID NO: 299 - DNA, 32 bp, synthetic construct
  source 1..32: mol_type = other DNA
actgttggagaggatccccgggtaccgctcac

SEQ ID NO: 300 - DNA, 21 bp, synthetic construct
  source 1..21: mol_type = other DNA
agggggatgtgcatgcaaatc

SEQ ID NO: 301 - DNA, 21 bp, synthetic construct
  source 1..21: mol_type = other DNA
gtgtctgtagtttgcagagca

SEQ ID NO: 302 - DNA, 26 bp, synthetic construct
  source 1..26: mol_type = other DNA
cgacttgcgggaggttttgccagcgc

SEQ ID NO: 303 - DNA, 28 bp, synthetic construct
  source 1..28: mol_type = other DNA
aacggtagtgaggccaccgagtcggcct

SEQ ID NO: 304 - DNA, 28 bp, synthetic construct
  source 1..28: mol_type = other DNA
gccaacaaatacgtggcacagtaaaaca

SEQ ID NO: 305 - DNA, 28 bp, synthetic construct
  source 1..28: mol_type = other DNA
gcttctgaatccttgaaaacagttgggt

SEQ ID NO: 306 - DNA, 28 bp, synthetic construct
  source 1..28: mol_type = other DNA
gagggttgtaccaggcggataaaagtat

SEQ ID NO: 307 - DNA, 28 bp, synthetic construct
  source 1..28: mol_type = other DNA
gtagcaaaaaacgaaagaggcttgtgtc

SEQ ID NO: 308 - DNA, 29 bp, synthetic construct
  source 1..29: mol_type = other DNA
tttaccctgaacaaagtcagaggagccta

SEQ ID NO: 309 - DNA, 33 bp, synthetic construct
  source 1..33: mol_type = other DNA
ttattagagaatcaatagccgaacaaagttacc

SEQ ID NO: 310 - DNA, 35 bp, synthetic construct
  source 1..35: mol_type = other DNA
gaaccctaaagggatgtagcgatgaggaagtttca

SEQ ID NO: 311 - DNA, 35 bp, synthetic construct
  source 1..35: mol_type = other DNA
ttcaacgcagtcaaatcaccaggctatcgcgccat

SEQ ID NO: 312 - DNA, 35 bp, synthetic construct
  source 1..35: mol_type = other DNA
ggagaaggaattaggatacatataactaaagtacg

SEQ ID NO: 313 - DNA, 35 bp, synthetic construct
  source 1..35: mol_type = other DNA
accattacaaaattaagcaatgtaatactgtgtac

SEQ ID NO: 314 - DNA, 35 bp, synthetic construct
  source 1..35: mol_type = other DNA
accagtaaccaatgcgattggaaagccaccagtaa

SEQ ID NO: 315 - DNA, 35 bp, synthetic construct
  source 1..35: mol_type = other DNA
tgcacgttatatttcattaacatccaataaaaatt

SEQ ID NO: 316 - DNA, 35 bp, synthetic construct
  source 1..35: mol_type = other DNA
tcagatataaaacagggaagcgagcgctaatattt

SEQ ID NO: 317 - DNA, 36 bp, synthetic construct
  source 1..36: mol_type = other DNA
tttgaagggcgatcggtgcgggcctcttccgccatt

SEQ ID NO: 318 - DNA, 44 bp, synthetic construct
  source 1..44: mol_type = other DNA
aaatagcccacaagaattgagttaagcccaatatttatacatct

SEQ ID NO: 319 - DNA, 46 bp, synthetic construct
  source 1..46: mol_type = other DNA
tttataaacagccatattaatttgccagttacaaatttatacatct

SEQ ID NO: 320 - DNA, 46 bp, synthetic construct
  source 1..46: mol_type = other DNA
ttttgaaaatagcagcctttgtttaacgtcaaaaatttatacatct

SEQ ID NO: 321 - DNA, 40 bp, synthetic construct
  source 1..40: mol_type = other DNA
ttttgccggagagggtagcctagctgtgaccctaaagcct

SEQ ID NO: 322 - DNA, 42 bp, synthetic construct
  source 1..42: mol_type = other DNA
cctgaaaagtattacgagaatagcggattgcatcaccagaac

SEQ ID NO: 323 - DNA, 42 bp, synthetic construct
  source 1..42: mol_type = other DNA
agtgatgaagggtagggcggtttttgcggatattcaaccgtt

SEQ ID NO: 324 - DNA, 42 bp, synthetic construct
  source 1..42: mol_type = other DNA
gaagggatagctctagtcccggagagatataagcaaatattt

SEQ ID NO: 325 - DNA, 42 bp, synthetic construct
  source 1..42: mol_type = other DNA
caaccggaatcataattactataccagctttcatcaacatta

SEQ ID NO: 326 - DNA, 42 bp, synthetic construct
  source 1..42: mol_type = other DNA
tcataaacactgttgccctgccccacgcaattattgggaggg

SEQ ID NO: 327 - DNA, 42 bp, synthetic construct
  source 1..42: mol_type = other DNA
cagcaaaagccagctcacggtgttcttccgctcacagtgtaa

SEQ ID NO: 328 - DNA, 42 bp, synthetic construct
  source 1..42: mol_type = other DNA
gtagattgaagttttgtagctgtcatttaggtcagttaattc

SEQ ID NO: 329 - DNA, 42 bp, synthetic construct
  source 1..42: mol_type = other DNA
gaatcgtggggtaagaaagattctacgtaacgtaaatcaaga

SEQ ID NO: 330 - DNA, 42 bp, synthetic construct
  source 1..42: mol_type = other DNA
aaggtaattcgcaatcgtcgcacgatgctgattgcgctcatt

SEQ ID NO: 331 - DNA, 42 bp, synthetic construct
  source 1..42: mol_type = other DNA
tacagagagaataacataagattgcacccagaaacgattttt

SEQ ID NO: 332 - DNA, 42 bp, synthetic construct
  source 1..42: mol_type = other DNA
ataagagcaacatggtccagcgctggtctggtcagctccggc

SEQ ID NO: 333 - DNA, 42 bp, synthetic construct
  source 1..42: mol_type = other DNA
aaaagataacagttccaaaataatgcagagaactgacgagaa

SEQ ID NO: 334 - DNA, 42 bp, synthetic construct
  source 1..42: mol_type = other DNA
cgtaatgctaaagatgatacctatcggtttatcagacgatct

SEQ ID NO: 335 - DNA, 42 bp, synthetic construct
  source 1..42: mol_type = other DNA
ccgtgaatttcttaaacagctctttttcgtcacgctgcgcgt

SEQ ID NO: 336 - DNA, 42 bp, synthetic construct
  source 1..42: mol_type = other DNA
tcatagttagcgtacttgctttcgagatgtaccgtacagccc

SEQ ID NO: 337 - DNA, 49 bp, synthetic construct
  source 1..49: mol_type = other DNA
taaatcgaaagttttgtcgtcttaattggatagttgcgccgaagaggct

SEQ ID NO: 338 - DNA, 49 bp, synthetic construct
  source 1..49: mol_type = other DNA
cacccaagtatgggattttgctcacgttaccgatatattcggagcagcg

SEQ ID NO: 339 - DNA, 49 bp, synthetic construct
  source 1..49: mol_type = other DNA
actacgtgaaccatattaaagaaagacatacactaaaacacttttgtat

SEQ ID NO: 340 - DNA, 49 bp, synthetic construct
  source 1..49: mol_type = other DNA
aggagcatatcatttatcaccgtcaccgacttgagagtttgagtaacat

SEQ ID NO: 341 - DNA, 49 bp, synthetic construct
  source 1..49: mol_type = other DNA
ggagcggacctaccagctgaaaaggttaatcggctatggaagggttaga

SEQ ID NO: 342 - DNA, 49 bp, synthetic construct
  source 1..49: mol_type = other DNA
cacgggaacggataaagttggtttttaatctccgtgggaacatgagggg

SEQ ID NO: 343 - DNA, 49 bp, synthetic construct
  source 1..49: mol_type = other DNA
aagacaaagaacgcataagaataaacgccagctttcaatcgcctatatg

SEQ ID NO: 344 - DNA, 49 bp, synthetic construct
  source 1..49: mol_type = other DNA
taaacagcgagcacaaggagcggggaaagtagcattccacagaacactg

SEQ ID NO: 345 - DNA, 49 bp, synthetic construct
  source 1..49: mol_type = other DNA
tgcatctgccagttaacggcgttaaatcagattgtctacaaatcaatat

SEQ ID NO: 346 - DNA, 49 bp, synthetic construct
  source 1..49: mol_type = other DNA
gccttcctgtagattaaaaactagcatgtcaatcagcgtctgtatataa

SEQ ID NO: 347 - DNA, 49 bp, synthetic construct
  source 1..49: mol_type = other DNA
gccgtttggtgccggtgcccctacactggagtacctttaaagaaagact

SEQ ID NO: 348 - DNA, 49 bp, synthetic construct
  source 1..49: mol_type = other DNA
acggagacatctttcaccctctcgctgagaataataattttttaaacaa

SEQ ID NO: 349 - DNA, 49 bp, synthetic construct
  source 1..49: mol_type = other DNA
cgagccataaagtattcagcttaagtccacgagcacttatcactcatcg

SEQ ID NO: 350 - DNA, 49 bp, synthetic construct
  source 1..49: mol_type = other DNA
gaagaaaattcatatggtttaaagccttaacgcgaggcgtttatcatta

SEQ ID NO: 351 - DNA, 49 bp, synthetic construct
  source 1..49: mol_type = other DNA
tttatcccaatccaaataagccgagcgttaaaagaattaagactcctta

SEQ ID NO: 352 - DNA, 49 bp, synthetic construct
  source 1..49: mol_type = other DNA
acggaatatacccaaaagaaccgaagcctgtagcgcgcagagagataac

SEQ ID NO: 353 - DNA, 49 bp, synthetic construct
  source 1..49: mol_type = other DNA
aatagctatcttactggcatgaacgcaaagacacccttaccattcatcg

SEQ ID NO: 354 - DNA, 49 bp, synthetic construct
  source 1..49: mol_type = other DNA
agcccaataggaacgaaccgcagggcgctatggttgctttgagaggccg

SEQ ID NO: 355 - DNA, 49 bp, synthetic construct
  source 1..49: mol_type = other DNA
gtcagacctttttaagaaaagtaacggaaagtttattcagagccaccac

SEQ ID NO: 356 - DNA, 50 bp, synthetic construct
  source 1..50: mol_type = other DNA
tttaaatttaatggtttgaaataccgaccttttagtgagactacgctgag

SEQ ID NO: 357 - DNA, 50 bp, synthetic construct
  source 1..50: mol_type = other DNA
tttataagagcaagaaacaatgttacgcaaaggtggcaacatactttcca

SEQ ID NO: 358 - DNA, 56 bp, synthetic construct
  source 1..56: mol_type = other DNA
gcttgacgggcgctcaccctcttagtaccgccaccccgtcgaattaaagggatttt

SEQ ID NO: 359 - DNA, 56 bp, synthetic construct
  source 1..56: mol_type = other DNA
aaagcacaaccaccttgaggaccactacgaaggcagctccatgagcccttcaccgc

SEQ ID NO: 360 - DNA, 56 bp, synthetic construct
  source 1..56: mol_type = other DNA
tacagggagacagggcagacggtcaatgctccattaaacgggtaaaataacgaggc

SEQ ID NO: 361 - DNA, 56 bp, synthetic construct
  source 1..56: mol_type = other DNA
cgagatagggttacccgccgcatcctgagttacttttgaaagaggacagacaccag

SEQ ID NO: 362 - DNA, 56 bp, synthetic construct
  source 1..56: mol_type = other DNA
aatagccctggcccgaaatccgtgtacagaccaggtaaggctgtcgggaaacctgt

SEQ ID NO: 363 - DNA, 56 bp, synthetic construct
  source 1..56: mol_type = other DNA
catcacgtgagtagtaagaggctattattctgaaatcagtgcagtaataaaaggga

SEQ ID NO: 364 - DNA, 56 bp, synthetic construct
  source 1..56: mol_type = other DNA
tgtttttagaacaaaacgagtcttatgcgattttaatacatactgaattcgtaatc

SEQ ID NO: 365 - DNA, 56 bp, synthetic construct
  source 1..56: mol_type = other DNA
aaaaacgcattctgcatagtaagagcggcatttcaactttaatcattgttacgagg

SEQ ID NO: 366 - DNA, 56 bp, synthetic construct
  source 1..56: mol_type = other DNA
taattgcgttgcttaccgccaaacccttacgccaagacgacgataaaaacagaaaa

SEQ ID NO: 367 - DNA, 56 bp, synthetic construct
  source 1..56: mol_type = other DNA
ctcacatatggtcattcaactagcgagaggcttttcctcaaagctgcggccagaat

SEQ ID NO: 368 - DNA, 56 bp, synthetic construct
  source 1..56: mol_type = other DNA
tggctatgaacgaataccgttgaatggaaagcgcatgaggcaaatcaacagttgaa

SEQ ID NO: 369 - DNA, 56 bp, synthetic construct
  source 1..56: mol_type = other DNA
gagagccaggaattcattgacaggaggtgtaccctgactattatagtcaccgccag

SEQ ID NO: 370 - DNA, 56 bp, synthetic construct
  source 1..56: mol_type = other DNA
ccagcgcttgctcgcctcagagaaccgccaccctctgacggaaaccagcttacggc

SEQ ID NO: 371 - DNA, 56 bp, synthetic construct
  source 1..56: mol_type = other DNA
atgccaatttcgtcatggtcaacaggcaaggcaaacctttattgccgccagcagtt

SEQ ID NO: 372 - DNA, 56 bp, synthetic construct
  source 1..56: mol_type = other DNA
atgaatatgctttgcaatagaaatgcagaacgcgcgtaccgatatatgtgagtgaa

SEQ ID NO: 373 - DNA, 56 bp, synthetic construct
  source 1..56: mol_type = other DNA
cagaaatttcattttttagaaaagggtgagaaagggagtctgtaacaatcggcgaa

SEQ ID NO: 374 - DNA, 56 bp, synthetic construct
  source 1..56: mol_type = other DNA
tgaaacataaccttaacggtaatcgtaacttgagtaatgtgtaggtaaaatcgatg

SEQ ID NO: 375 - DNA, 56 bp, synthetic construct
  source 1..56: mol_type = other DNA
acagcggatcaaattacctgatcgctatgagcaaacccggttgataatcgaacgcc

SEQ ID NO: 376 - DNA, 56 bp, synthetic construct
  source 1..56: mol_type = other DNA
cgcagaaacgtacaaggtcatccccaaaaacaggaagctcatgtaacgccagggtt

SEQ ID NO: 377 - DNA, 56 bp, synthetic construct
  source 1..56: mol_type = other DNA
attaagacctttttcgccatatcaacagtagggctgaaaaagaaataaggcgttaa

SEQ ID NO: 378 - DNA, 56 bp, synthetic construct
  source 1..56: mol_type = other DNA
taatttttaaatgcatcaaaagcgagtaacaacccggcctcacgcttctggtgccg

SEQ ID NO: 379 - DNA, 56 bp, synthetic construct
  source 1..56: mol_type = other DNA
aacgtggatcggcaaaatccccaagcggcatcgccgctggctgaccttccaaagct

SEQ ID NO: 380 - DNA, 56 bp, synthetic construct
  source 1..56: mol_type = other DNA
tggcgaagaaaccaacgacgacagtatcgtcggatccaatagagaaaagtgcctga

SEQ ID NO: 381 - DNA, 56 bp, synthetic construct
  source 1..56: mol_type = other DNA
tgagagaaccagtgagacgggctttccatgccctggctcattataccagtaccaca

SEQ ID NO: 382 - DNA, 56 bp, synthetic construct
  source 1..56: mol_type = other DNA
tccacgcgccagggtggttttcgtgccagctcattgttgggaagaaaaatcatcag

SEQ ID NO: 383 - DNA, 56 bp, synthetic construct
  source 1..56: mol_type = other DNA
aagaactaaatacctacatttcacgacccttgagttaataagttttaacctgaatt

SEQ ID NO: 384 - DNA, 56 bp, synthetic construct
  source 1..56: mol_type = other DNA
gctgcatagcctggggtgcctgtgaaatttgagataagttttgccagagcataaat

SEQ ID NO: 385 - DNA, 56 bp, synthetic construct
  source 1..56: mol_type = other DNA
taatgaaattgggctggtttgttccgaaactccaaatggcccctttcaacagtttc

SEQ ID NO: 386 - DNA, 56 bp, synthetic construct
  source 1..56: mol_type = other DNA
tagctgtgttgaggatccccgctgtggttgctttataagaggaagcccgccaccac

SEQ ID NO: 387 - DNA, 56 bp, synthetic construct
  source 1..56: mol_type = other DNA
tgttatcgcgtccgtgagcctgcggcggattcatttcaaatatcgcgttgattaga

SEQ ID NO: 388 - DNA, 56 bp, synthetic construct
  source 1..56: mol_type = other DNA
ccaccagaatgaaaaatctaagttggcaggtcagaaaaccatcgatagcgagccag

SEQ ID NO: 389 - DNA, 56 bp, synthetic construct
  source 1..56: mol_type = other DNA
aaatgcaatgccgtaaggcatcaattctactaataatattttgcagaggcgaatta

SEQ ID NO: 390 - DNA, 56 bp, synthetic construct
  source 1..56: mol_type = other DNA
ccctcatgtagtagtcatttggggcgcgatatcaattgcgtagattttcaaatcgc

SEQ ID NO: 391 - DNA, 56 bp, synthetic construct
  source 1..56: mol_type = other DNA
ctggtcaagcatcaaaatacctagtcttccagtcatgacgctacttgcccaaatta

SEQ ID NO: 392 - DNA, 56 bp, synthetic construct
  source 1..56: mol_type = other DNA
ctgtaaaggtttctagtgtcactgcgaccgcctgcaaaatatcaccaccagagccg

SEQ ID NO: 393 - DNA, 56 bp, synthetic construct
  source 1..56: mol_type = other DNA
gtgtgttcggttgcggtatgatggaggtttttaaatatgcatagttgctccttttg

SEQ ID NO: 394 - DNA, 56 bp, synthetic construct
  source 1..56: mol_type = other DNA
ccgaacgatcatattcctgatctgaatagtctttctgtagaaaccaatcttatcaa

SEQ ID NO: 395 - DNA, 56 bp, synthetic construct
  source 1..56: mol_type = other DNA
aataccaagaaaacaaaattaaaatcaacaaaagggtaataagagaatatgagaat

SEQ ID NO: 396 - DNA, 56 bp, synthetic construct
  source 1..56: mol_type = other DNA
aatcaggtctttctggaacactatcataaccctcgaatcaaagaggtgaggcggtc

SEQ ID NO: 397 - DNA, 56 bp, synthetic construct
  source 1..56: mol_type = other DNA
gaatcccgcaaaagttaggaatcaggaccagtgaacgcatagtgataaaaaaagaa

SEQ ID NO: 398 - DNA, 56 bp, synthetic construct
  source 1..56: mol_type = other DNA
agtacatattacatgcctgattacagtatatactttatcagaattaaatgaggatt

SEQ ID NO: 399 - DNA, 56 bp, synthetic construct
  source 1..56: mol_type = other DNA
aacctccctttttcaaatatagtgtgatcctgtttagtatcaccaacgctttaaca

SEQ ID NO: 400 - DNA, 56 bp, synthetic construct
  source 1..56: mol_type = other DNA
gagatggtttaatgagtcataagggaaccgaactgtgggctttgctggtaatatcc

SEQ ID NO: 401 - DNA, 56 bp, synthetic construct
  source 1..56: mol_type = other DNA
ccggcacggaagatcgcactcaatgtgaataattctatgtaccaagagagattcaa

SEQ ID NO: 402 - DNA, 56 bp, synthetic construct
  source 1..56: mol_type = other DNA
gcatcggacgcatagaaaatctccaaaaattttctatcaagttttttggagtttgg

SEQ ID NO: 403 - DNA, 56 bp, synthetic construct
  source 1..56: mol_type = other DNA
ccggagacaaggataaatcatataacctcgattgacattaaaaccctcagccgcca

SEQ ID NO: 404 - DNA, 56 bp, synthetic construct
  source 1..56: mol_type = other DNA
gaagcaagaccatatttaccaaaggaatgaattacagtaaataccaactagccgga

SEQ ID NO: 405 - DNA, 56 bp, synthetic construct
  source 1..56: mol_type = other DNA
gtactggaacagtgtcggaacctgagacttgctcagatataaaggaggtagaaccg

SEQ ID NO: 406 - DNA, 56 bp, synthetic construct
  source 1..56: mol_type = other DNA
atgaacggcgacctccaacctcggctaccaatgactccaaaaggagccttttccag

SEQ ID NO: 407 - DNA, 56 bp, synthetic construct
  source 1..56: mol_type = other DNA
acgttagtaaatgaaaaaggcaacaaccatcgcccaacgaggaacaagagtccact

SEQ ID NO: 408 - DNA, 56 bp, synthetic construct
  source 1..56: mol_type = other DNA
aatcaccggaacttagcccccccgcgcccaatagcttctaagaaatcaagattagt

SEQ ID NO: 409 - DNA, 56 bp, synthetic construct
  source 1..56: mol_type = other DNA
acgctaatacaattttatccttgctattaggcttatccggtaaagcaaagcatttt

SEQ ID NO: 410 - DNA, 56 bp, synthetic construct
  source 1..56: mol_type = other DNA
aggaaacgcaataataagcagagtttgcctttagccggtcatgtcacaatcaatat

SEQ ID NO: 411 - DNA, 57 bp, synthetic construct
  source 1..57: mol_type = other DNA
ttttttctccgtggtatcgacaggcctttcatcctcgcgtggtatcagcgccgggcg

SEQ ID NO: 412 - DNA, 57 bp, synthetic construct
  source 1..57: mol_type = other DNA
tttcggccagtgccaagctttcagaggtgttcccagatttttggattgaccgtaatg

SEQ ID NO: 413 - DNA, 69 bp, synthetic construct
  source 1..69: mol_type = other DNA
gtaaccgcaggctgcgcaactgttggtttcgtttctttgcttgtgtcttgtcttcgtcgtgcttgttcc

SEQ ID NO: 414 - DNA, 69 bp, synthetic construct
  source 1..69: mol_type = other DNA
gcattaatcacgacgttgtaaaacgatttcgtttctttgcttgtgtcttgtcttcgtcgtgcttgttcc

SEQ ID NO: 415 - DNA, 69 bp, synthetic construct
  source 1..69: mol_type = other DNA
tatttttgaatttgtgagagatagactttcgtttctttgcttgtgtcttgtcttcgtcgtgcttgttcc

SEQ ID NO: 416 - DNA, 69 bp, synthetic construct
  source 1..69: mol_type = other DNA
gaattgcggcttgcagggagttaaagtttcgtttctttgcttgtgtcttgtcttcgtcgtgcttgttcc

SEQ ID NO: 417 - DNA, 69 bp, synthetic construct
  source 1..69: mol_type = other DNA
gatagcaagtttcgtcaccagtacaatttcgtttctttgcttgtgtcttgtcttcgtcgtgcttgttcc

SEQ ID NO: 418 - DNA, 74 bp, synthetic construct
  source 1..74: mol_type = other DNA
tttggttgtaccaaaaacattaataaattaatttcgtttctttgcttgtgtcttgtcttcgtcgtgcttgttcc

SEQ ID NO: 419 - DNA, 77 bp, synthetic construct
  source 1..77: mol_type = other DNA
tttactacaacgcctgccggcgaacgtggcgagatttcgtttctttgcttgtgtcttgtcttcgtcgtgcttgttcc

SEQ ID NO: 420 - DNA, 78 bp, synthetic construct
  source 1..78: mol_type = other DNA
tttaccgtctatcagggcgcgtcaaagggcgaaaatttcgtttctttgcttgtgtcttgtcttcgtcgtgcttgttcc

SEQ ID NO: 421 - DNA, 78 bp, synthetic construct
  source 1..78: mol_type = other DNA
ttttcctgtttgatggtggccccagcaggcgaaaatttcgtttctttgcttgtgtcttgtcttcgtcgtgcttgttcc

SEQ ID NO: 422 - DNA, 78 bp, synthetic construct
  source 1..78: mol_type = other DNA
tttgagaggcggtttgcgttcggccaacgcgcgggtttcgtttctttgcttgtgtcttgtcttcgtcgtgcttgttcc

SEQ ID NO: 423 - DNA, 78 bp, synthetic construct
  source 1..78: mol_type = other DNA
tttcgagccggaagcataaaattccacacaacatatttcgtttctttgcttgtgtcttgtcttcgtcgtgcttgttcc

SEQ ID NO: 424 - DNA, 78 bp, synthetic construct
  source 1..78: mol_type = other DNA
tttgccagcacgcgtgcctcataccgggggtttcttttcgtttctttgcttgtgtcttgtcttcgtcgtgcttgttcc

SEQ ID NO: 425 - DNA, 78 bp, synthetic construct
  source 1..78: mol_type = other DNA
tttatgccgggttacctgctcgttaacggcatcagtttcgtttctttgcttgtgtcttgtcttcgtcgtgcttgttcc

SEQ ID NO: 426 - DNA, 78 bp, synthetic construct
  source 1..78: mol_type = other DNA
ttttttcgcactcaatccggggtcattgcaggcgctttcgtttctttgcttgtgtcttgtcttcgtcgtgcttgttcc

SEQ ID NO: 427 - DNA, 78 bp, synthetic construct
  source 1..78: mol_type = other DNA
tttgacttgtagaacgtcaataacggaacgtgccgtttcgtttctttgcttgtgtcttgtcttcgtcgtgcttgttcc

SEQ ID NO: 428 - DNA, 78 bp, synthetic construct
  source 1..78: mol_type = other DNA
tttaaaaagccgcacaggctaaaaaaatcccgtaatttcgtttctttgcttgtgtcttgtcttcgtcgtgcttgttcc

SEQ ID NO: 429 - DNA, 78 bp, synthetic construct
  source 1..78: mol_type = other DNA
ttttgtagatgggcgcatcggataggtcacgttggtttcgtttctttgcttgtgtcttgtcttcgtcgtgcttgttcc

SEQ ID NO: 430 - DNA, 78 bp, synthetic construct
  source 1..78: mol_type = other DNA
ttttattttgttaaaattcaaattgtaaacgttaatttcgtttctttgcttgtgtcttgtcttcgtcgtgcttgttcc

SEQ ID NO: 431 - DNA, 78 bp, synthetic construct
  source 1..78: mol_type = other DNA
ttttcttaccagtataaagtatgcgttatacaaattttcgtttctttgcttgtgtcttgtcttcgtcgtgcttgttcc

SEQ ID NO: 432 - DNA, 78 bp, synthetic construct
  source 1..78: mol_type = other DNA
ttttaggcagaggcattttacgccaacatgtaatttttcgtttctttgcttgtgtcttgtcttcgtcgtgcttgttcc

SEQ ID NO: 433 - DNA, 78 bp, synthetic construct
  source 1..78: mol_type = other DNA
tttaaaggaacaactaaagagcggagtgagaatagtttcgtttctttgcttgtgtcttgtcttcgtcgtgcttgttcc

SEQ ID NO: 434 - DNA, 78 bp, synthetic construct
  source 1..78: mol_type = other DNA
tttgacaataaacaacatgattctgtccagacgactttcgtttctttgcttgtgtcttgtcttcgtcgtgcttgttcc

SEQ ID NO: 435 - DNA, 78 bp, synthetic construct
  source 1..78: mol_type = other DNA
ttttatcccatcctaattttgaacaagaaaaataatttcgtttctttgcttgtgtcttgtcttcgtcgtgcttgttcc

SEQ ID NO: 436 - DNA, 78 bp, synthetic construct
  source 1..78: mol_type = other DNA
tttcaaggccggaaacgtcgcaccattaccattagtttcgtttctttgcttgtgtcttgtcttcgtcgtgcttgttcc

SEQ ID NO: 437 - DNA, 78 bp, synthetic construct
  source 1..78: mol_type = other DNA
tttcaaataaatcctcattccttgatattcacaaatttcgtttctttgcttgtgtcttgtcttcgtcgtgcttgttcc

SEQ ID NO: 438 - DNA, 78 bp, synthetic construct
  source 1..78: mol_type = other DNA
tttttgatgatacaggagtgcgtcatacatggctttttcgtttctttgcttgtgtcttgtcttcgtcgtgcttgttcc

SEQ ID NO: 439 - DNA, 78 bp, synthetic construct
  source 1..78: mol_type = other DNA
tttatgccccctgcctattcccgtataaacagttatttcgtttctttgcttgtgtcttgtcttcgtcgtgcttgttcc

SEQ ID NO: 440 - DNA, 78 bp, synthetic construct
  source 1..78: mol_type = other DNA
ttttaggattagcggggtttcctcaagagaaggattttcgtttctttgcttgtgtcttgtcttcgtcgtgcttgttcc

SEQ ID NO: 441 - DNA, 78 bp, synthetic construct
  source 1..78: mol_type = other DNA
tttgtgtatcaccgtactcgtatagcccggaatagtttcgtttctttgcttgtgtcttgtcttcgtcgtgcttgttcc

SEQ ID NO: 442 - DNA, 78 bp, synthetic construct
  source 1..78: mol_type = other DNA
tttcaccctcattttcaggccaccctcagagccactttcgtttctttgcttgtgtcttgtcttcgtcgtgcttgttcc

SEQ ID NO: 443 - DNA, 84 bp, synthetic construct
  source 1..84: mol_type = other DNA
tttaaggaagggaagaaagcgagtataacgtgctttcctcgtttcgtttctttgcttgtgtcttgtcttcgtcgtgcttgttcc

SEQ ID NO: 444 - DNA, 84 bp, synthetic construct
  source 1..84: mol_type = other DNA
tttttagaatcagagcgggagcaccgttgtagcaatacttctttcgtttctttgcttgtgtcttgtcttcgtcgtgcttgttcc

SEQ ID NO: 445 - DNA, 84 bp, synthetic construct
  source 1..84: mol_type = other DNA
ttttttgattagtaataacatccaatcgtctgaaatggatttttcgtttctttgcttgtgtcttgtcttcgtcgtgcttgttcc

SEQ ID NO: 446 - DNA, 84 bp, synthetic construct
  source 1..84: mol_type = other DNA
tttatttacattggcagattcataatgcgcgaactgatagctttcgtttctttgcttgtgtcttgtcttcgtcgtgcttgttcc

SEQ ID NO: 447 - DNA, 84 bp, synthetic construct
  source 1..84: mol_type = other DNA
tttcctaaaacatcgccattaaccttgctgaacctcaaatatttcgtttctttgcttgtgtcttgtcttcgtcgtgcttgttcc

SEQ ID NO: 448 - DNA, 84 bp, synthetic construct
  source 1..84: mol_type = other DNA
ttttcaaaccctcaatcaatattagaagtattagactttactttcgtttctttgcttgtgtcttgtcttcgtcgtgcttgttcc

SEQ ID NO: 449 - DNA, 84 bp, synthetic construct
  source 1..84: mol_type = other DNA
tttttcccaattctgcgaacgacagagcataaagctaaatctttcgtttctttgcttgtgtcttgtcttcgtcgtgcttgttcc

SEQ ID NO: 450 - DNA, 84 bp, synthetic construct
  source 1..84: mol_type = other DNA
tttaaacaattcgacaactcgttgatggcaattcatcaatatttcgtttctttgcttgtgtcttgtcttcgtcgtgcttgttcc

SEQ ID NO: 451 - DNA, 84 bp, synthetic construct
  source 1..84: mol_type = other DNA
ttttaattgctgaatataatgccattccatataacagttgatttcgtttctttgcttgtgtcttgtcttcgtcgtgcttgttcc

SEQ ID NO: 452 - DNA, 84 bp, synthetic construct
  source 1..84: mol_type = other DNA
ttttaatcctgattgtttggatacagtaccttttacatcggtttcgtttctttgcttgtgtcttgtcttcgtcgtgcttgttcc

SEQ ID NO: 453 - DNA, 84 bp, synthetic construct
  source 1..84: mol_type = other DNA
tttaccggaagcaaactccaacttgcggatggcttagagcttttcgtttctttgcttgtgtcttgtcttcgtcgtgcttgttcc

SEQ ID NO: 454 - DNA, 84 bp, synthetic construct
  source 1..84: mol_type = other DNA
tttgagaaacaataacggattcttaacaatttcatttgaattttcgtttctttgcttgtgtcttgtcttcgtcgtgcttgttcc

SEQ ID NO: 455 - DNA, 84 bp, synthetic construct
  source 1..84: mol_type = other DNA
tttgatagcgtccaatactgcggagcttcaaagcgaaccagtttcgtttctttgcttgtgtcttgtcttcgtcgtgcttgttcc

SEQ ID NO: 456 - DNA, 84 bp, synthetic construct
  source 1..84: mol_type = other DNA
ttttaccttttttaatggaaacaagagtcaatagtgaattttttcgtttctttgcttgtgtcttgtcttcgtcgtgcttgttcc

SEQ ID NO: 457 - DNA, 84 bp, synthetic construct
  source 1..84: mol_type = other DNA
tttaacaacattattacaggtatagtaaaatgtttagactgtttcgtttctttgcttgtgtcttgtcttcgtcgtgcttgttcc

SEQ ID NO: 458 - DNA, 84 bp, synthetic construct
  source 1..84: mol_type = other DNA
tttatcaaaatcataggtctgataatttcatcttctgaccttttcgtttctttgcttgtgtcttgtcttcgtcgtgcttgttcc

SEQ ID NO: 459 - DNA, 84 bp, synthetic construct
  source 1..84: mol_type = other DNA
tttgatattcattacccaaatctaataaaacgaactaacggtttcgtttctttgcttgtgtcttgtcttcgtcgtgcttgttcc

SEQ ID NO: 460 - DNA, 84 bp, synthetic construct
  source 1..84: mol_type = other DNA
tttaagcgcgaaacaaagtacagtaatcttgacaagaaccgtttcgtttctttgcttgtgtcttgtcttcgtcgtgcttgttcc

SEQ ID NO: 461 - DNA, 84 bp, synthetic construct
  source 1..84: mol_type = other DNA
tttgccgcttttgcgggatcgtgacccccagcgattatacctttcgtttctttgcttgtgtcttgtcttcgtcgtgcttgttcc

SEQ ID NO: 462 - DNA, 51 bp, synthetic construct
  source 1..51: mol_type = other DNA
cgcgtactggcaaggcccccgatttagatttacagttattagcccgcattt

SEQ ID NO: 463 - DNA, 51 bp, synthetic construct
  source 1..51: mol_type = other DNA
ataatcacgccagagcttaatgcgccgctttacagttattagcccgcattt

SEQ ID NO: 464 - DNA, 51 bp, synthetic construct
  source 1..51: mol_type = other DNA
tcttttcgttgcagttataaatcaaaagtttacagttattagcccgcattt

SEQ ID NO: 465 - DNA, 51 bp, synthetic construct
  source 1..51: mol_type = other DNA
ctcatggcaaactataaaagagtctgtctttacagttattagcccgcattt

SEQ ID NO: 466 - DNA, 51 bp, synthetic construct
  source 1..51: mol_type = other DNA
gcgtaaggagataggccattgcaacaggtttacagttattagcccgcattt

SEQ ID NO: 467 - DNA, 51 bp, synthetic construct
  source 1..51: mol_type = other DNA
cctcacattcctgtaatgagtgagctaatttacagttattagcccgcattt

SEQ ID NO: 468 - DNA, 51 bp, synthetic construct
  source 1..51: mol_type = other DNA
agcagcacagaagaacaatatttttgaatttacagttattagcccgcattt

SEQ ID NO: 469 - DNA, 51 bp, synthetic construct
  source 1..51: mol_type = other DNA
ctaacaagttatctaacagtgccacgcttttacagttattagcccgcattt

SEQ ID NO: 470 - DNA, 51 bp, synthetic construct
  source 1..51: mol_type = other DNA
gccgggtcatccctctgcatcagacgattttacagttattagcccgcattt

SEQ ID NO: 471 - DNA, 51 bp, synthetic construct
  source 1..51: mol_type = other DNA
aattatcttattaaccgtcaatagataatttacagttattagcccgcattt

SEQ ID NO: 472 - DNA, 51 bp, synthetic construct
  source 1..51: mol_type = other DNA
aaagaaaaattattagaaaccaccagaatttacagttattagcccgcattt

SEQ ID NO: 473 - DNA, 51 bp, synthetic construct
  source 1..51: mol_type = other DNA
aagttaatggcagccagcaaccgcaagatttacagttattagcccgcattt

SEQ ID NO: 474 - DNA, 51 bp, synthetic construct
  source 1..51: mol_type = other DNA
aacatcaagttacaaggtttaacgtcagtttacagttattagcccgcattt

SEQ ID NO: 475 - DNA, 51 bp, synthetic construct
  source 1..51: mol_type = other DNA
cccttagtaaatcggcaaaagaagatgatttacagttattagcccgcattt

SEQ ID NO: 476 - DNA, 51 bp, synthetic construct
  source 1..51: mol_type = other DNA
gagccgcgtttacccacggaaaaagagatttacagttattagcccgcattt

SEQ ID NO: 477 - DNA, 51 bp, synthetic construct
  source 1..51: mol_type = other DNA
acgagtgttgttccggtcgaggtgccgttttacagttattagcccgcattt

SEQ ID NO: 478 - DNA, 51 bp, synthetic construct
  source 1..51: mol_type = other DNA
gagaaaaggcttagtagcgatagcttagtttacagttattagcccgcattt

SEQ ID NO: 479 - DNA, 51 bp, synthetic construct
  source 1..51: mol_type = other DNA
tagctcactgcccgcaacagctgattagtttacagttattagcccgcattt

SEQ ID NO: 480 - DNA, 51 bp, synthetic construct
  source 1..51: mol_type = other DNA
accgcctgtgcactggtaccgagctcgatttacagttattagcccgcattt

SEQ ID NO: 481 - DNA, 51 bp, synthetic construct
  source 1..51: mol_type = other DNA
caacttaaatttctcgttccggcaaaaatttacagttattagcccgcattt

SEQ ID NO: 482 - DNA, 51 bp, synthetic construct
  source 1..51: mol_type = other DNA
tgtgcaaggcgattacctcaccggaaattttacagttattagcccgcattt

SEQ ID NO: 483 - DNA, 51 bp, synthetic construct
  source 1..51: mol_type = other DNA
ggcaaagcgccattgctattacgccagctttacagttattagcccgcattt

SEQ ID NO: 484 - DNA, 42 bp, synthetic construct
  source 1..42: mol_type = other DNA
tacatttcctttgccaaaatcagaacaagcaagccgttttta

SEQ ID NO: 485 - DNA, 45 bp, synthetic construct
  source 1..45: mol_type = other DNA
cagagccgccggtgaatttcggtggtgccatggctggtaatggtt

SEQ ID NO: 486 - DNA, 49 bp, synthetic construct
  source 1..49: mol_type = other DNA
gaggaagctaatagattagagttttaaaccatttgggaattaagcaccg

SEQ ID NO: 487 - DNA, 52 bp, synthetic construct
  source 1..52: mol_type = other DNA
gacattcaacgtttagcaacgcggtccgtttcggcagcaccgtgcggaacaa

SEQ ID NO: 488 - DNA, 22 bp, synthetic construct
  source 1..22: mol_type = other DNA
catcgtaggatagcgaacctcc

SEQ ID NO: 489 - DNA, 22 bp, synthetic construct
  source 1..22: mol_type = other DNA
cagtagcgaccgtttgccatct

SEQ ID NO: 490 - DNA, 23 bp, synthetic construct
  source 1..23: mol_type = other DNA
tttcataatcaacaaagacaaaa

SEQ ID NO: 491 - DNA, 23 bp, synthetic construct
  source 1..23: mol_type = other DNA
agaaggaaaccgcggaaccgcct

SEQ ID NO: 492 - DNA, 44 bp, synthetic construct
  source 1..44: mol_type = other DNA
ggtaattgcattagacgggagaattaactgaactttatacatct

SEQ ID NO: 493 - DNA, 78 bp, synthetic construct
  source 1..78: mol_type = other DNA
tttagaaaatacatacatagtatgttagcaaacgttttcactcacctccatctccactcctacccatccaactcccac

SEQ ID NO: 494 - DNA, 21 bp, synthetic construct
  source 1..21: mol_type = other DNA
taaccctatacactaaaacac

SEQ ID NO: 495 - DNA, 21 bp, synthetic construct
  source 1..21: mol_type = other DNA
ttaaacaaatctccaaaaaaa

SEQ ID NO: 496 - DNA, 21 bp, synthetic construct
  source 1..21: mol_type = other DNA
gcggccatgccccctgcctat

SEQ ID NO: 497 - DNA, 21 bp, synthetic construct
  source 1..21: mol_type = other DNA
gtagcatttgagccatttggg

SEQ ID NO: 498 - DNA, 84 bp, synthetic construct
  source 1..84: mol_type = other DNA
atttgtagcgcataaagataagagccagtttcactcacctccatctccactcctacccatccaactcccactttctagatgtat

SEQ ID NO: 499 - DNA, 84 bp, synthetic construct
  source 1..84: mol_type = other DNA
attgcgtatattcctaccgaatctaaagtttcactcacctccatctccactcctacccatccaactcccactttctagatgtat

SEQ ID NO: 500 - DNA, 84 bp, synthetic construct
  source 1..84: mol_type = other DNA
taccatactgattgttaatgcatcaatatttcactcacctccatctccactcctacccatccaactcccactttctagatgtat

SEQ ID NO: 501 - DNA, 28 bp, synthetic construct
  source 1..28: mol_type = other DNA
aggcaaagcaaggcaacagccatattat

SEQ ID NO: 502 - DNA, 32 bp, synthetic construct
  source 1..32: mol_type = other DNA
atttacattgggtgaggcggtgtacagaccag

SEQ ID NO: 503 - DNA, 32 bp, synthetic construct
  source 1..32: mol_type = other DNA
cgaacgtggcgttttagacctcagcagcgaaa

SEQ ID NO: 504 - DNA, 33 bp, synthetic construct
  source 1..33: mol_type = other DNA
ttgagggcaccgactaacatctcaattctacta

SEQ ID NO: 505 - DNA, 91 bp, synthetic construct
  source 1..91: mol_type = other DNA
acgcaatgtcaaatcaccatcagccccagttaaaatttcactcacctccatctccactcctacccatccaactcccactttctagatgtat

SEQ ID NO: 506 - DNA, 35 bp, synthetic construct
  source 1..35: mol_type = other DNA
aagcagaaaattaatgccggaactagcataaccaa

SEQ ID NO: 507 - DNA, 91 bp, synthetic construct
  source 1..91: mol_type = other DNA
aatatgatattcaaccgttctaccccggttgttaatttcactcacctccatctccactcctacccatccaactcccactttctagatgtat

SEQ ID NO: 508 - DNA, 91 bp, synthetic construct
  source 1..91: mol_type = other DNA
gagggtagctatttttgagagtcgatgaaaaataatttcactcacctccatctccactcctacccatccaactcccactttctagatgtat

SEQ ID NO: 509 - DNA, 35 bp, synthetic construct
  source 1..35: mol_type = other DNA
atcgtcgaaagaagagagcggaaagagtctgtcca

SEQ ID NO: 510 - DNA, 35 bp, synthetic construct
  source 1..35: mol_type = other DNA
aatcttgtgaattattttaagaactggctcattat

SEQ ID NO: 511 - DNA, 35 bp, synthetic construct
  source 1..35: mol_type = other DNA
aagaaacacaaacaactaacaactaatagattaga

SEQ ID NO: 512 - DNA, 35 bp, synthetic construct
  source 1..35: mol_type = other DNA
acattatattaaatatctaaaatatcttaccctca

SEQ ID NO: 513 - DNA, 35 bp, synthetic construct
  source 1..35: mol_type = other DNA
aattaaccgttgtaatccagaagtaacagtacctt

SEQ ID NO: 514 - DNA, 35 bp, synthetic construct
  source 1..35: mol_type = other DNA
cgggcgctagggcgtagaatcatgatgaaacaaac

SEQ ID NO: 515 - DNA, 35 bp, synthetic construct
  source 1..35: mol_type = other DNA
agtccactattaaaaatcaagaacatagcgatagc

SEQ ID NO: 516 - DNA, 35 bp, synthetic construct
  source 1..35: mol_type = other DNA
gagccggagcctcccagacgaaggtttcacgcaac

SEQ ID NO: 517 - DNA, 35 bp, synthetic construct
  source 1..35: mol_type = other DNA
ttaatgaatcggccgcggtcctaaatgctgatgca

SEQ ID NO: 518 - DNA, 35 bp, synthetic construct
  source 1..35: mol_type = other DNA
taagaggtcattttagaccggaggtgtatcaccgt

SEQ ID NO: 519 - DNA, 35 bp, synthetic construct
  source 1..35: mol_type = other DNA
tcacagttgaggattccacacctagaaaaagcctg

SEQ ID NO: 520 - DNA, 35 bp, synthetic construct
  source 1..35: mol_type = other DNA
ctggtaatgggtaatccagcgaggcagaggcattt

SEQ ID NO: 521 - DNA, 35 bp, synthetic construct
  source 1..35: mol_type = other DNA
ttacactggtgtgtttacctgaccgacaaaaggta

SEQ ID NO: 522 - DNA, 35 bp, synthetic construct
  source 1..35: mol_type = other DNA
ccattagatacattgaagtttttgaggcaggtcag

SEQ ID NO: 523 - DNA, 35 bp, synthetic construct
  source 1..35: mol_type = other DNA
ctccggccagagcaggtggtgaaaccaatcaataa

SEQ ID NO: 524 - DNA, 35 bp, synthetic construct
  source 1..35: mol_type = other DNA
gataaccgacggccctcaggagtaaccgatatttt

SEQ ID NO: 525 - DNA, 35 bp, synthetic construct
  source 1..35: mol_type = other DNA
acgtacagcgccattacatcgtatagaaggcttat

SEQ ID NO: 526 - DNA, 35 bp, synthetic construct
  source 1..35: mol_type = other DNA
tagactttctccgtttaaattagcgaacctcccga

SEQ ID NO: 527 - DNA, 91 bp, synthetic construct
  source 1..91: mol_type = other DNA
ggtgaagacgccaggcgcaacgtaacaactggccttttcactcacctccatctccactcctacccatccaactcccactttctagatgtat

SEQ ID NO: 528 - DNA, 36 bp, synthetic construct
  source 1..36: mol_type = other DNA
cactgcccgctttccgatggtgagcgtaacgatcta

SEQ ID NO: 529 - DNA, 44 bp, synthetic construct
  source 1..44: mol_type = other DNA
  modified_base 1: mod_base = OTHER; note = Biotin modified nucleotide
tttggggtttccggaataagcaaacgagcttcaaagcgaacgct

SEQ ID NO: 530 - DNA, 98 bp, synthetic construct
  source 1..98: mol_type = other DNA
gaatacccaaagacgccagtttgaggaaatatttaaattgtatttcactcacctccatctccactcctacccatccaactcccactttctagatgtat

SEQ ID NO: 531 - DNA, 42 bp, synthetic construct
  source 1..42: mol_type = other DNA
cgaggaattattttgcgcatcagatcgcactccagcgacgtt

SEQ ID NO: 532 - DNA, 95 bp, synthetic construct
  source 1..95: mol_type = other DNA
ttcaaaagggtgagaaaggccgtataagcaaataaaaattttcactcacctccatctccactcctacccatccaactcccactttctagatgtat

SEQ ID NO: 533 - DNA, 42 bp, synthetic construct
  source 1..42: mol_type = other DNA
tggtttacagtagcgtaaaactcaccggaaacaatcgtaaaa

SEQ ID NO: 534 - DNA, 45 bp, synthetic construct
  source 1..45: mol_type = other DNA
  modified_base 1: mod_base = OTHER; note = Biotin modified nucleotide
tttgatagcacgtttgcagtgatgaaggggcaaatggtcaataac

SEQ ID NO: 535 - DNA, 42 bp, synthetic construct
  source 1..42: mol_type = other DNA
aacgtcacaaaatcaaagccgtccggcaaacgcggcagcatc

SEQ ID NO: 536 - DNA, 42 bp, synthetic construct
  source 1..42: mol_type = other DNA
accgcctaaacaaaagcggggcgggtcactgttgcgcctgtg

SEQ ID NO: 537 - DNA, 45 bp, synthetic construct
  source 1..45: mol_type = other DNA
  modified_base 1: mod_base = OTHER; note = Biotin modified nucleotide
tttaccgttccagttaagaatgcggcgggcggatggcttagagct

SEQ ID NO: 538 - DNA, 42 bp, synthetic construct
  source 1..42: mol_type = other DNA
gaaagcgttcggaacactctgtctgccagcacgcggggtgcc

SEQ ID NO: 539 - DNA, 42 bp, synthetic construct
  source 1..42: mol_type = other DNA
attaagacaccctctaatgagaaacctgtcgtgcccagcagg

SEQ ID NO: 540 - DNA, 42 bp, synthetic construct
  source 1..42: mol_type = other DNA
gtgcctttttgatggcattgaccaccctgcattttgaatcaa

SEQ ID NO: 541 - DNA, 42 bp, synthetic construct
  source 1..42: mol_type = other DNA
accctcaaagttttcgaaaattagcccgagataggggaaccc

SEQ ID NO: 542 - DNA, 42 bp, synthetic construct
  source 1..42: mol_type = other DNA
tgaatttattgtattaaagggaagggaagaaagcgacaggag

SEQ ID NO: 543 - DNA, 42 bp, synthetic construct
  source 1..42: mol_type = other DNA
tgaatttgacagcagccgattaatcagtgaggccagctcatg

SEQ ID NO: 544 - DNA, 42 bp, synthetic construct
  source 1..42: mol_type = other DNA
tttttcagagtgagacgcctgacccatggtatagctgctcag

SEQ ID NO: 545 - DNA, 42 bp, synthetic construct
  source 1..42: mol_type = other DNA
cagaggctataccagaaatacaccagtcacacgacccagcag

SEQ ID NO: 546 - DNA, 98 bp, synthetic construct
  source 1..98: mol_type = other DNA
ttcattataatttcaccagtcaggacgtagcaccgcctgcaatttcactcacctccatctccactcctacccatccaactcccactttctagatgtat

SEQ ID NO: 547 - DNA, 42 bp, synthetic construct
  source 1..42: mol_type = other DNA
ccttaacatttgaggatttaggccgtcaatagataattgcga

SEQ ID NO: 548 - DNA, 42 bp, synthetic construct
  source 1..42: mol_type = other DNA
gtgttgacgctcaatcgtctgacagggccagaatcctgagaa

SEQ ID NO: 549 - DNA, 42 bp, synthetic construct
  source 1..42: mol_type = other DNA
tttttataaagggaagaaaggagcccccaaaagaacctgttt

SEQ ID NO: 550 - DNA, 42 bp, synthetic construct
  source 1..42: mol_type = other DNA
gatttagagcttgacggggctaagcaaaatcccttataaatc

SEQ ID NO: 551 - DNA, 42 bp, synthetic construct
  source 1..42: mol_type = other DNA
actcacattaattgcgttgcctgccgttttcacggtcatacc

SEQ ID NO: 552 - DNA, 42 bp, synthetic construct
  source 1..42: mol_type = other DNA
agctgcaaagcctgtgcctgtactgcgccctgcggaggtgtc

SEQ ID NO: 553 - DNA, 42 bp, synthetic construct
  source 1..42: mol_type = other DNA
aggcgctttcgcactcaattgtctaaagttaaacgatgctga

SEQ ID NO: 554 - DNA, 42 bp, synthetic construct
  source 1..42: mol_type = other DNA
agtgccaagctttcagaggtataggacgacgacagtatcggc

SEQ ID NO: 555 - DNA, 44 bp, synthetic construct
  source 1..44: mol_type = other DNA
atagtggagccgccacgggaacgggcctttcatcttttcataat

SEQ ID NO: 556 - DNA, 45 bp, synthetic construct
  source 1..45: mol_type = other DNA
ttcagcgcgttgaagttcagagaatccccctcaaatgaaagccgg

SEQ ID NO: 557 - DNA, 45 bp, synthetic construct
  source 1..45: mol_type = other DNA
cattaaacaaaagacgtttacgtaagagcaacactataatggatt

SEQ ID NO: 558 - DNA, 49 bp, synthetic construct
  source 1..49: mol_type = other DNA
acaaagtccctgaaaggtcactccggcaccgcttcacgccagggttttc

SEQ ID NO: 559 - DNA, 49 bp, synthetic construct
  source 1..49: mol_type = other DNA
tcttaccagataacgattctctcgccattcaggctctggcgaaaggggg

SEQ ID NO: 560 - DNA, 49 bp, synthetic construct
  source 1..49: mol_type = other DNA
gaagcgccaaaatagattaagagtcccggaatttggccagcagttgggc

SEQ ID NO: 561 - DNA, 49 bp, synthetic construct
  source 1..49: mol_type = other DNA
gagcgtcaatcagaacataaatttcgtctcgtcgccagcttacggctgg

SEQ ID NO: 562 - DNA, 49 bp, synthetic construct
  source 1..49: mol_type = other DNA
gcacccagcgtttttctgctcataacggaacgtgcaatgccaacggcag

SEQ ID NO: 563 - DNA, 49 bp, synthetic construct
  source 1..49: mol_type = other DNA
gtaggaacatgtagccatccctttgctcgtcataaggtgccccctgcat

SEQ ID NO: 564 - DNA, 49 bp, synthetic construct
  source 1..49: mol_type = other DNA
aagaaaagtaatttcagtgtctcttcgcgtccgtgaagcataaagtgta

SEQ ID NO: 565 - DNA, 49 bp, synthetic construct
  source 1..49: mol_type = other DNA
tgcagaaataaagtcagccagtaccgagctcgaataaattgttatccgc

SEQ ID NO: 566 - DNA, 49 bp, synthetic construct
  source 1..49: mol_type = other DNA
ttgagaaataattaaacatacggggagaggcggttgccctgagagagtt

SEQ ID NO: 567 - DNA, 49 bp, synthetic construct
  source 1..49: mol_type = other DNA
taaggcgctatatgacgctgggttgttccagtttgggtgccgtaaagca

SEQ ID NO: 568 - DNA, 49 bp, synthetic construct
  source 1..49: mol_type = other DNA
tgacctaacgcgagcccttcagactccaacgtcaacactacgtgaacca

SEQ ID NO: 569 - DNA, 49 bp, synthetic construct
  source 1..49: mol_type = other DNA
ttttaacccttgaattttttggtgtagcggtcacgcgtataacgtgctt

SEQ ID NO: 570 - DNA, 49 bp, synthetic construct
  source 1..49: mol_type = other DNA
acataaaacatttatgctttgttctttgattagtaactatcggccttgc

SEQ ID NO: 571 - DNA, 49 bp, synthetic construct
  source 1..49: mol_type = other DNA
attgtgtgatgaacggtcagtattaaatttaggaataccacaagattca

SEQ ID NO: 572 - DNA, 49 bp, synthetic construct
  source 1..49: mol_type = other DNA
tgctcatccgaacttgttactaaagaggcgggtaacagggagaaccatc

SEQ ID NO: 573 - DNA, 49 bp, synthetic construct
  source 1..49: mol_type = other DNA
acaaagctaaattgaaaaatctacgttaggtagaattcaactaggcata

SEQ ID NO: 574 - DNA, 49 bp, synthetic construct
  source 1..49: mol_type = other DNA
gaaaaacccgagtagagctaaaaaggagctaaatcgttgagttttgccc

SEQ ID NO: 575 - DNA, 49 bp, synthetic construct
  source 1..49: mol_type = other DNA
agccattgcaacagaaaagggacattctttaaaaatgattatcagatga

SEQ ID NO: 576 - DNA, 49 bp, synthetic construct
  source 1..49: mol_type = other DNA
tccgtttaaaatcccggcgaaccagtcaccagcttgttggtgtagatgg

SEQ ID NO: 577 - DNA, 49 bp, synthetic construct
  source 1..49: mol_type = other DNA
tggcagcggttgtggtttaccttgggtatggtgccgaccgtacattttt

SEQ ID NO: 578 - DNA, 56 bp, synthetic construct
  source 1..56: mol_type = other DNA
tcaccgtagggaagataaagggactccttgtgtaggtaaagatagaaccatttcaa

SEQ ID NO: 579 - DNA, 56 bp, synthetic construct
  source 1..56: mol_type = other DNA
agagccacaggaggcattccaactaaagtacggtgtcccgccgggcgcggttgcgg

SEQ ID NO: 580 - DNA, 56 bp, synthetic construct
  source 1..56: mol_type = other DNA
attttcacatagttgttccgaaatcgagcggattgcatcaaattatagtcagaagc

SEQ ID NO: 581 - DNA, 56 bp, synthetic construct
  source 1..56: mol_type = other DNA
taccgattcgtcaccaggaacggtactaatagtaaaatgtttgttttgccagaggg

SEQ ID NO: 582 - DNA, 56 bp, synthetic construct
  source 1..56: mol_type = other DNA
gaggcgaaatatacacaatatagagatagaaccctgatagccctaaaacacctcaa

SEQ ID NO: 583 - DNA, 56 bp, synthetic construct
  source 1..56: mol_type = other DNA
gcgaacttctgacctggtaatgcaatacacgagcactgcgcgtcacccagaacgtg

SEQ ID NO: 584 - DNA, 56 bp, synthetic construct
  source 1..56: mol_type = other DNA
taccgcctcacgcatcctcgtctggcaagggtcgagaacaaggcagcaaaacgcgc

SEQ ID NO: 585 - DNA, 56 bp, synthetic construct
  source 1..56: mol_type = other DNA
aagattatttaattctccaaccttttgataattgcatatgcatataacagttgatt

SEQ ID NO: 586 - DNA, 56 bp, synthetic construct
  source 1..56: mol_type = other DNA
ccgcctgtgcgtattcacaatccccgggcggtgccacatccccaccgtccatcctc

SEQ ID NO: 587 - DNA, 56 bp, synthetic construct
  source 1..56: mol_type = other DNA
agtcgggtgagctagggggtttggtgcttatgagctcattgcttgccgtcacaggc

SEQ ID NO: 588 - DNA, 56 bp, synthetic construct
  source 1..56: mol_type = other DNA
atttgcctgagagaatgtgctgcgccatcgtgggagccatcaacggtaatcgtaaa

SEQ ID NO: 589 - DNA, 27 bp, synthetic construct
  source 1..27: mol_type = other DNA
tttaaacgtagaaaagaccctgtattt

SEQ ID NO: 590 - DNA, 27 bp, synthetic construct
  source 1..27: mol_type = other DNA
tttattcattaaaggggcaaggcattt

SEQ ID NO: 591 - DNA, 27 bp, synthetic construct
  source 1..27: mol_type = other DNA
tttctgtagcgcgtttttcatttgttt

SEQ ID NO: 592 - DNA, 27 bp, synthetic construct
  source 1..27: mol_type = other DNA
tttaccaccagagcccccaattctttt

SEQ ID NO: 593 - DNA, 27 bp, synthetic construct
  source 1..27: mol_type = other DNA
tttataagttttaacaatgctgtattt

SEQ ID NO: 594 - DNA, 27 bp, synthetic construct
  source 1..27: mol_type = other DNA
tttgtcgagagggttgattagagattt

SEQ ID NO: 595 - DNA, 27 bp, synthetic construct
  source 1..27: mol_type = other DNA
tttgtcaccagtacagcccgaaagttt

SEQ ID NO: 596 - DNA, 27 bp, synthetic construct
  source 1..27: mol_type = other DNA
tttaggaattgcgaaataaatcaattt

SEQ ID NO: 597 - DNA, 27 bp, synthetic construct
  source 1..27: mol_type = other DNA
tttattcggtcgctgccaatactgttt

SEQ ID NO: 598 - DNA, 27 bp, synthetic construct
  source 1..27: mol_type = other DNA
tttaaggcaccaaccaaccaaaatttt

SEQ ID NO: 599 - DNA, 27 bp, synthetic construct
  source 1..27: mol_type = other DNA
tttacggtcaatcatatacataacttt

SEQ ID NO: 600 - DNA, 27 bp, synthetic construct
  source 1..27: mol_type = other DNA
tttctgacgagaaacgaactaacgttt

SEQ ID NO: 601 - DNA, 27 bp, synthetic construct
  source 1..27: mol_type = other DNA
tttctggtctggtcaacgggtattttt

SEQ ID NO: 602 - DNA, 27 bp, synthetic construct
  source 1..27: mol_type = other DNA
tttagagacgcagaagaggttttgttt

SEQ ID NO: 603 - DNA, 27 bp, synthetic construct
  source 1..27: mol_type = other DNA
ttttgcgggcctctttttgtttaattt

SEQ ID NO: 604 - DNA, 27 bp, synthetic construct
  source 1..27: mol_type = other DNA
tttcaacattaaatgcaataataattt

SEQ ID NO: 605 - DNA, 29 bp, synthetic construct
  source 1..29: mol_type = other DNA
aatagcaaaggctatcaggtcattgcttt

SEQ ID NO: 606 - DNA, 29 bp, synthetic construct
  source 1..29: mol_type = other DNA
gccgccaatacaggagtgtactggtattt

SEQ ID NO: 607 - DNA, 30 bp, synthetic construct
  source 1..30: mol_type = other DNA
ttttttagttaatttcgttatacaaatttt

SEQ ID NO: 608 - DNA, 30 bp, synthetic construct
  source 1..30: mol_type = other DNA
tttcttttttaatggtgagaagagtcattt

SEQ ID NO: 609 - DNA, 30 bp, synthetic construct
  source 1..30: mol_type = other DNA
ttttaatggaagggtacaataacggatttt

SEQ ID NO: 610 - DNA, 33 bp, synthetic construct
  source 1..33: mol_type = other DNA
tttgcgaacgagtagatttagtttgactgttta

SEQ ID NO: 611 - DNA, 38 bp, synthetic construct
  source 1..38: mol_type = other DNA
tttacgacgacaataaacaaagtaattctgtccagttt

SEQ ID NO: 612 - DNA, 38 bp, synthetic construct
  source 1..38: mol_type = other DNA
ttttatttttgaatggctatacgtggcacagacaattt

SEQ ID NO: 613 - DNA, 38 bp, synthetic construct
  source 1..38: mol_type = other DNA
tttgagtagaagaactcaaataacatcacttgcctttt

SEQ ID NO: 614 - DNA, 38 bp, synthetic construct
  source 1..38: mol_type = other DNA
tttcgctacagggcgcgtagccgcgcttaatgcgcttt

SEQ ID NO: 615 - DNA, 38 bp, synthetic construct
  source 1..38: mol_type = other DNA
ttttatcagggcgatggccagggcgaaaaaccgtcttt

SEQ ID NO: 616 - DNA, 38 bp, synthetic construct
  source 1..38: mol_type = other DNA
tttgtgagacgggcaacaggtttttcttttcaccattt

SEQ ID NO: 617 - DNA, 38 bp, synthetic construct
  source 1..38: mol_type = other DNA
tttagctgtttcctgtgtgtcgtaatcatggtcatttt

SEQ ID NO: 618 - DNA, 38 bp, synthetic construct
  source 1..38: mol_type = other DNA
tttggcatcagatgccgggtcagcaaatcgttaacttt

SEQ ID NO: 619 - DNA, 39 bp, synthetic construct
  source 1..39: mol_type = other DNA
tttttatgcctgagtaatattacgcagtatgttagcttt

SEQ ID NO: 620 - DNA, 39 bp, synthetic construct
  source 1..39: mol_type = other DNA
tttttctgagagtctggtcctgtagccagctttcatttt

SEQ ID NO: 621 - DNA, 40 bp, synthetic construct
  source 1..40: mol_type = other DNA
tttttgttgaaaggaattgagagttggcaaatcaacattt

SEQ ID NO: 622 - DNA, 40 bp, synthetic construct
  source 1..40: mol_type = other DNA
tttcggaatcgtcataaatattcattaaacgagctgacta

SEQ ID NO: 623 - DNA, 42 bp, synthetic construct
  source 1..42: mol_type = other DNA
tttttgaacaacattattacaataaaacaccagaacgagtag

SEQ ID NO: 624 - DNA, 43 bp, synthetic construct
  source 1..43: mol_type = other DNA
tgaaagcgtaagaattagtcttttggattatacttctgaattt

SEQ ID NO: 625 - DNA, 43 bp, synthetic construct
  source 1..43: mol_type = other DNA
taaccaccacacccctatggtacaatttcatttgaattacttt

SEQ ID NO: 626 - DNA, 43 bp, synthetic construct
  source 1..43: mol_type = other DNA
tgggcgccagggtgctgattgaaaactttttcaaatatatttt

SEQ ID NO: 627 - DNA, 54 bp, synthetic construct
  source 1..54: mol_type = other DNA
tttaaaccaagtaccgcactccaagagcagcaaccgcaagcggacttatcaaac

SEQ ID NO: 628 - DNA, 55 bp, synthetic construct
  source 1..55: mol_type = other DNA
tttgagcaagaaacaatgattaagcctgagcgatgttgggaagggcgatcggttt

SEQ ID NO: 629 - DNA, 55 bp, synthetic construct
  source 1..55: mol_type = other DNA
tttcgtcaaaaatgaaaatacgatttcgctattggatagctctcacggaaaattt

SEQ ID NO: 630 - DNA, 55 bp, synthetic construct
  source 1..55: mol_type = other DNA
tttaagccttaaatcaagacttgcggacagcgggtagaacgtcagcgtggtgttt

SEQ ID NO: 631 - DNA, 55 bp, synthetic construct
  source 1..55: mol_type = other DNA
tttgccaaaaggaattacgaatgcagaagggaatcagtgaataaggcttgccttt

SEQ ID NO: 632 - DNA, 55 bp, synthetic construct
  source 1..55: mol_type = other DNA
tttagcgagaggcttttgccgataaataaaacgtagccggaacgaggcgcagttt

SEQ ID NO: 633 - DNA, 55 bp, synthetic construct
  source 1..55: mol_type = other DNA
tttaaatcaggtctttaccaatgacctaataatgcccacgcataaccgatatttt

SEQ ID NO: 634 - DNA, 55 bp, synthetic construct
  source 1..55: mol_type = other DNA
tttacttcaaatatcgcgtagaggaaaactacaaatagaaaggaacaactaattt

SEQ ID NO: 635 - DNA, 55 bp, synthetic construct
  source 1..55: mol_type = other DNA
tttgtacctttaattgctcaggtcaggatataataccgtaacactgagtttcttt

SEQ ID NO: 636 - DNA, 55 bp, synthetic construct
  source 1..55: mol_type = other DNA
tttgctcaacatgttttaatgaatatggggtcataccaggcggataagtgccttt

SEQ ID NO: 637 - DNA, 55 bp, synthetic construct
  source 1..55: mol_type = other DNA
tttgggcgcgagctgaaaagctatatttcatcgcagagccgccaccagaaccttt

SEQ ID NO: 638 - DNA, 55 bp, synthetic construct
  source 1..55: mol_type = other DNA
tttaagaattagcaaaatttcatacatgaattagtttgcctttagcgtcagattt

SEQ ID NO: 639 - DNA, 55 bp, synthetic construct
  source 1..55: mol_type = other DNA
tttatacttttgcgggagaacattattacatacgtaaatattgacggaaattttt

SEQ ID NO: 640 - DNA, 57 bp, synthetic construct
  source 1..57: mol_type = other DNA
cagacgaaaaagaaagactggatagcgtaggcttgaatacgtaatgccactacgttt

SEQ ID NO: 641 - DNA, 57 bp, synthetic construct
  source 1..57: mol_type = other DNA
ggtggcacaataaaaagcaataccaaaaagcctttctcatatattttaaatgcattt

SEQ ID NO: 642 - DNA, 65 bp, synthetic construct
  source 1..65: mol_type = other DNA
ttttcttaccagtataaagccatttcgtttctttgcttgtgtcttgtcttcgtcgtgcttgttcc

SEQ ID NO: 643 - DNA, 65 bp, synthetic construct
  source 1..65: mol_type = other DNA
tttatagtgaatttatcaaaattttcgtttctttgcttgtgtcttgtcttcgtcgtgcttgttcc

SEQ ID NO: 644 - DNA, 65 bp, synthetic construct
  source 1..65: mol_type = other DNA
ttttcgcctgattgctttgaattttcgtttctttgcttgtgtcttgtcttcgtcgtgcttgttcc

SEQ ID NO: 645 - DNA, 65 bp, synthetic construct
  source 1..65: mol_type = other DNA
tttaattttaaaagtttgagtatttcgtttctttgcttgtgtcttgtcttcgtcgtgcttgttcc

SEQ ID NO: 646 - DNA, 71 bp, synthetic construct
  source 1..71: mol_type = other DNA
cgacattagaaacgcaaaagaactggcatttcgtttctttgcttgtgtcttgtcttcgtcgtgcttgttcc

SEQ ID NO: 647 - DNA, 71 bp, synthetic construct
  source 1..71: mol_type = other DNA
aacataaatcagaggaagccctttttaatttcgtttctttgcttgtgtcttgtcttcgtcgtgcttgttcc

SEQ ID NO: 648 - DNA, 71 bp, synthetic construct
  source 1..71: mol_type = other DNA
gtcacaatcaatcataccagaaggaaactttcgtttctttgcttgtgtcttgtcttcgtcgtgcttgttcc

SEQ ID NO: 649 - DNA, 71 bp, synthetic construct
  source 1..71: mol_type = other DNA
ccaatgaaaatcacccagcgccaaagactttcgtttctttgcttgtgtcttgtcttcgtcgtgcttgttcc

SEQ ID NO: 650 - DNA, 71 bp, synthetic construct
  source 1..71: mol_type = other DNA
ttaccaaccagttaattagacgggagaatttcgtttctttgcttgtgtcttgtcttcgtcgtgcttgttcc

SEQ ID NO: 651 - DNA, 71 bp, synthetic construct
  source 1..71: mol_type = other DNA
ttagttgataagaaagcagcctttacagtttcgtttctttgcttgtgtcttgtcttcgtcgtgcttgttcc

SEQ ID NO: 652 - DNA, 71 bp, synthetic construct
  source 1..71: mol_type = other DNA
gaaccgcttattaggcaccgtaatcagttttcgtttctttgcttgtgtcttgtcttcgtcgtgcttgttcc

SEQ ID NO: 653 - DNA, 71 bp, synthetic construct
  source 1..71: mol_type = other DNA
tttttatacgcgaggctacaattttatctttcgtttctttgcttgtgtcttgtcttcgtcgtgcttgttcc

SEQ ID NO: 654 - DNA, 71 bp, synthetic construct
  source 1..71: mol_type = other DNA
accggaaccagacattagcaaggccggatttcgtttctttgcttgtgtcttgtcttcgtcgtgcttgttcc

SEQ ID NO: 655 - DNA, 71 bp, synthetic construct
  source 1..71: mol_type = other DNA
cagtctctattcacccctcagagccgcctttcgtttctttgcttgtgtcttgtcttcgtcgtgcttgttcc

SEQ ID NO: 656 - DNA, 71 bp, synthetic construct
  source 1..71: mol_type = other DNA
gataagtttacgagtcattaccgcgccctttcgtttctttgcttgtgtcttgtcttcgtcgtgcttgttcc

SEQ ID NO: 657 - DNA, 71 bp, synthetic construct
  source 1..71: mol_type = other DNA
acatgttttatcattcatcgagaacaagtttcgtttctttgcttgtgtcttgtcttcgtcgtgcttgttcc

SEQ ID NO: 658 - DNA, 71 bp, synthetic construct
  source 1..71: mol_type = other DNA
ggattaggtataaacagtaagcgtcatatttcgtttctttgcttgtgtcttgtcttcgtcgtgcttgttcc

SEQ ID NO: 659 - DNA, 71 bp, synthetic construct
  source 1..71: mol_type = other DNA
acagtagagagaatcgcgcctgtttatctttcgtttctttgcttgtgtcttgtcttcgtcgtgcttgttcc

SEQ ID NO: 660 - DNA, 71 bp, synthetic construct
  source 1..71: mol_type = other DNA
cctattattctgatataaagccagaatgtttcgtttctttgcttgtgtcttgtcttcgtcgtgcttgttcc

SEQ ID NO: 661 - DNA, 71 bp, synthetic construct
  source 1..71: mol_type = other DNA
gagccacgtaccgcggctgagactcctctttcgtttctttgcttgtgtcttgtcttcgtcgtgcttgttcc

SEQ ID NO: 662 - DNA, 71 bp, synthetic construct
  source 1..71: mol_type = other DNA
gaccgtgcggaatctcgccatatttaactttcgtttctttgcttgtgtcttgtcttcgtcgtgcttgttcc

SEQ ID NO: 663 - DNA, 71 bp, synthetic construct
  source 1..71: mol_type = other DNA
caactttcagccctgggatagcaagccctttcgtttctttgcttgtgtcttgtcttcgtcgtgcttgttcc

SEQ ID NO: 664 - DNA, 71 bp, synthetic construct
  source 1..71: mol_type = other DNA
ctgagagacaaagaaatttaatggtttgtttcgtttctttgcttgtgtcttgtcttcgtcgtgcttgttcc

SEQ ID NO: 665 - DNA, 71 bp, synthetic construct
  source 1..71: mol_type = other DNA
gtcgtctttccaaattctcagaaccgcctttcgtttctttgcttgtgtcttgtcttcgtcgtgcttgttcc

SEQ ID NO: 666 - DNA, 71 bp, synthetic construct
  source 1..71: mol_type = other DNA
cttaaacgcctttatctgtatgggattttttcgtttctttgcttgtgtcttgtcttcgtcgtgcttgttcc

SEQ ID NO: 667 - DNA, 71 bp, synthetic construct
  source 1..71: mol_type = other DNA
ccttgctttagaatctccggcttaggtttttcgtttctttgcttgtgtcttgtcttcgtcgtgcttgttcc

SEQ ID NO: 668 - DNA, 71 bp, synthetic construct
  source 1..71: mol_type = other DNA
catgaggtgcgggaagttgcgccgacaatttcgtttctttgcttgtgtcttgtcttcgtcgtgcttgttcc

SEQ ID NO: 669 - DNA, 71 bp, synthetic construct
  source 1..71: mol_type = other DNA
tacaaaaattaatttcaatatatgtgagtttcgtttctttgcttgtgtcttgtcttcgtcgtgcttgttcc

SEQ ID NO: 670 - DNA, 71 bp, synthetic construct
  source 1..71: mol_type = other DNA
tcggaacgagggcactttgctttcgaggtttcgtttctttgcttgtgtcttgtcttcgtcgtgcttgttcc

SEQ ID NO: 671 - DNA, 71 bp, synthetic construct
  source 1..71: mol_type = other DNA
tcatcgccagcgattttgaggactaaagtttcgtttctttgcttgtgtcttgtcttcgtcgtgcttgttcc

SEQ ID NO: 672 - DNA, 71 bp, synthetic construct
  source 1..71: mol_type = other DNA
acagaaatcagatgattattcatttcaatttcgtttctttgcttgtgtcttgtcttcgtcgtgcttgttcc

SEQ ID NO: 673 - DNA, 71 bp, synthetic construct
  source 1..71: mol_type = other DNA
cccaaatgaggacacgaaatccgcgacctttcgtttctttgcttgtgtcttgtcttcgtcgtgcttgttcc

SEQ ID NO: 674 - DNA, 71 bp, synthetic construct
  source 1..71: mol_type = other DNA
cattttgtataatctcaaaattatttgctttcgtttctttgcttgtgtcttgtcttcgtcgtgcttgttcc

SEQ ID NO: 675 - DNA, 71 bp, synthetic construct
  source 1..71: mol_type = other DNA
ggctggctgacctcagagtacaacggagtttcgtttctttgcttgtgtcttgtcttcgtcgtgcttgttcc

SEQ ID NO: 676 - DNA, 71 bp, synthetic construct
  source 1..71: mol_type = other DNA
aactttaatcattgacaagaaccggatatttcgtttctttgcttgtgtcttgtcttcgtcgtgcttgttcc

SEQ ID NO: 677 - DNA, 71 bp, synthetic construct
  source 1..71: mol_type = other DNA
aagtattagactttcaccagaaggagcgtttcgtttctttgcttgtgtcttgtcttcgtcgtgcttgttcc

SEQ ID NO: 678 - DNA, 21 bp, synthetic construct
  source 1..21: mol_type = other DNA
ttaactgaaagaaaattcata

SEQ ID NO: 679 - DNA, 21 bp, synthetic construct
  source 1..21: mol_type = other DNA
aatagcaaggccaccaccgga

SEQ ID NO: 680 - DNA, 21 bp, synthetic construct
  source 1..21: mol_type = other DNA
aacgccaacaaacatgaaagt

SEQ ID NO: 681 - DNA, 21 bp, synthetic construct
  source 1..21: mol_type = other DNA
gggttatatgacgttagtaaa

SEQ ID NO: 682 - DNA, 21 bp, synthetic construct
  source 1..21: mol_type = other DNA
ttacctgagtagcaacggcta

SEQ ID NO: 683 - DNA, 21 bp, synthetic construct
  source 1..21: mol_type = other DNA
gaattatcattcatcaagagt

SEQ ID NO: 684 - DNA, 28 bp, synthetic construct
  source 1..28: mol_type = other DNA
tgattaatggcaacatataaacaaccga

SEQ ID NO: 685 - DNA, 28 bp, synthetic construct
  source 1..28: mol_type = other DNA
gaaaagtaattgagcgctaataaacagg

SEQ ID NO: 686 - DNA, 28 bp, synthetic construct
  source 1..28: mol_type = other DNA
aaaagggaattagagccagcaaaccatc

SEQ ID NO: 687 - DNA, 28 bp, synthetic construct
  source 1..28: mol_type = other DNA
agagaatttatcccaatccaactatttt

SEQ ID NO: 688 - DNA, 28 bp, synthetic construct
  source 1..28: mol_type = other DNA
agcgacacggtcatagccccccaccctc

SEQ ID NO: 689 - DNA, 28 bp, synthetic construct
  source 1..28: mol_type = other DNA
ctgaatcccggtattctaagatttcatc

SEQ ID NO: 690 - DNA, 28 bp, synthetic construct
  source 1..28: mol_type = other DNA
accctcaacgattggccttgatgaattt

SEQ ID NO: 691 - DNA, 28 bp, synthetic construct
  source 1..28: mol_type = other DNA
caagccgtcggctgtctttcccagctaa

SEQ ID NO: 692 - DNA, 28 bp, synthetic construct
  source 1..28: mol_type = other DNA
catggctgagtaacagtgcccgattagc

SEQ ID NO: 693 - DNA, 28 bp, synthetic construct
  source 1..28: mol_type = other DNA
aacaatatcgagccagtaataggcttaa

SEQ ID NO: 694 - DNA, 28 bp, synthetic construct
  source 1..28: mol_type = other DNA
aagagaaactcaggaggtttacaccctc

SEQ ID NO: 695 - DNA, 28 bp, synthetic construct
  source 1..28: mol_type = other DNA
acgctcatttagtatcatatgcatcttc

SEQ ID NO: 696 - DNA, 28 bp, synthetic construct
  source 1..28: mol_type = other DNA
aataggatagcattccacagacaacagt

SEQ ID NO: 697 - DNA, 28 bp, synthetic construct
  source 1..28: mol_type = other DNA
aaataccaatccaatcgcaagactacct

SEQ ID NO: 698 - DNA, 28 bp, synthetic construct
  source 1..28: mol_type = other DNA
tgctaaaaggctccaaaaggaagcttga

SEQ ID NO: 699 - DNA, 28 bp, synthetic construct
  source 1..28: mol_type = other DNA
cataggtttagattaagacgcaaacagt

SEQ ID NO: 700 - DNA, 28 bp, synthetic construct
  source 1..28: mol_type = other DNA
tgacaacttaaaggccgctttaagtttc

SEQ ID NO: 701 - DNA, 28 bp, synthetic construct
  source 1..28: mol_type = other DNA
tgaataaatcaagaaaacaaatcgcgca

SEQ ID NO: 702 - DNA, 28 bp, synthetic construct
  source 1..28: mol_type = other DNA
acttttttcatctttgaccccctgataa

SEQ ID NO: 703 - DNA, 28 bp, synthetic construct
  source 1..28: mol_type = other DNA
accaagtttacatcgggagaatagaacc

SEQ ID NO: 704 - DNA, 28 bp, synthetic construct
  source 1..28: mol_type = other DNA
tgctccagaccaactttgaaacaacgta

SEQ ID NO: 705 - DNA, 28 bp, synthetic construct
  source 1..28: mol_type = other DNA
acgtaaatggcaattcatcaacggaaca

SEQ ID NO: 706 - DNA, 28 bp, synthetic construct
  source 1..28: mol_type = other DNA
aaaacaggaagattggagacaaataacg

SEQ ID NO: 707 - DNA, 28 bp, synthetic construct
  source 1..28: mol_type = other DNA
tgtcaatcatatgtagctgattagccga

SEQ ID NO: 708 - DNA, 28 bp, synthetic construct
  source 1..28: mol_type = other DNA
agcaaacaagagaaatctacaatagcta

SEQ ID NO: 709 - DNA, 35 bp, synthetic construct
  source 1..35: mol_type = other DNA
accattaccatttccagagcctaatttgcgctaac

SEQ ID NO: 710 - DNA, 35 bp, synthetic construct
  source 1..35: mol_type = other DNA
taaatcctcattaatatcccatcctaatcctgaac

SEQ ID NO: 711 - DNA, 35 bp, synthetic construct
  source 1..35: mol_type = other DNA
agaaccgccaccaaataagaataaacactgataaa

SEQ ID NO: 712 - DNA, 35 bp, synthetic construct
  source 1..35: mol_type = other DNA
cggtttatcagcattaattaattttccctctgtaa

SEQ ID NO: 713 - DNA, 35 bp, synthetic construct
  source 1..35: mol_type = other DNA
agcgcgaaacaaattttcaggtttaacgtaaagaa

SEQ ID NO: 714 - DNA, 31 bp, synthetic construct
  source 1..31: mol_type = other DNA
  modified_base 1: mod_base = OTHER; note = Biotin modified nucleotide
ttttcagttgtgggaagggcttgagatggtt

SEQ ID NO: 715 - DNA, 31 bp, synthetic construct
  source 1..31: mol_type = other DNA
  modified_base 1: mod_base = OTHER; note = Biotin modified nucleotide
tttcagcaaatgaaaaacgaaccacagtaat

SEQ ID NO: 716 - DNA, 31 bp, synthetic construct
  source 1..31: mol_type = other DNA
  modified_base 1: mod_base = OTHER; note = Biotin modified nucleotide
tttatatcaataggagcattcgacaactcgt

SEQ ID NO: 717 - DNA, 31 bp, synthetic construct
  source 1..31: mol_type = other DNA
  modified_base 1: mod_base = OTHER; note = Biotin modified nucleotide
tttttcgcattaaatttttgataatcagaaa

SEQ ID NO: 718 - DNA, 31 bp, synthetic construct
  source 1..31: mol_type = other DNA
  modified_base 1: mod_base = OTHER; note = Biotin modified nucleotide
ttttaggaacacaaacggcggattggaaacc

SEQ ID NO: 719 - DNA, 38 bp, synthetic construct
  source 1..38: mol_type = other DNA
  modified_base 1: mod_base = OTHER; note = Biotin modified nucleotide
tttcgcaagggctaaatcggttgtaaagcctcagagca

SEQ ID NO: 720 - DNA, 31 bp, synthetic construct
  source 1..31: mol_type = other DNA
  modified_base 1: mod_base = OTHER; note = Biotin modified nucleotide
tttcatcaccttgctgaatcgccaggccaac

SEQ ID NO: 721 - DNA, 31 bp, synthetic construct
  source 1..31: mol_type = other DNA
  modified_base 1: mod_base = OTHER; note = Biotin modified nucleotide
tttaacgttatgcatctaccacggaataagt

SEQ ID NO: 722 - DNA, 31 bp, synthetic construct
  source 1..31: mol_type = other DNA
  modified_base 1: mod_base = OTHER; note = Biotin modified nucleotide
tttatcagctatgggatcaaagtcagagggt

SEQ ID NO: 723 - DNA, 31 bp, synthetic construct
  source 1..31: mol_type = other DNA
  modified_base 1: mod_base = OTHER; note = Biotin modified nucleotide
tttttcgcgtcccgtcgccacaagaattgag

SEQ ID NO: 724 - DNA, 38 bp, synthetic construct
  source 1..38: mol_type = other DNA
  modified_base 1: mod_base = OTHER; note = Biotin modified nucleotide
tttcagtgccacgctgaaacagagcagattcctacatt

SEQ ID NO: 725 - DNA, 39 bp, synthetic construct
  source 1..39: mol_type = other DNA
  modified_base 1: mod_base = OTHER; note = Biotin modified nucleotide
ttttctggtcgaaggttcctttgcccgaacgttattttt

SEQ ID NO: 726 - DNA, 31 bp, synthetic construct; identical to SEQ ID NO: 714
  source 1..31: mol_type = other DNA

SEQ ID NO: 727 - DNA, 31 bp, synthetic construct; identical to SEQ ID NO: 715
  source 1..31: mol_type = other DNA

SEQ ID NO: 728 - DNA, 31 bp, synthetic construct; identical to SEQ ID NO: 716
  source 1..31: mol_type = other DNA

SEQ ID NO: 729 - DNA, 31 bp, synthetic construct; identical to SEQ ID NO: 717
  source 1..31: mol_type = other DNA

SEQ ID NO: 730 - DNA, 31 bp, synthetic construct; identical to SEQ ID NO: 718
  source 1..31: mol_type = other DNA

SEQ ID NO: 731 - DNA, 38 bp, synthetic construct; identical to SEQ ID NO: 719
  source 1..38: mol_type = other DNA

SEQ ID NO: 732 - DNA, 31 bp, synthetic construct; identical to SEQ ID NO: 720
  source 1..31: mol_type = other DNA

SEQ ID NO: 733 - DNA, 31 bp, synthetic construct; identical to SEQ ID NO: 721
  source 1..31: mol_type = other DNA

SEQ ID NO: 734 - DNA, 31 bp, synthetic construct; identical to SEQ ID NO: 722
  source 1..31: mol_type = other DNA

SEQ ID NO: 735 - DNA, 31 bp, synthetic construct; identical to SEQ ID NO: 723
  source 1..31: mol_type = other DNA

SEQ ID NO: 736 - DNA, 38 bp, synthetic construct; identical to SEQ ID NO: 724
  source 1..38: mol_type = other DNA

SEQ ID NO: 737 - DNA, 39 bp, synthetic construct; identical to SEQ ID NO: 725
  source 1..39: mol_type = other DNA

SEQ ID NO: 738 - protein, 4 aa, synthetic construct
FRRF

SEQ ID NO: 739 - protein, 5 aa, synthetic construct
GGGGS

(19) United States
(12) Patent Application Publication
(10) Pub. No.: US 2024/0426839 A1
(43) Pub. Date: Dec. 26, 2024

(54) COMPOSITIONS AND METHODS FOR IMPROVING AFFINITY REAGENT AVIDITY

(71) Applicant: Nautilus Subsidiary, Inc., Seattle, WA (US)

(72) Inventors: Ahana DUTTA, San Jose, CA (US); Michael Augusto DARCY, Fremont, CA (US); Torri Elise RINKER, San Francisco, CA (US); Katherine D. LAZARUK, Redwood City, CA (US); Gajalakshmi DAKSHINAMOORTHY, Redwood City, CA (US); Elvis IKWA, San Leandro, CA (US); Pengyu HAO, Belmont, CA (US); Kaitlyn BURKE, Belmont, CA (US); Maureen NEWMAN, Redwood City, CA (US); Sheri Kay WILCOX, Longmont, CO (US); Subramanian SANKAR, Danville, CA (US); Sujal M. PATEL, Seattle, WA (US); Parag MALLICK, San Mateo, CA (US); Tural AKSEL, Redwood City, CA (US)

(21) Appl. No.: 18/748,558

(22) Filed: Jun. 20, 2024

Related U.S. Application Data
(60) Provisional application No. 63/509,488, filed on Jun. 21, 2023.
(60) Provisional application No. 63/610,265, filed on Dec. 14, 2023.

(51) Int. Cl. G01N 33/68; G01N 33/53

(52) U.S. Cl. CPC G01N 33/6878; G01N 33/5306

(57) ABSTRACT

The present disclosure provides a method of processing an analyte, including steps of (a) providing an analyte including an epitope and a docker; (b) providing an affinity reagent, wherein the affinity reagent includes a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes the step of (d) detecting association of the affinity reagent with the analyte, thereby identifying the analyte.

### CROSS-REFERENCE TO RELATED APPLICATIONS

This application claims priority to U.S. Provisional Application No. 63/509,488, filed on Jun. 21, 2023, and U.S. Provisional Application No. 63/610,265, filed on Dec. 14, 2023, each of which is incorporated herein by reference in its entirety.

### SEQUENCE LISTING

This application contains a Sequence Listing which has been submitted electronically in XML file format and is hereby incorporated by reference in its entirety. Said XML copy, created on Jun. 17, 2024, is named 50109_4019_US_SL.xml and is 685,805 bytes in size.

### BACKGROUND

Affinity reagents include a broad class of chemical reagents that form detectable interactions with analytes and other molecules. Affinity reagents can be configured to form temporary or reversible bound complexes with analytes. The observation of binding between an affinity reagent and analyte may be utilized to characterize the structure and properties of the analyte based on known recognition properties of the affinity reagent. Substantial resources and effort are typically invested into producing affinity reagents that bind to analytes of interest with sufficient specificity to distinguish the analytes in complex mixtures, such as biopsy samples which can contain nearly a full complement of proteins, nucleic acids or other biochemical analytes from the biopsied patient. Moreover, the specificity and strength of binding between an affinity reagent and analyte can be heavily influenced by small changes in conditions used for the assay such as changes in temperature, ionic strength, pH, and concentration of affinity reagent and analyte. This combination of factors can constrain the design of multiplex assays in which a large number of different analytes are to be evaluated, in parallel, for binding to affinity reagents. The present disclosure provides compositions and methods that improve binding assays and provide advantages that extend to multiplexed formats. Other advantages are provided as well.

### SUMMARY

The present disclosure provides a method of processing an analyte. The method can include the steps of (a) providing an analyte including an epitope and a docker; (b) providing an affinity reagent, wherein the affinity reagent includes a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes the step of (d) detecting association of the affinity reagent with the analyte, thereby identifying the analyte. In another option, the analyte is present in a sample including other analytes and the method further includes the step of (d) separating the analyte from the other analytes via the association of the affinity reagent with the analyte.

Also provided is a method of identifying an analyte, including steps of (a) providing an array of analytes, wherein each of the analytes is attached to a unique identifier in the array, wherein a docker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further includes a tether that recognizes the docker; (c) contacting the array with the affinity reagent, whereby the affinity reagent associates with the first unique identifier via binding of the paratope to the epitope and via binding of the tether to the docker; and (d) detecting association of the affinity reagent with the first unique identifier, thereby identifying the analyte at the first unique identifier.

Optionally, a method of identifying an analyte can include steps of (a) providing an array of analytes, wherein each of the analytes is attached to a unique identifier in the array, wherein a docker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further includes a tether that recognizes the docker; (c) contacting the array with the affinity reagent, whereby the affinity reagent associates with the first unique identifier via binding of the paratope to the epitope and via binding of the tether to the docker; (d) detecting association of the affinity reagent with the first unique identifier, thereby identifying the analyte at the first unique identifier; (e) removing the affinity reagent from the array; (f) contacting the array with a second affinity reagent, wherein the second affinity reagent includes a second paratope that recognizes a second epitope of the analyte at the first unique identifier of the array, wherein the second paratope preferentially recognizes the analyte compared to other analytes in the array, wherein the second affinity reagent further includes a second tether, whereby the second affinity reagent associates with the first unique identifier via binding of the second paratope to the second epitope and via binding of the second tether to the docker; and (g) detecting association of the second affinity reagent with the first unique identifier, thereby identifying the analyte at the first unique identifier.

The present disclosure further provides a method of identifying an analyte, including steps of (a) providing an array of analytes, wherein each of the analytes is attached to a unique identifier in the array, wherein a docker is attached to each unique identifier in the array, wherein the docker comprises an epitope of an analyte in the array, or an analog of the epitope; (b) providing an affinity reagent, wherein the affinity reagent includes a plurality of paratopes that independently recognize the epitope at a first unique identifier of the array, wherein the paratopes preferentially recognize the analyte compared to other analytes in the array; (c) contacting the array with the affinity reagent, whereby the affinity reagent associates with the first unique identifier via binding of at least one of the paratopes to the epitope and via binding of at least one of the paratopes to the docker; and (d) detecting association of the affinity reagent with the first unique identifier, thereby identifying the analyte at the first unique identifier.

In some configurations, a method of identifying an analyte can include steps of (a) providing an array of analytes, wherein each of the analytes is attached to a unique identifier in the array, wherein a linker is attached to each unique identifier in the array; (b) providing an affinity reagent, wherein the affinity reagent includes a plurality of paratopes that independently recognize an epitope of an analyte at a first unique identifier of the array, wherein the paratopes preferentially recognize the analyte compared to other analytes in the array; (c) attaching a docker to the linker that is attached to each unique identifier in the array, wherein the docker comprises the epitope or analog thereof; (d) contacting the array with the affinity reagent, whereby the affinity reagent associates with the first unique identifier via binding of at least one of the paratopes to the epitope and via binding of at least one of the paratopes to the docker; and (e) detecting association of the affinity reagent with the first unique identifier, thereby identifying the analyte at the first unique identifier.

Optionally, a method of identifying an analyte can include steps of (a) providing an array of analytes, wherein each of the analytes is attached to a unique identifier in the array, wherein a linker is attached to each unique identifier in the array; (b) providing an affinity reagent, wherein the affinity reagent includes a plurality of paratopes that independently recognize an epitope of an analyte at a first unique identifier of the array, wherein the paratopes preferentially recognize the analyte compared to other analytes in the array; (c) attaching a docker to the linker that is attached to each unique identifier in the array, wherein the docker comprises the epitope or analog thereof; (d) contacting the array with the affinity reagent, whereby the affinity reagent associates with the first unique identifier via binding of at least one of the paratopes to the epitope and via binding of at least one of the paratopes to the docker; (e) detecting association of the affinity reagent with the first unique identifier, thereby identifying the analyte at the first unique identifier; (f) removing the affinity reagent and the docker from the array; (g) attaching a second docker to the linker that is attached to each unique identifier in the array, wherein the second docker includes the second epitope or analog thereof, (h) contacting the array with a second affinity reagent including a plurality of second paratopes that independently recognize a second epitope of the analyte at the first identifier, wherein the second epitope differs from the first epitope, whereby the second affinity reagent associates with the first unique identifier via binding of at least one of the second paratopes to the second epitope and via binding of at least one of the second paratopes to the second docker; and (i) detecting association of the second affinity reagent with the first unique identifier, thereby identifying the analyte at the first unique identifier. Typically, the paratopes preferentially recognize the epitope compared to the second epitope.

In some configurations of the methods and compositions set forth herein, dockers and tethers can form covalent attachments to each other. Accordingly, a method of identifying an analyte can include steps of (a) providing an array of analytes, wherein each of the analytes is attached to a unique identifier in the array, wherein a docker is attached to each unique identifier in the array, wherein the docker comprises a first reactive moiety; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further comprises a second reactive moiety; (c) contacting the array with the affinity reagent, whereby the affinity reagent associates with the first unique identifier via binding of at least one of the paratopes to the epitope and via the covalently linked product of reaction between the first reactive moiety with the second reactive moiety; and (d) detecting association of the affinity reagent with the first unique identifier, thereby identifying the analyte at the first unique identifier.

Optionally, a method of identifying an analyte can include steps of (a) providing an array of analytes, wherein each of the analytes is attached to a unique identifier in the array, wherein a linker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further comprises a first reactive moiety; (c) attaching a second reactive moiety to the linker, wherein the second reactive moiety is reactive toward the first reactive moiety to form a covalently linked product; (d) contacting the array with the affinity reagent, whereby the affinity reagent associates with the first unique identifier via binding of at least one of the paratopes to the epitope and via the covalently linked product of reaction between the first reactive moiety with the second reactive moiety; and (e) detecting association of the affinity reagent with the first unique identifier, thereby identifying the analyte at the first unique identifier.

A method that utilizes covalent bonding of dockers to tethers can be configured to serially contact an analyte with affinity reagents. Accordingly, a method of identifying an analyte, can include steps of (a) providing an array of analytes, wherein each of the analytes is attached to a unique identifier in the array, wherein a linker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further comprises a first reactive moiety; (c) attaching a second reactive moiety to the linker, wherein the second reactive moiety is reactive toward the first reactive moiety to form a covalently linked product; (d) contacting the array with the affinity reagent, whereby the affinity reagent associates with the first unique identifier via binding of at least one of the paratopes to the epitope and via the covalently linked product of reaction between the first reactive moiety with the second reactive moiety; and (e) detecting association of the affinity reagent with the first unique identifier, thereby identifying the analyte at the first unique identifier (f) removing the affinity reagent and the covalently linked product from the array; (g) attaching a third reactive moiety to the linker, wherein the third reactive moiety is reactive toward a fourth reactive moiety to form a covalently linked product; (h) contacting the array with a second affinity reagent, wherein the second affinity reagent includes a paratope that recognizes a second epitope of the analyte at the first unique identifier of the array, wherein the paratope preferentially recognizes the analyte compared to other analytes in the array, wherein the affinity reagent further includes the fourth reactive moiety, whereby the affinity reagent associates with the first unique identifier via binding of at least one of the paratopes to the epitope and via the covalently linked product of reaction between the third reactive moiety with the fourth reactive moiety; and (i) detecting association of the second affinity reagent with the first unique identifier, thereby identifying the analyte at the first unique identifier.

Also provided is an assay composition, including (a) an analyte; and (b) an affinity reagent; wherein the analyte includes an epitope attached to a docker; and wherein the affinity reagent includes a paratope that preferentially recognizes the epitope compared to the docker, and a tether that preferentially recognizes the docker compared to the epitope.

An affinity reagent for an analyte is provided and can include, (a) a paratope having affinity for an epitope of the analyte; and (b) a tether having affinity for a docker, wherein the docker is attached to the analyte, and wherein the paratope has greater affinity for the epitope compared to the affinity of the tether for the docker.

The present disclosure further provides an assay composition including (a) an analyte attached to a first particle, wherein the analyte includes an epitope, and wherein a set of dockers is attached to fixed positions on the first particle; and (b) an affinity reagent attached to a second particle, wherein the affinity reagent includes a paratope, wherein a set of tethers is attached to fixed positions on the second particle, wherein the paratope recognizes the epitope, wherein the tethers recognize the dockers, and wherein the fixed positions of the set of dockers on the first particle and the fixed positions of the set of tethers on the second particle prevent at least one of the tethers from contacting the set of dockers while (i) the paratope is bound to the epitope and (ii) a subset of the tethers is simultaneously bound to a subset of the dockers.

Further provided is a method including (a) providing an analyte attached to a first particle, wherein the analyte includes an epitope, and wherein a set of dockers is attached to fixed positions on the first particle; (b) contacting the analyte with an affinity reagent, wherein the affinity reagent is attached to a second particle, wherein the affinity reagent includes a paratope, wherein a set of tethers is attached to fixed positions on the second particle, wherein the paratope binds to the epitope, wherein a subset of the tethers binds to a subset of the dockers, and wherein the fixed positions of the set of dockers on the first particle and the fixed positions of the set of tethers on the second particle prevent at least one of the tethers from contacting the set of dockers while (i) the paratope is bound to the epitope and (ii) the subset of the tethers is simultaneously bound to the subset of the dockers, thereby forming a complex including the analyte and the affinity reagent.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1A provides a diagrammatic representation of an affinity reagent reversibly binding to an analyte in an equilibrium state.

FIG. 1B provides a diagrammatic representation of equilibrium binding between an affinity reagent having a tether and an analyte having a docker.

FIG. 2 shows a diagrammatic representation of equilibrium binding between an affinity reagent having two nucleic acid tethers and a protein analyte having six nucleic acid dockers.

FIG. 3A shows a diagrammatic representation of equilibrium binding between an affinity reagent having six nucleic acid tethers and a protein analyte having six nucleic acid dockers.

FIG. 3B shows a diagrammatic representation of equilibrium binding between a protein analyte having six nucleic acid dockers and an affinity reagent having two nucleic acid tethers and three antibody components.

FIG. 4 shows a diagrammatic representation of equilibrium binding between a protein analyte having four subsets of two nucleic acid dockers and an affinity reagent having four sets of two nucleic acid tethers.

FIG. 5A shows a diagram of a reaction for attaching epitope dockers to a unique identifier in an array. The figure discloses “GGHSPGG” as SEQ ID NO: 1.

FIG. 5B shows a diagrammatic representation of equilibrium binding between the unique identifier of FIG. 5A and an affinity reagent having three antibody components that recognize the epitope. The figure discloses “GGHSPGG” as SEQ ID NO: 1.

FIG. 5C shows a diagrammatic representation of equilibrium binding between (1) a unique identifier having a protein analyte with a target epitope and secondary epitopes as dockers and (2) an affinity reagent having an antibody component for the target epitope and two antibody components that bind the secondary epitope. The figure discloses “GGHSPGG” as SEQ ID NO: 1.

FIG. 6A shows a diagram of a reaction for attaching a unique identifier in an array to a docker having an antibody receptor.

FIG. 6B shows a diagrammatic representation of equilibrium binding between the unique identifier of FIG. 6A and an affinity reagent having three antibodies that recognize the HSP epitope of the protein analyte.

FIG. 7A shows a diagram of a reaction for attaching a unique identifier in an array to a docker having reactive moiety R.

FIG. 7B shows a diagrammatic representation of equilibrium binding between the unique identifier of FIG. 7A and an affinity reagent having an antibody component that binds to the HSP epitope of the protein analyte and tethers including reactive moiety R followed by activation of R to covalently bond with the affinity reagent.

FIG. 7C shows a diagrammatic representation of covalent reaction between the unique identifier of FIG. 7A and an affinity reagent.

FIG. 7D shows a diagrammatic representation of covalent reaction between an affinity reagent and unique identifier having dockers with scissile linkages and reactive moieties.

FIG. 8 shows a diagram of an array of addresses arranged in a rectilinear grid, wherein the array includes two overlapping patterns of addresses, the addresses in the respective patterns being indicated by black and grey circles.

FIG. 9A shows a diagram of sequence regions for a docker.

FIG. 9B shows a diagram of the docker hybridized to a region of a SNAP origami structure.

FIG. 10A shows a diagram of sequence regions for a v1tether.

FIG. 10B shows a diagram of the v1 tether hybridized to a region of a Lobe origami structure.

FIG. 11A shows a diagram of sequence regions for a v2 tether.

FIG. 11B shows a diagram of the v1 tether hybridized to a region of a Lobe origami structure.

FIG. 12 shows an image of the fluorescently detected gel used in a gel shift experiment.

FIG. 13 shows results of a fluorescent-based binding experiment, plotting the Lobe to SNAP colocalization rate observed for each lane of a flow cell.

FIG. 14 shows results of a fluorescent-based binding experiment, plotting the Lobe to SNAP colocalization rate observed for each lane of a flow cell.

FIG. 15 shows plots of colocalization measured for various Lobes with various SNAPs after forming complexes and removing unbound Lobes.

FIG. 16 shows plots of colocalization measured for various Lobes with various SNAPs after forming complexes, removing unbound Lobes and stripping bound Lobes.

FIG. 17 shows plots of colocalization measured for various Lobes with various SNAPs after forming complexes and removing unbound Lobes.

FIG. 18 shows plots of colocalization measured for various Lobes with various SNAPs after forming complexes, removing unbound Lobes and stripping bound Lobes.

FIG. 19 shows a system containing a luminescent enzyme and an enzymatic cofactor as a docker/tether system. The figure discloses SEQ ID NO: 739.

### DETAILED DESCRIPTION

The present disclosure provides compositions and methods for binding of affinity reagents to analytes. Binding of affinity reagents to analytes can be used to characterize the analytes in an analytical method based on known or expected properties of the affinity reagents. For example, observed binding between an analyte and an affinity reagent having known specificity for a given molecular structure (i.e. a known epitope) indicates that the analyte has the molecular structure. The presence of the molecular structure can provide useful information for identifying the analyte. Binding of affinity reagents to analytes can also be used to capture the analytes, for example, in a preparative method. For example, an immobilized affinity reagent can be bound to an analyte in a mixture, thereby forming an immobilized complex, and the immobilized complex can be separated from the mixture to isolate the analyte from other components of the mixture.

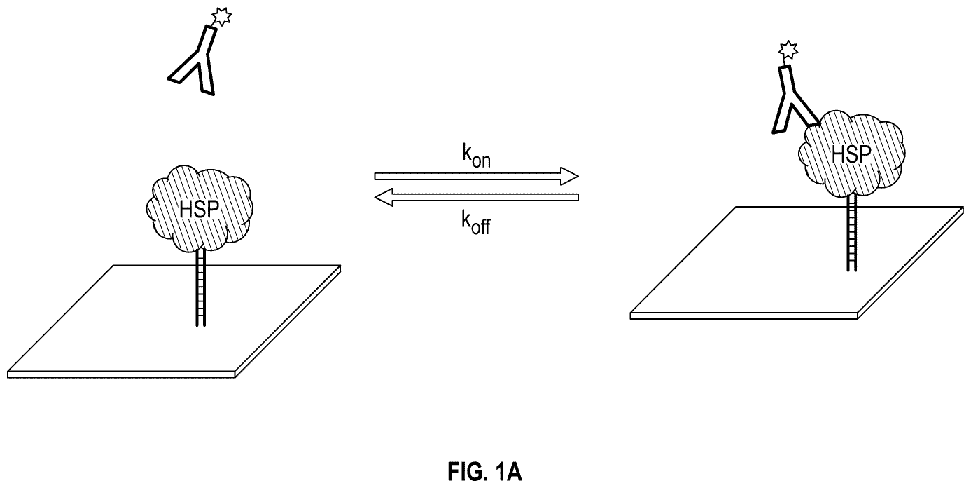

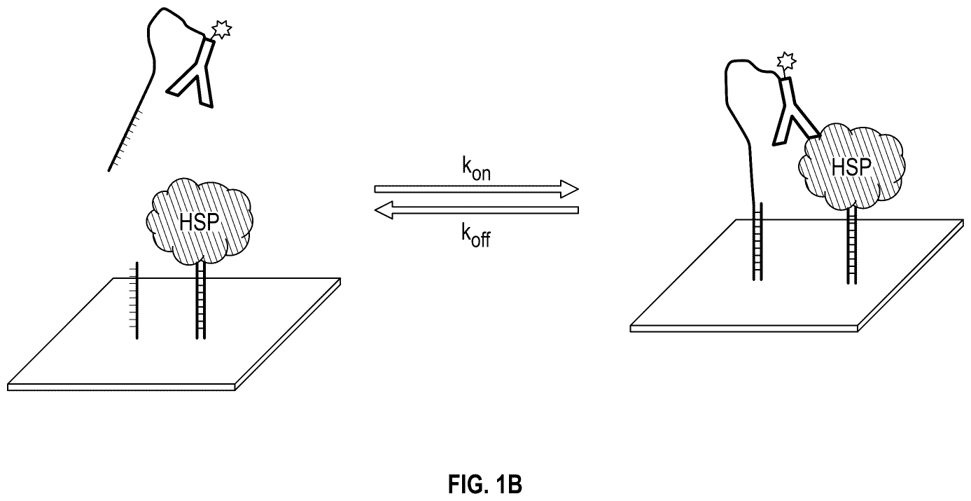

Binding of affinity reagents to analytes can be understood in terms of equilibrium binding kinetics. FIG. 1A provides a diagrammatic representation of an affinity reagent (Y-shape) reversibly binding to an analyte (stippled globule) in an equilibrium state. In this example, the affinity reagent is an antibody having a detectable label (7-point star) and the analyte is a protein immobilized at an address (grey rectangle) of an array. The protein includes an epitope (the amino acid sequence HSP) that is recognized by a paratope of the labeled antibody. At equilibrium, the affinity reagent and analyte can reversibly toggle between a dissociated state (left side of the equation) and associated state (right side of the equation). The relative distribution of species between the two states is quantifiable as the ratio of the association rate (represented by the association rate constant kₒₙ) and dissociation rate (represented by the dissociation rate constant k_{off}). As such, the probability that an analyte is complexed with an affinity reagent at any given time can be increased by increasing the association rate (i.e. increasing affinity) and/or decreasing the dissociation rate (i.e. increasing avidity).

The present disclosure provides compositions and methods for improving binding of analytes to affinity reagents by increasing avidity. In particular embodiments, avidity between an analyte and affinity reagent can be increased by addition of a docker to the analyte and addition of a tether to the affinity reagent. The docker and tether recognize each other and can thus bind to each other. FIG. 1B provides a diagrammatic representation of equilibrium binding between an affinity reagent (Y-shape) having a tether (comb structure attached to the affinity reagent) and an analyte (stippled globule) having a docker (comb structure attached to the address of the analyte). In this example, the tether is a nucleic acid having a nucleotide sequence that is complementary to a nucleotide sequence of the docker. Avidity of the interaction between the affinity reagent and analyte is a function not only of recognition between the paratope and epitope, but also recognition between the docker and tether. As will be set forth in further detail below herein, appropriate choice of docker and tether can increase stability of binding between an analyte and affinity reagent. Surprisingly, the addition of the docker and tether does not adversely impact the ability to characterize analytes based on known or expected specificity of affinity reagents for the analytes.

Accordingly, the present disclosure provides a method of processing an analyte. The method can include the steps of (a) providing an analyte comprising an epitope and a docker; (b) providing an affinity reagent, wherein the affinity reagent comprises a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes the step of (d) detecting association of the affinity reagent with the analyte, thereby identifying the analyte. In another option, the analyte is present in a sample comprising other analytes and the method further includes the step of (d) separating the analyte from the other analytes via the association of the affinity reagent with the analyte.

The compositions and methods of the present disclosure are particularly well suited for detecting analytes using affinity reagents in non-equilibrium conditions. A typical binding assay that is configured to detect an analyte will employ an excess amount of affinity reagent. This is done to drive the reaction to form a complex between the analyte and affinity reagent. However, the excess affinity reagent can produce unwanted background or artifacts, for example, when an immobilized analyte is to be detected by virtue of a label on the affinity reagent to which it binds and when the large excess of labels in solution overwhelms signal produced by the bound affinity reagent. One option is to remove excess affinity reagent from the environment of the immobilized complex and rapidly detect the complex before it dissociates under the non-equilibrium condition resulting from absence of the excess affinity reagent. The use of tethers and dockers can increase the half-life of the complex under the non-equilibrium condition, thereby improving detectability of analyte-affinity reagent complexes. This can be particularly advantageous for multiplex methods, for example, when detection of multiple analytes occurs over a substantial duration of time.

The compositions and methods of the present disclosure are particularly well suited for detecting analytes at single-molecule resolution. Detection of an analyte at single-molecule resolution can be stochastic, for example, when a bound complex is unobserved due to being dissociated during the brief period of time that a signal is being acquired by a detector. Increasing avidity of the bound complex can reduce the frequency and duration of dissociation events, thereby improving detectability of the complex.

As set forth in further detail below, a variety of different dockers and tethers can be employed to increase avidity of binding between an analyte and affinity reagent. The type of docker and tether that is to be used in combination with a particular analyte and affinity reagent pair can be selected based on known or expected affinity of the affinity reagent for the analyte. For example, a method that employs a first affinity reagent having relatively strong affinity for a particular analyte can utilize a docker and tether pair having relatively weak affinity, whereas a method that employs a second affinity reagent having weaker affinity for the analyte can utilize a docker and tether pair having higher affinity compared to the pair used for the first affinity reagent. Accordingly, the probability of forming a complex and duration of the complex can be tuned by appropriate choice of docker species and tether species. A further variable that can be employed to tune binding between an analyte and affinity reagent is the number of dockers associated with the analyte and/or the number of tethers associated with the affinity reagent. For example, a method that employs a first affinity reagent having relatively strong affinity for an analyte can utilize a relatively low number of docker-tether pairs, whereas a method that employs a second affinity reagent having weaker affinity for the analyte can utilize a greater number of docker-tether pairs compared to the number(s) used for the first affinity reagent.

Of course, a binding event can be tuned via a combination of the number and type of docker-tether pairs used. This can be illustrated in the context of nucleic acid dockers and tethers having complementary nucleotide sequences. For example, the maintenance of a complex between an analyte and affinity reagent can be increased by increasing the number of dockers and tethers present in the complex and also by increasing the avidity of each docker for its complementary tether. The avidity of binding between a nucleic acid docker and tether can be increased, for example, by increasing the length of the complementary sequences, increasing the GC content of the complementary sequences, or otherwise increasing the melting temperature (Tₘ) of the duplex formed by the complementary sequences. Tuning can also be achieved by altering solution conditions for the binding event. When using pairs of dockers and tethers that have orthogonal binding properties compared to binding properties of the epitopes and paratopes with which they are associated, conditions that are selected for alteration can be those known or suspected to have a greater impact on the docker and tether pair compared to the paratope and epitope pair. For example, when using nucleic acid dockers and tethers in association with protein-based analytes and antibody-based affinity reagents, conditions that have a greater impact on nucleic acid hybridization than on protein-antibody binding can be modified to adjust avidity.

A binding event can also be tuned via geometric constraints on the positions of dockers relative to the positions of tethers to which they will interact. Optionally, a set of dockers can be geometrically constrained via attachment to fixed positions on a particle or solid support. The dockers can be geometrically constrained with respect to the position at which an analyte is attached to the particle or solid support. Moreover, the dockers in the set can be geometrically constrained with respect to each other. Similarly, a set of tethers can be geometrically constrained via attachment to fixed positions on a particle or solid support. The tethers can be geometrically constrained with respect to the position at which a paratope is attached to the particle or solid support. Optionally, the tethers in the set can be geometrically constrained with respect to each other. The arrangements of the set of dockers and the set of tethers can be configured to accommodate the possibility that an affinity reagent may include fewer tethers than expected and/or the possibility that an analyte may include fewer dockers than expected. Such a situation can occur due to inefficient synthesis of the affinity reagent or analyte, or due to post-synthesis degradation of the affinity reagent or analyte. In these cases, the geometries for the set of dockers and set of tethers can be configured to provide redundant combinations for tether-docker interactions. As such, absence of a particular tether from an affinity reagent can be recompensed by presence of another tether. The present tether can be a member of a redundant subset of tethers that can replace a subset of tethers that was intended to include the absent tether, and the redundant subset can bind to dockers that would have otherwise bound to a subset that included the absent tether. Similarly, absence of a particular docker from an affinity reagent can be recompensed by presence of another docker that forms a subset of dockers that is not reliant on presence of the absent docker.

Multiplex methods, in which a plurality of different analytes is processed in parallel, can employ universal dockers. The dockers are referred to as “universal” because they have identical structures that interact with tethers. For example, an array can include a plurality of addresses, each of the addresses being attached to an analyte that differs from other analytes in the array and each of the addresses being attached to a docker that is the same as other dockers in the array. A plurality of different analytes that are associated with universal dockers can be contacted with a plurality of different affinity reagents that are associated with tethers. Some or all the different affinity reagents can have the same tether structure. As such, the avidity effect of the dockers and tethers can be substantially uniform. Alternatively, each of the different affinity reagents can have a unique tether structure, for example, a tether that is tuned to the relative affinity or avidity of the affinity reagent compared to other affinity reagents in the plurality. Similarly, affinity reagents can differ with respect to the number of tethers associated with each affinity reagent or, alternatively, the same number of tethers can be associated with each of the affinity reagents. By configuring an array of analytes to have an excess number of dockers at each analyte address, the avidity of individual affinity reagents can be tuned by adjusting the number of tethers to be in a range from zero to the number of dockers at each address.

Methods that employ multiple different affinity reagents can employ universal tethers. The tethers are referred to as ‘universal’ because they have identical structures that interact with dockers. For example, an array of analytes can be contacted with a plurality of different affinity reagents, each of the affinity reagents having a paratope that differs from other affinity reagents in the plurality and each of the affinity reagents being attached to a tether that is the same as other tethers in the plurality. The different affinity reagents can be present in a mixture that is simultaneously in contact with the array or, alternatively, the different affinity reagents can be serially contacted with the array.

Multiplex methods, in which a plurality of different analytes is processed in parallel, can employ indexed dockers. A set of indexed dockers can include at least two subsets of dockers, wherein dockers in different subsets have different structures that interact with different tethers, respectively, and wherein the dockers within each subset have identical structures that interact with the same tethers. For example, an array can include a plurality of addresses, each of the addresses being attached to an analyte that differs from other analytes in the array, a first subset of addresses being attached to a docker that binds to a first subset of tethers, and a second subset of addresses being attached to a docker that binds to a second subset of tethers, wherein the first subset of dockers does not substantially bind to the second subset of tethers and the second subset of dockers does not substantially bind to the first subset of tethers.

Methods that employ multiple different affinity reagents can employ indexed tethers. A set of indexed tethers can include at least two subsets of tethers, wherein tethers in different subsets have different structures that interact with different dockers, and wherein the tethers within each subset have identical structures that interact with the same dockers. For example, an array of analytes can be contacted with a first subset of affinity reagents, each of the affinity reagents in the first subset having a paratope that differs from other affinity reagents in the first subset and each of the affinity reagents in the first subset being attached to a tether that is the same as other tethers in the first subset. Continuing with the example, the array of analytes can also be contacted with a second subset of affinity reagents, each of the affinity reagents in the second subset having a paratope that differs from other affinity reagents in the second subset and each of the affinity reagents in the second subset being attached to a tether that is the same as other tethers in the second subset. Because the tethers in the first subset of affinity reagents do not cross-react with dockers recognized by the second subset and, conversely, the tethers in the second subset of affinity reagents do not cross-react with dockers recognized by the first subset, the affinity reagents in the respective subsets can be distinguished as can the addresses (or other unique identifiers) with which they associate. The different subsets of affinity reagents can be present in a mixture that is simultaneously in contact with the array or, alternatively, the different subsets of affinity reagents can be serially contacted with the array.

Terms used herein will be understood to take on their ordinary meaning in the relevant art unless specified otherwise. Several terms used herein and their meanings are set forth below.

As used herein, the term “address,” when used in reference to an array, means a location in an array occupied by, or configured to be occupied by, a particular molecule or analyte such as a protein, nucleic acid, structured nucleic acid particle or reactive moiety. An address can contain a single molecule, or it can contain a population of several molecules of the same species (i.e. an ensemble of the molecules). Alternatively, an address can include a population of molecules that are different species. Addresses of an array are typically discrete. The discrete sites can be contiguous, or they can have interstitial spaces between each other. An array useful herein can have, for example, addresses that are separated by less than 100 microns, 10 microns, 1 micron, 0.5 micron, 0.1 micron, 0.01 micron or less. Alternatively or additionally, an array can have addresses that are separated by at least 0.01 micron, 0.1 micron, 0.5 micron, 1 micron, 10 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁷, 1×10⁸, 1×10⁹, 1×10¹⁰, 1×10¹¹, 1×10¹², or more addresses, some or all of which are occupied by analytes or molecules. An address can also be referred to herein as a “site.”

As used herein, the term “affinity reagent” refers to a molecule or other substance that is capable of specifically or reproducibly binding to an analyte (e.g. protein) or moiety (e.g. post-translational modification of a protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), aptamers, affibodies, affilins, affimers, affitins, alphabodies, anticalins, avimers, miniproteins, DARPins, monobodies, nanoCLAMPs, lectins, or functional fragments thereof. The term “affinity agent” is intended to be synonymous with the term “affinity reagent.”

As used herein, the term “antibody” refers to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

As used herein, the term “array” refers to a population of analytes (e.g. proteins) that are associated with unique identifiers such that the analytes can be distinguished from each other. A unique identifier can be a solid support (e.g. particle or bead), structured nucleic acid particle (SNAP), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent or non-covalent bonds. An array can include different analytes that are each attached to different unique identifiers. An array can include different unique identifiers that are attached to the same or similar analytes. An array can include separate solid supports, or separate sites on the same solid support, that each bear a different analyte, wherein the different analytes can be identified according to the locations of the solid supports or sites. Analytes that can be included in an array can be, for example, nucleic acids such as structured nucleic acid particles, proteins, enzymes, glycans, affinity reagents, ligands, or receptors.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “bioorthogonal reaction” refers to a chemical reaction that can occur within a biological system (in vitro and/or in vivo) without interfering with some or all native biological processes, functions, or activities of the biological system. A bioorthogonal reaction may be further characterized as being inert to components of a biological system other than those targeted by the bioorthogonal reaction. A bioorthogonal reaction may include a click reaction. A bioorthogonal reaction may utilize an enzymatic approach, such as attachment between a first molecule and a second molecule by an enzyme such as a sortase, a ligase, or a subtiligase. A bioorthogonal reaction may utilize an irreversible peptide capture system, such as SpyCatcher/SpyTag, SnoopCatcher/SnoopTag, or SdyCatcher/SdyTag. A “bioorthogonal reactant” or “bioorthogonal moiety” is capable of being converted to a different product in a bioorthogonal reaction.

As used herein, the term “click reaction” refers to a single-step, thermodynamically-favorable conjugation reactions utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary click reactions may include Staudinger ligation, copper-free click reaction, metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, quadricyclane ligation and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference. A “click reactant” or “click moiety” is capable of being converted to a different product in a click reaction.

The term “comprising” is intended herein to be open-ended, including not only the recited elements, but further encompassing any additional elements.

As used herein, the term “docker” refers to a molecule or moiety that is configured to interact with a tether or that is interacting with a tether. A docker can be a moiety of a substance, object, molecule, solid support, address, particle, or bead. A docker can include a polymer, nucleic acid strand, nucleic acid duplex, nucleotide sequence, protein, affinity reagent, epitope, paratope, receptor, ligand or the like. A docker can interact with a tether via covalent or non-covalent bonding.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to a molecule or part of a molecule, which is recognized by or binds specifically to an affinity reagent or paratope. Epitopes may include amino acid sequences that are sequentially adjacent in the primary structure of a protein or amino acids that are structurally adjacent in the secondary, tertiary or quaternary structure of a protein. An epitope can optionally be recognized by or bound to an antibody. However, an epitope need not necessarily be recognized by any antibody, for example, instead being recognized by an aptamer, miniprotein or other affinity reagent. An epitope can optionally bind an antibody to elicit an immune response. However, an epitope need not necessarily participate in, nor be capable of, eliciting an immune response.

As used herein, the term “exogenous,” when used in reference to a moiety of a molecule, means a moiety that is not present in a natural analog of the molecule. For example, an exogenous label of an amino acid is a label that is not present on a naturally occurring amino acid. Similarly, an exogenous label that is present on an antibody is not found on the antibody in its native milieu.

As used herein, the term “fluid-phase,” when used in reference to a molecule or particle, means the molecule or particle is in a state wherein it is mobile in a fluid, for example, being capable of diffusing through the fluid. A fluid-phase molecule or particle is not in a state of immobilization on a solid-phase support.

As used herein, the term “immobilized,” when used in reference to a molecule or particle that is in contact with a fluid phase, refers to the molecule or particle being prevented from diffusing in the fluid phase. For example, immobilization can occur due to confinement at, or attachment to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “paratope” refers to a molecule or part of an affinity reagent, which recognizes or binds specifically to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “protein” refers to a molecule including two or more amino acids joined by a peptide bond. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications.

As used herein, the term “recognize” refers to the capability of two or more molecules to interact through non-covalent bonding such as hydrogen bonding, metal coordination, hydrophobic forces, van der Waals forces, π-π interactions, halogen bonding, or resonant interaction effects.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass, modified or functionalized glass, plastics (including acrylics, polystyrene and copolymers of styrene and other materials, polypropylene, polyethylene, polybutylene, polyurethanes, Teflon™ cyclic olefins, polyimides etc.), nylon, ceramics, resins, Zeonor™, silica or silica-based materials including silicon and modified silicon, carbon, metals, inorganic glasses, optical fiber bundles, and polymers.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure (i.e. the helical twist or direction of the polynucleotide strand) of a SNAP can be configured to be more dense than a nucleic acid molecule of similar length in a random coil or other non-structured state. A SNAP can optionally be modified to permit attachment of additional molecules to the SNAP. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “tether” refers to a molecule or moiety that is configured to interact with a docker or that is interacting with a docker. A tether can be a moiety of a substance, object, molecule, solid support, address, particle, or bead. A tether can include a polymer, nucleic acid strand, nucleic acid duplex, nucleotide sequence, protein, affinity reagent, epitope, paratope, receptor, ligand or the like. A tether can interact with a docker via covalent or non-covalent bonding.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; a site in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

The embodiments set forth below and recited in the claims can be understood in view of the above definitions.

The present disclosure provides a method of identifying an analyte. The method can include steps of (a) providing an array of analytes, wherein each of the analytes is attached to a unique identifier in the array, wherein a docker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further includes a tether that recognizes the docker; (c) contacting the array with the affinity reagent, whereby the affinity reagent associates with the first unique identifier via binding of the paratope to the epitope and via binding of the tether to the docker; and (d) detecting association of the affinity reagent with the first unique identifier, thereby identifying the analyte at the first unique identifier.

Any of a variety of analytes can be used in a method or composition set forth herein. For ease of explanation, various methods and compositions will be exemplified herein in the context of using proteins. It will be understood that the exemplified methods and compositions can be extended to other analytes. Exemplary analytes include, but are not limited to, a tissue, cell, organelle, virus, nucleic acid (e.g. DNA or RNA), carbohydrate (e.g. monosaccharide, oligosaccharide or polysaccharide), vitamin, enzyme cofactor, hormone, small molecule such as a candidate therapeutic agent, metabolite, nucleotide, nucleoside, amino acid, sugar, lipid, or the like. In some configurations, a composition or method set forth herein can lack one or more of the analytes set forth herein.

A composition or method set forth herein can be configured for a single analyte or for a plurality of different analytes. A plurality of analytes can include, for example, a proteome, or substantial fraction thereof, including a variety of different proteins; a genome, or substantial fraction thereof, including a variety of different DNA sequences; a transcriptome, or substantial fraction thereof, including a variety of different RNA sequences; a metabolome, or substantial fraction thereof, including a variety of different metabolites; or a microbiome, or substantial fraction thereof, including a variety of different microbes. These and other analytes known in the art can be used in compositions and methods set forth herein.

A protein or other analyte can be derived from a natural or synthetic source. Exemplary sources include, but are not limited to a biological tissue, fluid, cell or subcellular compartment (e.g. organelle). For example, a sample can be derived from a tissue biopsy, biological fluid (e.g. blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or protein synthesis reaction. A primary source for a cancer biomarker protein may be a tumor biopsy sample. Other sources include environmental samples or forensic samples.

Exemplary organisms from which a protein or other analyte can be derived include, but are not limited to, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A protein can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A protein or other analyte can be derived from a homogeneous culture or population of the above organisms or alternatively from a collection of several different organisms, for example, in a community or ecosystem.

In some cases, a protein or other analyte can be derived from an organism that is collected from a host organism. A protein or other analyte may be derived from a parasitic, pathogenic, symbiotic, or latent organism collected from a host organism. A protein or other analyte can be derived from an organism, tissue, cell or biological fluid that is known or suspected of being associated with a disease state or disorder (e.g., an oncogenic virus). Alternatively, a protein or other analyte can be derived from an organism, tissue, cell or biological fluid that is known or suspected of not being associated with a particular disease state or disorder. For example, one or more proteins isolated from such a source can be used as a control for comparison to results acquired from a source that is known or suspected of being associated with the particular disease state or disorder. A sample may include a microbiome. A sample may include a plurality of proteins or other analytes of interest contributed by microbiome constituents. In some cases, one or more proteins (or other analytes) used in a method, composition or apparatus set forth herein may be obtained from a single organism (e.g. an individual human), single cell, single organelle, or single protein-containing particle (e.g., a viral particle).

One or more analytes can optionally be separated or isolated from other components of the source for the analyte(s). For example, one or more proteins can be separated or isolated from lipids, nucleic acids, hormones, enzyme cofactors, vitamins, metabolites, microtubules, organelles (e.g. nucleus, mitochondria, chloroplast, endoplasmic reticulum, vesicle, cytoskeleton, vacuole, lysosome, cell membrane, cytosol or Golgi apparatus), other proteins or the like. Protein separation can be carried out using methods known in the art such as centrifugation (e.g. to separate membrane fractions from soluble fractions), density gradient centrifugation (e.g. to separate different types of organelles), precipitation, affinity capture, adsorption, liquid-liquid extraction, solid-phase extraction, chromatography (e.g. affinity chromatography, ion exchange chromatography, reverse phase chromatography, size exclusion chromatography, electrophoresis (e.g. polyacrylamide gel electrophoresis) or the like. Useful protein separation methods are set forth in Scopes, Protein Purification Principles and Practice, Springer; 3rd edition (1993).

A protein that is used in a composition or method set forth herein can be in a native or denatured conformation. For example, a protein can be in a native conformation, whereby it is capable of performing native function(s) such as catalysis of its natural substrate(s) or binding to its natural substrate(s). Alternatively, a protein can be in a denatured conformation whereby it is incapable of performing certain native function(s) such as catalysis of its natural substrate(s) or binding to its natural substrate(s). A protein can be in a native conformation for some manipulations set forth herein and in a denatured conformation for other manipulations set forth herein. A protein may be denatured at any stage during manipulation, including for example, upon removal from a native milieu or at a later stage of processing such as a stage where the protein is separated from other cellular components, fractionated from other proteins, functionalized to include a reactive moiety, attached to a particle or solid support, contacted with an affinity reagent, detected, or other manipulation. Any of a variety of denaturants can be used such as heat (e.g. temperatures greater than about 40° C., 60° C., 80° C. or higher), excessive pH (e.g. pH lower than 4.0, 3.0 or 2.0; or pH greater than 10.0, 11.0 or 12.0); chaotropic agents (e.g. urea, guanidinium chloride, or sodium dodecyl sulfate), organic solvent (e.g. chloroform or ethanol), physical agitation (e.g. sonication) or radiation. A denatured protein may be refolded, for example, reverting to a native state for one or more steps of a process set forth herein.

An analyte used in a composition or method of the present disclosure can be associated with a docker. The docker can be any molecule or moiety that is capable of attaching to a tether. A particularly useful docker is a nucleic acid strand having a nucleotide sequence that complements a nucleotide sequences of a tether strand. In some cases, a docker may comprise a nucleic acid strand containing two or more nucleotide sequences. For example, a docker nucleic acid strand may include a first nucleotide sequence that is configured to hybridize to a first tether nucleic acid strand and a second nucleotide sequence that is configured to hybridize to a first tether nucleic acid strand. In another example, a docker nucleic acid strand may include a first nucleotide sequence that is configured to hybridize to a first tether nucleic acid strand and a second nucleotide sequence that is configured to facilitate binding of a toehold displacement oligonucleotide, thereby facilitating dissociation of a docker from a tether. A docker nucleic acid strand may comprise two or more nucleotide sequences, in which a first nucleotide sequence and a second nucleotide sequence are overlapping (e.g., comprise one or more common or shared nucleotides). A docker nucleic acid strand may comprise two or more nucleotide sequences, in which a first nucleotide sequence and a second nucleotide sequence are non-overlapping. A nucleic acid strand that is used as a docker can include a sequence of at least 3, 4, 5, 6, 7, 8, 9, 10, 12, 14, 16, 18, 20, 25 or more nucleotides. Alternatively or additionally, a nucleic acid strand that is used as a docker can include a sequence of at most 25, 20, 18, 16, 14, 12, 10, 9, 8, 7, 6, 5, 4, 3 or fewer nucleotides.

Other useful dockers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary dockers include, but are not limited to, an antibody, Fab′ fragment, F(ab′)₂ fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a docker can be a protein that recognizes a nucleic acid sequence such as a DNA binding protein or RNA binding protein. Exemplary nucleic acid-binding proteins, which can be used as dockers, and the nucleic acid moieties to which they bind, which can be used as tethers, include a Toll-Like Receptor (TLR) which binds to DNA having a CpG moiety, transcription factor which binds to a specific nucleic acid sequence, or histone protein(s) which binds to DNA. Further examples are provided in the Eukaryotic nucleic acid binding protein database (ENPD). See Leung et al. Nucleic Acids Res. 47(Database issue): D322-D329 (2019), which is incorporated herein by reference.

A docker can be associated with an analyte via covalent and/or non-covalent attachment of the docker to the analyte. Exemplary attachment chemistries include those set forth herein in the context of attaching analytes and affinity reagents to solid supports and particles. In some configurations, a docker can be attached to a particle (e.g. structured nucleic acid particle) or solid support to which an analyte is attached. Optionally, one or more dockers can be associated with an analyte via attachment to an address or other unique identifier to which the analyte is attached.

An analyte can be associated with a single docker or, alternatively, with a plurality of dockers. For example, an analyte can be associated with at least 2, 3, 4, 5, 6, 7, 8, 9, 10, 15, 20, 25, 50 or more dockers. Alternatively or additionally, an analyte can be associated with at most 50, 25, 20, 15, 10, 9, 8, 7, 6, 5, 4, 3, 2 or fewer dockers. The dockers can be substantially identical to each other, thereby recognizing the same tethers. Alternatively, a plurality of dockers can include dockers that differ from each other. A plurality of dockers can comprise a first set of dockers having a first common sequence, and a second set of dockers having a second common sequence, in which the first set of dockers and the second set of dockers contain equal quantities of dockers (e.g., 4 first dockers and 4 second dockers). Alternatively, a plurality of dockers can comprise a first set of dockers having a first common sequence, and a second set of dockers having a second common sequence, in which the first set of dockers and the second set of dockers contain differing quantities of dockers (e.g., 4 first dockers and 8 second dockers). In some cases, the different dockers will recognize different tethers. It is also possible for the different dockers to recognize the same tethers. In some configurations, an analyte and the docker with which it is associated will have binding characteristics that are orthogonal to each other. As such, a paratope of an affinity reagent that recognizes or binds to the analyte will not recognize or bind to the docker, and a tether that recognizes or binds to the docker will not recognize or bind to the analyte. In alternative configurations, an analyte and the docker with which it is associated can recognize or bind the same paratope or the same epitope. Similarly, the analyte and the docker with which it is associated can be configured to recognize or bind the same tether.

The number of dockers associated with an analyte can be selected to accommodate any number of tethers that will be associated with an affinity reagent to which the analyte is expected to bind. For example, a particle or unique identifier to which an analyte is attached can include at least as many dockers as the number of tethers associated with an affinity reagent that will be contacted with the analyte. Optionally, an analyte may be contacted with a plurality of affinity reagents, wherein the range of tethers associated with each affinity reagent is variable. In such configurations, the number of dockers associated with the analyte can be greater than or equal to the upper end of the range. As such, all affinity reagents in the plurality can benefit from avidity improvements imparted by the full set of tethers with which they are associated.

A plurality of dockers can be configured as a set of dockers that is geometrically constrained relative to an analyte with which the set is associated. For example, the analyte can be attached to a fixed position on a particle or sold support, and the dockers in the set can be attached at fixed positions relative to the position of analyte attachment. As a further option, individual dockers in the set can each be attached to the particle or solid support at fixed positions relative to other dockers in the set. As set forth in further detail herein, the number, type and/or position of the dockers in a set can be configured to accommodate interaction with a particular number, type or geometric arrangement of tethers that are associated with an affinity reagent. In some configurations, a set of dockers can be configured to include one or more subsets of dockers, wherein the dockers within each subset are geometrically constrained with respect to their relative attachment positions on a particle or solid support. The number, type and/or position of the dockers in a given subset of dockers can be configured to accommodate interaction with a particular number, type or geometric arrangement of tethers that are associated with an affinity reagent. In some cases, multiple subsets of dockers are attached to a particle or solid support, and each of the subsets has a substantially identical configuration. As such, the multiple subsets provide redundant opportunities for interaction with a plurality of tethers that are associated with an affinity reagent. Alternatively, multiple subsets of dockers that are attached to a particle or solid support have configurations that differ from each other. As such, the subsets can be selective for different affinity reagents according to differences in the number, type and/or positions of tethers that are associated with the respective affinity reagents.

An analyte can be attached to a particle, solid support or other substance. A particularly useful particle is a structured nucleic acid particle. Structured nucleic acid particles can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. No. 11,203,612 or 11,505,796; US Pat. App. Pub. No. 2022/0162684 A1, or U.S. patent application Ser. No. 18/058,000, each of which is incorporated herein by reference. Alternatively, an analyte can be attached to a solid support by a particle that is substantially devoid of nucleic acids, such as a synthetic polymeric particle, a carbon nanoparticle, or an inorganic nanoparticle.

A structured nucleic acid particle may have any of a variety of sizes and shapes to accommodate use in a desired application. For example, a structured nucleic acid particle can have a regular or symmetric shape or, alternatively, a structured nucleic acid particle can have an irregular or asymmetric shape. The shape can be rigid or pliable. The size or shape of a structured nucleic acid particle can be characterized with respect to length, area (i.e. footprint), or volume. The size or shape of a structured nucleic acid particle can be smaller than an address in an array to which it will associate or attach. Optionally, the size or shape of individual structured nucleic acid particles in a population of structured nucleic acid particles is configured to preclude more than one of the particles from occupying an address in an array.

Optionally, a structured nucleic acid particle or population thereof can have a minimum, maximum or average length of at least about 50 nm, 100 nm, 250 nm, 500 nm, 1 micron, 5 micron or more. Alternatively or additionally, a structured nucleic acid particle or population thereof can have a minimum, maximum or average length of no more than about 5 micron, 1 micron, 500 nm, 250 nm, 100 nm, 50 nm, or less.

Optionally, a structured nucleic acid particle or population thereof can have a minimum, maximum or average volume of at least about 1 micron³, 10 micron³, 100 micron³, 1 mm³ or more. Alternatively or additionally, a structured nucleic acid particle or population thereof can have a minimum, maximum or average volume of no more than about 1 mm³, 100 micron³, 10 micron³, 1 micron³ or less.

Optionally, the minimum, maximum or average area (i.e. footprint) for a structured nucleic acid particle can be at least about 10 nm², 100 nm², 1 micron², 10 micron², 100 micron², 1 mm² or more. Alternatively or additionally, the minimum, maximum or average area for a structured nucleic acid particle footprint can be at most about 1 mm², 100 micron², 10 micron², 1 micron², 100 nm², 10 nm², or less. The footprint of a structured nucleic acid particle may have a regular shape or an approximately regular shape, such as triangular, square, rectangular, circular, ovoid, or polygonal shape.

A structured nucleic acid particle (e.g. having origami or nanoball structures) may include regions of single-stranded nucleic acid, regions of double-stranded nucleic acid, or combinations thereof. For example, a structured nucleic acid particle can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. For example, the moiety can include an analyte (e.g. protein), paratope, affinity moiety (e.g. antibody), organic linker, inorganic ion, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near the 5′ end of the oligonucleotide, at or near the 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Optionally, an analyte of the present disclosure can be attached to a unique identifier in an array of unique identifiers. An array can include a number or variety of unique identifiers, for example, to accommodate a desired sample complexity. In some configurations, the array is configured for single-molecule resolution. For example, individual addresses (or other unique identifiers) of an array can each be attached to no more than one analyte. Alternatively, individual addresses of the array can each be attached to an ensemble of analytes.

The addresses of an array can optionally be optically observable and, in some configurations, adjacent addresses can be optically distinguishable when detected. Addresses of an array are typically discrete. The discrete addresses can be contiguous, or they can have interstitial spaces between each other. An array can have, for example, addresses that are separated by less than 100 microns, 10 microns, 1 micron, 500 nm, 100 nm, 10 nm or less. Alternatively or additionally, an array can have addresses that are separated by at least 10 nm, 100 nm, 500 nm, 1 micron, 5 microns, 10 microns, 50 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁸, 1×10¹⁰, 1×10¹², or more addresses. Although exemplified for addresses, it will be understood that other unique identifiers can be similarly configured in an array.

An array can have a size and complexity that is sufficient to accommodate a plurality of proteins such as those exemplified below. Arrays of other analytes or arrays of affinity reagents can also have size and complexity exemplified below for proteins. It will be understood that the pluralities of proteins set forth below need not be limited to array configurations.

A plurality of proteins, whether present in an array or other composition set forth herein, can be characterized in terms of total protein mass. The total mass of protein in a liter of plasma has been estimated to be 70 g and the total mass of protein in a human cell has been estimated to be between 100 μg and 500 pg depending upon cells type. See Wisniewski et al. Molecular & Cellular Proteomics 13:10.1074/mcp.M113.037309, 3497-3506 (2014), which is incorporated herein by reference. A plurality of proteins can include at least 1 μg, 10 pg, 100 μg, 1 ng, 10 ng, 100 ng, 1 ug, 10 ug, 100 ug, 1 mg, 10 mg, 100 mg or more protein by mass. Alternatively or additionally, a plurality of proteins may contain at most 100 mg, 10 mg, 1 mg, 100 ug, 10 ug, 1 ug, 100 ng, 10 ng, 1 ng, 100 μg, 10 pg, 1 μg or less protein by mass.

A plurality of proteins can include or be obtained from a proteomic sample. A proteomic sample can include substantially all proteins from a given source or a substantial fraction thereof. For example, a proteomic sample may contain at least 60%, 75%, 90%, 95%, 99%, 99.9% or more of the total protein mass present in the source from which the sample was derived. Alternatively or additionally, a proteomic sample may contain at most 99.9%, 99%, 95%, 90%, 75%, 60% or less of the total protein mass present in the source from which the sample was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein molecules, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length amino acid sequences in the plurality. For example, the variety of full-length amino acid sequences in a plurality of proteins can be equated with the number of different protein-encoding genes in the source for the plurality of proteins. Whether or not the proteins are derived from a known genome or from any genome at all, the variety of full-length amino acid sequences can be counted independent of presence or absence of post translational modifications in the proteins. A human proteome is estimated to have about 20,000 different protein-encoding genes such that a plurality of proteins derived from a human can include up to about 20,000 different full-length amino acid sequences. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity that includes substantially all different native-length amino acid sequences from a given source. A proteome or subfraction can have a complexity of at least 2, 5, 10, 100, 1×10³, 1×10⁴, 2×10⁴, 3×10⁴ or more different native-length amino acid sequences. Alternatively or additionally, a proteome or subfraction can have a complexity that is at most 3×10⁴, 2×10⁴, 1×10⁴, 1×10³, 100, 10, 5, 2 or fewer different native-length amino acid sequences.

The diversity of a proteomic sample can include at least one representative for substantially all proteins encoded by a source from which the sample was derived or a substantial fraction thereof. For example, a proteomic sample may contain at least one representative for at least 60%, 75%, 90%, 95%, 99%, 99.9% or more of the proteins encoded by a source from which the sample was derived. Alternatively or additionally, a proteomic sample may contain a representative for at most 99.9%, 99%, 95%, 90%, 75%, 60% or less of the proteins encoded by a source from which the sample was derived.

A plurality of proteins can be characterized in terms of the variety of full-length amino acid sequences in the plurality including transcribed splice variants. The human proteome has been estimated to include about 70,000 different full-length amino acid sequences when splice variants ae included. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different full-length amino acid sequences. Alternatively or additionally, a plurality of proteins can have a complexity that is at most 1×10⁶, 1×10⁵, 7×10⁴, 1×10⁴, 1×10³, 100, 10, 5, 2 or fewer different full-length amino acid sequences.

A plurality of proteins can be characterized in terms of the variety of protein structures therein including, for example, different full-length amino acid sequences or different proteoforms among those sequences. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the plurality. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A sample used herein, whether containing proteins or other analytes, need not be from a biological source and can instead be from an artificial source, such as a library from a combinatorial synthesis or a library from an in vitro synthesis that exploits biological components. An artificial sample can have a range of complexity similar to those set forth herein for proteomes. A method set forth herein can detect, identify or characterize some or all proteins in a proteome or other sample including, for example, at least about 1%, 5%, 10%, 25%, 50%, 75%, 90% or 99% of the proteins in the sample.

An array of analytes can include a plurality of unique identifiers (e.g. addresses) each of which is associated with a different analyte. For example, the unique identifiers in an array can each be attached to a different analyte, such as a different protein. Each unique identifier in an array can also be attached to at least one docker. Optionally, the array utilizes universal dockers, whereby the dockers are substantially the same for some or all unique identifiers in the array. As such, each unique identifier in an array can be associated with an analyte that differs from analytes at other unique identifiers in the array and a universal docker that is the same as universal dockers at other unique identifiers in the array. In an exemplary configuration, addresses in an array are each attached to a different protein selected from a protein sample, and the universal docker that is attached to each of the addresses is the same. Accordingly, individual addresses can be distinguished based upon the unique structure of the attached analyte but the universal docker provides a structure that is universal across the addresses.

In some configurations, an array can include indexed dockers, wherein unique identifiers in one subset are distinguishable from unique identifiers in another subset based on the binding properties of the indexed dockers with which they are respectively associated. Accordingly, an array can include at least 1, 2, 3, 4, 5, 10 or more subsets of indexed dockers, wherein each subset of indexed dockers is associated with a respective subset of unique identifiers in the array. An array need not use universal nor indexed dockers. For example, individual addresses (or other unique identifiers) in an array can be attached to unique dockers, wherein the addresses are distinguishable based on the structure of the attached docker.

An analyte that is used in a composition or method set forth herein can be in a fluid (i.e. a fluid-phase analyte). As such, an analyte can be diffusible in the fluid. A fluid-phase analyte can be associated with a docker. In some configurations, the analyte can be attached to a particle, such as a structured nucleic acid particle. The particle-attached analyte can include an associated docker, for example, via attachment of the docker to the particle.

Any of a variety of affinity reagents can be used in a composition or method set forth herein. Particularly useful affinity reagents include, but are not limited to, antibodies or functional fragments thereof (e.g., Fab′ fragments, F(ab′)₂ fragments, single-chain variable fragments (scFv), di-scFv, tri-scFv, or microantibodies), aptamers, affibodies, affilins, affimers, affitins, alphabodies, anticalins, avimers, miniproteins, DARPins, monobodies, nanoCLAMPs, lectins, or functional fragments thereof. The exemplified affinity reagents can be used individually. Alternatively, an affinity reagent set forth herein can be used as a paratope or moiety of an affinity reagent having a plurality of paratopes or moieties. For example, an affinity reagent set forth herein can provide one paratope (or a subset of paratopes) of an affinity reagent having a plurality of paratopes. In some configurations, a composition or method set forth herein can lack one or more of the affinity reagents set forth herein.

An antibody is a particularly useful affinity reagent for use in a composition or method set forth herein. The antibody can be any antigen-binding molecule or molecular complex having at least one complementarity determining region (CDR) that binds to a particular epitope with high affinity. An antibody can include four polypeptide chains: two heavy chains (HC1 and HC2) and two light chains (LC1 and LC2). HC1 and HC2 can be covalently connected by one, two or more disulfide bonds. HC1 can be covalently connected to LC1 by at least one disulfide bond. HC2 can be covalently connected to LC2 by at least one disulfide bond. Each heavy chain can include a heavy chain variable region (V_{H}) and a heavy chain constant region (C_{H}). The heavy chain constant region can include three domains, C_{H1}, C_{H2} and C_{H3}. Each light chain can include a light chain variable region (V_{L}) and a light chain constant region (C_{L}). The V_{H} and V_{L} regions can further include regions of hypervariability, termed complementarity determining regions (CDRs), interspersed with regions that are more conserved, termed framework regions (FR). Each V_{H} and V_{L} can include three CDRs and four FRs, arranged from amino-terminus to carboxy-terminus in the following order: FR1, CDR1, FR2, CDR2, FR3, CDR3, FR4.

An antibody can include all elements of a full-length antibody, such as those enumerated above. However, an antibody need not be full length and functional fragments can be particularly useful for many uses. The term “antibody” as used herein encompasses full length antibodies and functional fragments thereof. A functional fragment can be naturally occurring, enzymatically obtainable, synthetic, or genetically engineered. An antibody can be obtained using any suitable technique such as proteolytic digestion or recombinant genetic engineering techniques involving the manipulation and expression of DNA encoding one or more antibody domains. Such DNA is readily available, for example, from commercial sources, DNA libraries (e.g., phage-antibody libraries), or can be synthesized. The DNA may be manipulated chemically or by using molecular biology techniques, for example, to arrange one or more variable and/or constant domains into a suitable configuration, or to introduce codons, introduce cysteine residues, remove cysteine residues, modify, add or delete other amino acids, etc.

A functional fragment of an antibody can include any fragment that is capable of binding to an epitope with a detectable affinity, such as a Fab, Fab′, F(ab′)₂, Fd, Fv, dAb, single-chain variable (scFv), di-scFv, tri-scFv, microantibody, or minimal recognition unit consisting of the amino acid residues that mimic the hypervariable region of an antibody (e.g., an isolated complementarity determining region (CDR) such as a CDR3 peptide). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains can also be useful.

A functional fragment of an antibody will typically include at least one variable domain. The variable domain may be of any of a variety of sizes or amino acid compositions and will generally include at least one CDR which is adjacent to or in frame with one or more framework sequences. For antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, a functional fragment of an antibody may contain a monomeric V_{H} or V_{L} domain.

In particular configurations, a functional fragment of an antibody contains at least one variable domain covalently connected to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (v) V_{H}-C_{H1}-C_{H2}-C_{H3}; (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly connected to one another or may be connected by a full or partial hinge or linker region. A hinge region may consist of at least 2 (e.g., at least 5, 10, 15, 20, 40, 60 or more) amino acids which result in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

An affinity reagent used in a composition or method of the present disclosure can be associated with a tether. The tether can be any molecule or moiety that is capable of interacting with a docker. A particularly useful tether is a nucleic acid strand having a nucleotide sequence that complements a nucleotide sequence of a docker strand. In some cases, a tether may comprise a nucleic acid strand containing two or more nucleotide sequences. For example, a tether nucleic acid strand may include a first nucleotide sequence that is configured to hybridize to a first tether nucleic acid strand and a second nucleotide sequence that is configured to facilitate binding of a toehold displacement oligonucleotide, thereby facilitating dissociation of the tether from a docker. A tether nucleic acid strand may comprise two or more nucleotide sequences, in which a first nucleotide sequence and a second nucleotide sequence are overlapping (e.g., comprise one or more common or shared nucleotides). A tether nucleic acid strand may comprise two or more nucleotide sequences, in which a first nucleotide sequence and a second nucleotide sequence are non-overlapping. A nucleic acid strand that is used as a tether can include a sequence of at least 3, 4, 5, 6, 7, 8, 9, 10, 12, 14, 16, 18, 20, 25 or more nucleotides. Alternatively or additionally, a nucleic acid strand that is used as a tether can include a sequence of at most 25, 20, 18, 16, 14, 12, 10, 9, 8, 7, 6, 5, 4, 3 or fewer nucleotides.

Other useful tethers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary tethers include, but are not limited to, an antibody, Fab′ fragment, F(ab′)₂ fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a tether can be a protein that recognizes a nucleic acid sequence such as a DNA binding protein or RNA binding protein. Exemplary nucleic acid-binding proteins, which can be used as tethers, and the nucleic acid moieties to which they bind, which can be used as dockers, include Toll-Like Receptor (TLR) which binds to DNA having a CpG moiety, transcription factors which bind to specific nucleic acid sequences, or histones which bind to DNA. Further examples are provided in the Eukaryotic nucleic acid binding protein database (ENPD). See Leung et al. Nucleic Acids Res. 47(Database issue): D322-D329 (2019), which is incorporated herein by reference.

A tether can be associated with an affinity reagent via covalent and/or non-covalent attachment of the tether to the affinity reagent. Exemplary attachment chemistries include those set forth herein in the context of attaching analytes and affinity reagents to solid supports and particles. In some configurations, a tether can be attached to a particle (e.g. structured nucleic acid particle) or solid support to which an affinity reagent is attached. For example, one or more tethers can be associated with an affinity reagent via attachment to a structured nucleic acid particle to which the affinity reagent is attached.

An affinity reagent can be associated with a plurality of tethers. For example, an affinity reagent can be associated with at least 2, 3, 4, 5, 6, 7, 8, 9, 10, 15, 20, 25, 50 or more tethers. Alternatively or additionally, an affinity reagent can be associated with at most 50, 25, 20, 15, 10, 9, 8, 7, 6, 5, 4, 3, 2 or fewer tethers. The tethers can be substantially identical to each other, thereby recognizing the same dockers. Alternatively, a plurality of tethers can include tethers that differ from each other. A plurality of tether can comprise a first set of tethers having a first common sequence, and a second set of tethers having a second common sequence, in which the first set of tethers and the second set of tethers contain equal quantities of tethers (e.g., 4 first tethers and 4 second tethers). Alternatively, a plurality of tethers can comprise a first set of tethers having a first common sequence, and a second set of tethers having a second common sequence, in which the first set of tethers and the second set of tethers contain differing quantities of tethers (e.g., 4 first tethers and 8 second tethers). In some cases, the different tethers will recognize different dockers. It is also possible for the different tethers to recognize the same dockers. In some configurations, an affinity reagent and the tether with which it is associated will have orthogonal binding recognition. As such, an analyte that recognizes or binds to a paratope of the affinity reagent will not recognize or bind to the tether, and a docker that recognizes or binds to the tether will not recognize or bind to the paratope. Alternatively, an affinity reagent and the tether with which it is associated can be configured to recognize or bind the same analyte. Similarly, the affinity reagent and the tether with which it is associated can be configured to recognize or bind the same docker.

A plurality of tethers can be configured as a set of tethers that is geometrically constrained relative to an affinity reagent with which the set is associated. For example, a paratope of the affinity reagent can be attached to a fixed position on a particle or sold support, and the tethers in the set can be attached at fixed positions relative to the position of paratope attachment. As a further option, individual tethers in the set can each be attached to the particle or solid support at fixed positions relative to other tethers in the set. As set forth in further detail herein, the number, type and/or position of the tethers in a set can be configured to accommodate interaction with a particular number, type or geometric arrangement of dockers that are associated with an analyte. In some configurations, a set of tethers can be configured to include one or more subsets of tethers, wherein the tethers within each subset are geometrically constrained with respect to their relative attachment positions on a particle or solid support. The number, type and/or position of the tethers in a given subset of tethers can be configured to accommodate interaction with a particular number, type or geometric arrangement of dockers that are associated with an analyte. In some cases, multiple subsets of tethers are attached to a particle or solid support, and each of the subsets has a substantially identical configuration. As such, the multiple subsets provide redundant opportunities for interaction with a plurality of dockers that are associated with an analyte. Alternatively, multiple subsets of tethers that are attached to a particle or solid support can have configurations that differ from each other. As such, the subsets can be selective for different analytes according to differences in the number, type and/or positions of dockers that are associated with the respective analytes.

Optionally, an affinity reagent can be attached to a solid support or particle. A particularly useful particle is a structured nucleic acid particle (e.g. nucleic acid origami), for example, having structural or functional characteristics set forth herein in the context of attachment to proteins and other analytes. An analyte, affinity reagent, docker, tether, label or other moiety can be attached to a nucleic acid origami via a scaffold component or oligonucleotide component. For example, the scaffold or oligonucleotide can include a nucleotide analog that forms a covalent or non-covalent bond.

Any of a variety of chemistries can be used to attach an analyte, affinity reagent, docker, tether or other moiety to a solid support or particle (e.g. structured nucleic acid particle). The attachment can be covalent. Exemplary covalent chemistries include, but are not limited to, click chemistries or chemistries set forth in U.S. Pat. No. 11,203,612 or 11,505,796; or US Pat. App. Pub. No. 2022/0162684 A1, each of which is incorporated herein by reference. Another example is the SpyTag/SpyCatcher system (See, Zakeri et al. Proceedings Natl Acad. Sciences USA. 109 (12): E690-7 (2012)). In this system, a 13 amino acid tag polypeptide (Spy Tag) forms a first coupling handle, with a 12.3 kDa protein (Spy-Catcher) forming the other coupling handle. The SpyCatcher can function by irreversibly bonding to a SpyTag through an isopeptide bond. Any of a variety of non-covalent bonds can be used to attach an analyte, affinity reagent or other moiety to a solid support or particle (e.g. structured nucleic acid particle). Receptors and their ligands can be particularly useful. Examples include, but are not limited to, antibodies, antigens, (strept)avidin (or analogs thereof), biotin (or analogs thereof), affibodies, affilins, affimers, affitins, alphabodies, anticalins, avimers, DARPins, monobodies, nanoCLAMPs, nucleic acids, peptide nucleic acids, polypeptides, nucleic acid aptamers, protein aptamers, lectins (or analogs thereof), carbohydrates or functional fragments thereof. Complementary nucleic acids can be used to non-covalently attach a functional moiety to a solid support or particle (e.g. structured nucleic acid particle). Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Reagents and techniques that can be used to non-covalently attach an affinity reagent or other moiety to a particle (e.g. structured nucleic acid particle) are set forth in U.S. Pat. No. 11,203,612 or 11,505,796; or US Pat. App. Pub. No. 2022/0162684 A1, each of which is incorporated herein by reference.

A structured nucleic acid particle that is made or used in a method set forth herein can be suspended in a fluid, immobilized on a solid support, or immobilized in another material such as a gel or solid support material. This can be the case before, during or after being attached to a moiety of interest, such as a tether, docker, handle, linker, label, analyte or affinity moiety. For example, a population of structured nucleic acid particles can be colloidal for some, or all steps of a method set forth herein. Alternatively, a population of structured nucleic acid particles can be immobilized in, or on a solid support for some, or all steps of a method set forth herein. For example, analytes or affinity reagents can be attached to addresses (or other unique identifiers) of an array via structured nucleic acid molecules.

A particle need not be composed primarily of nucleic acid and, in some cases, may be devoid of nucleic acids. For example, an analyte, affinity reagent, docker or tether can be attached to an artificial polymer that is configured to form a particle. In other examples, a particle can be composed of a solid support material, such as those set forth herein, glass, silicon, silica, carbon, cellulose, polyethylene glycol (PEG), upconversion nanocrystal, or a quantum dot.

An affinity reagent of the present disclosure can include one or more paratopes. For example, an affinity reagent can include at least 1, 2, 3, 4, 5, 10, 15, 20, 25 or more paratopes. Alternatively or additionally, an affinity reagent can include at most 25, 20, 15, 10, 5, 4, 3, 2, or 1 paratopes. Multiple paratopes that are present in an affinity reagent can have the same function or different functions compared to each other. For example, the multiple paratopes can each have affinity for the same epitope or same set of epitopes. In some cases, the strength and/or specificity of the affinity can be substantially the same, for example, in cases where the paratopes have the same structure. In other cases, the strength and/or specificity of the affinity of two or more paratopes for a given epitope or set of epitopes can overlap despite some differences in the functional or structural characteristics of the two or more paratopes. In some configurations, multiple paratopes of a given affinity reagent can have affinity for different epitopes. This can be the case, whether the different epitopes are found in the same analyte (e.g. two different amino acid trimer epitopes present in a given protein analyte) or in different analytes (e.g. a first trimer epitope being found in a first protein that lacks a second trimer epitope, and a second trimer epitope being found in a second protein that lacks the first trimer epitope).

An affinity reagent of the present disclosure can include one or more labels. For example, an affinity reagent can include at least 1, 2, 3, 4, 5, 10, 15, 20 or more labels. Alternatively or additionally, an affinity reagent can include at most 20, 15, 10, 5, 4, 3, 2, or 1 labels. In some configurations, an affinity reagent can be attached to one or more labels. For example, an affinity reagent can include a particle (e.g. structured nucleic acid particle) that is attached to at least one paratope and further attached to at least one label.

Multiple labels that are present in an affinity reagent can have the same structure as each other or they can differ structurally from each other. Optionally, multiple labels can have different detectable characteristics. For example, two or more optical labels can differ in terms of luminescence lifetime, luminescence polarity, extinction coefficient, quantum yield of luminescence, spectral region for absorbance, spectral region for excitation or spectral region for emission. Alternatively, two or more optical labels can have overlapping detectable characteristics, for example, in terms of luminescence lifetime, luminescence polarity, extinction coefficient, quantum yield of luminescence, spectral region for absorbance, spectral region for excitation or spectral region for emission. This can result from the two or more optical labels having the same structure, but in some cases two or more labels can have the same or overlapping detection properties despite having different structures.

A wide variety of labels can be used in a composition or method set forth herein including, for example, optically detectable labels, such as luminophores (e.g. fluorophores), enzymes (e.g. enzymes which catalyze reactions with colored reagents or products), electrochemical labels (e.g. highly charged moieties). Magnetic contrast imaging moieties can be used such as gadolinium-diethylenetriaminepentacetate (Gd-DTPA), gadolinium-dodecane tetraacetic acid (Gd-DOTA) or others used for magnetic resonance techniques. Moieties that are detected through subsequent processing, such as nucleic acid barcode labels, can also be useful. Nucleic acids can be detected or identified via nucleic acid amplification, sequencing or hybridization assays.

Any of a variety of luminophores may be used herein. Luminophores may include labels that emit in the ultraviolet, visible, or infrared region of the spectrum. In some cases, the luminophore may be selected from the group consisting of FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, R-Phycoerythrin, Allophcocyanin (APC). In some cases, the label may be an Atto dye, for example Atto 390, Atto 425, Atto 430, Atto 465, Atto 488, Atto 490, Atto 495, Atto 514, Atto 520, Atto 532, Atto 540, Atto 550, Atto 565, Atto 580, Atto 590, Atto 594, Atto 610, Atto 611, Atto 612, Atto 620, Atto 633, Atto 635, Atto 647, Atto 655, Atto 680, Atto 700, Atto 725, Atto 740, Atto MB2, Atto Oxa12, Atto Rho101, Atto Rho12, Atto Rho13, Atto Rho14, Atto Rho3B, Atto Rho6G, or Atto Thio12. In some cases, the luminophore may be a fluorescent protein such as green fluorescent protein (GFP), cyan fluorescent protein (CFP), red fluorescent protein (RFP), blue fluorescent protein (BFP), orange fluorescent protein (OFP), and yellow fluorescent protein (YFP). A wide range of effective luminophores are commercially available, for example, from the Molecular Probes division of ThermoFisher Scientific and/or generally described in the Molecular Probes Handbook (11^{th} Edition) which is hereby incorporated by reference. Label components may also include intercalation dyes, such as ethidium bromide, propidium bromide, crystal violet, 4′,6-diamidino-2-phenylindole (DAPI), 7-aminoactinomycin D (7-AAD), Hoescht 33258, Hoescht 33342, Hoescht 34580, YOYO-1, DiYO-1, TOTO-1, DiTO-1, or combinations thereof.

A method of the present disclosure can include a step of contacting a docker-associated analyte with a tether-associated affinity reagent, whereby the affinity reagent binds to the analyte, for example, via at least one paratope of the affinity reagent interacting with an epitope of the analyte and via at least one tether interacting with at least one docker. For example, an array of analytes can be contacted with an affinity reagent, wherein a first analyte is attached to a unique identifier in the array, wherein a docker is attached to the unique identifier, wherein the affinity reagent has a paratope that recognizes an epitope of the analyte, and wherein the affinity reagent further comprises a tether that recognizes the docker. In this configuration, the affinity reagent can associate with the unique identifier via binding of the paratope to the epitope and via binding of the tether to the docker.

Avidity of association between an affinity reagent and an analyte and a docker and tether may be balanced according to the respective binding strengths or equilibria of the affinity reagent/analyte interaction and the docker/tether interaction. In some cases, a docker/tether pair or a plurality thereof may be chosen to have a larger dissociation constant than the dissociation constant of an affinity agent/analyte pair (e.g., a docker/tether pair having a 1 μM dissociation constant and an affinity reagent/analyte pair having a 10 nM dissociation constant). Accordingly, an affinity reagent may be expected to have a faster association rate to a target analyte than the association rate of a tether to its complementary docker. In some cases, an affinity reagent may be provided with a plurality of tethers, in which each tether forms a binding interaction with a docker having a larger dissociation constant than the dissociation constant of an affinity reagent/analyte pair. Alternatively, a docker/tether pair or a plurality thereof may be chosen to have a smaller dissociation constant than the dissociation constant of an affinity agent/analyte pair. In some cases, an affinity reagent may be provided with a plurality of tethers, in which each tether forms a binding interaction with a docker having a smaller dissociation constant than the dissociation constant of an affinity reagent/analyte pair. Methods of measuring dissociation constants, binding on-rate, and/or binding off-rate are known in the art and can include methods such as equilibrium dialysis, ELISA, and TIRF. Once dissociation constants of affinity reagent/analyte pairs and docker/tethers pair are known, it may be possible to empirically determine a suitable docker/tether pair and titrate for an optimal quantity of dockers and/or tethers provided to achieve a given effective dissociation constant of the analyte/affinity reagent/dockers/tethers complex.

A difference in dissociation constant between an affinity reagent/analyte pair and a docker/tether pair may be at least about 2-fold (e.g., 10 nM vs 20 nM; 20 nM vs 10 nM), 3-fold, 5-fold, 10-fold, 20-fold, 50-fold, 100-fold, 200-fold, 500-fold, 1000-fold, or more than 1000-fold. Alternatively or additionally, a difference in dissociation constant between an affinity reagent/analyte pair and a docker/tether pair may be no more than about 1000-fold, 500-fold, 200-fold, 100-fold, 50-fold, 20-fold, 10-fold, 5-fold, 3-fold, 2-fold, or less than 2-fold.

Methods of the present disclosure may involve a step of measuring a signal from a detectable label of an affinity reagent, thereby determining association between an analyte and the affinity reagent at a unique identifier associated with the analyte. To increase the chances of observing coupling of the affinity reagent to the analyte, it may be preferable to provide sets of binding interactions (e.g., affinity reagent/analyte or docker/tether) that are likely to become associated within a certain incubation time and/or remain associated for the duration of a detection event (e.g., a scan of a fluorescent microscope).

Accordingly, an affinity reagent may be observed to bind to an analyte and a tether attached to the affinity reagent may be observed to bind to a docker co-located with the analyte after an incubation time of no more than about 60 minutes (mins), 30 mins, 20 mins, 15 mins, 10 mins, 5 mins, 3 mins, 2 mins, 1 min, or less than 1 min. Alternatively or additionally, an affinity reagent may be observed to bind to an analyte and a tether attached to the affinity reagent may be observed to bind to a docker co-located with the analyte after an incubation time of at least about 1 min, 2 mins, 3 mins, 5 mins, 10 mins, 15 mins, 20 mins, 30 mins, 60 mins, or more than 60 mins. Likewise, affinity agents and associated tethers may be observed to bind to a percentage of target analytes and associated dockers (e.g., at least about 10%, 25%, 50%, 75%, 90%, 95%, 99%, or more than 99% of target analytes and associated dockers) after an incubation time of no more than about 60 minutes, 30 mins, 20 mins, 15 mins, 10 mins, 5 mins, 3 mins, 2 mins, 1 min, or less than 1 min. Alternatively or additionally, affinity agents and associated tethers may be observed to bind to a percentage of target analytes and associated dockers (e.g., at least about 10%, 25%, 50%, 75%, 90%, 95%, 99%, or more than 99% of target analytes and associated dockers) after an incubation time of at least about 1 min, 2 mins, 3 mins, 5 mins, 10 mins, 15 mins, 20 mins, 30 mins, 60 mins, or more than 60 mins.

Accordingly, an affinity reagent may be observed to bind to an analyte and a tether attached to the affinity reagent may be observed to bind to a docker co-located with the analyte when a detection event occurs at least about 1 s, 15 s, 30 s, 1 min, 2 mins, 3 mins, 5 mins, 10 mins, 15 mins, 20 mins, 30 mins, 60 mins, or more than 60 mins after associating the affinity reagent/analyte/docker/tether complex together. Alternatively or additionally, an affinity reagent may be observed to be bound to an analyte and a tether attached to the affinity reagent may be observed to be bound to a docker co-located with the analyte when a detection event occurs no more than about 60 minutes, 30 mins, 20 mins, 15 mins, 10 mins, 5 mins, 3 mins, 2 mins, 1 min, 30 s, 15 s, Is, or less than 1 s after associating the affinity reagent/analyte/docker/tether complex together. Likewise, affinity agents and associated tethers may be observed to be bound to a percentage of target analytes and associated dockers (e.g., at least about 10%, 25%, 50%, 75%, 90%, 95%, 99%, or more than 99% of target analytes and associated dockers) when a detection event occurs at least about 1 min, 2 mins, 3 mins, 5 mins, 10 mins, 15 mins, 20 mins, 30 mins, 60 mins, or more than 60 mins after associating the affinity reagent/analyte/docker/tether complex together. Alternatively or additionally, affinity agents and associated tethers may be observed to be bound to a percentage of target analytes and associated dockers (e.g., at least about 10%, 25%, 50%, 75%, 90%, 95%, 99%, or more than 99% of target analytes and associated dockers) when a detection event occurs no more than about 60 minutes, 30 mins, 20 mins, 15 mins, 10 mins, 5 mins, 3 mins, 2 mins, 1 min, or less than 1 min after associating the affinity reagent/analyte/docker/tether complex together.

In some configurations, an analyte is associated with an orthogonal docker. The docker can be orthogonal to the analyte due to differences between the structures of the docker and the analyte and/or due to differences in binding function. For example, a protein analyte can be associated with a docker consisting of a nucleic acid, carbohydrate, small molecule ligand or other non-proteinaceous moiety. In some configurations, an analyte is associated with an analogous docker. The docker can be analogous to the analyte due to structural similarities or similar binding function. For example, a protein analyte can be associated with a proteinaceous docker. In some configurations, a docker that is associated with a protein analyte can include an epitope that is the same or biosimilar to a target epitope in the protein analyte.

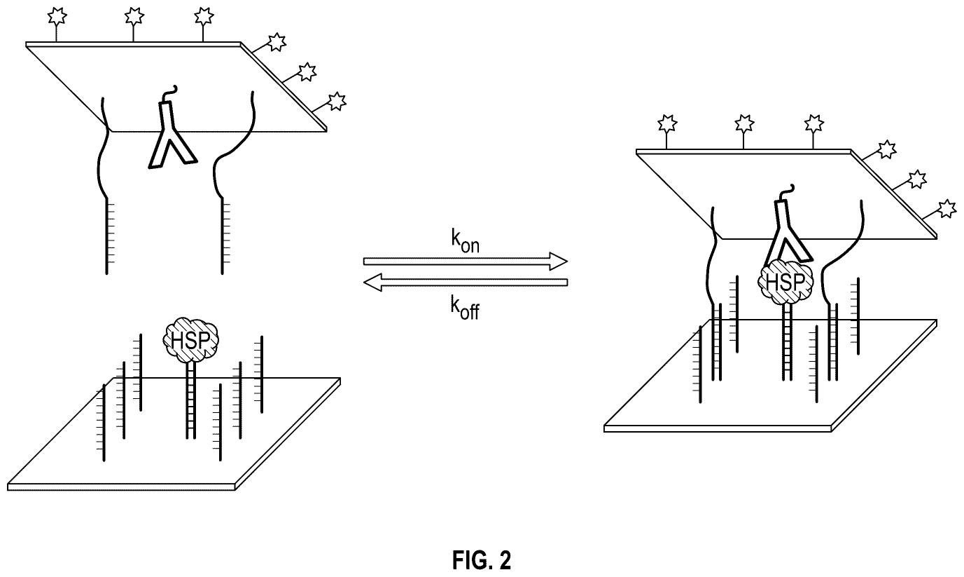

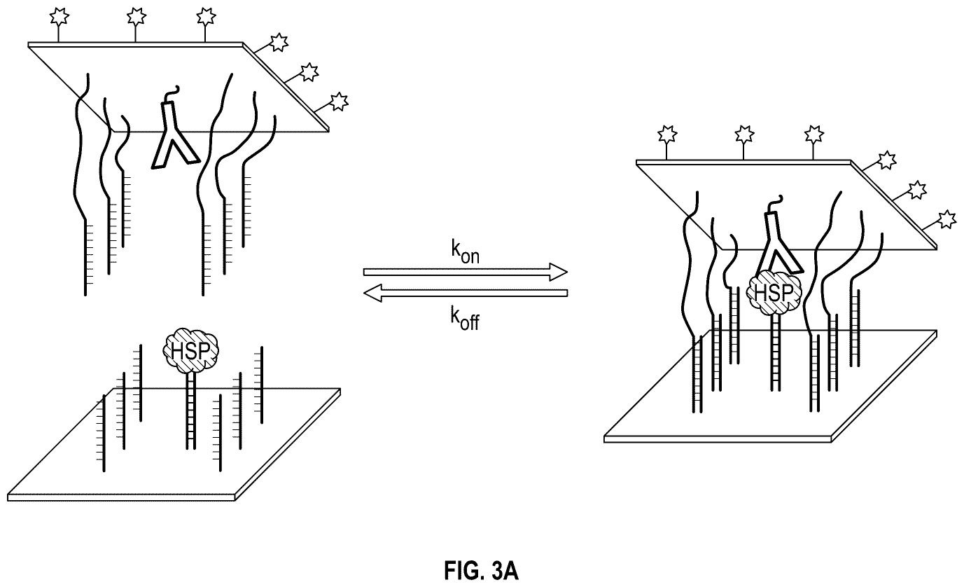

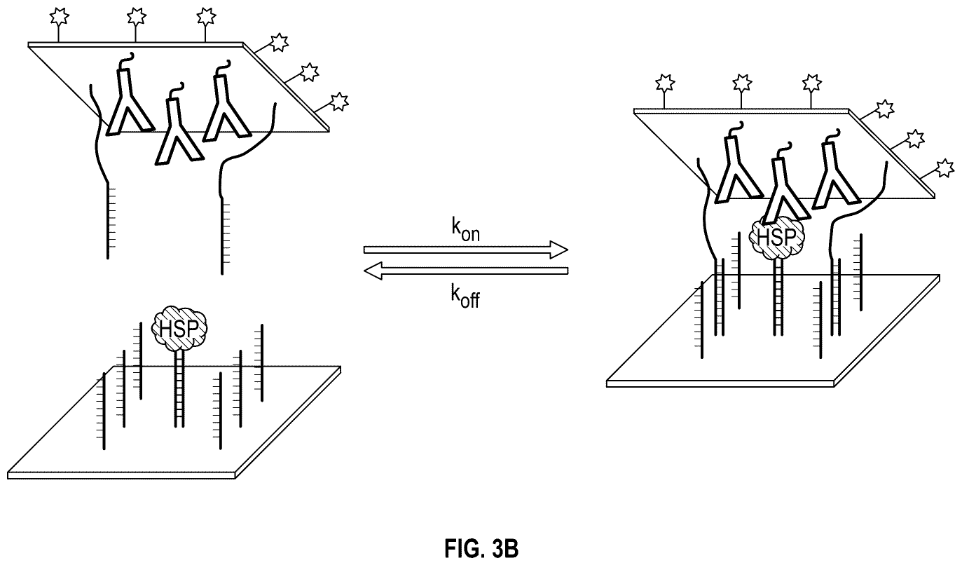

Nucleic acids are particularly useful as dockers and tethers. FIG. 2 shows a diagrammatic representation of equilibrium binding between an affinity reagent associated with two nucleic acid tethers and a protein analyte associated with six nucleic acid dockers. The affinity reagent includes a structured nucleic acid particle (grey tile) attached to an antibody component (Y-shape), six labels (7-pointed stars) and two nucleic acid tethers (comb structures). The protein analyte (stippled globule) is attached to a unique identifier (grey rectangle) to which six nucleic acid dockers (comb structures) are attached. The affinity reagent and analyte are in a dissociated relationship on the left side of the equation and an associated state on the right side. Association is mediated by binding of the paratope of the antibody component to the epitope (HSP amino acid sequence) of the protein and by hybridization of two dockers of the unique identifier to the two tethers of the affinity reagent. The number of tethers and/or dockers can be altered to tune avidity of the complex formed between an affinity reagent and analyte. FIG. 3A shows a diagrammatic representation of equilibrium binding between an affinity reagent associated with six nucleic acid tethers and a protein analyte associated with six nucleic acid dockers. The complex shown in FIG. 3A would be expected to have increased avidity compared to the complex in FIG. 2 due to the increased number of docker-tether interactions. The unique identifiers shown in FIGS. 2 and 3A each include six nucleic acid dockers and are thus configured to bind with at least 1, 2, 3, 4, 5 or 6 complementary tethers of an affinity reagent. Thus, the unique identifier can provide a range of avidities for multiple different affinity reagents.

As exemplified above, an analyte (e.g. protein analyte) can be associated with any number of nucleic acid dockers. One, some or all nucleic acid dockers that are associated with the analyte can hybridize to a complementary tether that is associated with an affinity reagent bound to the analyte. For example, at least 2, 3, 4, 5, 6, 7, 8, 9, 10, 15, 20, 25, 50 or more analyte-associated dockers can hybridize to tethers that are associated with an affinity reagent. Alternatively or additionally, at most 50, 25, 20, 15, 10, 9, 8, 7, 6, 5, 4, 3, 2 or fewer analyte-associated dockers can hybridize to tethers that are associated with an affinity reagent. Independent of the number of hybridized dockers and tethers in a complex, the length of the hybrids can be at least 2, 3, 4, 5, 6, 7, 8, 9, 10, 12, 14, 16, 18, 20, 25 or more nucleotides. Alternatively or additionally, the length of a hybridized region of a docker and tether can be at most 25, 20, 18, 16, 14, 12, 10, 9, 8, 7, 6, 5, 4, 3 or fewer nucleotides. A nucleic acid docker can bind to a protein tether or, conversely, a protein docker can bind to a nucleic acid tether. The combination of the number of nucleic acid duplexes formed by docker-tether pairs and the length of the duplexes can be tuned to produce a desired level of avidity. Exemplary titrations of number and length of such duplexes are demonstrated in Examples III and IV, herein.

Avidity of an affinity reagent can also be modified by changing the number of paratopes present. FIG. 3A shows a diagrammatic representation of equilibrium binding between a protein analyte having six nucleic acid dockers and an affinity reagent having two nucleic acid tethers and six paratopes (i.e. three antibodies each having two paratopes). By comparison, the complex formed in FIG. 3A has the same number of docker-tether interactions as the complex formed in FIG. 2 but a greater number of paratopes. Due to the added paratopes, the complex formed in FIG. 3A would be expected to have increased avidity compared to the complex formed in FIG. 2. Although not wishing to be limited by mechanism, and purely for purposes of explanation, increased avidity can be attributed to the increased local concentration of paratopes which, in turn, would be expected to decrease the overall off rate at equilibrium. Methods and compositions that utilize a plurality of affinity moieties to increase avidity, and which can be used in combination with the present disclosure, are set forth in US Pat. App. Pub. No. 2022/0162684 A1, which is incorporated herein by reference.

Vagaries in the synthesis and handling of analyte-bearing particles or array sites can yield populations having variable distributions of attached dockers. Looking to the example of nucleic acid origami structures, it is not unusual for staples and other oligonucleotide components to achieve incorporation efficiencies of approximately 90%. For a nominal analyte composition designed to have four dockers attached via oligonucleotide assembly, 90% incorporation would be expected to produce a distribution of origami products whereby less than 1% have no dockers, about 1% have one docker, about 5% have two dockers, about 29% have three dockers and about 65% have four dockers. Thus, 65% of the origami products will have the desired four dockers, whilst the other 35% of the origami products will have fewer than the desired number of dockers. The distribution of products is not necessarily problematic for many uses of the compositions, systems and methods set forth herein. However, in some configurations, the presence of origami having an incomplete complement of dockers can adversely impact assay results.

One solution is to design the nucleic acid origami component of an analyte to include an excess quantity of dockers. This can be coupled with a strategy of designing affinity reagents to include a limiting quantity of tethers. Returning to the example of a configuration in which four docker-tether interactions is desired, an analyte component can be designed to include five dockers and an affinity reagent component can be designed to include four tethers. For some configurations, this may be effective. However, in other configurations, the vagaries of synthesis and handling may also impact synthetic yields of the affinity reagent component. As such, a population of affinity reagents that is designed to include four tethers may, in actuality, include a distribution of products including those having a full complement of four tethers and those having fewer than four tethers.

One approach to mitigate complications arising from the combined heterogeneity in the distribution of dockers associated with analytes and in the distribution of tethers associated with affinity reagents is to separate the desired species (i.e. species having the full complement of dockers or tethers) from the undesired species (i.e. species having less than the full complement of dockers or tethers). The species can be separated using methods known in the art such as techniques for separating macromolecules based on differences in mass, charge, charge-to-mass ratio or the like. Another option is to exploit the binding properties of the tethers or dockers by separating the various species based on differences in binding strength resulting from differences in the numbers of the tethers or dockers. For example, affinity chromatography can employ chromatography media having binding partners for the dockers or tethers, or solid-phase extraction utilize solid-phase materials having binding partners for the dockers or tethers.

Another approach to mitigate complications arising from the combined heterogeneity in the distribution of dockers associated with analytes and in the distribution of tethers associated with affinity reagents, is to configure sets of tethers and sets of dockers to have fixed geometries. The geometries can be designed to produce a desired number of subsets of dockers and/or subsets of tethers that interact when the affinity reagents bind to the analytes. The presence of multiple subsets of geometrically configured dockers on a particle or solid support to which an analyte is attached can provide redundant combinations for tether-docker interactions. As such, absence of a particular tether in a subset that is associated with an affinity reagent can be recompensed by presence of another complete subset of tethers. Yet the geometric constraints on the subsets of tethers inhibits more than the desired number of tether subsets from interacting with dockers when the affinity reagent is bound to the analyte. Similarly, analytes can be associated with multiple subsets of dockers which are designed to provide this redundancy and geometric constraint for desired binding interactions with affinity reagents having dockers.

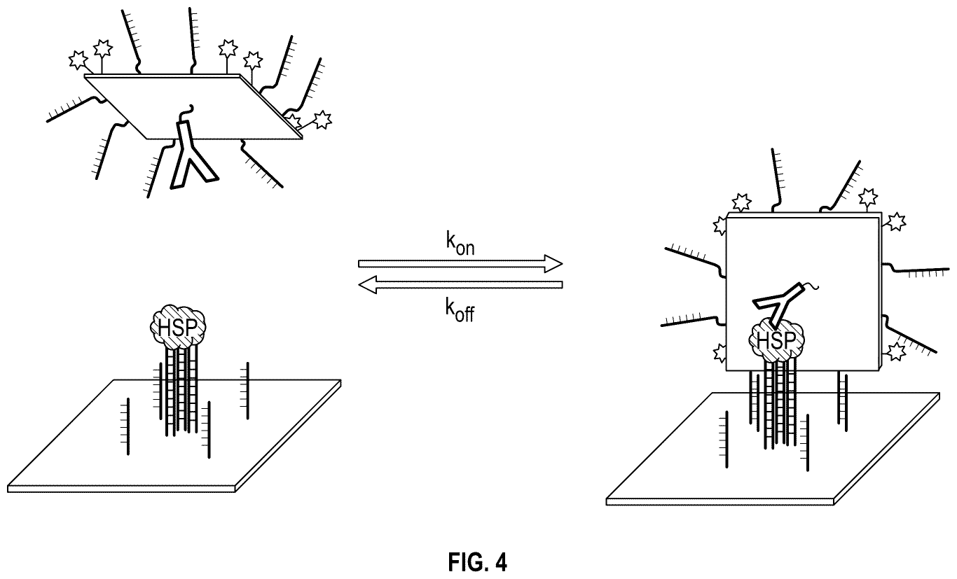

FIG. 4 shows a diagrammatic representation of equilibrium binding between a protein (stippled globule shape) having a set of four nucleic acid dockers (comb shapes) and an affinity reagent having a set of eight nucleic acid tethers (comb shapes) and an antibody component (Y shape). The protein is attached to a structured nucleic acid tile (e.g. composed of a nucleic acid origami and shown as a rectangle shape) via a rigid post composed of multiple nucleic acid helices (ladder shapes). The affinity reagent also includes a structured nucleic acid particle (e.g. composed of a nucleic acid origami and shown as a tile shape) to which the antibody component and tether components are attached. The affinity reagent also includes labels (star shapes) attached to the structured nucleic acid tile.

In the exemplary system of FIG. 4, the set of dockers is geometrically arranged into four subsets of dockers, wherein each of the subsets including a pair of adjacent dockers on the same face of the tile as the protein. In the configuration shown, each docker is a member of two subsets. The set of tethers is geometrically arranged into four subsets of tethers, each subset including two tethers located on an edge of the tile. The affinity reagent tile and protein-bearing tile are configured to constrain the dockers and tethers such that only two of the dockers can bind to respective tethers when the antibody component is bound to the protein. Rigidity of the structured nucleic acid particles contribute to this constraint. The shape of the structured nucleic acid particles also contributes to the constraint. For example, the rigid post creates an obstacle that prevents certain pairs of dockers from simultaneously interacting with two tethers. More specifically, tethers that are positioned diagonally on the protein-bearing structured nucleic acid particle are prevented from simultaneously contacting any pair of dockers due to the obstacle-based constraint provided by the rigid post. It will be understood that a variety of other configurations can utilize one or more of particle rigidity, particle shape, number of dockers, number of tethers, geometric arrangement of dockers, geometric arrangement of tethers, and obstacle-based constraints to facilitate stabilizing effects of docker-tether interactions.

In the configuration exemplified by FIG. 4, up to two dockers can be missing from the protein bearing tile without loss of the stabilizing effect of the interaction between a pair of dockers and a pair of tethers. In other words, so long as one subset of dockers is present, the stabilizing effects of interactions between dockers and tethers can be achieved. Looking to the affinity reagent, up to six tethers can be missing without loss of the stabilizing effect of the interaction between a pair of dockers and a pair of tethers. As such, at least one subset of tethers is present, thereby facilitating the stabilizing effects of interactions between dockers and tethers. It is further notable that the smaller size of the affinity reagent tile compared to the protein-bearing tile and the locations of the tethers on different edges of the tile limit the binding interaction to no more than two pairs of tethers and dockers even though more than two pairs of dockers and/or more than two pairs of tethers may be present. Furthermore, the location of the protein bearing post, in this example being located between several of the dockers, also creates a constraint to limit the binding interaction to no more than two pairs of tethers and dockers even though more than two pairs of dockers and/or more than two pairs of tethers may be present.

The present disclosure provides an assay composition including (a) an analyte attached to a first particle, wherein the analyte includes an epitope, and wherein a set of dockers is attached to fixed positions on the first particle; and (b) an affinity reagent attached to a second particle, wherein the affinity reagent includes a paratope, wherein a set of tethers is attached to fixed positions on the second particle, wherein the paratope recognizes the epitope, wherein the tethers recognize the dockers, and wherein the fixed positions of the set of dockers on the first particle and the fixed positions of the set of tethers on the second particle prevent at least one of the tethers from contacting the set of dockers while (i) the paratope is bound to the epitope and (ii) a subset of the tethers is simultaneously bound to a subset of the dockers.

As exemplified for the above composition, the analyte can be attached to a particle. In some configurations, the particle can be attached to a solid support. For example, the particle can be attached to an address of an array. An analyte and its associated dockers can be attached to a solid support, for example, at an address, without necessarily being attached to a particle. For configurations in which the analyte is immobilized on a solid support, such as an address of an array, the affinity reagent can be present in a fluid phase that is in contact with the solid support. The affinity reagent can bind to the solid support via its paratope binding to an epitope of the analyte along with at least one tether binding to at least one docker. In an alternative configuration, the affinity reagent and its associated tethers can be immobilized on a solid support, such as an address of an array, and the analyte and its associated dockers can be present in a fluid phase that is in contact with the solid support. An affinity reagent and its associated tethers can be attached to a solid support via a particle, but a particle need not mediate attachment of the affinity reagent and its associated tethers with a solid support or with an address of an array. It will be understood that configurations exemplified herein in the context of analytes and associated dockers can be applied to affinity reagents and associated tethers, and vice versa.

The analyte and affinity reagent of the above assay composition can be associated with each other or dissociated. For example, the analyte and affinity reagent can be associated, thereby being in the form of a complex. The complex can include the analyte being bound to the affinity reagent via (i) the paratope being bound to the epitope and (ii) at least one of the tethers being simultaneously bound to at least one of the dockers. Alternatively, the analyte and affinity reagent can be dissociated from each other. Whether the analyte and affinity reagent are in an associated state (i.e. a complex) or dissociated state, they can both be present in a vessel such as a flow cell or test tube. Dissociated analyte and affinity reagent can, nonetheless, be capable of diffusion and in fluid communication with each other. In some configurations, the affinity reagent and analyte can be present in separate vessels, and thus incapable of contact via diffusion. For example, the affinity reagent and analyte can be present in respective vessels of a kit, reagent cartridge or detection system.

In some configurations of a composition set forth herein, a set of dockers that is associated with an analyte is in molar excess compared to a set of tethers that is associated with an affinity reagent. Alternatively, a set of tethers that is associated with an affinity reagent can be in molar excess compared to a set of dockers that is associated with an analyte. In yet another configuration, a set of dockers that is associated with an analyte can have the same stoichiometry as a set of tethers that is associated with an affinity reagent. It will be understood that the above comparative sizes for sets of dockers and tethers can refer to the design for the analyte and affinity reagent or they can refer to the manufactured product.

A set of dockers, or a subset of dockers, can include at least 2, 3, 4, 5, 6, 7, 8, 9, 10, 15, 20, 25 or more dockers. Alternatively or additionally, a set of dockers, or a subset of dockers can include at most 50, 25, 20, 15, 10, 9, 8, 7, 6, 5, 4, 3, 2 or fewer. Typically, a set or subset of dockers will be homogenous with respect to composition. For example, all members of a set or subset of nucleic acid dockers can have a common nucleotide sequence. Alternatively, the dockers in a set or subset can have different nucleotide sequences. Dockers in a set or subset can have a common position with respect to an analyte. The length of nucleic acid dockers in a set or subset can be the same or different. The lengths or compositions of dockers in a set or subset can be selected from those set forth elsewhere herein.

A set of tethers, or a subset of tethers, can include at least 2, 3, 4, 5, 6, 7, 8, 9, 10, 15, 20, 25 or more tethers. Alternatively or additionally, a set of tethers, or a subset of tethers can include at most 50, 25, 20, 15, 10, 9, 8, 7, 6, 5, 4, 3, 2 or fewer. Typically, a set or subset of tethers will be homogenous with respect to composition. For example, all members of a set or subset of nucleic acid tethers can have a common nucleotide sequence. Tethers in a set or subset can have a common position with respect to an affinity agent. Alternatively, the tethers in a set or subset can have different nucleotide sequences. The length of nucleic acid tethers in a set or subset can be the same or different. The lengths or compositions of tethers in a set or subset can be selected from those set forth elsewhere herein.

An analyte can be attached to, or otherwise associated with, a set of dockers that includes at least 1, 2, 3, 4, 5, 6, 7, 8, 9, 10 or more subsets of the dockers. Alternatively or additionally, an analyte can be attached to, or otherwise associated with, a set of dockers that includes at most 10, 9, 8, 7, 6, 5, 4, 3, 2 or 1 subsets of the dockers. The number of subsets in a set of tethers can fall in one or both of these ranges.

A subset of dockers or tethers can be identified according to relative geometry between members. Exemplary configurations will be set forth below for subsets of dockers, but can be applied to subsets of tethers. For example, dockers in a subset can be equidistant from each other. Not all dockers in a subset need be equidistant from each other. For example, a subset of dockers can include three members, one of which is equidistant from the other two members. The distance between dockers can be measured according to the positions where the dockers are attached to a particle or solid support. For example, a set of dockers can include a first docker, a second docker and a third docker, wherein the fixed position for the second docker is equidistant from the fixed position for the first docker and from the fixed position for the third docker. Another measure of distance between dockers can be based on the volume of space that each docker can occupy based on position of attachment and degree of conformational freedom. For example, a set of dockers can include a first docker, a second docker and a third docker, wherein the volume of space accessible to the second docker is equidistant from the volume of space accessible to the first docker and from the volume of space accessible to the third docker. In some configurations, members of a subset of dockers can have at least partially overlapping volumes of space. Alternatively, members of a subset of dockers are not capable of accessing overlapping volumes of space. Generally, dockers in a first subset will not be capable of accessing a volume of space that is accessible to a docker from a second subset of dockers. A subset of dockers may be attached within a common area with respect to an analyte or a unique identifier. For example, an analyte may be attached to a surface of a particle, in which the surface of the particle is divided into two or more regions, and in which a subset of dockers is located only within one of the regions.

Separate subsets of dockers or tethers can be distinguished according to the relative geometry for members within a single subset compared to relative geometry between members of a different subset. This will be exemplified for dockers but applies to tethers as well. For example, a first subset of dockers can include members that are separated by a distance that is less than the distance separating any of those members from any member of a second subset of dockers. Looking to the example of a first subset of dockers that includes members that are equidistant from each other, the members of a second subset of dockers can be separated from the members of the first subset by a distance that is greater than the equidistance. Again, the distances can be measured from the positions at which the dockers are attached to a particle or solid support, or the distances can be measured between the volumes of space that are accessible to the dockers when attached to a particle or solid support.

Separate subsets of dockers or tethers can be distinguished according to composition of the members. Turning to dockers as an example (which can be extended to tethers as well), members of a first subset of nucleic acid dockers can have the same nucleotide sequence or length as each other, whereas members of a second subset of nucleic acid dockers differ in nucleotide sequence or length compared to the members of the first subset of dockers.

Separate subsets of dockers or tethers can be distinguished according to the contours or features of a particle or solid support to which they are attached. An example is provided by the subsets of tethers in the affinity reagent shown in FIG. 4. The tethers of each subset are located on one of four edges of the tile-shaped particle, and the subsets can be distinguished due to members of different subsets being attached to different edges. More generally, a particle can have multiple faces and each of the faces can be attached to a subset of tethers or dockers that is distinct from tethers attached to other faces, the other tethers being members of other subsets. Particles or solid supports can include other contours or features that delineate separate subsets of tethers or dockers including, for example, posts, pits, channels, ridges, corners, sides, or the like.

Separate subsets of dockers can be distinguished according to the geometric grouping of tethers to which they will bind. Conversely, subsets of tethers can be distinguished according to the geometric grouping of dockers to which they will bind. For example, a first subset of dockers that is associated with an analyte can be arranged in a geometry that is complementary to an arrangement of tethers that is associated with an affinity reagent that recognizes the analyte. In this example, a second subset of dockers can be distinguished from the first subset of dockers due to the dockers of the second subset being incapable of binding to the affinity reagent so long as the first subset of dockers is bound to the affinity reagent. Similarly, a first subset of tethers that is associated with an affinity reagent can be arranged in a geometry that is complementary to an arrangement of dockers that is associated with an analyte that is recognized by the affinity reagent. In this example, a second subset of tethers can be distinguished from the first subset of tethers due to the tethers of the second subset being incapable of binding to the analyte so long as the first subset of tethers is bound to the analyte.

Separate subsets of dockers that are attached to an analyte can be distinguished according to the relative distance between an epitope of the analyte and the subset of dockers. One or more dockers, for example in a subset of dockers, can be separated from an epitope by at least 10 nm, 20 nm, 30 nm, 40 nm, 50 nm, 60 nm, 70 nm, 80 nm, 90 nm, 100 nm or more. Alternatively or additionally, one or more dockers, for example in a subset of dockers, can be separated from an epitope by at most 100 nm, 90 nm, 80 nm, 70 nm, 60 nm, 50 nm, 40 nm, 300 nm, 20 nm, 10 nm or less. Optionally, members of a subset of dockers can be equidistant from an epitope of an analyte.

Separate subsets of tethers that are attached to an affinity reagent can be distinguished according to the relative distance between a paratope of the affinity reagent and the subset of tethers. One or more tethers, for example in a subset of tethers, can be separated from a paratope by at least 10 nm, 20 nm, 30 nm, 40 nm, 50 nm, 60 nm, 70 nm, 80 nm, 90 nm, 100 nm or more. Alternatively or additionally, one or more tethers, for example in a subset of tethers, can be separated from a paratope by at most 100 nm, 90 nm, 80 nm, 70 nm, 60 nm, 50 nm, 40 nm, 300 nm, 20 nm, 10 nm or less. Optionally, members of a subset of tethers can be equidistant from a paratope of an affinity reagent.

A method of the present disclosure can include a step of detecting association of an affinity reagent with a unique identifier, thereby identifying an analyte at the unique identifier. Association of affinity reagents with analytes can be detected using any of a variety of techniques that are appropriate to the assay components used. For example, an affinity reagent can be detected at a unique identifier by acquiring a signal from a label attached to the affinity reagent when bound to an analyte at the unique identifier. In some configurations, a complex between an affinity reagent and analyte need not be directly detected, for example, in formats where a nucleic acid tag or other moiety is created or modified as a result of binding. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary detection techniques and apparatus are set forth in U.S. Pat. No. 10,473,654 or US Pat. App. Pub. No. 2022/0162684 A1, each of which is incorporated herein by reference. A detection technique used in a method set forth herein can be configured to resolve addresses (or other unique identifiers) of an array. For example, a detection technique can be configured for single-molecule resolution of analytes.

A method of the present disclosure can be configured to contact an analyte with a series of affinity reagents. This can be facilitated by reversibility of binding interactions between docker-associated analytes and tether-associated affinity reagents. A complex, once formed between a docker-associated analyte and tether-associated affinity reagent, can be dissociated due to reversible binding of an epitope of the analyte with a paratope of the affinity reagent and reversible binding of the docker to the tether. As such, a method set forth herein can be configured to (i) form a complex between a docker-associated analyte and a first tether-associated affinity reagent, (ii) optionally detect the complex, (iii) dissociate the complex and optionally wash away the first tether-associated affinity reagent, and (iv) repeat steps (i) through (iii) using the analyte and a second tether-associated affinity reagent instead of the first tether-associated affinity reagent. A docker-associated analyte can be serially contacted with any number of tether-associated affinity reagents including, for example, at least 2, 3, 4, 5, 10, 25, 50, 100, 200, 300 tether-associated affinity reagents or more. Alternatively or additionally, a docker-associated analyte can be serially contacted with at most 300, 200, 100, 50, 25, 10, 5, 4, 3, or 2 tether-associated affinity reagents. The series of tether-associated affinity reagents that is contacted with a docker-associated analyte can include different tether-associated affinity reagents for each cycle (e.g. the paratopes and/or tethers can differ from one cycle to the next). In some configurations, one or more of the cycles can be repeated using the same species of tether-associated affinity reagent (e.g. the paratope and tether can be the same from one cycle to the next).

Optionally, a method of identifying an analyte can include steps of (a) providing an array of analytes, wherein each of the analytes is attached to a unique identifier in the array, wherein a docker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further includes a tether that recognizes the docker; (c) contacting the array with the affinity reagent, whereby the affinity reagent associates with the first unique identifier via binding of the paratope to the epitope and via binding of the tether to the docker; (d) detecting association of the affinity reagent with the first unique identifier, thereby identifying the analyte at the first unique identifier; (e) removing the affinity reagent from the array; (f) contacting the array with a second affinity reagent, wherein the second affinity reagent includes a second paratope that recognizes a second epitope of the analyte at the first unique identifier of the array, wherein the second paratope preferentially recognizes the analyte compared to other analytes in the array, wherein the second affinity reagent further includes a second tether, whereby the second affinity reagent associates with the first unique identifier via binding of the second paratope to the second epitope and via binding of the second tether to the docker; and (g) detecting association of the second affinity reagent with the first unique identifier, thereby identifying the analyte at the first unique identifier.

An affinity reagent can be dissociated from an analyte, or unique identifier with which it is associated, using reagents or conditions that disrupt interactions between docker and tether and that disrupt interactions between paratope and epitope. For example, dissociation can be achieved by increasing temperature; increasing pH (e.g. by adding NaOH); decreasing pH (e.g. by adding HCl); adding chemical denaturants such as dimethylsulfoxide (DMSO), formamide or alcohol (e.g. ethanol or isopropanol); adding detergent; increasing ionic strength (e.g. increasing concentration of salts such as NaCl or KCl); and/or physical perturbations such as sonication. Dissociation can also be achieved by use of a binding competitor for the paratope-epitope interaction or for the docker-tether interaction. For example, a paratope-epitope interaction can be dissociated by competitive binding with another copy of the epitope or with an analog of the epitope. The paratope-epitope interaction can also be dissociated by competition with a copy of the paratope or with another paratope that binds to the epitope. The docker-tether interaction can be similarly dissociated by competition with a copy of the docker, a copy of the tether or analogs thereof. Nucleic acid-based tethers and dockers can be dissociated by toehold mediated strand displacement or branch migration using a nucleic acid complement of either the tether or docker. A nucleic acid that is used as a tether or docker can include a nucleotide sequence region that functions as a toehold. The toehold region can be upstream or downstream of a region that hybridizes to form a docker-tether duplex. Accordingly, a method may comprise a step of contacting a complex comprising an affinity agent bound to an analyte by coupling of a docker and tether with a nucleic acid strand that is configured to perform toehold mediated strand displacement or branch migration.

A docker-associated analyte that is to be contacted with a plurality of different tether-associated affinity reagents can include a number of dockers that is at least as large as the greatest number of tethers that will be present in any of the tether-associated affinity reagents. As such, in some cycles, the number of dockers that is associated with the analyte can exceed the number of tethers associated with an affinity reagent to which the analyte is bound. In other cycles, the number of dockers can be the same as the number of tethers associated with an affinity reagent to which the analyte is bound. Furthermore, a first affinity reagent that is contacted with an analyte can be associated with an equal, greater or lesser number of tethers compared to the number of tethers associated with a second affinity reagent that is contacted with the analyte.

When using a nucleic acid-based docker and tether, the docker that is associated with a given analyte can have a nucleotide sequence length that is at least as long as the longest hybrid region that is to be formed between the docker and any tether that is associated with an affinity reagent to which the analyte will bind. In a first cycle, a nucleic acid docker can hybridize with a complementary tether associated with a first affinity reagent to form a duplex having a first length. Then, in a second cycle the docker can hybridizes with a complementary tether associated with a second affinity reagent to form a duplex having a second length that is shorter or longer than the first duplex. The different duplex lengths can be selected to achieve a desired level of avidity that is customized for each paratope-epitope pair. For example, a paratope-epitope pair having relatively weak affinity can be associated with a first docker-tether pair having a relatively long duplex, whereas a second docker-tether pair having a shorter duplex can be associated with a higher affinity paratope-epitope pair. Thus, the lengths of the duplexes formed between a given docker and tether can vary from cycle to cycle. However, the lengths of the duplexes need not vary for all cycles and can instead be the same from one cycle to another.

When using a nucleic acid-based docker and tether, the docker that is associated with a given analyte can have a nucleotide sequence that is capable of hybridizing with tethers having any of a variety of different sequences. For example, a nucleic acid docker that is associated with a given analyte can hybridize with a first tether associated with a first affinity reagent to form a first duplex having no mismatches. Then, in a second cycle the docker can hybridizes with a second tether associated with a second affinity reagent to form a second duplex having one or more internal mismatch. The mismatch can be due to the presence of non-complementary nucleotides at positions that would form a base pair if they had been complementary. The mismatch can also be due to an abasic nucleotide or non-naturally occurring base analog in one or both strands of the duplex. The presence of a mismatch will reduce the avidity of the second duplex compared to the first. As such, avidity can be adjusted for a given affinity reagent by designing the duplexes to have different numbers or locations of mismatches. For example, a first paratope-epitope pair having relatively weak affinity can be associated with a first docker-tether pair having a duplex with no mismatches or fewer mismatches compared to the duplex formed by a second docker-tether pair that is associated with a second paratope-epitope pair having stronger affinity for each other. Thus, the presence or location of mismatches in the duplexes formed between a given docker and tether can vary from cycle to cycle.

Generally, any variability in the structures of duplexes formed between dockers and tethers that results in a substantial increase or decrease of duplex melting temperature (Tₘ) can be employed in a method of the present disclosure. Thus, a nucleic acid docker that is associated with a given analyte can hybridize with a first tether associated with a first affinity reagent to form a first duplex having a first Tₘ. Then, in a second cycle the docker can hybridizes with a second tether associated with a second affinity reagent to form a second duplex having a second Tₘ that is lower than the first Tₘ or higher than the first Tₘ. When using nucleic acid-based dockers and tethers, the affinity reagents used from one cycle to another can differ with respect to one or more of the number of docker-tether duplexes formed, the length of one or more docker-tether duplexes formed, the number of mismatches in one or more docker-tether duplexes formed, the location of mismatches in one or more docker-tether duplexes formed, the Tₘ of one or more docker-tether duplexes formed or a combination of at least two of the foregoing.

Hybridization between dockers and tethers can be modulated by the presence of sequences that are capable of forming intra-strand secondary structures to compete with inter-strand hybridization between the dockers and tethers. For example, a palindromic sequence can be configured to form a hairpin at a first Tₘ and to hybridize to a second strand at a second Tₘ. Those skilled in the art will be able to determine appropriate sequence contents and lengths, as well as an ambient temperature and fluid composition to achieve a desired level of competition between a docker intra-strand secondary structure (e.g. hairpin) and a complementary tether or, alternatively, between a tether intra-strand secondary structure (e.g. hairpin) and a complementary docker. The level of competition can be selected to reduce the rate or level of binding between an affinity reagent-associated tether and an analyte-associated docker in the absence of engagement between the paratope and the epitope. In contrast, when the paratope and epitope are bound to each other, the prolonged proximity between docker and tether can provide sufficient time for intra-strand secondary structure in the docker or tether to dissociate, thereby allowing inter-strand hybridization between the docker and tether. In this way, off-target binding of the affinity reagent to the analyte is reduced and avidity of on-target complexes is increased via binding of the tether to the docker. Generally, only one member of a docker and tether pair will contain a sequence that is capable of forming an intra-strand secondary structure (e.g. a palindromic sequence that forms a hairpin); however, it is also possible to include sequences in both the docker and tether that are capable of forming respective intra-strand secondary structures under the conditions used. Any of a variety of models can be used to determine an appropriate balance of dissociation rates for intra-strand secondary structures and association rates for duplex formation including, for example, those set forth in Hertel et al., Nucl. Acids Res. 50:7829-7841 (2022), which is incorporated herein by reference.

A nucleic acid-based tether or docker can include a palindromic sequence that is capable of forming an intra-strand secondary structure (e.g. hairpin structure). The palindromic sequence and intra-strand secondary structure of a tether can be positioned to compete with binding to a complementary docker or, in the case of a docker the palindromic sequence and intra-strand secondary structure can be positioned to compete with binding to a complementary tether. For example, the sequence region of a tether that complements a docker sequence can be present, at least partially, in the palindromic sequence that forms the intra-strand secondary structure. In some configurations, the intra-strand secondary structure can form in a region of the tether that creates a steric hindrance, thereby competing for binding of the tether to a complementary docker. For example, the intra-strand secondary structure can form in a palindromic sequence that is at or near the distal end of the tether (i.e. the end of the tether furthest from the position where the tether attaches to a particle, solid support, or other entity), whereas the region that complements the docker is located at a more proximal region of the tether. The sequence configurations exemplified above for a tether that is capable of interacting with a docker can be present in a docker, thereby mediating hybridization to a complementary tether. The length of a sequence that forms a hairpin or other secondary structure can be at least 2, 3, 4, 5, 6, 7, 8, 9, 10, 12, 14, 16, 18, 20, 25 or more nucleotides. Alternatively or additionally, the length of the sequence can be at most 25, 20, 18, 16, 14, 12, 10, 9, 8, 7, 6, 5, 4, 3 or fewer nucleotides. A nucleotide sequence that forms an intra-strand secondary structure can include multiple nucleation sites, for example, in the form of repeat sequences. The length of a loop region that results from a hairpin can be designed to favor or disfavor formation of a hairpin or other intra-strand secondary structure. For example, a loop region can include at least 4, 5, 6, 7, 8, 9, 10 or more nucleotides. Alternatively or additionally, a loop region can include at most 10, 9, 8, 7, 6, 5, 4 or fewer nucleotides.

The nucleotide composition of a sequence region that is intended to form a hybrid structure can have a larger proportion of A, T or U nucleotides (relative to the number of G and C nucleotides). This can favor faster hybridization at lower temperatures. Alternatively, the nucleotide composition of a sequence region that is intended to form a hybrid structure can have a larger proportion of G or C nucleotides (relative to the number of A, T and U nucleotides). This can favor faster hybridization at higher temperatures. Accordingly, a region of a nucleotide sequence that is intended to form intra-strand or inter-strand base pairs can include, for example, at least 60%, 70%, 80%, 90% or 100% G and/or C nucleotides. Alternatively, a region of a nucleotide sequence that is intended to form an intra-strand or inter-strand base pairs can include, for example, at least 60%, 70%, 80%, 90% or 100% A, T and/or U nucleotides. Moreover, the nucleotide composition of a loop region can be designed to favor or disfavor formation of a hairpin or other intra-strand secondary structure. For example, a region of a nucleotide sequence that is intended to occur in a loop when an intra-strand secondary structure forms can include, for example, at least 60%, 70%, 80%, 90% or 100% G and/or C nucleotides. Alternatively, a region of a nucleotide sequence that is intended to occur in a loop when an intra-strand secondary structure forms can include, for example, at least 60%, 70%, 80%, 90% or 100% A, T and/or U nucleotides.

The dockers or tethers that are used in a method configured for delivery of a plurality of affinity reagents can be universal dockers or universal tethers. The use of universal dockers or tethers can benefit multiplexed methods, thereby allowing a plurality of docker-associated analytes to be processed in parallel even when the analytes differ substantially in their binding to various affinity reagents. Universal dockers or tethers can also facilitate cyclical assays by allowing the same docker or tether to be used for different affinity reagents used in respective cycles. The dockers or tethers that are used in a method configured for delivery of a plurality of affinity reagents can be indexed dockers or indexed tethers. Thus, the methods can be configured to distinguishably process or detect subpopulations of analytes, each subpopulation being effectively tagged by a given docker. Similarly, indexed dockers or tethers can be used for selective binding and/or detection of subsets of affinity reagents to respective analytes based on known or expected affinity of different subsets in the indexed set.

A method for binding a docker-associated analyte with a tether-associated affinity reagent via interaction of a primary paratope with a target epitope of the analyte can utilize an additional epitope and additional paratope as a docker-tether pair. For example, an analyte having a target epitope can be associated with a docker having a copy of the target epitope, and an affinity reagent can have multiple copies of a paratope that recognizes the target epitope. In some cases, the additional epitope need not be identical to the target epitope. For example, the additional epitope can be an analog of the target epitope, whereby the additional epitope and target epitope are recognized by the same paratope. Analogs of peptide epitopes or amino acid epitopes include, for example, those understood in the art to be similar for example according to the BLOSUM Matrix or other similarity score.

Accordingly, the present disclosure further provides a method of identifying an analyte, including steps of (a) providing an array of analytes, wherein each of the analytes is attached to a unique identifier in the array, wherein a docker is attached to each unique identifier in the array, wherein the docker comprises an epitope of an analyte in the array, or an analog of the epitope; (b) providing an affinity reagent, wherein the affinity reagent includes a plurality of paratopes that independently recognize the epitope at a first unique identifier of the array, wherein the paratopes preferentially recognize the analyte compared to other analytes in the array; (c) contacting the array with the affinity reagent, whereby the affinity reagent associates with the first unique identifier via binding of at least one of the paratopes to the epitope and via binding of at least one of the paratopes to the docker; and (d) detecting association of the affinity reagent with the first unique identifier, thereby identifying the analyte at the first unique identifier.

In some configurations, a method of identifying an analyte can include steps of (a) providing an array of analytes, wherein each of the analytes is attached to a unique identifier in the array, wherein a linker is attached to each unique identifier in the array; (b) providing an affinity reagent, wherein the affinity reagent includes a plurality of paratopes that independently recognize an epitope of an analyte at a first unique identifier of the array, wherein the paratopes preferentially recognize the analyte compared to other analytes in the array; (c) attaching a docker to the linker that is attached to each unique identifier in the array, wherein the docker comprises the epitope or analog thereof; (d) contacting the array with the affinity reagent, whereby the affinity reagent associates with the first unique identifier via binding of at least one of the paratopes to the epitope and via binding of at least one of the paratopes to the docker; and (e) detecting association of the affinity reagent with the first unique identifier, thereby identifying the analyte at the first unique identifier.

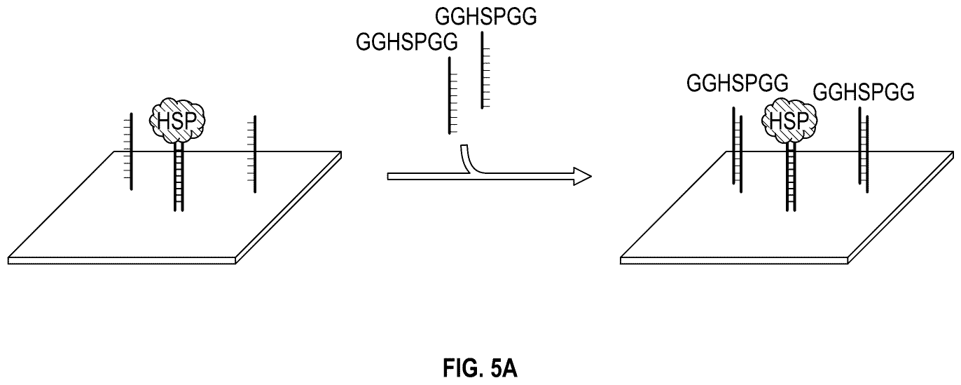

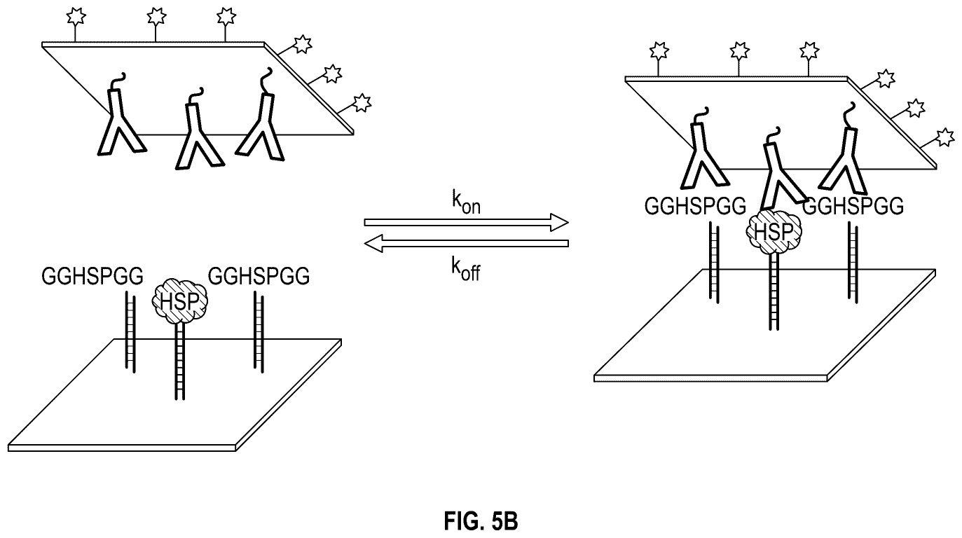

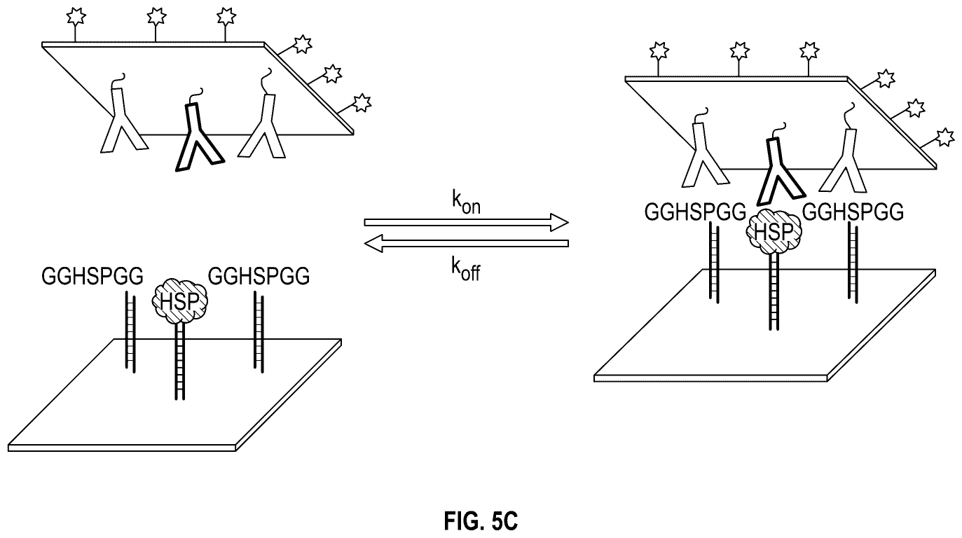

FIG. 5A shows a diagram of a reaction for attaching epitope dockers to a unique identifier in an array. The unique identifier (grey rectangle) is attached to a protein analyte (stippled globule) via a double-stranded nucleic acid linker (ladder) and is also attached to two single-stranded linkers (combs). The unique identifier is contacted with dockers having (1) a peptide sequence (GGHSPGG)(SEQ ID NO: 1), the HSP epitope being present in the protein analyte and the docker, and (2) a nucleic acid strand that is complementary to the single-stranded linkers. As a result, the dockers attach to the unique identifiers, thereby providing two additional copies of the target epitope. FIG. 5B shows a diagrammatic representation of equilibrium binding between the unique identifier of FIG. 5A and an affinity reagent having three antibody components that recognize the HSP epitope.

A linker that is associated with an analyte can be reversibly attached to a docker. As such, the linker can be reused for serial attachment of different dockers. For example, a first epitope docker can be attached to an analyte-associated linker and then the attached epitope docker can be bound to a paratope of a first affinity reagent. The first affinity reagent can then be removed from the analyte (or from the unique identifier to which the analyte and docker are associated) followed by attachment of a second epitope docker to the linker. Then, the second epitope docker can be bound to a paratope of a second affinity reagent. The epitopes of the first and second dockers can be specific for one or more paratopes that are associated with the first affinity reagent and second affinity reagent, respectively. In this way, analyte-associated linkers can allow addition and removal of dockers that are customized for use with a particular affinity reagent. Although the use of linkers is exemplified above in the context of epitope dockers, it will be understood that other dockers can be similarly added and removed from a linker-associated analyte including, for example, a docker that forms a nucleic acid duplex with a tether, docker having a paratope that binds to an epitope tether, a docker having a reactive moiety that covalently bonds to a tether or the like.

The use of linkers can facilitate customization of dockers to accommodate variable assay conditions and/or to accommodate variable affinity reagent characteristics. For example, a pairing of analyte and docker(s) that is effectively detected in a first assay condition may not be satisfactorily detected in a second assay condition. In this situation, the analyte can be paired with a first docker and assayed under a first condition. Then, the first docker can be replaced with a second docker for assay under the second condition. As such, a first docker or first set of dockers can be associated with an analyte for assay under a first condition set forth herein or known in the art, and then the analyte can be associated with a second docker or second set of dockers for assay under a second condition set forth herein or known in the art. Exemplary conditions that can differ for an analyte that is associated with different dockers include, but are not limited to pH, ionic strength, polarity, temperature, duration, buffer composition, concentration of particular assay reagents, or presence of particular assay reagents. In another example, an analyte-docker pair that is accurately detected by a first set of affinity reagents may not be suitably detected by a second set of affinity reagents. In this situation, the analyte can be paired with a first docker for binding assay with a first affinity reagent or with a first set of affinity reagents. Then the first docker can then be replaced with a second docker for assay with a second affinity reagent or with a second set of affinity reagents. For example, a first set of at least 1, 2, 5, 10, 25, 50, 100 or more different affinity reagents can be contacted with an analyte that is paired with a first docker, and a second set of at least 1, 2, 5, 10, 25, 50, 100 or more different affinity reagents can be contacted with an analyte that is paired with a second docker. The different affinity reagents can be delivered in parallel or in series. Alternatively or additionally, to changing the composition of the docker, the number of dockers can be changed in the examples above. Similarly, the number and/or type of tethers that are associated with affinity reagents can be changed as exemplified above for dockers.

Optionally, a method of identifying an analyte can include steps of (a) providing an array of analytes, wherein each of the analytes is attached to a unique identifier in the array, wherein a linker is attached to each unique identifier in the array; (b) providing an affinity reagent, wherein the affinity reagent includes a plurality of paratopes that independently recognize an epitope of an analyte at a first unique identifier of the array, wherein the paratopes preferentially recognize the analyte compared to other analytes in the array; (c) attaching a docker to the linker that is attached to each unique identifier in the array, wherein the docker comprises the epitope or analog thereof; (d) contacting the array with the affinity reagent, whereby the affinity reagent associates with the first unique identifier via binding of at least one of the paratopes to the epitope and via binding of at least one of the paratopes to the docker; (e) detecting association of the affinity reagent with the first unique identifier, thereby identifying the analyte at the first unique identifier; (f) removing the affinity reagent and the docker from the array; (g) attaching a second docker to the linker that is attached to each unique identifier in the array, wherein the second docker includes the second epitope or analog thereof, (h) contacting the array with a second affinity reagent including a plurality of second paratopes that independently recognize a second epitope of the analyte at the first identifier, wherein the second epitope differs from the first epitope, whereby the second affinity reagent associates with the first unique identifier via binding of at least one of the second paratopes to the second epitope and via binding of at least one of the second paratopes to the second docker; and (i) detecting association of the second affinity reagent with the first unique identifier, thereby identifying the analyte at the first unique identifier. Typically, the paratopes preferentially recognize the epitope compared to the second epitope.

An epitope docker that is associated with an analyte can differ from a target epitope of the analyte that is recognized by a primary paratope of an affinity reagent. Thus, the docker can include a secondary epitope that is not substantially recognized by the primary paratope of the affinity reagent. Optionally, the affinity reagent can include a secondary paratope that recognizes the secondary epitope of the docker but does not substantially recognize the primary epitope of the analyte. For example, an analyte having a target epitope can be associated with a docker having a secondary epitope, and an affinity reagent can have a first paratope that recognizes the target epitope and a second paratope that recognizes the secondary epitope. FIG. 5C shows a diagrammatic representation of equilibrium binding between (1) a unique identifier attached to a protein analyte with a target epitope (HSP) and attached to two dockers having secondary epitopes (DTR), and (2) an affinity component (black Y-shape) having a paratope that binds the target epitope and two affinity components (grey Y-shape) that bind the secondary epitopes. It will be understood that the use of a secondary epitope as docker and secondary paratope as tether in FIGS. 5A through 5C is exemplary. Alternatively, a secondary paratope can be used as a docker and a secondary epitope can be used as a tether.

Any number of epitopes and paratopes can be used as dockers and tethers. For example, at least 1, 2, 3, 4, 5, 6, 7, 8, 9, 10, 12, 15, 20 or more epitopes can bind to paratopes to improve avidity of a complex. Alternatively or additionally, at most 20, 15, 12, 10, 9, 8, 7, 6, 5, 4, 3, 2 or 1 epitopes can bind to paratopes to improve avidity of a complex. The epitopes can be copies of target epitopes that are present in an analyte that is associated with the dockers or secondary epitopes.

In some configurations of the methods and compositions set forth herein, an analyte can be associated with a docker that binds to an endogenous moiety of an affinity reagent. Thus, the affinity reagent itself can function as a tether. For example, an analyte can be associated with an antibody binding protein that functions as a docker. Exemplary antibody binding proteins that can be used as a docker include, but are not limited to, a gamma Fc receptor, protein A, protein L, protein G or ‘secondary’ antibody raised to recognize the antibody component. Similarly, a nucleic acid docker can be associated with an analyte and the nucleic acid docker can bind to an endogenous nucleotide sequence of a nucleic acid aptamer moiety of an affinity reagent. Other dockers and tethers include pairs of affinity tags and their receptors used for affinity purification of recombinant proteins. For example, an antibody or other recombinant protein that is present in an affinity reagent can include an affinity tag such as polyhistidine, glutathione-S-transferase (GST), maltose binding protein (MBP), strep-tag, Green Fluorescent Protein (or wavelength-shifted variant thereof), Calmodulin-Binding Protein (CBP), biotinylation tag, or others known in the art. A SNAP can include a docker having a moiety that binds to the tag such as an antibody, glutathione, maltose, streptavidin, avidin, calmodulin, Ni²⁺ (e.g. Ni(NTA)) or the like.

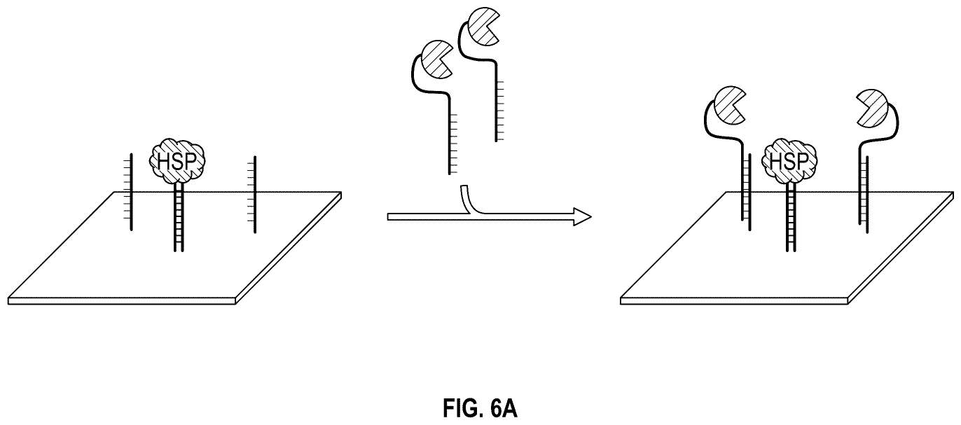

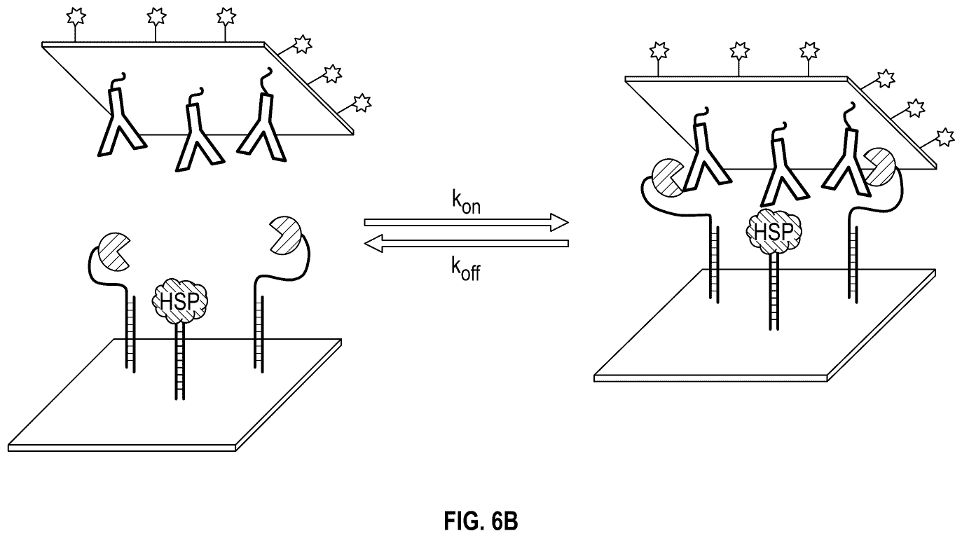

FIG. 6A shows a diagram of a reaction for attaching a unique identifier in an array to a docker having an antibody receptor (e.g. Protein A or a secondary antibody that binds to the antibody). The unique identifier (grey rectangle) is attached to a protein analyte (stippled globule) via a double-stranded nucleic acid linker (ladder) and is also attached to two single-stranded linkers (combs). The unique identifier is contacted with dockers having an antibody receptor (striped circle with cut-out), and further including a nucleic acid strand that is complementary to the single-stranded linkers. As a result, the dockers attach to the unique identifier so as to display two antibody receptors. FIG. 6B shows a diagrammatic representation of equilibrium binding between the unique identifier of FIG. 6A and an affinity reagent having three antibodies that recognize the HSP epitope of the protein analyte. Avidity of the complex is enhanced by binding of the antibody receptors to the antibody components of the affinity reagent.

In some configurations of the methods and compositions set forth herein, dockers and tethers can form covalent attachments to each other. Accordingly, a method of identifying an analyte can include steps of (a) providing an array of analytes, wherein each of the analytes is attached to a unique identifier in the array, wherein a docker is attached to each unique identifier in the array, wherein the docker comprises a first reactive moiety; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further comprises a second reactive moiety; (c) contacting the array with the affinity reagent, whereby the affinity reagent associates with the first unique identifier via binding of at least one of the paratopes to the epitope and via the covalently linked product of reaction between the first reactive moiety with the second reactive moiety; and (d) detecting association of the affinity reagent with the first unique identifier, thereby identifying the analyte at the first unique identifier.

Optionally, a method of identifying an analyte can include steps of (a) providing an array of analytes, wherein each of the analytes is attached to a unique identifier in the array, wherein a linker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further comprises a first reactive moiety; (c) attaching a second reactive moiety to the linker, wherein the second reactive moiety is reactive toward the first reactive moiety to form a covalently linked product; (d) contacting the array with the affinity reagent, whereby the affinity reagent associates with the first unique identifier via binding of at least one of the paratopes to the epitope and via the covalently linked product of reaction between the first reactive moiety with the second reactive moiety; and (e) detecting association of the affinity reagent with the first unique identifier, thereby identifying the analyte at the first unique identifier.

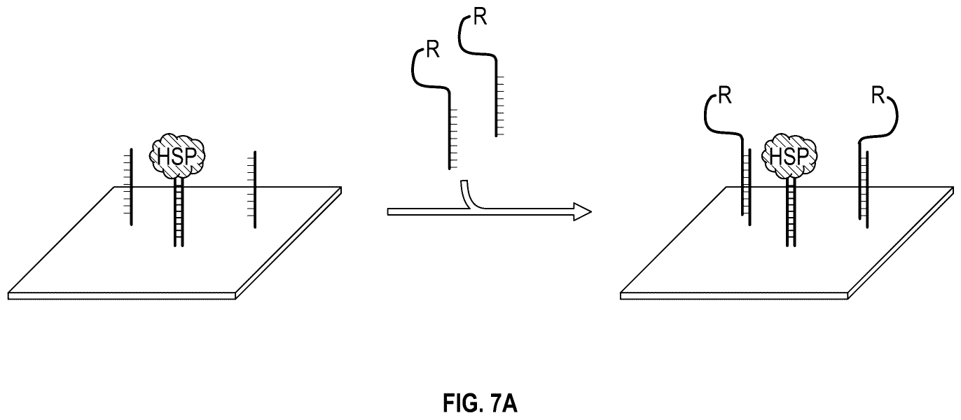

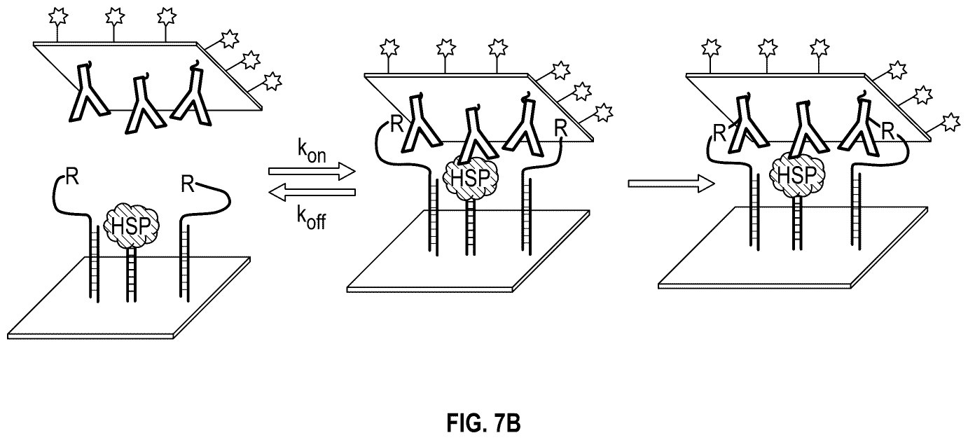

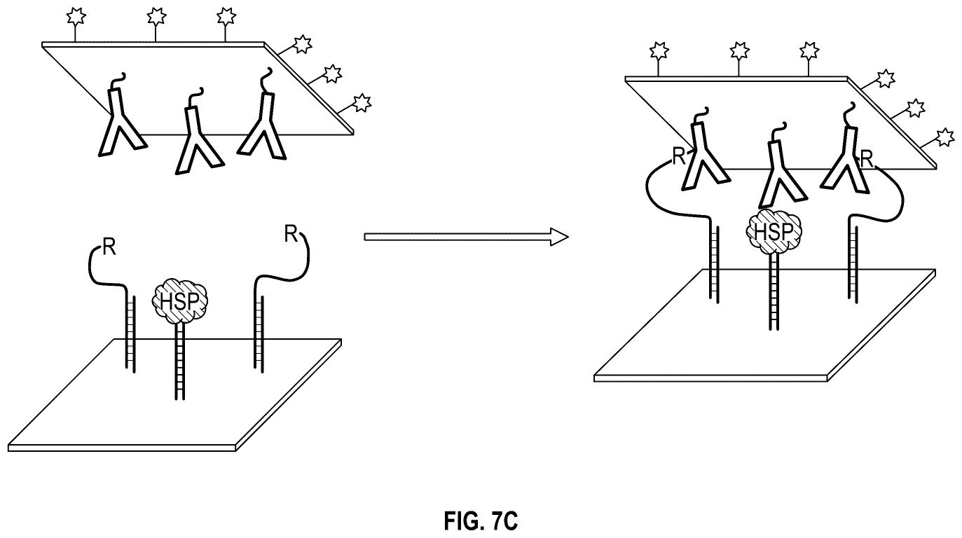

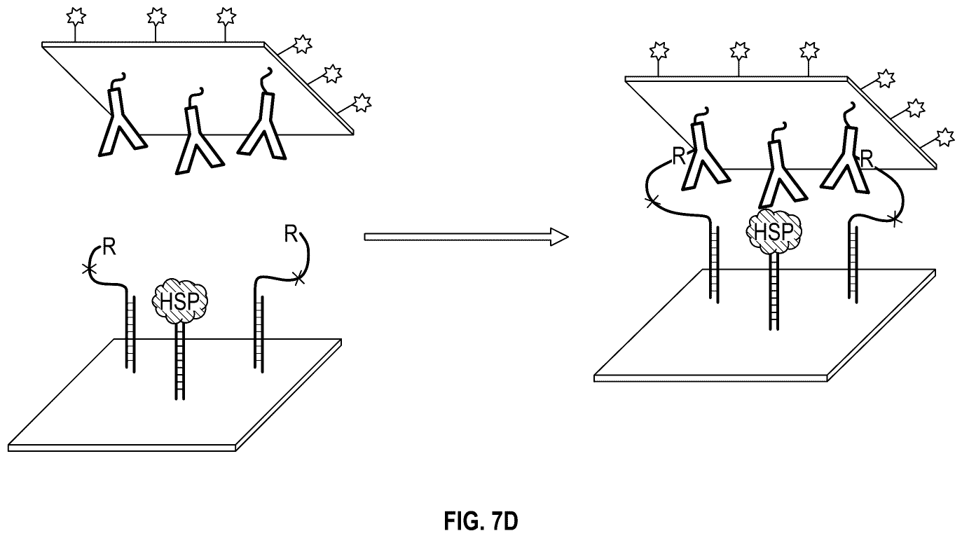

FIG. 7A shows a diagram of a reaction for attaching a unique identifier to a docker having a reactive moiety. The unique identifier (grey rectangle) is attached to a protein analyte (stippled globule) via a double-stranded nucleic acid linker (ladder) and is also attached to two single-stranded linkers (combs). The unique identifier is contacted with dockers having reactive moiety R, each of the dockers further including a nucleic acid strand that is complementary to the single-stranded linkers. As a result, the dockers attach to the unique identifier, thereby displaying two copies of reactive moiety R.

FIG. 7B shows a diagrammatic representation of two steps of a reaction for attaching the unique identifier of FIG. 7A to an affinity reagent. The first reaction is equilibrium binding between the unique identifier and the affinity via non-covalent interaction of an antibody moiety of the affinity reagent with the HSP epitope of the protein analyte. In the second reaction the two copies of reactive moiety R bond covalently to antibody moieties of the affinity reagent. Reactive moiety R can react with an endogenous amino acid of the antibody or with a reactive moiety of a functionalized amino acid of the antibody, such as an amino acid having a click moiety.

FIG. 7C shows a diagrammatic representation of a single step reaction for attaching an affinity reagent to a unique identifier having a protein analyte of interest. A first antibody moiety of the affinity reagent binds non-covalently to the HSP epitope of the protein and reactive moieties (R) of the unique identifier form covalent linkages with second and third antibody moieties of the affinity reagent. The reactive moieties and HSP epitope need not react with different antibody moieties of the affinity reagent. For example, at least one of the reactive moieties can covalently bond to an antibody that is also bound to the HSP epitope.

Any of a variety of reactive moieties can be used to form a covalent bond between a docker and tether. Those skilled in the art will know or be able to readily determine chemistries appropriate to the reagents in use. Particularly useful reactive moieties include reactive amino acid side chains, bioorthogonal moieties and click-type moieties. Examples of useful crosslinking reactions and reactive moieties include, but are not limited to, those set forth in U.S. Pat. No. 11,203,612 or 11,505,796; or US Pat. App. Pub. No. 2022/0162684 A1, each of which is incorporated herein by reference. Particularly useful linkers have nucleotide sequences that are complementary to nucleotide sequences attached to dockers.

A method that utilizes covalent bonding of dockers to tethers can be configured to serially contact an analyte with affinity reagents. Accordingly, a method of identifying an analyte, can include steps of (a) providing an array of analytes, wherein each of the analytes is attached to a unique identifier in the array, wherein a linker is attached to each unique identifier in the array; (b) providing an affinity reagent, wherein the affinity reagent includes a paratope that recognizes an epitope of an analyte at a first unique identifier of the array, wherein the paratope preferentially recognizes the analyte compared to other analytes in the array, wherein the affinity reagent further comprises a first reactive moiety; (c) attaching a second reactive moiety to the linker, wherein the second reactive moiety is reactive toward the first reactive moiety to form a covalently linked product; (d) contacting the array with the affinity reagent, whereby the affinity reagent associates with the first unique identifier via binding of at least one of the paratopes to the epitope and via the covalently linked product of reaction between the first reactive moiety with the second reactive moiety; and (e) detecting association of the affinity reagent with the first unique identifier, thereby identifying the analyte at the first unique identifier (f) removing the affinity reagent and the covalently linked product from the array; (g) attaching a third reactive moiety to the linker, wherein the third reactive moiety is reactive toward a fourth reactive moiety to form a covalently linked product; (h) contacting the array with a second affinity reagent, wherein the second affinity reagent includes a paratope that recognizes a second epitope of the analyte at the first unique identifier of the array, wherein the paratope preferentially recognizes the analyte compared to other analytes in the array, wherein the affinity reagent further includes the fourth reactive moiety, whereby the affinity reagent associates with the first unique identifier via binding of at least one of the paratopes to the epitope and via the covalently linked product of reaction between the third reactive moiety with the fourth reactive moiety; and (i) detecting association of the second affinity reagent with the first unique identifier, thereby identifying the analyte at the first unique identifier.

Optionally, reactive moieties used on dockers and/or tethers are inert until activated. This can prevent the reactive moieties from reacting during association of the paratope and epitope, thereby not substantially influencing association of the affinity reagent with the analyte or its unique identifier. Reaction can be activated after association between the paratope and epitope has occurred, for example, after binding equilibrium has been achieved. Photo-reactive agents and moieties, such as photo-crosslinkers, are particularly useful. For example, a docker or tether can be derivatized with a photo-crosslinker which can be activated by irradiation with light of an appropriate wavelength to form crosslinks that covalently attach the docker and tether. In some configurations, a docker that is derivatized with a photo-crosslinker can be irradiated to form a crosslink with a protein moiety of an affinity reagent such as an amino acid present in an antibody. Other methods for activating reactions will be known to those skilled in the art in view of the characteristics of the reactive moieties used and can include, for example, changes in pH, changes in temperature, delivery of cofactors, uncaging of cofactors, delivery of catalysts or uncaging of catalysts.

Another useful type of reactive moiety is a substrate for an enzyme. For example, peroxidases can catalyze oxidation of various organic substrates by hydrogen peroxide to form highly reactive species that can covalently bond to nearby moieties. For example, horseradish peroxidase or ascorbate peroxidase can convert phenols to free radicals which are highly reactive with atoms found in nucleic acids or proteins. A docker or tether can be derivatized with a moiety that functions as a peroxidase substrate and the moiety can be contacted with peroxidase and hydrogen peroxide to form crosslinks that covalently attach the docker and tether. In some configurations, a docker that is derivatized with a peroxidase substrate can be reacted with peroxidase and hydrogen peroxide to form a crosslink with a protein moiety of an affinity reagent such as an amino acid present in an antibody.

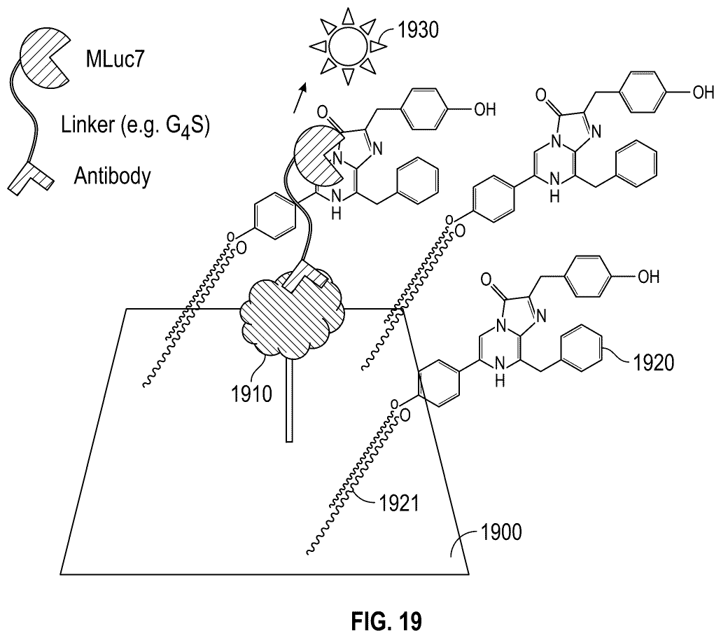

Other binding ligands may be useful as components of a docker/tether system. Binding ligands could include allosteric regulators that bind to a complementary protein. Likewise, certain cofactors that bind to a complementary protein may be useful. A particularly useful cofactor may include a cofactor for a luminescent protein. For example, certain luminescent luciferase enzymes emit light when bound to a cofactor molecule such as coelenterazine. FIG. 19 illustrates a system incorporating a luminescent enzyme (e.g., MLuc7) as a tether and a cofactor 1920 (e.g., coelenterazine) for the luminescent enzyme as a component of a docker strand. An analyte 1910 is attached to a particle 1900 (e.g., a nucleic acid nanoparticle). The particle is further attached to a plurality of cofactors 1920 that are immobilized by optional linking moieties 1921 (e.g., oligonucleotides, PEG moieties, etc.) In some cases, a linking moiety may comprise a plurality of attached cofactors 1920, thereby facilitating an increased intensity or time length of a luminescent signal 1930. For example, a linking moiety may comprise a branched or dendrimeric polymer attached to a plurality of cofactors for the luminescent enzyme. An affinity reagent is provided, in which the affinity reagent is attached to the luminescent enzyme by an optional linking moiety. In the depicted configuration of FIG. 19, the affinity reagent (e.g., an antibody or aptamer) is bound to the analyte 1910 and the cofactor 1920 is bound to the luminescent enzyme, thereby facilitating increased avidity of the affinity reagent for the analyte and/or providing a detectable signal 1930 that facilitates detection of the affinity reagent bound to the analyte 1910.

An affinity reagent that is attached to an analyte via a covalently linked docker-tether pair, can be removed from the analyte using any of a variety of methods. For example, denaturation conditions can be used to dissociate the paratope from the epitope and to dissociate the dockers from the linkers. Alternatively or additionally, the docker or tether can include a scissile linkage and reagents that break the scissile linkage can be contacted with the complex. For example, FIG. 7D shows dockers having scissile linkages as indicated by an ‘X’. Exemplary scissile linkages include, but are not limited to, thioethers which can be reductively cleaved, peptide linkers or carbohydrate linkers which can be enzymatically cleaved, photoreactive moieties which can be photolyzed, nucleic acid linkers which can be cleaved by endonucleases, duplex nucleic acid linkers which can be disconnected via denaturation, or the like. Scissile linkers and reagents for their cleavage can be obtained from commercial sources such as BroadPharm (San Diego, CA), ThermoFisher (Waltham, MA) or Sigma Aldrich (St. Louis, MO). In some cases, dissociation can occur due to denaturation of a duplex formed between a nucleic acid linker and docker.

An array-based method of the present disclosure can be configured to use indexed dockers, wherein a first subset of unique identifiers in the array is associated with a first subset of dockers and a second subset of unique identifiers in the array is associated with a second subset of dockers. Members of the first subset of unique identifiers can be distinguished from members of the second subset of unique identifiers based on differential binding of the first and second docker subsets to various affinity reagents. Differential binding of first and second affinity reagents can result from the first affinity reagent being associated with a first tether that preferentially binds to the first subset of dockers and the second affinity reagent being associated with a second tether that preferentially binds to the second subset of dockers.

Optionally, an array can include at least 2, 3, 4, 5, 10, 25 or more subsets of indexed dockers, wherein each subset of indexed dockers is associated with a respective subset of unique identifiers in the array. The availability of different subsets of unique identifiers can facilitate sample multiplexing, whereby each subset of unique identifiers is attached to analytes from a respective sample. For example, a first subset of unique identifiers can be attached to proteins from a first biological sample and a second subset of unique identifiers can be attached to proteins from a second biological sample.

Different subsets of unique identifiers can also provide super-resolution detection of analytes on an array surface. For example, addresses can be present on the surface of an array at a density that exceeds the resolution of a detector, but a detection event can be configured to observe signal from a first subset of the addresses while a second subset of addresses does not produce detectable signal. This can effectively reduce the density of addresses observed during the detection event. The second subset of addresses can then be observed in a second detection event while the first subset of addresses does not produce signal. Optimal spacing can be achieved by interleaving the addresses of the two subsets in a way that nearest neighbor addresses in the overall array belong to different subsets of addresses.

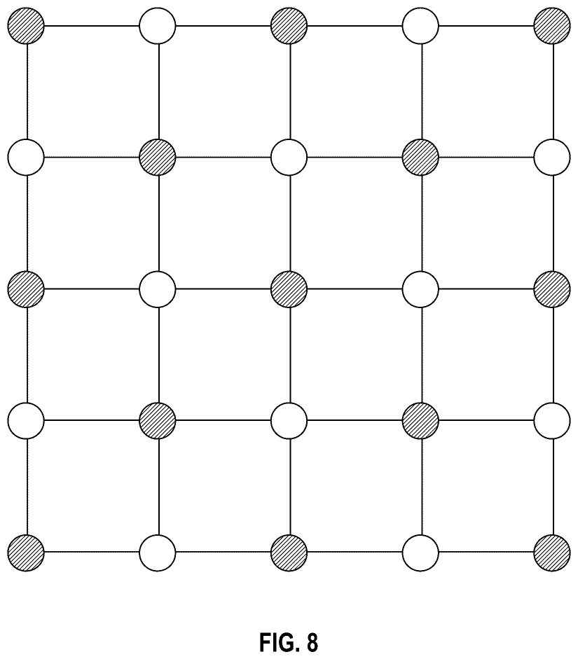

FIG. 8 shows a diagram of an array of addresses arranged in a rectilinear grid. The grid is indicated by the lines and the addresses are indicated by circles at the vertices of the grid. Two subsets of addresses are present in the array, the black circles indicating addresses attached to a first subset of indexed dockers and the grey circles indicating addresses attached to a second subset of indexed dockers. In this example, a detector that is not able to resolve nearest neighbor addresses from each other may nonetheless be capable of resolving each of the black addresses from all other black addresses in the array and may also be capable of resolving each of the grey addresses from all other grey addresses in the array. This resolution can be achieved by contacting the array with affinity reagents that selectively bind to only one subset of addresses, for example, by virtue of having tethers that selectively recognize dockers associated with only one subset of addresses. The use of a rectilinear array is exemplary. Other grids can be used such as hexagonal grids. In some cases, an array of randomly spaced addresses can be resolved using random distribution of addresses from different subsets, for example, by loading the array according to an acceptable probability that addresses from a given subset will not occur as nearest neighbors in the array.

Accordingly, a method of identifying analytes can include steps of (a) providing an array of analytes, wherein each of the analytes is attached to a unique identifier in the array, wherein a first docker is attached to a first subset of unique identifiers in the array and a second docker is attached to a second subset of unique identifiers in the array; (b) contacting the array with a first set of affinity reagents, wherein the first set of affinity reagents is attached to first tethers that bind selectively to the first docker compared to the second docker, whereby the first set of affinity reagents associate with the first subset of unique identifiers via binding of the paratope to the epitope and via binding of the tether to the docker; (c) detecting association of a first affinity reagent with a unique identifier of the first subset, thereby identifying an analyte at a first unique identifier; (d) contacting the array with a second set of affinity reagents, wherein the second set of affinity reagents is attached to second tethers that bind selectively to the second docker compared to the first docker, whereby the second set of affinity reagents associate with the second subset of unique identifiers via binding of the paratope to the epitope and via binding of the tether to the docker; and (e) detecting association of a second affinity reagent with a unique identifier of the second subset, thereby identifying an analyte at a second unique identifier.

A method of the present disclosure can employ affinity reagents that are associated with multiple subsets of tethers and/or analytes that are associated with multiple subsets of dockers. For example, a method of the present disclosure can include steps of (a) providing an analyte attached to a first particle or solid support, wherein the analyte includes an epitope, and wherein a set of dockers is attached to fixed positions on the first particle or solid support; (b) contacting the analyte with an affinity reagent, wherein the affinity reagent is attached to a second particle, wherein the affinity reagent includes a paratope, wherein a set of tethers is attached to fixed positions on the second particle, wherein the paratope binds to the epitope, wherein a subset of the tethers binds to a subset of the dockers, and wherein the fixed positions of the set of dockers on the first particle or solid support and the fixed positions of the set of tethers on the second particle prevent at least one of the tethers from contacting the set of dockers while (i) the paratope is bound to the epitope and (ii) the subset of the tethers is simultaneously bound to the subset of the dockers, thereby forming a complex including the analyte and the affinity reagent. Exemplary compositions for the affinity reagents, analytes and their component parts include those set forth previously herein. Some are set forth below.

In particular configurations of the above method, the first particle or solid support is immobilized in a flow cell and step (b) is carried out by delivering the affinity reagent to the flow cell. It will be understood that the affinity reagent can be immobilized instead of the analyte. For example, the affinity reagent can be immobilized in a flow cell and the analyte can be delivered in fluid phase to the flow cell. The analyte can be attached to a particle when delivered to the flow cell.

Optionally, the above method can further include a step of dissociating the affinity reagent from the analyte and removing the affinity reagent from contact with the first particle or solid support. For example, the affinity reagent can be removed from a flow cell to which the analyte is immobilized. For configurations in which the affinity reagent is immobilized, the analyte can be removed from contact with the affinity reagent, for example by removal from a flow cell to which the affinity reagent is immobilized. An affinity reagent or analyte that is removed can be replaced with another affinity reagent or analyte respectively. For example, the other affinity reagent or analyte can be delivered to the flow cell. An iterative process can be carried out in which a series of affinity reagents or analytes is delivered and removed. For example, the iterative process can include detection steps carried out between the addition of reagents and their removal. Whether the process is iterative or not, a method set forth herein can include a step of detecting a complex formed between an affinity reagent and analyte.

A method set forth above can be carried out in a fluid and can further include a step of separating an analyte-affinity reagent complex from the fluid. Optionally, the separated complex can be processed to dissociate the analyte from the affinity reagent. Accordingly, a method set forth herein can be used to prepare an analyte or affinity reagent for further use. A method that is used to prepare an analyte or affinity reagent can optionally include a detection step, but need not necessarily include a detection step.

Any of a variety of assay formats can be used to detect an analyte. Several methods will be exemplified below in the context of detecting proteins but can readily be extended to other analytes by modifications that will be apparent to those skilled in the art. The exemplified methods can utilize tether-associated affinity reagent(s) and/or docker-associated analyte(s).

A protein can be detected using one or more affinity reagents having binding affinity for the protein. The affinity reagent and the protein can bind each other to form a complex and the complex can be detected during or after formation. The complex can be stabilized by interaction of docker(s) and tether(s). The complex can be detected directly, for example, due to a label that is present on the affinity reagent or protein. In some configurations, the complex need not be directly detected, for example, in formats where the complex is formed and then the affinity reagent, protein, or a label component that was present in the complex is subsequently detected.

Many protein assays, such as enzyme linked immunosorbent assay (ELISA), achieve high-confidence characterization of one or more proteins in a sample by exploiting high specificity binding of affinity reagents to the protein(s) and detecting the binding event while ignoring all other proteins in the sample. Binding assays can be carried out by detecting affinity reagents and/or proteins that are immobilized in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FLEXMAP 3D®.

Exemplary assay formats that can be performed at a variety of plexity scales up to and including proteome scale are set forth in U.S. Pat. No. 10,473,654 or US Pat. App. Pub. Nos. 2020/0318101 A1 or 2020/0286584 A1; U.S. patent application Ser. No. 18/045,036, or Egertson et al., BioRxiv (2021), DOI: 10.1101/2021.10.11.463967, each of which is incorporated herein by reference. A plurality of proteins can be assayed for binding to affinity reagents, for example, on single-molecule resolved protein arrays. Proteins can be in a denatured state or native state when manipulated or detected in a method set forth herein.

Turning to the example of an array-based assay configuration, the identity of an extant protein at any given address is typically not known prior to performing the assay. The assay can be used to identify extant proteins at one or more addresses in the array. A plurality of affinity reagents, optionally labeled (e.g. with fluorophores), can be contacted with the array, and the presence of affinity reagents can be detected at individual addresses to determine binding outcomes. A plurality of different affinity reagents can be delivered to the array and detected serially, such that each cycle detects binding outcomes for a given type of affinity reagent (e.g. a type of affinity reagent having affinity for a particular epitope) at each address. The assay can include a step of dissociating affinity reagents from the array after detecting the binding outcomes, such that the next affinity reagent can be delivered to the flow cell and detected. In some configurations, a plurality of different affinity reagents can be detected in parallel, for example, when different affinity reagents are distinguishably labeled.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers can be expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. No. 10,473,654; US Pat. App. Pub. No. 2020/0318101 A1; U.S. patent application Ser. No. 18/045,036, or Egertson et al., BioRxiv (2021), DOI: 10.1101/2021.10.11.463967, each of which is incorporated herein by reference.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g. the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g. technology developed by SomaLogic or Palamedrix) may also be useful for detecting proteins. Accordingly, a method of detecting a terminal amino acid or a derivative thereof utilizing an affinity agent may be combined with methods of dockers and tethers set forth herein.

Cyclical removal of terminal amino acids from a protein can be carried out using an Edman-type sequencing reaction. In some configurations, an Edman-type sequencing reaction can involve reaction of a phenyl isothiocyanate with an N-terminal amino group of a protein under mildly alkaline conditions (e.g., about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

Edman-type processes can be carried out in a multiplex format to detect, characterize or identify a plurality of proteins. A method of detecting a protein can include steps of (i) exposing a terminal amino acid on a protein at an address of an array, where the address of the array optionally comprises a docker; (ii) binding an affinity agent to the terminal amino acid, where the affinity agent includes a nucleic acid tag and optionally a tether that binds to the docker, and where a primer nucleic acid is present at the address; (iii) extending the primer nucleic acid in the presence of the nucleic acid tag, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. The extending of a primer can be carried out, for example, by polymerase-based extension of the primer, using the nucleic acid tag as a template. Alternatively, the extending of a primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g., PCR-based detection, or rolling circle amplification-based detection) or nuclei acid sequencing (e.g., cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. Optionally, the contacting may include a docker-tether interaction as set forth herein, thereby facilitating association of the first protein to the second protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. In each case, the other molecules may be provided with a tether that facilitates association of the other molecule to a protein that is co-located with a docker. For example, a protein that participates in a signal transduction pathway can be identified as a particular candidate protein by detecting binding to a second protein that is known to be a binding partner for the candidate protein in the pathway.

One or more compositions set forth herein can be present in an apparatus or vessel. For example, a tether-associated affinity reagent and/or docker-associated analyte of the present disclosure can be present in a vessel, such as a flow cell. In some cases, the tether is associated with the affinity reagent via a particle (e.g. structured nucleic acid particle). Similarly, the docker can be associated with the analyte via a particle (e.g. structured nucleic acid particle). As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. The one or more reservoirs can contain tether-associated affinity reagents that recognize one or more docker-associated analytes in the vessel. Optionally, a detection apparatus can be configured to include a waste receptacle to which waste from the vessel is collected. For example, tether-associated affinity reagents can be delivered from the apparatus through an ingress of a flow cell and waste can be removed through an egress of the flow cell to the apparatus.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

### Example I

### Structured Nucleic Acid Particles Having Dockers and Labelled Probes Having Tethers; Tile-Shaped Origami Particles

This example describes structured nucleic acid particles (SNAPs) having nucleic acid dockers. Labelled probes (Lobes) having nucleic acid tethers are also described.

The origami structure of the SNAP is composed of the p7249 scaffold (Bayou Biolabs, LA) and the oligonucleotides listed in Table 1. The origami SNAP folds to produce a tile-shaped structure.

TABLE 1

| | | SEQ |
|---|---|---|
| Sequence | | ID |
| Name | Sequence | NO |
| 1-Reg- | TCATTTGCTAATAGTAGTAGCATT | 2 |
| TIR01C6 | | |
| 2-Reg- | ACCCTCATTCAGGGATAGCAAGCC | 3 |
| T2R01C6 | | |
| 3-Reg- | AGAATATCAGACGACGACAATAAA | 4 |
| T3R01C6 | | |
| 4-Reg- | TCATATGCGTTATACAAAGGCGTT | 5 |
| T3R03C5 | | |
| 5-Reg- | CGGGAGAATTTAATGGAAACAGTA | 6 |
| T3R09C2 | | |
| 6-Reg- | GCGCGTACTTTCCTCGTTAGAATC | 7 |
| T4R01C6 | | |
| 7-Reg- | TTAAGAGGGTCCAATACTGCGGATAGCGAG | 8 |
| TIR06C3 | | |
| 8-Reg- | AGGCTTTTCAGGTAGAAAGATTCAATTACC | 9 |
| TIR08C3 | | |
| 9-Reg- | TTATGCGATTGACAAGAACCGGAGGTCAAT | 10 |
| TIR10C3 | | |
| 10-Reg- | CATAAGGGACACTAAAACACTCACATTAAA | 11 |
| T1R12C3 | | |
| 11-Reg- | CGGGTAAAATTCGGTCGCTGAGGAATGACA | 12 |
| T1R14C3 | | |
| 12-Reg- | GTCTCTGACACCCTCAGAGCCACATCAAAA | 13 |
| T2R06C3 | | |
| 13-Reg- | TCACCGGAAACGTCACCAATGAATTATTCA | 14 |
| T2R08C3 | | |
| 14-Reg- | TTAAAGGTACATATAAAAGAAACAAACGCA | 15 |
| T2R10C3 | | |
| 15-Reg- | ATAATAACTCAGAGAGATAACCCGAAGCGC | 16 |
| T2R12C3 | | |
| 16-Reg- | ATTAGACGGAGCGTCTTTCCAGAGCTACAA | 17 |
| T2R14C3 | | |
| 17-Reg- | TATATAACGTAAATCGTCGCTATATTTGAA | 18 |
| T3R06C3 | | |
| 18-Reg- | TTACCTTTACAATAACGGATTCGCAAAATT | 19 |
| T3R08C3 | | |
| 19-Reg- | ATTTGCACCATTTTGCGGAACAAATTTGAG | 20 |
| T3R10C3 | | |
| 20-Reg- | GATTTAGATTGCTGAACCTCAAAGTATTAA | 21 |
| T3R12C3 | | |
| 21-Reg- | CACCGCCTGAAAGCGTAAGAATACATTCTG | 22 |
| T3R14C3 | | |
| 22-Reg- | TGAGTGTTCAGCTGATTGCCCTTGCGCGGG | 23 |
| T4R06C3 | | |
| 23-Reg- | GAGAGGCGACAACATACGAGCCGCTGCAGG | 24 |
| T4R08C3 | | |
| 24-Reg- | TCGACTCTGAAGGGCGATCGGTGCGGCCTC | 25 |
| T4R10C3 | | |
| 25-Reg- | AGGAAGATCATTAAATGTGAGCGTTTTTAA | 26 |
| T4R12C3 | | |
| 26-Reg- | CCAATAGGAAACTAGCATGTCAAGGAGCAA | 27 |
| T4R14C3 | | |
| 27-Reg- | AAGCAAACCTATTATAGTCAGAAGATTGAAT | 28 |
| T1R05C5 | C | |
| 28-Reg- | AAATATTCCAAAGCGGATTGCATCGAGCTTCA | 29 |
| T1R07C4 | | |
| 29-Reg- | CCCCTCAACCTCGTTTACCAGACGGAATACCA | 30 |
| T1R07C5 | | |
| 30-Reg- | AGATTTAGACGATAAAAACCAAAAATCGTCA | 31 |
| T1R09C4 | T | |
| 31-Reg- | AGTCAGGACATAGGCTGGCTGACCTTTGAAA | 32 |
| TIR10C1 | G | |
| 32-Reg- | ACCCAAATAACTTTAATCATTGTGATCAGTTG | 33 |
| TIR11C4 | | |
| 33-Reg- | CAAAGCTGTCCATGTTACTTAGCCATTATACC | 34 |
| T1R11C5 | | |
| 34-Reg- | CCCCAGCGGGAACGAGGCGCAGACTATTCAT | 35 |
| T1R13C4 | T | |
| 35-Reg- | AAGCGCGAGAGGACTAAAGACTTTGGCCGCT | 36 |
| TIR13C5 | T | |
| 36-Reg- | GAGTTAAATTCATGAGGAAGTTTCTCTTTGAC | 37 |
| T1R15C4 | | |
| 37-Reg- | TTTCACGTCGATAGTTGCGCCGACCTTGCAGG | 38 |
| TIR17C4 | | |
| 38-Reg- | GAGCCGCCTTAAAGCCAGAATGGAGATGATA | 39 |
| T2R07C4 | C | |
| 39-Reg- | CACCACCAGCCCCCTTATTAGCGTCCGTAATC | 40 |
| T2R07C5 | | |
| 40-Reg- | AGTAGCGAATTGAGGGAGGGAAGGATAAGTT | 41 |
| T2R09C5 | T | |
| 41-Reg- | GCCATTTGCAAACGTAGAAAATACCTGGCAT | 42 |
| T2R10C1 | G | |
| 42-Reg- | ATTTTGTCAGCCGAACAAAGTTACGCCCAATA | 43 |
| T2R11C5 | | |
| 43-Reg- | ATAAGAGCAGCCTTTACAGAGAGAACAAAAT | 44 |
| T2R13C5 | A | |
| 44-Reg- | CAAATCAGTGCTATTTTGCACCCAGCCTAATT | 45 |
| T2R17C4 | | |
| 45-Reg- | AAATAAGAACTTTTTCAAATATATCTGAGAGA | 46 |
| T3R04C5 | | |
| 46-Reg- | CTACCTTTAGAATCCTTGAAAACAAGAAAAC | 47 |
| T3R06C5 | A | |
| 47-Reg- | TTTCCCTTTTAACCTCCGGCTTAGCAAAGAAC | 48 |
| T3R07C4 | | |
| 48-Reg- | AAATTAATACCAAGTTACAAAATCCTGAATA | 49 |
| T3R08C5 | A | |
| 49-Reg- | CTTTGAATTACATTTAACAATTTCTAATTAAT | 50 |
| T3R09C4 | | |
| 50-Reg- | GTAGATTTGTTATTAATTTTAAAAAACAATTC | 51 |
| T3R10C1 | | |
| 51-Reg- | TGGAAGGGAGCGGAATTATCATCAACTAATA | 52 |
| T3R10C5 | G | |
| 52-Reg- | AACATTATGTAAAACAGAAATAAATTTTACAT | 53 |
| T3R11C2 | | |
| 53-Reg- | CCAGAAGGTTAGAACCTACCATATCCTGATTG | 54 |
| T3R11C4 | | |
| 54-Reg- | ATTAGAGCAATATCTGGTCAGTTGCAGCAGA | 55 |
| T3R12C5 | A | |
| 55-Reg- | GCATCACCAGTATTAGACTTTACAGTTTGAGT | 56 |
| T3R13C2 | | |
| 56-Reg- | CCTCAATCCGTCAATAGATAATACAGAAACC | 57 |
| T3R13C4 | A | |
| 57-Reg- | GATAAAACTTTTTGAATGGCTATTTTCACCAG | 58 |
| T3R14C5 | | |
| 58-Reg- | AGACAATAAGAGGTGAGGCGGTCATATCAAA | 59 |
| T3R15C4 | C | |
| 59-Reg- | TCACACGATGCAACAGGAAAAACGGAAGAAC | 60 |
| T3R16C5 | T | |
| 60-Reg- | CCAGCCATCCAGTAATAAAAGGGACGTGGCA | 61 |
| T3R17C4 | C | |
| 61-Reg- | AAAAACCGAAATCCCTTATAAATCGAGAGTT | 62 |
| T4R05C5 | G | |
| 62-Reg- | GGCCCTGAAAAAGAATAGCCCGAGCGTGGAC | 63 |
| T4R07C4 | T | |
| 63-Reg- | CAGCAAGCGTCGTGCCAGCTGCATAGCCTGG | 64 |
| T4R07C5 | G | |
| 64-Reg- | AAGTGTAATAATGAATCGGCCAACCACCGCC | 65 |
| T4R09C4 | T | |
| 65-Reg- | GAATTCGTGCCATTCGCCATTCAGTTCCGGCA | 66 |
| T4R10C1 | | |
| 66-Reg- | TTCGCTATTGCCAAGCTTGCATGCGAAGCATA | 67 |
| T4R11C4 | | |
| 67-Reg- | CTGGCGAACATCTGCCAGTTTGAGATTCTCCG | 68 |
| T4R11C5 | | |
| 68-Reg- | CCCGTCGGGGGACGACGACAGTATCGGGCCT | 69 |
| T4R13C4 | C | |
| 69-Reg- | TGGGAACAATTCGCATTAAATTTTTGATAATC | 70 |
| T4R13C5 | | |
| 70-Reg- | ACCCCGGTTGTTAAATCAGCTCATAGTAACAA | 71 |
| T4R15C4 | | |
| 71-Reg- | AGACAGTCCATTGCCTGAGAGTCTTCATATGT | 72 |
| T4R17C4 | | |
| 72-Reg- | CAACTAAAGTACGGTGGGATGGCTTAGAGCT | 73 |
| T1R03C5 | TCAGACCGG | |
| 73-Reg- | ACTGGCTCCGGAACAACATTATTAGCAAAAG | 74 |
| T1R10C2 | AAGTTTTGC | |
| 74-Reg- | TTAGGATTAGCGGGGTGGAACCTATTATTCTG | 75 |
| T2R03C5 | ACTGGTAA | |
| 75-Reg- | ACCGTCACCATTAGCAAGGCCGGAACCAGAG | 76 |
| T2R10C2 | CCACCACCG | |
| 76-Reg- | AAAGCCGGCGAACGTGTGCCGTAAAGCACTA | 77 |
| T4R03C5 | AAAAGGGCG | |
| 77-Reg- | CCCGGGTACCGCTCACAATTCCACGTTTGCGT | 78 |
| T4R10C2 | ATTGGGCG | |
| 78-Reg- | TTGCGGGATTAAACAGCTTGATACTGAAAATC | 79 |
| T1R15C5 | TCCAAAAAAGCGGAGT | |
| 79-Reg- | GACGGAAAACCATCGATAGCAGCATTGCCAT | 80 |
| T2R10C4 | CTTTTCATACACCCTCA | |
| 80-Reg- | TGCCAGTTATAACATAAAAACAGGACAAGAA | 81 |
| T2R15C4 | TTGAGTTAACAGAAGGA | |
| 81-Reg- | AACAGCCATTAAATCAAGATTAGTATATAGA | 82 |
| T2R15C5 | AGGCTTATCCAAGCCGT | |
| 82-Reg- | AGAAAAGCACAAAGGCTATCAGGTAAATCAC | 83 |
| T4R15C5 | CATCAATATCAATGCCT | |
| 83-Edg- | TTTGGTGGCATCAATTCTAGGGCGCGAGCTGA | 84 |
| T1R00C7- | AAATTTCGTTTCTTTGCTTGTGTCTTGTCTTCG | |
| T3.5p-T3. | TCGTGCTTGTTCC | |
| 3p-DYE40. | | |
| 22.9.3p | | |
| 84-Edg- | TTTTCCCAATTCTGCGAACCCATATAACAGTT | 85 |
| T1R02C7- | GATTTTCGTTTCTTTGCTTGTGTCTTGTCTTCG | |
| T3.5p-T3. | TCGTGCTTGTTCC | |
| 3p-DYE40. | | |
| 22.9.3p | | |
| 85-Edg- | TTTATTGCTCCTTTTGATATTAGAGAGTACCTT | 86 |
| T1R04C7- | TATTTCGTTTCTTTGCTTGTGTCTTGTCTTCGT | |
| T3.5p-T3. | CGTGCTTGTTCC | |
| 3p-DYE40. | | |
| 22.9.3p | | |
| 86-Edg- | TTTCCATAAATCAAAAATCCAGAAAACGAGA | 87 |
| T1R06C7- | ATGATTTCGTTTCTTTGCTTGTGTCTTGTCTTC | |
| T3.5p-T3. | GTCGTGCTTGTTCC | |
| 3p-DYE40. | | |
| 22.9.3p | | |
| 87-Edg- | TTTCGAGGCATAGTAAGAGACGCCAAAAGGA | 88 |
| T1R08C7- | ATTATTTCGTTTCTTTGCTTGTGTCTTGTCTTC | |
| T3.5p-T3. | GTCGTGCTTGTTCC | |
| 3p-DYE40. | | |
| 22.9.3p | | |
| 88-Edg- | TTTGAAACACCAGAACGAGAGGCTTGCCCTG | 89 |
| TIR10C7- | ACGATTTCGTTTCTTTGCTTGTGTCTTGTCTTC | |
| T3.5p-T3. | GTCGTGCTTGTTCC | |
| 3p-DYE40. | | |
| 22.9.3p | | |
| 89-Edg- | TTTCTGATAAATTGTGTCGAGATTTGTATCAT | 90 |
| TIR12C7- | CGCTTTCGTTTCTTTGCTTGTGTCTTGTCTTCG | |
| T3.5p-T3. | TCGTGCTTGTTCC | |
| 3p-DYE40. | | |
| 22.9.3p | | |
| 90-Edg- | TTTGAACGAGGGTAGCAACGCGAAAGACAGC | 91 |
| T1R14C7- | ATCGTTTCGTTTCTTTGCTTGTGTCTTGTCTTC | |
| T3.5p-T3. | GTCGTGCTTGTTCC | |
| 3p-DYE40. | | |
| 22.9.3p | | |
| 91-Edg- | TTTGGTTTATCAGCTTGCTAGCCTTTAATTGTA | 92 |
| T1R16C7- | TCTTTCGTTTCTTTGCTTGTGTCTTGTCTTCGTC | |
| T3.5p-T3. | GTGCTTGTTCC | |
| 3p-DYE40. | | |
| 22.9.3p | | |
| 92-Edg- | TTTGGGATTTTGCTAAACAAATGAATTTTCTG | 93 |
| T1R18C7- | TATTTTCGTTTCTTTGCTTGTGTCTTGTCTTCGT | |
| T3.5p-T3. | CGTGCTTGTTCC | |
| 3p-DYE40. | | |
| 22.9.3p | | |
| 93-Edg- | TTTACAAACTACAACGCCTGAGTTTCGTCACC | 94 |
| T1R20C7- | AGTTTTCGTTTCTTTGCTTGTGTCTTGTCTTCG | |
| T3.5p-T3. | TCGTGCTTGTTCC | |
| 3p-DYE40. | | |
| 22.9.3p | | |
| 94-Edg- | TTTAGCCACCACCCTCATTGAACCGCCACCCT | 95 |
| T2R00C7- | CAGTTTCGTTTCTTTGCTTGTGTCTTGTCTTCG | |
| T3.5p-T3. | TCGTGCTTGTTCC | |
| 3p-DYE40. | | |
| 22.9.3p | | |
| 95-Edg- | TTTGAGAGGGTTGATATAAGCGGATAAGTGC | 96 |
| T2R02C7- | CGTCTTTCGTTTCTTTGCTTGTGTCTTGTCTTC | |
| T3.5p-T3. | GTCGTGCTTGTTCC | |
| 3p-DYE40. | | |
| 22.9.3p | | |
| 96-Edg- | TTTGTATAAACAGTTAATGTTGAGTAACAGTG | 97 |
| T2R04C7- | CCCTTTCGTTTCTTTGCTTGTGTCTTGTCTTCG | |
| T3.5p-T3. | TCGTGCTTGTTCC | |
| 3p-DYE40. | | |
| 22.9.3p | | |
| 97-Edg- | TTTGCAGGTCAGACGATTGTTGACAGGAGGTT | 98 |
| T2R06C7- | GAGTTTCGTTTCTTTGCTTGTGTCTTGTCTTCG | |
| T3.5p-T3. | TCGTGCTTGTTCC | |
| 3p-DYE40. | | |
| 22.9.3p | | |
| 98-Edg- | TTTTAGCGCGTTTTCATCGCTTTAGCGTCAGA | 99 |
| T2R08C7- | CTGTTTCGTTTCTTTGCTTGTGTCTTGTCTTCG | |
| T3.5p-T3. | TCGTGCTTGTTCC | |
| 3p-DYE40. | | |
| 22.9.3p | | |
| 99-Edg- | TTTGCGCCAAAGACAAAAGTTCATATGGTTTA | 100 |
| T2R10C7- | CCATTTCGTTTCTTTGCTTGTGTCTTGTCTTCG | |
| T3.5p-T3. | TCGTGCTTGTTCC | |
| 3p-DYE40. | | |
| 22.9.3p | | |
| 100-Edg- | TTTCCGAAGCCCTTTTTAAAGCAATAGCTATC | 101 |
| T2R12C7- | TTATTTCGTTTCTTTGCTTGTGTCTTGTCTTCGT | |
| T3.5p-T3. | CGTGCTTGTTCC | |
| 3p-DYE40. | | |
| 22.9.3p | | |
| 101-Edg- | TTTTTTTTTGTTTAACGTCTCCAAATAAGAAAC | 102 |
| T2R14C7- | GATTTCGTTTCTTTGCTTGTGTCTTGTCTTCGT | |
| T3.5p-T3. | CGTGCTTGTTCC | |
| 3p-DYE40. | | |
| 22.9.3p | | |
| 102-Edg- | TTTAACCTCCCGACTTGCGGCGAGGCGTTTTA | 103 |
| T2R16C7- | GCGTTTCGTTTCTTTGCTTGTGTCTTGTCTTCG | |
| T3.5p-T3. | TCGTGCTTGTTCC | |
| 3p-DYE40. | | |
| 22.9.3p | | |
| 103-Edg- | TTTTAAACCAAGTACCGCATTCCAAGAACGGG | 104 |
| T2R18C7- | TATTTTCGTTTCTTTGCTTGTGTCTTGTCTTCGT | |
| T3.5p-T3. | CGTGCTTGTTCC | |
| 3p-DYE40. | | |
| 22.9.3p | | |
| 104-Edg- | TTTAGATAAGTCCTGAACACCTGTTTATCAAC | 105 |
| T2R20C7- | AATTTTCGTTTCTTTGCTTGTGTCTTGTCTTCG | |
| T3.5p-T3. | TCGTGCTTGTTCC | |
| 3p-DYE40. | | |
| 22.9.3p | | |
| 105-Edg- | TTTGTAAAGTAATTCTGTCAAAGTACCGACAA | 106 |
| T3R00C7- | AAGTTTCGTTTCTTTGCTTGTGTCTTGTCTTCG | |
| T3.5p-T3. | TCGTGCTTGTTCC | |
| 3p-DYE40. | | |
| 22.9.3p | | |
| 106-Edg- | TTTAGTAGGGCTTAATTGAAAAGCCAACGCTC | 107 |
| T3R02C7- | AACTTTCGTTTCTTTGCTTGTGTCTTGTCTTCG | |
| T3.5p-T3. | TCGTGCTTGTTCC | |
| 3p-DYE40. | | |
| 22.9.3p | | |
| 107-Edg- | TTTAATGGTTTGAAATACCCTTCTGACCTAAA | 108 |
| T3R04C7- | TTTTTTCGTTTCTTTGCTTGTGTCTTGTCTTCGT | |
| T3.5p-T3. | CGTGCTTGTTCC | |
| 3p-DYE40. | | |
| 22.9.3p | | |
| 108-Edg- | TTTAGTCAATAGTGAATTTTTAAGACGCTGAG | 109 |
| T3R06C7- | AAGTTTCGTTTCTTTGCTTGTGTCTTGTCTTCG | |
| T3.5p-T3. | TCGTGCTTGTTCC | |
| 3p-DYE40. | | |
| 22.9.3p | | |
| 109-Edg- | TTTTGAGCAAAAGAAGATGATTCATTTCAATT | 110 |
| T3R08C7- | ACCTTTCGTTTCTTTGCTTGTGTCTTGTCTTCG | |
| T3.5p-T3. | TCGTGCTTGTTCC | |
| 3p-DYE40. | | |
| 22.9.3p | | |
| 110-Edg- | TTTCAATATAATCCTGATTGATGATGGCAATT | 111 |
| T3R10C7- | CATTTTCGTTTCTTTGCTTGTGTCTTGTCTTCG | |
| T3.5p-T3. | TCGTGCTTGTTCC | |
| 3p-DYE40. | | |
| 22.9.3p | | |
| 111-Edg- | TTTGTTATCTAAAATATCTAAAGGAATTGAGG | 112 |
| T3R12C7- | AAGTTTCGTTTCTTTGCTTGTGTCTTGTCTTCG | |
| T3.5p-T3. | TCGTGCTTGTTCC | |
| 3p-DYE40. | | |
| 22.9.3p | | |
| 112-Edg- | TTTACATCGCCATTAAAAAAACTGATAGCCCT | 113 |
| T3R14C7- | AAATTTCGTTTCTTTGCTTGTGTCTTGTCTTCG | |
| T3.5p-T3. | TCGTGCTTGTTCC | |
| 3p-DYE40. | | |
| 22.9.3p | | |
| 113-Edg- | TTTTCGTCTGAAATGGATTACATTTTGACGCT | 114 |
| T3R16C7- | CAATTTCGTTTCTTTGCTTGTGTCTTGTCTTCG | |
| T3.5p-T3. | TCGTGCTTGTTCC | |
| 3p-DYE40. | | |
| 22.9.3p | | |
| 114-Edg- | TTTTTGATTAGTAATAACATTGTAGCAATACT | 115 |
| T3R18C7- | TCTTTTCGTTTCTTTGCTTGTGTCTTGTCTTCGT | |
| T3.5p-T3. | CGTGCTTGTTCC | |
| 3p-DYE40. | | |
| 22.9.3p | | |
| 115-Edg- | TTTAGGAACGGTACGCCAGTAAAGGGATTTTA | 116 |
| T3R20C7- | GACTTTCGTTTCTTTGCTTGTGTCTTGTCTTCG | |
| T3.5p-T3. | TCGTGCTTGTTCC | |
| 3p-DYE40. | | |
| 22.9.3p | | |
| 116-Edg- | TTTGAGCACGTATAACGTGCTATGGTTGCTTT | 117 |
| T4R00C7- | GACTTTCGTTTCTTTGCTTGTGTCTTGTCTTCG | |
| T3.5p-T3. | TCGTGCTTGTTCC | |
| 3p-DYE40. | | |
| 22.9.3p | | |
| 117-Edg- | TTTCGGGCGCTAGGGCGCTAAGAAAGCGAAA | 118 |
| T4R02C7- | GGAGTTTCGTTTCTTTGCTTGTGTCTTGTCTTC | |
| T3.5p-T3. | GTCGTGCTTGTTCC | |
| 3p-DYE40. | | |
| 22.9.3p | | |
| 118-Edg- | TTTATCACCCAAATCAAGTGCCCACTACGTGA | 119 |
| T4R04C7- | ACCTTTCGTTTCTTTGCTTGTGTCTTGTCTTCG | |
| T3.5p-T3. | TCGTGCTTGTTCC | |
| 3p-DYE40. | | |
| 22.9.3p | | |
| 119-Edg- | TTTATCCTGTTTGATGGTGGCCCCAGCAGGCG | 120 |
| T4R06C7- | AAATTTCGTTTCTTTGCTTGTGTCTTGTCTTCG | |
| T3.5p-T3. | TCGTGCTTGTTCC | |
| 3p-DYE40. | | |
| 22.9.3p | | |
| 120-Edg- | TTTGCTCACTGCCCGCTTTACATTAATTGCGTT | 121 |
| T4R08C7- | GCTTTCGTTTCTTTGCTTGTGTCTTGTCTTCGT | |
| T3.5p-T3. | CGTGCTTGTTCC | |
| 3p-DYE40. | | |
| 22.9.3p | | |
| 121-Edg- | TTTGTAACGCCAGGGTTTTAAGGCGATTAAGT | 122 |
| T4R10C7- | TGGTTTCGTTTCTTTGCTTGTGTCTTGTCTTCG | |
| T3.5p-T3. | TCGTGCTTGTTCC | |
| 3p-DYE40. | | |
| 22.9.3p | | |
| 122-Edg- | TTTCGTTGGTGTAGATGGGGTAATGGGATAGG | 123 |
| T4R12C7- | TCATTTCGTTTCTTTGCTTGTGTCTTGTCTTCG | |
| T3.5p-T3. | TCGTGCTTGTTCC | |
| 3p-DYE40. | | |
| 22.9.3p | | |
| 123-Edg- | TTTTTTAAATTGTAAACGTATTGTATAAGCAA | 124 |
| T4R14C7- | ATATTTCGTTTCTTTGCTTGTGTCTTGTCTTCG | |
| T3.5p-T3. | TCGTGCTTGTTCC | |
| 3p-DYE40. | | |
| 22.9.3p | | |
| 124-Edg- | TTTGCCGGAGAGGGTAGCTTAGCTGATAAATT | 125 |
| T4R16C7- | AATTTTCGTTTCTTTGCTTGTGTCTTGTCTTCG | |
| T3.5p-T3. | TCGTGCTTGTTCC | |
| 3p-DYE40. | | |
| 22.9.3p | | |
| 125-Edg- | TTTAAATTTTTAGAACCCTTTCAACGCAAGGA | 126 |
| T4R18C7- | TAATTTCGTTTCTTTGCTTGTGTCTTGTCTTCG | |
| T3.5p-T3. | TCGTGCTTGTTCC | |
| 3p-DYE40. | | |
| 22.9.3p | | |
| 126-Edg- | TTTTAAGCAATAAAGCCTCAAAGAATTAGCA | 127 |
| T4R20C7- | AAATTTTCGTTTCTTTGCTTGTGTCTTGTCTTC | |
| T3.5p-T3. | GTCGTGCTTGTTCC | |
| 3p-DYE40. | | |
| 22.9.3p | | |
| 127-Bri- | TGCCACTACTTTTTTTGCCACCCTC | 128 |
| T1R14C2 | | |
| 128-Bri- | AACTGAACATTTTTTTTGAATAACC | 129 |
| T2R14C2 | | |
| 129-Bri- | GCCACGCTGTTTTTTTACCAGTGAG | 130 |
| T3R14C2 | | |
| 130-Bri- | CAAAAATAATTTTTTTTGTTTAGAC | 131 |
| T4R14C2 | | |
| 131-Bri- | GATACATTTCGCTTTTTTGACCCTGTAAT | 132 |
| T1R02C5 | | |
| 132-Bri- | ACCGTACTCAGGTTTTTGATCTAAAGTTT | 133 |
| T2R02C5 | | |
| 133-Bri- | AACATGTAATTTTTTTTGAAACCAATCAA | 134 |
| T3R02C5 | | |
| 134-Bri- | GCGTAACCACCATTTTTGAGTAAAAGAGT | 135 |
| T4R02C5 | | |
| 135-Bri- | CAGAGGGGGTTTTGCCTTCCTGTAGCCAGCT | 136 |
| T1R08C1 | | |
| 136-Bri- | GAACCGCCTCTTTACCTAAAACGAAAGAGGC | 137 |
| T2R08C1 | | |
| 137-Bri- | CATAAATCAATTTAGTCAGAGGGTAATTGAG | 138 |
| T3R08C1 | | |
| 138-Bri- | CCAGGGTGGTTTTGCAAATGAAAAATCTAAA | 139 |
| T4R08C1 | | |
| 139-Bri- | ACAACCATTTTTTCATACATGGCTTTTAAGCG | 140 |
| T1R16C3 | CA | |
| 140-Bri- | TTTTATCTTTTTTATCCAATCGCAAGAGTTGGG | 141 |
| T2R16C3 | T | |
| 141-Bri- | GCCAACATTTTTTCCACTATTAAAGAAATAGG | 142 |
| T3R16C3 | GT | |
| 142-Bri- | ACAAGAGTTTTTTTCGCGTTTTAATTCAAAAA | 143 |
| T4R16C3 | GA | |
| 143-Bri- | TGGATAGCAAGCCCGATTTTTAATCGTAAACG | 144 |
| T1R07C2 | CCAT | |
| 144-Bri- | AGAACCGCATTTACCGTTTTACCGATATATAC | 145 |
| T2R07C2 | GTAA | |
| 145-Bri- | TTGCTTCTTATATGTATTTTACGCTAACGGAG | 146 |
| T3R07C2 | AATT | |
| 146-Bri- | ACGGGCAAGTTCCAGTTTTTTCTGACCTGCAA | 147 |
| T4R07C2 | CAGT | |
| 147-Bri- | GAGAATAGAAAGGAACAACTATTTTCTCAAG | 148 |
| T1R18C5 | AGAAGGA | |
| 148-Bri- | TTTTATTTTCATCGTAGGAATTTTTAGCCTGTT | 149 |
| T2R18C5 | TAGTA | |
| 149-Bri- | CAAACTATCGGCCTTGCTGGTTTTTGAGCTTG | 150 |
| T3R18C5 | ACGGGG | |
| 150-Bri- | GAGTAATGTGTAGGTAAAGATTTTTTGTTTTA | 151 |
| T4R18C5 | AATATG | |
| 151-Bri- | AGGACAGATGATTTTTTCACCAGTAGCACCAT | 152 |
| TIR12C1 | TACCGACTTGA | |
| 152-Bri- | ATTAAGACTCCTTTTTAATATACAGTAACAGT | 153 |
| T2R12C1 | ACCGAAATTGC | |
| 153-Bri- | GACAACTCGTATTTTTTCCTGTGTGAAATTGTT | 154 |
| T3R12C1 | ATCCGAGCTC | |
| 154-Bri- | CCGCTTCTGGTTTTTTCGTTAATAAAACGAAC | 155 |
| T4R12C1 | TAAATTATACC | |
| 155-Bri- | TGTCGTCTCAGCCCTCATATTTTTTTCGCCACC | 156 |
| T1R19C5 | CTCAGGTGTATC | |
| 156-Bri- | TAATCGGCCATCCTAATTTTTTTTTTTTTTCGA | 157 |
| T2R19C5 | GCCAACAACGCC | |
| 157-Bri- | CTGTCCATTTTTATAATCATTTTTTTCTTAATG | 158 |
| T3R19C5 | CGCCCACGCTGC | |
| 158-Bri- | ACTTTTGCATCGGTTGTACTTTTTTTAACCTGT | 159 |
| T4R19C5 | TTAGGACCATTA | |
| 159-Bri- | AAGCGAACAATTGCTGAATATAATGCTGTATT | 160 |
| T1R05C4 | TTTTTGTGAGAAAGGCCGG | |
| 160-Bri- | AGGAGTGTAAACATGAAAGTATTAAGAGGCT | 161 |
| T2R05C4 | TTTTTTGCGAATAATAATTT | |
| 161-Bri- | GCGAGAAAATAAACACCGGAATCATAATTAT | 162 |
| T3R05C4 | TTTTTTCGCCCAATAGCAAG | |
| 162-Bri- | CCAACGTCATCGGAACCCTAAAGGGAGCCCT | 163 |
| T4R05C4 | TTTTTTGAACAATATTACCG | |
| 163-Bri- | GGAATTAGAGCTTTTTTTTCAGACCAGGCGCG | 164 |
| T2R10C0 | TTGGGAAGATTTTTTTTCCAGGCAAAGC | |
| 164-Bri- | AATCATGGTCATTTTTTTTTTTGCCCGAACTCA | 165 |
| T4R10C0 | GGTTTAACTTTTTTTTCAGTATGTTAG | |
| 165-Reg- | GTCAGGAAGAGGTCATTTTTGCTCTGGAAGTT | 166 |
| T1R05C6- | TACAGTTATTAGCCCGCATTT | |
| T3.3p- | | |
| JF20.22.9.3p | | |
| 166-Reg- | ATACATACAACACTATCATAACATGCTTTATT | 167 |
| T1R09C6- | TACAGTTATTAGCCCGCATTT | |
| T3.3p- | | |
| JF20.22.9.3p | | |
| 167-Reg- | ACAACGGAAATCCGCGACCTGCCTCATTCATT | 168 |
| T1R13C6- | TACAGTTATTAGCCCGCATTT | |
| T3.3p- | | |
| JF20.22.9.3p | | |
| 168-Reg- | CAAAAGGTTCGAGGTGAATTTCTCGTCACCTT | 169 |
| TIR17C6- | TACAGTTATTAGCCCGCATTT | |
| T3.3p- | | |
| JF20.22.9.3p | | |
| 169-Reg- | CAGTGCCCCCCCTGCCTATTTCTTTGCTCATTT | 170 |
| T2R05C6- | ACAGTTATTAGCCCGCATTT | |
| T3.3p- | | |
| JF20.22.9.3p | | |
| 170-Reg- | AGTTTGCGCATTTTCGGTCATAGAGCCGCCTT | 171 |
| T2R09C6- | TACAGTTATTAGCCCGCATTT | |
| T3.3p- | | |
| JF20.22.9.3p | | |
| 171-Reg- | ATGAAATGAAAAGTAAGCAGATACAATCAAT | 172 |
| T2R13C6- | TTACAGTTATTAGCCCGCATTT | |
| T3.3p- | | |
| JF20.22.9.3p | | |
| 172-Reg- | TAAGAACGGAGGTTTTGAAGCCTATTATTTTT | 173 |
| T2R17C6- | TACAGTTATTAGCCCGCATTT | |
| T3.3p- | | |
| JF20.22.9.3p | | |
| 173-Reg- | ATTTCATGACCGTGTGATAAATAATTCTTATT | 174 |
| T3R05C6- | TACAGTTATTAGCCCGCATTT | |
| T3.3p- | | |
| JF20.22.9.3p | | |
| 174-Reg- | GCGAATTATGAAACAAACATCATAGCGATAT | 175 |
| T3R09C6- | TTACAGTTATTAGCCCGCATTT | |
| T3.3p- | | |
| JF20.22.9.3p | | |
| 175-Reg- | ACAGTTGTTAGGAGCACTAACATATTCCTGTT | 176 |
| T3R13C6- | TACAGTTATTAGCCCGCATTT | |
| T3.3p- | | |
| JF20.22.9.3p | | |
| 176-Reg- | AATACCTATTTACATTGGCAGAAGTCTTTATT | 177 |
| T3R17C6- | TACAGTTATTAGCCCGCATTT | |
| T3.3p- | | |
| JF20.22.9.3p | | |
| 177-Reg- | GGCGATGTTTTTGGGGTCGAGGGCGAGAAAT | 178 |
| T4R05C6- | TTACAGTTATTAGCCCGCATTT | |
| T3.3p- | | |
| JF20.22.9.3p | | |
| 178-Reg- | CTAACTCCCAGTCGGGAAACCTGGTCCACGTT | 179 |
| T4R09C6- | TACAGTTATTAGCCCGCATTT | |
| T3.3p- | | |
| JF20.22.9.3p | | |
| 179-Reg- | ATTGACCCGCATCGTAACCGTGAGGGGGATTT | 180 |
| T4R13C6- | TACAGTTATTAGCCCGCATTT | |
| T3.3p- | | |
| JF20.22. | | |
| 9.3p | | |
| 180-Reg- | ACCGTTCATTTTTGAGAGATCTCCCAAAAATT | 181 |
| T4R17C6- | TACAGTTATTAGCCCGCATTT | |
| T3.3p- | | |
| JF20.22. | | |
| 9.3p | | |
| 181-Reg- | CCATGTACCGTAACACTGTAGCATTCCACAGA | 182 |
| T1R21C5- | TTCCAGACTTTACAGTTATTAGCCCGCATTT | |
| T3.3p- | | |
| JF20.22. | | |
| 9.3p | | |
| 182-Reg- | AGCTAATGCAGAACGCGAGAAAAATAATATC | 183 |
| T2R21C5- | CTGTCTTTCTTTACAGTTATTAGCCCGCATTT | |
| T3.3p- | | |
| JF20.22. | | |
| 9.3p | | |
| 183-Reg- | CTAAACAGGAGGCCGATAATCCTGAGAAGTG | 184 |
| T3R21C5- | TCACGCAAATTTACAGTTATTAGCCCGCATTT | |
| T3.3p- | | |
| JF20.22. | | |
| 9.3p | | |
| 184-Reg- | AATCATACAGGCAAGGCAGAGCATAAAGCTA | 185 |
| T4R21C5- | AGGGAGAAGTTTACAGTTATTAGCCCGCATTT | |
| T3.3p- | | |
| JF20.22. | | |
| 9.3p | | |
| 185-Reg- | TTTCATTGAGTAGATTTAGTTTCTATATTT | 186 |
| T1R03C6 | | |
| 186-Reg- | AACAGTTAGGTCTTTACCCTGATCCAACAG | 187 |
| T1R07C6 | | |
| 187-Reg- | GTGAATATAGTAAATTGGGCTTTAATGCAG | 188 |
| T1R11C6 | | |
| 188-Reg- | CTCAGCAGGCTACAGAGGCTTTAACAAAGT | 189 |
| T1R15C6 | | |
| 189-Reg- | GTTAGTAACTTTCAACAGTTTCAAAGGCTC | 190 |
| T1R19C6 | | |
| 190-Reg- | GTACCAGGTATAGCCCGGAATAGAACCGCC | 191 |
| T2R03C6 | | |
| 191-Reg- | GCCAGCAGCCTTGATATTCACAAACGGGGT | 192 |
| T2R07C6 | | |
| 192-Reg- | TAGAAAAGGCGACATTCAACCGCAGAATCA | 193 |
| T2R11C6 | | |
| 193-Reg- | ATCCCAAAAAAATGAAAATAGCAAGAAACA | 194 |
| T2R15C6 | | |
| 194-Reg- | CTTATCACTCATCGAGAACAAGCGGTATTC | 195 |
| T2R19C6 | | |
| 195-Reg- | CCAGTATGAATCGCCATATTTAGTAATAAG | 196 |
| T3R03C6 | | |
| 196-Reg- | GCTTAGAATCAAAATCATAGGTTTTAGTTA | 197 |
| T3R07C6 | | |
| 197-Reg- | ATTATCAGTTTGGATTATACTTGCGCAGAG | 198 |
| T3R11C6 | | |
| 198-Reg- | ATGCGCGTACCGAACGAACCACGCAAATCA | 199 |
| T3R15C6 | | |
| 199-Reg- | TTAACCGTCACTTGCCTGAGTACTCATGGA | 200 |
| T3R19C6 | | |
| 200-Reg- | GGAAGGGGGCAAGTGTAGCGGTGCTACAGG | 201 |
| T4R03C6 | | |
| 201-Reg- | CTGGTTTGTTCCGAAATCGGCATCTATCAG | 202 |
| T4R07C6 | | |
| 202-Reg- | GTGCTGCCCCAGTCACGACGTTTGAGTGAG | 203 |
| T4R11C6 | | |
| 203-Reg- | CAGGAAGTAATATTTTGTTAAAAACGGCGG | 204 |
| T4R15C6 | | |
| 204-Reg- | CCTTTATCATATATTTTAAATGGATATTCA | 205 |
| T4R19C6 | | |
| 205-Reg- | AAAAGAATAACCGAACTGACCAACTTCATCA | 206 |
| T1R13C2 | AGAGTAATCTTTTAAGA | |
| 206-Reg- | CGCTAATAGGAATACCCAAAAGAAATACATA | 207 |
| T2R13C2 | AAGGTGGCAGAATTATC | |
| 207-Reg- | TTCATCAACGCACTCCAGCCAGCTGCTGCGCA | 208 |
| T4R13C2 | ACTGTTGGAGAGGATC | |
| 214-Reg- | AAAAGAATAACCGAACTGACCAACTTCATCA | 209 |
| T1R13C2- | AGAGTAATCTTTTAAGATTTATACATCT | |
| T3.3p- | | |
| RJ8.RC.1.3p | | |
| 215-Reg- | CGCTAATAGGAATACCCAAAAGAAATACATA | 210 |
| T2R13C2- | AAGGTGGCAGAATTATCTTTATACATCT | |
| T3.3p- | | |
| RJ8.RC.1.3p | | |
| 216-Reg- | TTCATCAACGCACTCCAGCCAGCTGCTGCGCA | 211 |
| T4R13C2- | ACTGTTGGAGAGGATCTTTATACATCT | |
| T3.3p- | | |
| RJ8.RC.1.3p | | |
| 217-Reg- | AAAAGAATAACCGAACTGACCAACTTCATCA | 212 |
| T1R13C2- | AGAGTAATCTTTTAAGATTTATACATCTAG | |
| T3.3p- | | |
| RJ10.RC.3p | | |
| 218-Reg- | CGCTAATAGGAATACCCAAAAGAAATACATA | 213 |
| T2R13C2- | AAGGTGGCAGAATTATCTTTATACATCTAG | |
| T3.3p- | | |
| RJ10.RC.3p | | |
| 219-Reg- | TTCATCAACGCACTCCAGCCAGCTGCTGCGCA | 214 |
| T4R13C2- | ACTGTTGGAGAGGATCTTTATACATCTAG | |
| T3.3p- | | |
| RJ10.RC.3p | | |
| 220-Reg- | CATTCAACGAGATGGTTTAATTTCCAACGTAA | 215 |
| TIR09C5 | | |
| 221-Reg- | TAAGTTTTAACAAATAAATCCTCAACCAGAAC | 216 |
| T2R05C5 | | |
| 222-Reg- | GTGCCTAAGTAAAACGACGGCCAGTACGCCA | 217 |
| T4R09C5 | G | |
| 229-Reg- | CATTCAACGAGATGGTTTAATTTCCAACGTAA | 218 |
| T1R09C5- | TTTATACATCT | |
| T3.3p- | | |
| RJ8.RC.1.3p | | |
| 230-Reg- | TAAGTTTTAACAAATAAATCCTCAACCAGAAC | 219 |
| T2R05C5- | TTTATACATCT | |
| T3.3p- | | |
| RJ8.RC.1.3p | | |
| 231-Reg- | GTGCCTAAGTAAAACGACGGCCAGTACGCCA | 220 |
| T4R09C5- | GTTTATACATCT | |
| T3.3p- | | |
| RJ8.RC.1.3p | | |
| 232-Reg- | CATTCAACGAGATGGTTTAATTTCCAACGTAA | 221 |
| T1R09C5- | TTTATACATCTAG | |
| T3.3p- | | |
| RJ10.RC.3p | | |
| 233-Reg- | TAAGTTTTAACAAATAAATCCTCAACCAGAAC | 222 |
| T2R05C5- | TTTATACATCTAG | |
| T3.3p- | | |
| RJ10.RC.3p | | |
| 234-Reg- | GTGCCTAAGTAAAACGACGGCCAGTACGCCA | 223 |
| T4R09C5- | GTTTATACATCTAG | |
| T3.3p- | | |
| RJ10.RC.3p | | |
| 235-Reg- | AACCGAGGGCAAAGACACCACGGATAAATAT | 224 |
| T2R12C4- | TTTTCACTCACCTCCATCTCCACTCCTACCCAT | |
| T3.3p- | CCAACTCCCAC | |
| APT40.2.3p | | |

The SNAP has a nucleic acid origami structure that forms a rectangular tile. A post protrudes from the planar surface of the tile and is configured to attach analytes such as proteins or short peptide epitopes. The SNAP further includes Alexa-488 dyes attached to twenty handles located near the perimeter of the tile (sequences with the word “DYE” in the first column of Table 1). Eleven dockers also protrude from the surface of the SNAP tile. The dockers include the sequences including an 8 nucleotide sequence region (ATACATCT) at the 3′ end.

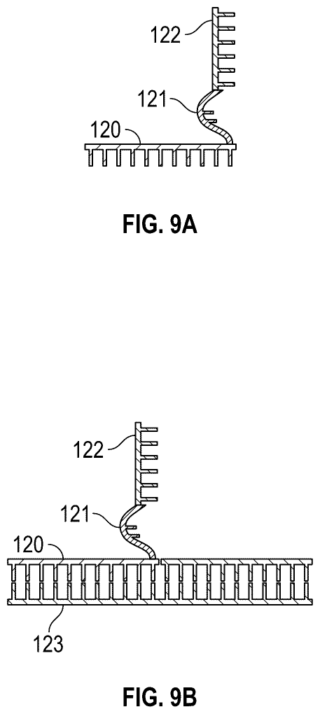

FIG. 9 shows a diagram of sequence regions for a SNAP docker including an origami binding region 120, a swivel region 121 and tether binding region 122. Exemplary tether binding regions include, but are not limited to, the RJ-8 nt sequence (ATACATCT), the RJ-9 nt sequence (ATACATCTA), the RJ-10 nt sequence (ATACATCTAG), the MD-8 nt sequence (ATCTGTCT), the MD-9 nt sequence (ATCTGTCTT), and the MD-10 nt sequence (ATCTGTCTTA). Shorter regions of the foregoing sequences can be used as well if desired. The swivel region is designed to allow the docker to have a wide range of rotational freedom when bound to a tether and will generally remain single stranded when the docker is bound to the tether. A sequence of 3 thymines is useful, but other sequences can be used. FIG. 9B shows a diagram of the SNAP docker hybridized to a region 123 of the SNAP origami structure. Exemplary sequences for origami binding regions for the SNAP are set forth in Table 2.

TABLE 2

| | Hybridization Region | SEQ |
|---|---|---|
| Location in SNAP | Sequence | ID NO |
| 1 (205-Reg-T1R13C2) | AAAAGAATAACCGAACTGACCAAC | 206 |
| | TTCATCAAGAGTAATCTTTTAAGA | |
| 2 (206-Reg-T2R13C2) | CGCTAATAGGAATACCCAAAAGAA | 207 |
| | ATACATAAAGGTGGCAGAATTATC | |
| 3 (207-Reg-T4R13C2) | TTCATCAACGCACTCCAGCCAGCT | 208 |
| | GCTGCGCAACTGTTGGAGAGGATC | |
| 4 (220-Reg-T1R09C5) | CATTCAACGAGATGGTTTAATTTC | 215 |
| | CAACGTAA | |
| 5 (221-Reg-T2R05C5) | TAAGTTTTAACAAATAAATCCTCA | 216 |
| | ACCAGAAC | |
| 6 (222-Reg-T2R09C5) | GTGCCTAAGTAAAACGACGGCCAG | 217 |
| | TACGCCAG | |
| 7 (52-Reg-T3R11C2) | AACATTATGTAAAACAGAAATAAA | 53 |
| | TTTTACAT | |
| 8 (53-Reg-T3R11C4) | CCAGAAGGTTAGAACCTACCATAT | 54 |
| | CCTGATTG | |
| 9 (78-Reg-T1R15C5) | TTGCGGGATTAAACAGCTTGATAC | 79 |
| | TGAAAATCTCCAAAAAAGCGGAGT | |
| 10 (81-Reg-T2R15C5) | AACAGCCATTAAATCAAGATTAGT | 82 |
| | ATATAGAAGGCTTATCCAAGCCGT | |
| 11 (82-Reg-T4R15C5) | AGAAAAGCACAAAGGCTATCAGGT | 83 |
| | AAATCACCATCAATATCAATGCCT | |

The Lobe has a nucleic acid origami structure with twelve locations for attachment of tethers. The origami structure of the Lobe is formed using the p7249 scaffold and the oligonucleotides listed in Table 3. The Lobe also includes Alexa 647 fluorescent labels attached near the perimeter of the origami structure. The origami Lobe folds to produce a tile-shaped structure.

TABLE 3

| Sequence Name | Sequence | SEQ ID NO |
|---|---|---|
| 1-Reg-T1R01C6 | TCATTTGCTAATAGTAGTAGCATT | 2 |
| 2-Reg-T1R03C5 | CAACTAAAGTACGGTGGGATGGCT | 227 |
| 3-Reg-T1R09C2 | CATTATTAGCAAAAGAAGTTTTGC | 228 |
| 4-Reg-T2R01C6 | ACCCTCATTCAGGGATAGCAAGCC | 3 |
| 5-Reg-T2R03C5 | TTAGGATTAGCGGGGTGGAACCTA | 229 |
| 6-Reg-T2R09C2 | AGGCCGGAACCAGAGCCACCACCG | 230 |
| 7-Reg-T3R01C6 | AGAATATCAGACGACGACAATAAA | 4 |
| 8-Reg-T3R03C5 | TCATATGCGTTATACAAAGGCGTT | 5 |
| 9-Reg-T3R09C2 | CGGGAGAATTTAATGGAAACAGTA | 6 |
| 10-Reg-T4R01C6 | GCGCGTACTTTCCTCGTTAGAATC | 7 |
| 11-Reg-T4R03C5 | AAAGCCGGCGAACGTGTGCCGTAA | 231 |
| 12-Reg-T4R09C2 | AATTCCACGTTTGCGTATTGGGCG | 232 |
| 13-Reg-T1R03C6 | TTTCATTGAGTAGATTTAGTTTCTATATTT | 186 |
| 14-Reg-T1R06C3 | TTAAGAGGGTCCAATACTGCGGATAGCGAG | 8 |
| 15-Reg-T1R07C6 | AACAGTTAGGTCTTTACCCTGATCCAACAG | 187 |
| 16-Reg-T1R08C3 | AGGCTTTTCAGGTAGAAAGATTCAATTACC | 9 |
| 17-Reg-T1R10C3 | TTATGCGATTGACAAGAACCGGAGGTCAAT | 10 |
| 18-Reg-T1R11C6 | GTGAATATAGTAAATTGGGCTTTAATGCAG | 188 |
| 19-Reg-T1R12C3 | CATAAGGGACACTAAAACACTCACATTAAA | 11 |
| 20-Reg-T1R14C3 | CGGGTAAAATTCGGTCGCTGAGGAATGACA | 12 |
| 21-Reg-T1R15C6 | CTCAGCAGGCTACAGAGGCTTTAACAAAGT | 189 |
| 22-Reg-T1R19C6 | GTTAGTAACTTTCAACAGTTTCAAAGGCTC | 190 |
| 23-Reg-T2R03C6 | GTACCAGGTATAGCCCGGAATAGAACCGCC | 191 |
| 24-Reg-T2R06C3 | GTCTCTGACACCCTCAGAGCCACATCAAAA | 13 |
| 25-Reg-T2R07C6 | GCCAGCAGCCTTGATATTCACAAACGGGGT | 192 |
| 26-Reg-T2R08C3 | TCACCGGAAACGTCACCAATGAATTATTCA | 14 |
| 27-Reg-T2R10C3 | TTAAAGGTACATATAAAAGAAACAAACGCA | 15 |
| 28-Reg-T2R11C6 | TAGAAAAGGCGACATTCAACCGCAGAATCA | 193 |
| 29-Reg-T2R12C3 | ATAATAACTCAGAGAGATAACCCGAAGCGC | 16 |
| 30-Reg-T2R14C3 | ATTAGACGGAGCGTCTTTCCAGAGCTACAA | 17 |
| 31-Reg-T2R15C6 | ATCCCAAAAAAATGAAAATAGCAAGAAACA | 194 |
| 32-Reg-T2R19C6 | CTTATCACTCATCGAGAACAAGCGGTATTC | 195 |
| 33-Reg-T3R03C6 | CCAGTATGAATCGCCATATTTAGTAATAAG | 196 |
| 34-Reg-T3R06C3 | TATATAACGTAAATCGTCGCTATATTTGAA | 18 |
| 35-Reg-T3R07C6 | GCTTAGAATCAAAATCATAGGTTTTAGTTA | 197 |
| 36-Reg-T3R08C3 | TTACCTTTACAATAACGGATTCGCAAAATT | 19 |
| 37-Reg-T3R10C3 | ATTTGCACCATTTTGCGGAACAAATTTGAG | 20 |
| 38-Reg-T3R11C6 | ATTATCAGTTTGGATTATACTTGCGCAGAG | 198 |
| 39-Reg-T3R12C3 | GATTTAGATTGCTGAACCTCAAAGTATTAA | 21 |
| 40-Reg-T3R14C3 | CACCGCCTGAAAGCGTAAGAATACATTCTG | 22 |
| 41-Reg-T3R15C6 | ATGCGCGTACCGAACGAACCACGCAAATCA | 199 |
| 42-Reg-T3R19C6 | TTAACCGTCACTTGCCTGAGTACTCATGGA | 200 |
| 43-Reg-T4R03C6 | GGAAGGGGGCAAGTGTAGCGGTGCTACAGG | 201 |
| 44-Reg-T4R06C3 | TGAGTGTTCAGCTGATTGCCCTTGCGCGGG | 23 |
| 45-Reg-T4R07C6 | CTGGTTTGTTCCGAAATCGGCATCTATCAG | 202 |
| 46-Reg-T4R08C3 | GAGAGGCGACAACATACGAGCCGCTGCAGG | 24 |
| 47-Reg-T4R10C3 | TCGACTCTGAAGGGCGATCGGTGCGGCCTC | 25 |
| 48-Reg-T4R11C6 | GTGCTGCCCCAGTCACGACGTTTGAGTGAG | 203 |
| 49-Reg-T4R12C3 | AGGAAGATCATTAAATGTGAGCGTTTTTAA | 26 |
| 50-Reg-T4R14C3 | CCAATAGGAAACTAGCATGTCAAGGAGCAA | 27 |
| 51-Reg-T4R15C6 | CAGGAAGTAATATTTTGTTAAAAACGGCGG | 204 |
| 52-Reg-T4R19C6 | CCTTTATCATATATTTTAAATGGATATTCA | 205 |
| 53-Reg-T1R04C5 | TAGAGCTTCAGACCGGAAGCAAACCTATTAT | 233 |
| | A | |
| 54-Reg-T1R06C5 | GTCAGAAGATTGAATCCCCCTCAACCTCGTTT | 234 |
| 55-Reg-T1R07C4 | AAATATTCCAAAGCGGATTGCATCGAGCTTCA | 29 |
| 56-Reg-T1R08C5 | ACCAGACGGAATACCACATTCAACGAGATGG | 235 |
| | T | |
| 57-Reg-T1R10C1 | AGTCAGGACATAGGCTGGCTGACCTTTGAAA | 32 |
| | G | |
| 58-Reg-T1R10C5 | TTAATTTCCAACGTAACAAAGCTGTCCATGTT | 236 |
| 59-Reg-T1R11C4 | ACCCAAATAACTTTAATCATTGTGATCAGTTG | 33 |
| 60-Reg-T1R12C5 | ACTTAGCCATTATACCAAGCGCGAGAGGACT | 237 |
| | A | |
| 61-Reg-T1R13C2 | AAAAGAATAACCGAACTGACCAACTTCATCA | 238 |
| | A | |
| 62-Reg-T1R13C4 | CCCCAGCGGGAACGAGGCGCAGACTATTCAT | 35 |
| | T | |
| 63-Reg-T1R14C5 | AAGACTTTGGCCGCTTTTGCGGGATTAAACAG | 239 |
| 64-Reg-T1R16C5 | CTTGATACTGAAAATCTCCAAAAAAGCGGAG | 240 |
| | T | |
| 65-Reg-T1R17C4 | TTTCACGTCGATAGTTGCGCCGACCTTGCAGG | 38 |
| 66-Reg-T2R04C5 | TTATTCTGACTGGTAATAAGTTTTAACAAATA | 241 |
| 67-Reg-T2R06C5 | AATCCTCAACCAGAACCACCACCAGCCCCCTT | 242 |
| 68-Reg-T2R07C4 | GAGCCGCCTTAAAGCCAGAATGGAGATGATA | 39 |
| | C | |
| 69-Reg-T2R08C5 | ATTAGCGTCCGTAATCAGTAGCGAATTGAGG | 243 |
| | G | |
| 70-Reg-T2R10C1 | GCCATTTGCAAACGTAGAAAATACCTGGCAT | 42 |
| | G | |
| 71-Reg-T2R10C5 | AGGGAAGGATAAGTTTATTTTGTCAGCCGAAC | 244 |
| 72-Reg-T2R11C4 | ACCACGGATAAATATTGACGGAAAACCATCG | 245 |
| | A | |
| 73-Reg-T2R12C5 | AAAGTTACGCCCAATAATAAGAGCAGCCTTT | 246 |
| | A | |
| 74-Reg-T2R13C2 | CGCTAATAGGAATACCCAAAAGAAATACATA | 247 |
| | A | |
| 75-Reg-T2R13C4 | TGAGTTAACAGAAGGAAACCGAGGGCAAAGA | 248 |
| | C | |
| 76-Reg-T2R14C5 | CAGAGAGAACAAAATAAACAGCCATTAAATC | 249 |
| | A | |
| 77-Reg-T2R16C5 | AGATTAGTATATAGAAGGCTTATCCAAGCCGT | 250 |
| 78-Reg-T2R17C4 | CAAATCAGTGCTATTTTGCACCCAGCCTAATT | 45 |
| 79-Reg-T3R04C5 | AAATAAGAACTTTTTCAAATATATCTGAGAGA | 46 |
| 80-Reg-T3R06C5 | CTACCTTTAGAATCCTTGAAAACAAGAAAAC | 47 |
| | A | |
| 81-Reg-T3R07C4 | TTTCCCTTTTAACCTCCGGCTTAGCAAAGAAC | 48 |
| 82-Reg-T3R08C5 | AAATTAATACCAAGTTACAAAATCCTGAATA | 49 |
| | A | |
| 83-Reg-T3R10C1 | GTAGATTTGTTATTAATTTTAAAAAACAATTC | 51 |
| 84-Reg-T3R10C5 | TGGAAGGGAGCGGAATTATCATCAACTAATA | 52 |
| | G | |
| 85-Reg-T3R11C4 | CCAGAAGGTTAGAACCTACCATATCCTGATTG | 54 |
| 86-Reg-T3R12C5 | ATTAGAGCAATATCTGGTCAGTTGCAGCAGA | 55 |
| | A | |
| 87-Reg-T3R13C2 | GCATCACCAGTATTAGACTTTACAGTTTGAGT | 56 |
| 88-Reg-T3R13C4 | CCTCAATCCGTCAATAGATAATACAGAAACC | 57 |
| | A | |
| 89-Reg-T3R14C5 | GATAAAACTTTTTGAATGGCTATTTTCACCAG | 58 |
| 90-Reg-T3R16C5 | TCACACGATGCAACAGGAAAAACGGAAGAAC | 60 |
| | T | |
| 91-Reg-T3R17C4 | CCAGCCATCCAGTAATAAAAGGGACGTGGCA | 61 |
| | C | |
| 92-Reg-T4R04C5 | AGCACTAAAAAGGGCGAAAAACCGAAATCCC | 251 |
| | T | |
| 93-Reg-T4R06C5 | TATAAATCGAGAGTTGCAGCAAGCGTCGTGC | 252 |
| | C | |
| 94-Reg-T4R07C4 | GGCCCTGAAAAAGAATAGCCCGAGCGTGGAC | 63 |
| | T | |
| 95-Reg-T4R08C5 | AGCTGCATAGCCTGGGGTGCCTAAGTAAAAC | 253 |
| | G | |
| 96-Reg-T4R10C1 | GAATTCGTGCCATTCGCCATTCAGTTCCGGCA | 66 |
| 97-Reg-T4R10C5 | ACGGCCAGTACGCCAGCTGGCGAACATCTGC | 254 |
| | C | |
| 98-Reg-T4R11C4 | TTCGCTATTGCCAAGCTTGCATGCGAAGCATA | 67 |
| 99-Reg-T4R12C5 | AGTTTGAGATTCTCCGTGGGAACAATTCGCAT | 255 |
| 100-Reg-T4R13C2 | TTCATCAACGCACTCCAGCCAGCTGCTGCGCA | 256 |
| 101-Reg-T4R13C4 | CCCGTCGGGGGACGACGACAGTATCGGGCCT | 69 |
| | C | |
| 102-Reg-T4R14C5 | TAAATTTTTGATAATCAGAAAAGCACAAAGG | 257 |
| | C | |
| 103-Reg-T4R16C5 | TATCAGGTAAATCACCATCAATATCAATGCCT | 258 |
| 104-Reg-T4R17C4 | AGACAGTCCATTGCCTGAGAGTCTTCATATGT | 72 |
| 105-Reg-T2R21C5 | AGCTAATGCAGAACGCGAGAAAAATAATATC | 259 |
| | CTGTCTTTC | |
| 106-Reg-T4R21C5 | AATCATACAGGCAAGGCAGAGCATAAAGCTA | 260 |
| | AGGGAGAAG | |
| 107-Edg-T1R00C7- | TTTGGTGGCATCAATTCTAGGGCGCGAGCTGA | 84 |
| T3.5p-T3.3p- | AAATTTCGTTTCTTTGCTTGTGTCTTGTCTTCG | |
| DYE40.22.9.3p | TCGTGCTTGTTCC | |
| 108-Edg-T1R02C7- | TTTTCCCAATTCTGCGAACCCATATAACAGTT | 85 |
| T3.5p-T3.3p- | GATTTTCGTTTCTTTGCTTGTGTCTTGTCTTCG | |
| DYE40.22.9.3p | TCGTGCTTGTTCC | |
| 109-Edg-T1R04C7- | TTTATTGCTCCTTTTGATATTAGAGAGTACCTT | 86 |
| T3.5p-T3.3p- | TATTTCGTTTCTTTGCTTGTGTCTTGTCTTCGT | |
| DYE40.22.9.3p | CGTGCTTGTTCC | |
| 110-Edg-T1R06C7- | TTTCCATAAATCAAAAATCCAGAAAACGAGA | 87 |
| T3.5p-T3.3p- | ATGATTTCGTTTCTTTGCTTGTGTCTTGTCTTC | |
| DYE40.22.9.3p | GTCGTGCTTGTTCC | |
| 111-Edg-T1R08C7- | TTTCGAGGCATAGTAAGAGACGCCAAAAGGA | 88 |
| T3.5p-T3.3p- | ATTATTTCGTTTCTTTGCTTGTGTCTTGTCTTC | |
| DYE40.22.9.3p | GTCGTGCTTGTTCC | |
| 112-Edg-T1R10C7- | TTTGAAACACCAGAACGAGAGGCTTGCCCTG | 89 |
| T3.5p-T3.3p- | ACGATTTCGTTTCTTTGCTTGTGTCTTGTCTTC | |
| DYE40.22.9.3p | GTCGTGCTTGTTCC | |
| 113-Edg-T1R12C7- | TTTCTGATAAATTGTGTCGAGATTTGTATCAT | 90 |
| T3.5p-T3.3p- | CGCTTTCGTTTCTTTGCTTGTGTCTTGTCTTCG | |
| DYE40.22.9.3p | TCGTGCTTGTTCC | |
| 114-Edg-T1R14C7- | TTTGAACGAGGGTAGCAACGCGAAAGACAGC | 91 |
| T3.5p-T3.3p- | ATCGTTTCGTTTCTTTGCTTGTGTCTTGTCTTC | |
| DYE40.22.9.3p | GTCGTGCTTGTTCC | |
| 115-Edg-T1R16C7- | TTTGGTTTATCAGCTTGCTAGCCTTTAATTGTA | 92 |
| T3.5p-T3.3p- | TCTTTCGTTTCTTTGCTTGTGTCTTGTCTTCGTC | |
| DYE40.22.9.3p | GTGCTTGTTCC | |
| 116-Edg-T1R18C7- | TTTGGGATTTTGCTAAACAAATGAATTTTCTG | 93 |
| T3.5p-T3.3p- | TATTTTCGTTTCTTTGCTTGTGTCTTGTCTTCGT | |
| DYE40.22.9.3p | CGTGCTTGTTCC | |
| 117-Edg-T1R20C7- | TTTACAAACTACAACGCCTGAGTTTCGTCACC | 94 |
| T3.5p-T3.3p- | AGTTTTCGTTTCTTTGCTTGTGTCTTGTCTTCG | |
| DYE40.22.9.3p | TCGTGCTTGTTCC | |
| 118-Edg-T2R00C7- | TTTAGCCACCACCCTCATTGAACCGCCACCCT | 95 |
| T3.5p-T3.3p- | CAGTTTCGTTTCTTTGCTTGTGTCTTGTCTTCG | |
| DYE40.22.9.3p | TCGTGCTTGTTCC | |
| 119-Edg-T2R02C7- | TTTGAGAGGGTTGATATAAGCGGATAAGTGC | 96 |
| T3.5p-T3.3p- | CGTCTTTCGTTTCTTTGCTTGTGTCTTGTCTTC | |
| DYE40.22.9.3p | GTCGTGCTTGTTCC | |
| 120-Edg-T2R04C7- | TTTGTATAAACAGTTAATGTTGAGTAACAGTG | 97 |
| T3.5p-T3.3p- | CCCTTTCGTTTCTTTGCTTGTGTCTTGTCTTCG | |
| DYE40.22.9.3p | TCGTGCTTGTTCC | |
| 121-Edg-T2R06C7- | TTTGCAGGTCAGACGATTGTTGACAGGAGGTT | 98 |
| T3.5p-T3.3p- | GAGTTTCGTTTCTTTGCTTGTGTCTTGTCTTCG | |
| DYE40.22.9.3p | TCGTGCTTGTTCC | |
| 122-Edg-T2R08C7- | TTTTAGCGCGTTTTCATCGCTTTAGCGTCAGA | 99 |
| T3.5p-T3.3p- | CTGTTTCGTTTCTTTGCTTGTGTCTTGTCTTCG | |
| DYE40.22.9.3p | TCGTGCTTGTTCC | |
| 123-Edg-T2R10C7- | TTTGCGCCAAAGACAAAAGTTCATATGGTTTA | 100 |
| T3.5p-T3.3p- | CCATTTCGTTTCTTTGCTTGTGTCTTGTCTTCG | |
| DYE40.22.9.3p | TCGTGCTTGTTCC | |
| 124-Edg-T2R12C7- | TTTCCGAAGCCCTTTTTAAAGCAATAGCTATC | 101 |
| T3.5p-T3.3p- | TTATTTCGTTTCTTTGCTTGTGTCTTGTCTTCGT | |
| DYE40.22.9.3p | CGTGCTTGTTCC | |
| 125-Edg-T2R14C7- | TTTTTTTTTGTTTAACGTCTCCAAATAAGAAAC | 102 |
| T3.5p-T3.3p- | GATTTCGTTTCTTTGCTTGTGTCTTGTCTTCGT | |
| DYE40.22.9.3p | CGTGCTTGTTCC | |
| 126-Edg-T2R16C7- | TTTAACCTCCCGACTTGCGGCGAGGCGTTTTA | 103 |
| T3.5p-T3.3p- | GCGTTTCGTTTCTTTGCTTGTGTCTTGTCTTCG | |
| DYE40.22.9.3p | TCGTGCTTGTTCC | |
| 127-Edg-T2R18C7- | TTTTAAACCAAGTACCGCATTCCAAGAACGGG | 104 |
| T3.5p-T3.3p- | TATTTTCGTTTCTTTGCTTGTGTCTTGTCTTCGT | |
| DYE40.22.9.3p | CGTGCTTGTTCC | |
| 128-Edg-T2R20C7- | TTTAGATAAGTCCTGAACACCTGTTTATCAAC | 105 |
| T3.5p-T3.3p- | AATTTTCGTTTCTTTGCTTGTGTCTTGTCTTCG | |
| DYE40.22.9.3p | TCGTGCTTGTTCC | |
| 129-Edg-T3R00C7- | TTTGTAAAGTAATTCTGTCAAAGTACCGACAA | 106 |
| T3.5p-T3.3p- | AAGTTTCGTTTCTTTGCTTGTGTCTTGTCTTCG | |
| DYE40.22.9.3p | TCGTGCTTGTTCC | |
| 130-Edg-T3R02C7- | TTTAGTAGGGCTTAATTGAAAAGCCAACGCTC | 107 |
| T3.5p-T3.3p- | AACTTTCGTTTCTTTGCTTGTGTCTTGTCTTCG | |
| DYE40.22.9.3p | TCGTGCTTGTTCC | |
| 131-Edg-T3R04C7- | TTTAATGGTTTGAAATACCCTTCTGACCTAAA | 108 |
| T3.5p-T3.3p- | TTTTTTCGTTTCTTTGCTTGTGTCTTGTCTTCGT | |
| DYE40.22.9.3p | CGTGCTTGTTCC | |
| 132-Edg-T3R06C7- | TTTAGTCAATAGTGAATTTTTAAGACGCTGAG | 109 |
| T3.5p-T3.3p- | AAGTTTCGTTTCTTTGCTTGTGTCTTGTCTTCG | |
| DYE40.22.9.3p | TCGTGCTTGTTCC | |
| 133-Edg-T3R08C7- | TTTTGAGCAAAAGAAGATGATTCATTTCAATT | 110 |
| T3.5p-T3.3p- | ACCTTTCGTTTCTTTGCTTGTGTCTTGTCTTCG | |
| DYE40.22.9.3p | TCGTGCTTGTTCC | |
| 134-Edg-T3R10C7- | TTTCAATATAATCCTGATTGATGATGGCAATT | 111 |
| T3.5p-T3.3p- | CATTTTCGTTTCTTTGCTTGTGTCTTGTCTTCG | |
| DYE40.22.9.3p | TCGTGCTTGTTCC | |
| 135-Edg-T3R12C7- | TTTGTTATCTAAAATATCTAAAGGAATTGAGG | 112 |
| T3.5p-T3.3p- | AAGTTTCGTTTCTTTGCTTGTGTCTTGTCTTCG | |
| DYE40.22.9.3p | TCGTGCTTGTTCC | |
| 136-Edg-T3R14C7- | TTTACATCGCCATTAAAAAAACTGATAGCCCT | 113 |
| T3.5p-T3.3p- | AAATTTCGTTTCTTTGCTTGTGTCTTGTCTTCG | |
| DYE40.22.9.3p | TCGTGCTTGTTCC | |
| 137-Edg-T3R16C7- | TTTTCGTCTGAAATGGATTACATTTTGACGCT | 114 |
| T3.5p-T3.3p- | CAATTTCGTTTCTTTGCTTGTGTCTTGTCTTCG | |
| DYE40.22.9.3p | TCGTGCTTGTTCC | |
| 138-Edg-T3R18C7- | TTTTTGATTAGTAATAACATTGTAGCAATACT | 115 |
| T3.5p-T3.3p- | TCTTTTCGTTTCTTTGCTTGTGTCTTGTCTTCGT | |
| DYE40.22.9.3p | CGTGCTTGTTCC | |
| 139-Edg-T3R20C7- | TTTAGGAACGGTACGCCAGTAAAGGGATTTTA | 116 |
| T3.5p-T3.3p- | GACTTTCGTTTCTTTGCTTGTGTCTTGTCTTCG | |
| DYE40.22.9.3p | TCGTGCTTGTTCC | |
| 140-Edg-T4R00C7- | TTTGAGCACGTATAACGTGCTATGGTTGCTTT | 117 |
| T3.5p-T3.3p- | GACTTTCGTTTCTTTGCTTGTGTCTTGTCTTCG | |
| DYE40.22.9.3p | TCGTGCTTGTTCC | |
| 141-Edg-T4R02C7- | TTTCGGGCGCTAGGGCGCTAAGAAAGCGAAA | 118 |
| T3.5p-T3.3p- | GGAGTTTCGTTTCTTTGCTTGTGTCTTGTCTTC | |
| DYE40.22.9.3p | GTCGTGCTTGTTCC | |
| 142-Edg-T4R04C7- | TTTATCACCCAAATCAAGTGCCCACTACGTGA | 119 |
| T3.5p-T3.3p- | ACCTTTCGTTTCTTTGCTTGTGTCTTGTCTTCG | |
| DYE40.22.9.3p | TCGTGCTTGTTCC | |
| 143-Edg-T4R06C7- | TTTATCCTGTTTGATGGTGGCCCCAGCAGGCG | 120 |
| T3.5p-T3.3p- | AAATTTCGTTTCTTTGCTTGTGTCTTGTCTTCG | |
| DYE40.22.9.3p | TCGTGCTTGTTCC | |
| 144-Edg-T4R08C7- | TTTGCTCACTGCCCGCTTTACATTAATTGCGTT | 121 |
| T3.5p-T3.3p- | GCTTTCGTTTCTTTGCTTGTGTCTTGTCTTCGT | |
| DYE40.22.9.3p | CGTGCTTGTTCC | |
| 145-Edg-T4R10C7- | TTTGTAACGCCAGGGTTTTAAGGCGATTAAGT | 122 |
| T3.5p-T3.3p- | TGGTTTCGTTTCTTTGCTTGTGTCTTGTCTTCG | |
| DYE40.22.9.3p | TCGTGCTTGTTCC | |
| 146-Edg-T4R12C7- | TTTCGTTGGTGTAGATGGGGTAATGGGATAGG | 123 |
| T3.5p-T3.3p- | TCATTTCGTTTCTTTGCTTGTGTCTTGTCTTCG | |
| DYE40.22.9.3p | TCGTGCTTGTTCC | |
| 147-Edg-T4R14C7- | TTTTTTAAATTGTAAACGTATTGTATAAGCAA | 124 |
| T3.5p-T3.3p- | ATATTTCGTTTCTTTGCTTGTGTCTTGTCTTCG | |
| DYE40.22.9.3p | TCGTGCTTGTTCC | |
| 148-Edg-T4R16C7- | TTTGCCGGAGAGGGTAGCTTAGCTGATAAATT | 125 |
| T3.5p-T3.3p- | AATTTTCGTTTCTTTGCTTGTGTCTTGTCTTCG | |
| DYE40.22.9.3p | TCGTGCTTGTTCC | |
| 149-Edg-T4R18C7- | TTTAAATTTTTAGAACCCTTTCAACGCAAGGA | 126 |
| T3.5p-T3.3p- | TAATTTCGTTTCTTTGCTTGTGTCTTGTCTTCG | |
| DYE40.22.9.3p | TCGTGCTTGTTCC | |
| 150-Edg-T4R20C7- | TTTTAAGCAATAAAGCCTCAAAGAATTAGCA | 127 |
| T3.5p-T3.3p- | AAATTTTCGTTTCTTTGCTTGTGTCTTGTCTTC | |
| DYE40.22.9.3p | GTCGTGCTTGTTCC | |
| 151-Bri-T1R14C2 | TGCCACTACTTTTTTTGCCACCCTC | 128 |
| 152-Bri-T2R14C2 | AACTGAACATTTTTTTTGAATAACC | 129 |
| 153-Bri-T3R14C2 | GCCACGCTGTTTTTTTACCAGTGAG | 130 |
| 154-Bri-T4R14C2 | CAAAAATAATTTTTTTTGTTTAGAC | 131 |
| 155-Bri-T1R02C5 | GATACATTTCGCTTTTTTGACCCTGTAAT | 132 |
| 156-Bri-T2R02C5 | ACCGTACTCAGGTTTTTGATCTAAAGTTT | 133 |
| 157-Bri-T3R02C5 | AACATGTAATTTTTTTTGAAACCAATCAA | 134 |
| 158-Bri-T4R02C5 | GCGTAACCACCATTTTTGAGTAAAAGAGT | 135 |
| 159-Bri-T1R08C1 | CAGAGGGGGTTTTGCCTTCCTGTAGCCAGCT | 136 |
| 160-Bri-T2R08C1 | GAACCGCCTCTTTACCTAAAACGAAAGAGGC | 137 |
| 161-Bri-T3R08C1 | CATAAATCAATTTAGTCAGAGGGTAATTGAG | 138 |
| 162-Bri-T4R08C1 | CCAGGGTGGTTTTGCAAATGAAAAATCTAAA | 139 |
| 163-Bri-T1R16C3 | ACAACCATTTTTTCATACATGGCTTTTAAGCG | 140 |
| | CA | |
| 164-Bri-T2R16C3 | TTTTATCTTTTTTATCCAATCGCAAGAGTTGGG | 141 |
| | T | |
| 165-Bri-T3R16C3 | GCCAACATTTTTTCCACTATTAAAGAAATAGG | 142 |
| | GT | |
| 166-Bri-T4R16C3 | ACAAGAGTTTTTTTCGCGTTTTAATTCAAAAA | 143 |
| | GA | |
| 167-Bri-T1R07C2 | TGGATAGCAAGCCCGATTTTTAATCGTAAACG | 144 |
| | CCAT | |
| 168-Bri-T2R07C2 | AGAACCGCATTTACCGTTTTACCGATATATAC | 145 |
| | GTAA | |
| 169-Bri-T3R07C2 | TTGCTTCTTATATGTATTTTACGCTAACGGAG | 146 |
| | AATT | |
| 170-Bri-T4R07C2 | ACGGGCAAGTTCCAGTTTTTTCTGACCTGCAA | 147 |
| | CAGT | |
| 171-Bri-T1R18C5 | GAGAATAGAAAGGAACAACTATTTTCTCAAG | 148 |
| | AGAAGGA | |
| 172-Bri-T2R18C5 | TTTTATTTTCATCGTAGGAATTTTTAGCCTGTT | 149 |
| | TAGTA | |
| 173-Bri-T3R18C5 | CAAACTATCGGCCTTGCTGGTTTTTGAGCTTG | 150 |
| | ACGGGG | |
| 174-Bri-T4R18C5 | GAGTAATGTGTAGGTAAAGATTTTTTGTTTTA | 151 |
| | AATATG | |
| 175-Bri-T1R12C1 | AGGACAGATGATTTTTTCACCAGTAGCACCAT | 152 |
| | TACCGACTTGA | |
| 176-Bri-T2R12C1 | ATTAAGACTCCTTTTTAATATACAGTAACAGT | 153 |
| | ACCGAAATTGC | |
| 177-Bri-T3R12C1 | GACAACTCGTATTTTTTCCTGTGTGAAATTGTT | 154 |
| | ATCCGAGCTC | |
| 178-Bri-T4R12C1 | CCGCTTCTGGTTTTTTCGTTAATAAAACGAAC | 155 |
| | TAAATTATACC | |
| 179-Bri-T1R19C5 | TGTCGTCTCAGCCCTCATATTTTTTTCGCCACC | 156 |
| | CTCAGGTGTATC | |
| 180-Bri-T2R19C5 | TAATCGGCCATCCTAATTTTTTTTTTTTTTCGA | 157 |
| | GCCAACAACGCC | |
| 181-Bri-T3R19C5 | CTGTCCATTTTTATAATCATTTTTTTCTTAATG | 158 |
| | CGCCCACGCTGC | |
| 182-Bri-T4R19C5 | ACTTTTGCATCGGTTGTACTTTTTTTAACCTGT | 159 |
| | TTAGGACCATTA | |
| 183-Bri-T1R05C4 | AAGCGAACAATTGCTGAATATAATGCTGTATT | 160 |
| | TTTTTGTGAGAAAGGCCGG | |
| 184-Bri-T2R05C4 | AGGAGTGTAAACATGAAAGTATTAAGAGGCT | 161 |
| | TTTTTTGCGAATAATAATTT | |
| 185-Bri-T3R05C4 | GCGAGAAAATAAACACCGGAATCATAATTAT | 162 |
| | TTTTTTCGCCCAATAGCAAG | |
| 186-Bri-T4R05C4 | CCAACGTCATCGGAACCCTAAAGGGAGCCCT | 163 |
| | TTTTTTGAACAATATTACCG | |
| 187-Bri-T2R10C0 | GGAATTAGAGCTTTTTTTTCAGACCAGGCGCG | 164 |
| | TTGGGAAGATTTTTTTTCCAGGCAAAGC | |
| 188-Bri-T4R10C0 | AATCATGGTCATTTTTTTTTTTGCCCGAACTCA | 165 |
| | GGTTTAACTTTTTTTTCAGTATGTTAG | |
| 213-Reg-T1R09C4- | AGATTTAGACGATAAAAACCAAAAATCGTCA | 261 |
| T3.3p-APT40.2.3p- | TTTTCACTCACCTCCATCTCCACTCCTACCCAT | |
| T3.3p-RJ10.3p | CCAACTCCCACTTTCTAGATGTAT | |
| 214-Reg-T1R11C2- | GAGTAATCTTTTAAGAACTGGCTCCGGAACAA | 262 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 215-Reg-T1R15C4- | GAGTTAAATTCATGAGGAAGTTTCTCTTTGAC | 263 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 216-Reg-T2R09C4- | TAGCAGCATTGCCATCTTTTCATACACCCTCA | 264 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 217-Reg-T2R11C2- | AGGTGGCAGAATTATCACCGTCACCATTAGCA | 265 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 218-Reg-T2R15C4- | TGCCAGTTATAACATAAAAACAGGACAAGAA | 266 |
| T3.3p-APT40.2.3p- | TTTTCACTCACCTCCATCTCCACTCCTACCCAT | |
| T3.3p-RJ10.3p | CCAACTCCCACTTTCTAGATGTAT | |
| 219-Reg-T3R09C4- | CTTTGAATTACATTTAACAATTTCTAATTAATT | 267 |
| T3.3p-APT40.2.3p- | TTCACTCACCTCCATCTCCACTCCTACCCATCC | |
| T3.3p-RJ10.3p | AACTCCCACTTTCTAGATGTAT | |
| 220-Reg-T3R11C2- | AACATTATGTAAAACAGAAATAAATTTTACAT | 268 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 221-Reg-T3R15C4- | AGACAATAAGAGGTGAGGCGGTCATATCAAA | 269 |
| T3.3p-APT40.2.3p- | CTTTCACTCACCTCCATCTCCACTCCTACCCAT | |
| T3.3p-RJ10.3p | CCAACTCCCACTTTCTAGATGTAT | |
| 222-Reg-T4R09C4- | AAGTGTAATAATGAATCGGCCAACCACCGCC | 270 |
| T3.3p-APT40.2.3p- | TTTTCACTCACCTCCATCTCCACTCCTACCCAT | |
| T3.3p-RJ10.3p | CCAACTCCCACTTTCTAGATGTAT | |
| 223-Reg-T4R11C2- | ACTGTTGGAGAGGATCCCCGGGTACCGCTCAC | 271 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 224-Reg-T4R15C4- | ACCCCGGTTGTTAAATCAGCTCATAGTAACAA | 272 |
| T3.3p-APT40.2.3p- | TTTCACTCACCTCCATCTCCACTCCTACCCATC | |
| T3.3p-RJ10.3p | CAACTCCCACTTTCTAGATGTAT | |
| 225-Reg-T1R05C6- | GTCAGGAAGAGGTCATTTTTGCTCTGGAAGTT | 273 |
| T3.3p-BIO.3p | T/3Bio/ | |
| 226-Reg-T1R13C6- | ACAACGGAAATCCGCGACCTGCCTCATTCATT | 274 |
| T3.3p-BIO.3p | T/3Bio/ | |
| 227-Reg-T2R09C6- | AGTTTGCGCATTTTCGGTCATAGAGCCGCCTT | 275 |
| T3.3p-BIO.3p | T/3Bio/ | |
| 228-Reg-T2R17C6- | TAAGAACGGAGGTTTTGAAGCCTATTATTTTT | 276 |
| T3.3p-BIO.3p | T/3Bio/ | |
| 229-Reg-T3R05C6- | ATTTCATGACCGTGTGATAAATAATTCTTATT | 277 |
| T3.3p-BIO.3p | T/3Bio/ | |
| 230-Reg-T3R13C6- | ACAGTTGTTAGGAGCACTAACATATTCCTGTT | 278 |
| T3.3p-BIO.3p | T/3Bio/ | |
| 231-Reg-T4R09C6- | CTAACTCCCAGTCGGGAAACCTGGTCCACGTT | 279 |
| T3.3p-BIO.3p | T/3Bio/ | |
| 232-Reg-T4R17C6- | ACCGTTCATTTTTGAGAGATCTCCCAAAAATT | 280 |
| T3.3p-BIO.3p | T/3Bio/ | |
| 233-Reg-T1R21C5- | CCATGTACCGTAACACTGTAGCATTCCACAGA | 281 |
| T3.3p-BIO.3p | TTCCAGACTTT/3Bio/ | |
| 234-Reg-T3R21C5- | CTAAACAGGAGGCCGATAATCCTGAGAAGTG | 282 |
| T3.3p-BIO.3p | TCACGCAAATTT/3Bio/ | |
| 235-Reg-T1R09C6 | ATACATACAACACTATCATAACATGCTTTA | 283 |
| 236-Reg-T1R17C6 | CAAAAGGTTCGAGGTGAATTTCTCGTCACC | 284 |
| 237-Reg-T2R05C6 | CAGTGCCCCCCCTGCCTATTTCTTTGCTCA | 285 |
| 238-Reg-T2R13C6 | ATGAAATGAAAAGTAAGCAGATACAATCAA | 286 |
| 239-Reg-T3R09C6 | GCGAATTATGAAACAAACATCATAGCGATA | 287 |
| 240-Reg-T3R17C6 | AATACCTATTTACATTGGCAGAAGTCTTTA | 288 |
| 241-Reg-T4R05C6 | GGCGATGTTTTTGGGGTCGAGGGCGAGAAA | 289 |
| 242-Reg-T4R13C6 | ATTGACCCGCATCGTAACCGTGAGGGGGAT | 290 |

The Lobe has a nucleic acid origami structure with 12 locations for attachment of tethers. The tethers include sequences having an 8 nucleotide sequence region (AGATGTAT) at the 3′ end. The 8 nucleotide sequence at the 3′ end of the tethers is complementary to the 8 nucleotide sequence at the 3′ end of the dockers. The Lobe also includes Alexa 647 fluorescent labels attached at 44 locations (sequences with the word “DYE” in the first column of Table 3). The Lobe also includes 10 biotinylated oligonucleotides (indicated “/3Bio/” in Table 3) for attachment of streptavidin modified antibodies or other affinity components.

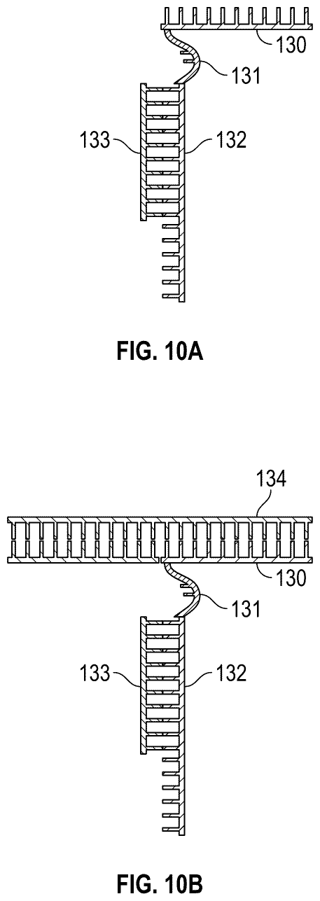

FIG. 10A shows a diagram of sequence regions for a v1 tether including an origami binding region 130, a swivel region 131, a docker binding region 132 and a stiffening region 133. Exemplary docker binding regions include, but are not limited to, the RJ sequence (CTAGATGTAT (SEQ ID NO: 291)) which is sufficiently long and complementary to any one of the RJ-8 nt, RJ-9 nt, RJ-10 nt sequences. Further examples of docker binding regions include the MD sequence (TAAGACAGAT (SEQ ID NO: 292)) which is sufficiently long and complementary to any one of the MD-8 nt, MD-9 nt or MD-10 nt sequences. Shorter regions of the RJ or MD sequences can be used as well, if desired. The swivel region is designed to allow the tether to have a wide range of rotational freedom when bound to a docker and can have similar structures to the docker swivel region exemplified above. The stiffener 133 functions to form a double stranded region, thereby increasing the end-to-end distance between the origami surfaces that are attached via interaction of tether and docker. This can prevent steric hinderance between the SNAP and Lobe. Stiffener 133 can have the sequence GTGGATGGGTAGGAGTGGAGATGGAGGTGAGTG (SEQ ID NO: 293) and can bind to a region of the v1 tether having the sequence CACTCACCTCCATCTCCACTCCTACCCATCCAAC (SEQ ID NO: 294). FIG. 10B shows a diagram of the v1 tether hybridized to a region 134 of the Lobe origami structure. Exemplary sequences for origami binding regions for a v1 tether are set forth in Table 4.

TABLE 4

| | | SEQ ID |
|---|---|---|
| Location in Lobe | Nucleotide Sequence in-structure | NO |
| 1 (189-Reg-T1R09C4) | AGATTTAGACGATAAAAACCAAAAATCGTCAT | 31 |
| 2 (190-Reg-T1R11C2) | GAGTAATCTTTTAAGAACTGGCTCCGGAACAA | 295 |
| 3 (191-Reg-T1R15C4) | GAGTTAAATTCATGAGGAAGTTTCTCTTTGAC | 37 |
| 4 (192-Reg-T2R09C4) | TAGCAGCATTGCCATCTTTTCATACACCCTCA | 296 |
| 5 (193-Reg-T2R11C2) | AGGTGGCAGAATTATCACCGTCACCATTAGCA | 297 |
| 6 (194-Reg-T2R15C4) | TGCCAGTTATAACATAAAAACAGGACAAGAAT | 298 |
| 7 (195-Reg-T3R09C4) | CTTTGAATTACATTTAACAATTTCTAATTAAT | 50 |
| 8 (196-Reg-T3R11C2) | AACATTATGTAAAACAGAAATAAATTTTACAT | 53 |
| 9 (197-Reg-T3R15C4) | AGACAATAAGAGGTGAGGCGGTCATATCAAAC | 59 |
| 10 (198-Reg- | AAGTGTAATAATGAATCGGCCAACCACCGCCT | 65 |
| T4R09C4) | | |
| 11 (199-Reg- | ACTGTTGGAGAGGATCCCCGGGTACCGCTCAC | 299 |
| T4R11C2) | | |
| 12 (200-Reg- | ACCCCGGTTGTTAAATCAGCTCATAGTAACAA | 71 |
| T4R15C4) | | |

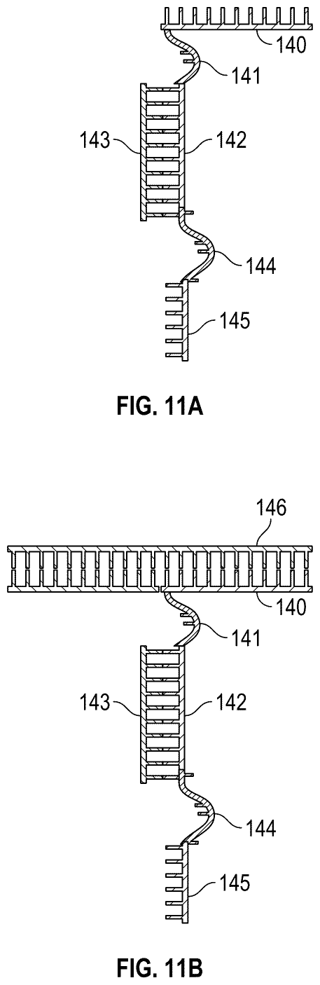

FIG. 11A shows a diagram of sequence regions for a v2 tether including an origami binding region 140, a swivel region 141, a stiffener binding region 142, a stiffener 143, a second swivel region 144 and a docker binding region 145. Exemplary docker binding regions include, but are not limited to, the RJ sequence and MD sequence set forth above in connection with the v1tether. Both swivel regions are designed to allow the tether to have a wide range of rotational freedom. However, the second swivel region 144 further functions to reduce base stacking between the stiffener and the duplex formed when the docker binding region 145 is bound to a docker sequence. The v1 tether interacts with a docker by Watson-Crick base pairing and base stacking. The v2 tether interacts with the docker via Watson-Crick base pairing, but the second swivel region reduces base-stacking in the duplex formed between v2 tether and docker. The stiffener has structure and function as set forth in the context of the v1 tether. FIG. 11B shows a diagram of the v1 tether hybridized to a region 134 of the Lobe origami structure. Exemplary sequences for origami binding regions for a Lobe are set forth in Table 4.

SNAPS and Lobes were folded using various dockers and tethers and evaluated using gel electrophoresis. A master mix was formed including 120 μl of 500 nM staple oligonucleotides (Table 1 or 3, respectively), 60 μl of 1 μM tethers or dockers (Table 2 or 4, respectively), 40 μl of 100 nM p7249 m13 ssDNA scaffold, 40 μl of 10×FOB (50 mM NaCl, 10 mM EDTA, 50 mM Tris-HC1 pH 8.0), 25 μl of 200 mM MgCl₂ and 115 μl water. The master mix was folded in strip tubes in a PCR instrument using the following cycle: 2 min at 95° C. followed by cooling from 90° C. to 20° C. at a rate of 1° C. per minute. Lobes having v2 tethers were not observed to fold well under the conditions tested. However, Lobes having vi tethers were observed to fold well as were the SNAPs.

### Example II

### Binding of Docker-Bearing SNAPs to Tether-Bearing Lobes

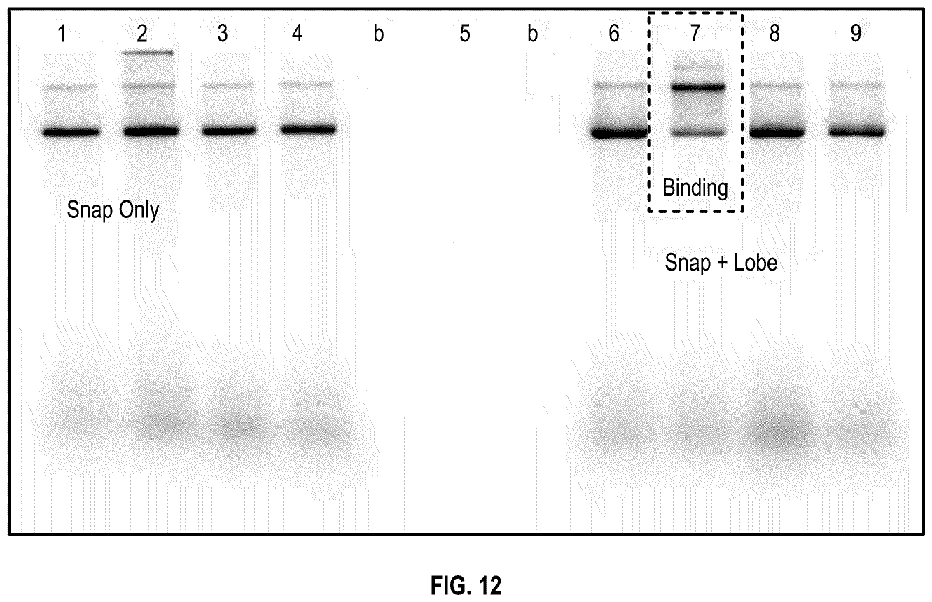

A gel-shift assay was performed as follows to detect binding between SNAPs and Lobes having structures set forth in Example I. The Lobes had v1 tethers. Four binding reactions were set up in which 100 nM Lobe (with RJ tether) was incubated with one of (1) 10 nM Null SNAP (i.e. the SNAP does not have any dockers), (2) 10 nM SNAP having 6 dockers with a 10 nucleotide complement of the RJ tether, (3) 10 nM SNAP having 1 docker with an 8 nucleotide complement of the RJ tether, (4) 10 nM SNAP having 1 docker with a 10 nucleotide complement of the RJ tether. Incubation occurred for 1 hour at room temperature, with shaking on a benchtop thermal shaker at 600 rpm. Then the samples were loaded on to a 2% Agarose gel as follows. Aliquots of the SNAPs used for each of the binding reactions were loaded in lanes 1 through 4 and binding reaction mixtures (1) through (4) were loaded in lanes 6 through 9. The gel was run for 3 hours on an ice bath at 70V. Gel Running Buffer was 0.5×TBE Buffer with 11 mM MgCl₂. The gels were then imaged to detect fluorescence from the Alexa-488 dyes on the SNAPs. FIG. 12 shows an image of the fluorescently detected gel. The shift observed for lane 7 indicated that the Lobe bearing the RJ tethers was able to bind to the SNAP having 6 dockers with a 10 nucleotide complement of the RJ tether. However, the SNAPs with only 1 docker did not demonstrate binding as compared to the SNAP having no docker.

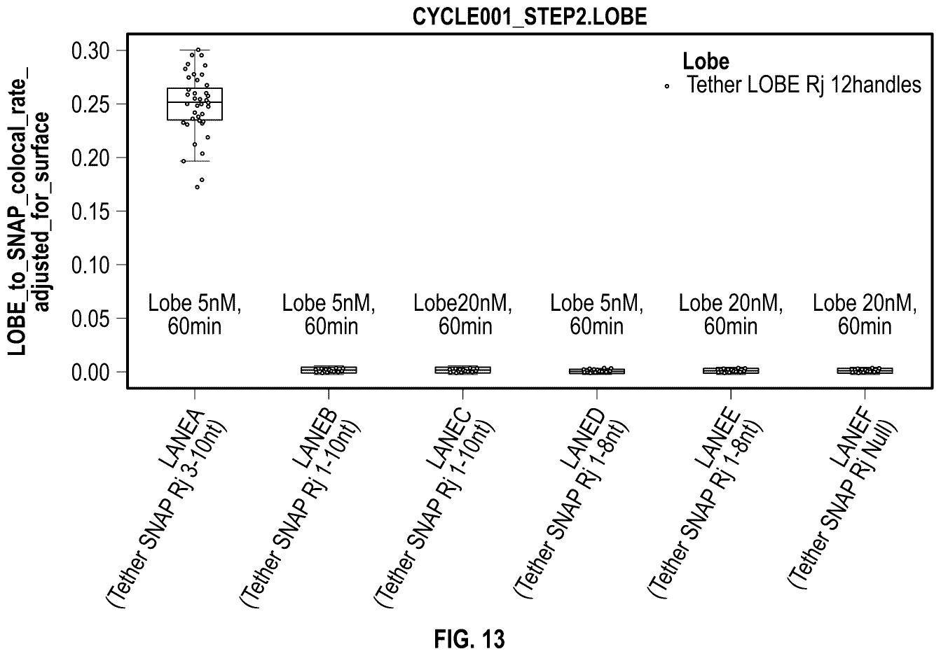

Binding between the Lobe and various SNAPs was measured on a custom fluorescence microscope equipped with a six-lane flow cell. Flow cells included an array of pads, each pad having an active surface formed by photolithographic patterning, CVD deposition of 3-aminopropyltrimethoxysilane, conjugation with NHS-PEG-azide and reaction with DBCO-derivatized oligonucleotides. Pads were surrounded by inert HMDS. The flow cells were assembled using a 25 mm×75 mm glass backer with the patterned chip joined by a film of pressure-sensitive adhesive. The surface oligonucleotides at the pads were complementary to single stranded handles on SNAPs and SNAPs were sized to exclude more than one SNAP from attaching per pad. SNAPs were attached to proteins prior to deposition. Each lane of the flow cell was loaded with one of six different SNAPs under conditions for attaching the SNAPs to the surface of the flow cell. The lanes were loaded as follows: (A) SNAP having 3 dockers with a 10 nucleotide complement of the Lobe tether, (B) SNAP having 1 docker with a 10 nucleotide complement of the Lobe tether, (C) duplicate of (B), (D) SNAP having 1 docker with an 8 nucleotide complement of the Lobe tether, (E) duplicate of (D), and (F) SNAP having no tether. The lanes were then incubated with 5 nM Lobe for 60 min. Unbound Lobe was washed away and fluorescence was detected from Alexa 647 dyes on Lobes bound to SNAPs. FIG. 13 shows results of the flow cell binding experiment, plotting the Lobe to SNAP colocalization rate observed for each lane (n.b. ‘rate’ in this context refers to the fraction of sites where colocalization was observed). The SNAP having 3 tethers with a 10 nucleotide complement of the Lobe tether produced a 25% colocalization rate which was undesirable given the absence of a target epitope on the SNAP and a primary paratope on the Lobe. As such, the interaction of the docker and tether would be at risk of producing a high proportion of false positive binding observations. The other SNAP-Lobe combinations did not appear to have the same risk.

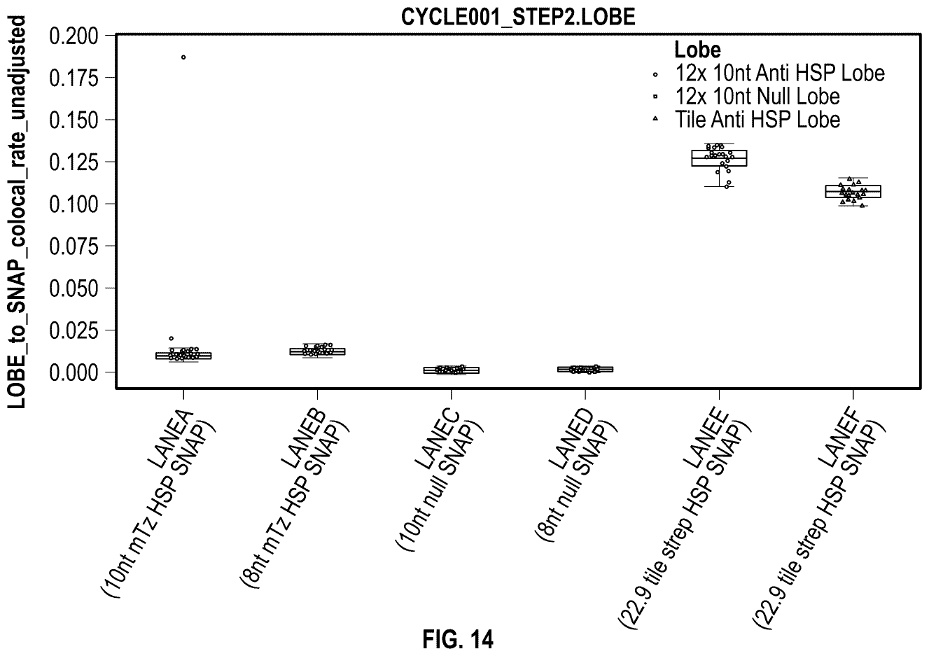

The custom fluorescence microscope was used to measure binding between (a) Lobes having various combinations of epitopes and dockers and (b) SNAPs having various combinations of paratopes and tethers. The assay was carried out as set forth above in connection with FIG. 13 with lanes treated as follows: (A) loaded with SNAPs having one HSP epitope and one docker, contacted with Lobes having anti-HSP antibodies and 12 tethers with 10 nucleotide complement of the docker, (B) loaded with SNAPs having one HSP epitope and one docker, contacted with Lobes having anti-HSP antibodies and 12 tethers with 8 nucleotide complement of the docker, (C) loaded with SNAPs having no epitope and one docker, contacted with Lobes having no antibody and 12 tethers with 10 nucleotide complement of the docker, (D) loaded with SNAPs having no epitope and one docker, contacted with Lobes having no antibody and 12 tethers with 10 nucleotide complement of the docker, (E) loaded with SNAPs having up to three HSP epitopes but no docker, contacted with Lobes having anti-HSP antibody and 12 tethers with 10 nucleotide complement of the docker, and (F) loaded with SNAPs having up to three HSP epitopes but no docker, contacted with Lobes having anti-HSP antibody and no tethers. The results, shown in FIG. 14, indicated that the presence of a single docker on epitope-bearing SNAPs did not have a substantial impact on binding to Lobes bearing antibodies that recognize the epitope. Lanes E and F are experimental controls indicating the flow cell and process was valid.

### Example III

### Removable Single-Epitope Peptide Detection

SNAPs and Lobes having various combinations of epitope-paratope pairs and tether-docker pairs were evaluated for their binding properties. Tether-docker pairs were designed to have weak interactions that increase the likelihood that the Lobe bearing a paratope will remain in close proximity to a SNAP having a target epitope for the paratope. The goal is to increase avidity of a Lobe for its target SNAP. However, another aspect of the goal is to modulate the binding strength between the docker and tether to minimize the risk of the tether and docker pair driving false-positive binding.

A titration experiment was carried out to evaluate binding between SNAPs loaded with 1 to 6 dockers and Lobes loaded with 12 tethers. The SNAPs had the structure set forth in Example L. The Lobes had the structure set forth Example I and vi tethers. Complementary regions between the dockers and tethers were 8 or 10 nucleotides in length. The SNAPs were attached to a peptide having one of two different amino acid timer epitopes (DTR or HSP) and the Lobes were attached to an S-DTR or anti-HSP antibodies. Binding was evaluated using the custom fluorescence microscope as set forth in Example IL. The results of the titration experiment are presented in Table 5.

TABLE 5

| | | | On-Target | Off-Target |
|---|---|---|---|---|
| SNAP | SNAP | Lobe | Binding | Binding |
| Dockers | Epitope | Antibody | Proportion | Proportion |
| 1x 10 nt | DTR | antiDTR | 0.02 | 0 |
| 1x 10 nt | HSP | antiHSP | 0 | 0 |
| 1x 8 nt | DTR | antiDTR | 0.01 | 0 |
| 1x 8 nt | HSP | antiHSP | 0.01 | 0 |
| 1x 9 nt | DTR | antiDTR | 0 | 0 |
| 1x 9 nt | HSP | antiHSP | 0 | 0 |
| 2x 10 nt | DTR | antiDTR | 0.37 | 0.08 |
| 2x 10 nt | HSP | antiHSP | 0.22 | 0.04 |
| 2x 8 nt | DTR | antiDTR | 0.01 | 0 |
| 2x 8 nt | HSP | antiHSP | 0.01 | 0 |
| 2x 9 nt | DTR | antiDTR | 0.07 | 0 |
| 2x 9 nt | HSP | antiHSP | 0.03 | 0 |
| 3x 10 nt | HSP | antiHSP | 0.4 | 0.15 |
| 3x 10 nt | DTR | antiDTR | 0.49 | 0.27 |
| 3x 8 nt | DTR | antiDTR | 0.01 | 0 |
| 3x 8 nt | HSP | antiHSP | 0.01 | 0 |
| 3x 9 nt | DTR | antiDTR | 0.37 | 0.12 |
| 3x 9 nt | HSP | antiHSP | 0.2 | 0.05 |
| 4x 8 nt | DTR | antiDTR | 0.03 | 0 |
| 4x 8 nt | HSP | antiHSP | 0.02 | 0 |
| 4x 9 nt | HSP | antiHSP | 0.28 | 0.09 |
| 4x 9 nt | DTR | antiDTR | 0.41 | 0.25 |
| 5x 8 nt | DTR | antiDTR | 0.08 | 0 |
| 5x 8 nt | HSP | antiHSP | 0.04 | 0 |
| 5x 9 nt | HSP | antiHSP | 0.42 | 0.15 |
| 5x 9 nt | DTR | antiDTR | 0.53 | 0.31 |
| 6x 8 nt | HSP | antiHSP | 0.08 | 0 |
| 6x 8 nt | DTR | antiDTR | 0.12 | 0.01 |
| 6x 9 nt | HSP | antiHSP | 0.45 | 0.17 |
| 6x 9 nt | DTR | antiDTR | 0.55 | 0.33 |

Based on results of the titration, further experiments were run to evaluate binding between (a) SNAPs having DTR or HSP epitopes and 1, 2 or 3 dockers and (b) Lobes having anti-DTR or anti-HSP antibodies and 12 v1 tethers. The dockers had a 10 nucleotide tether complementing region. The experiments were performed on the custom fluorescence microscope as set forth in Example II.

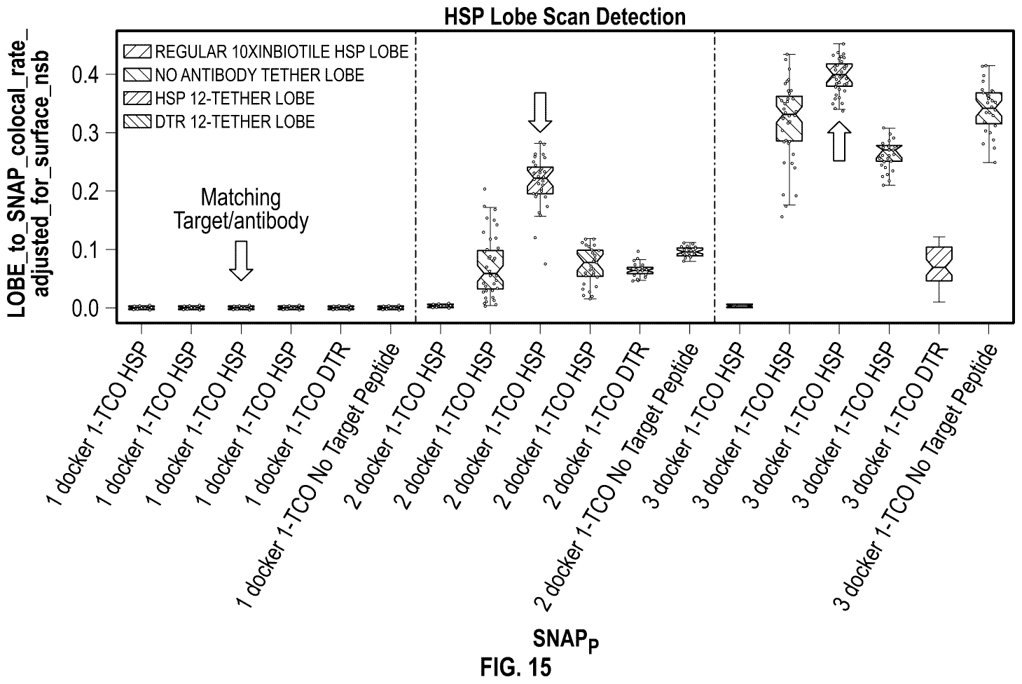

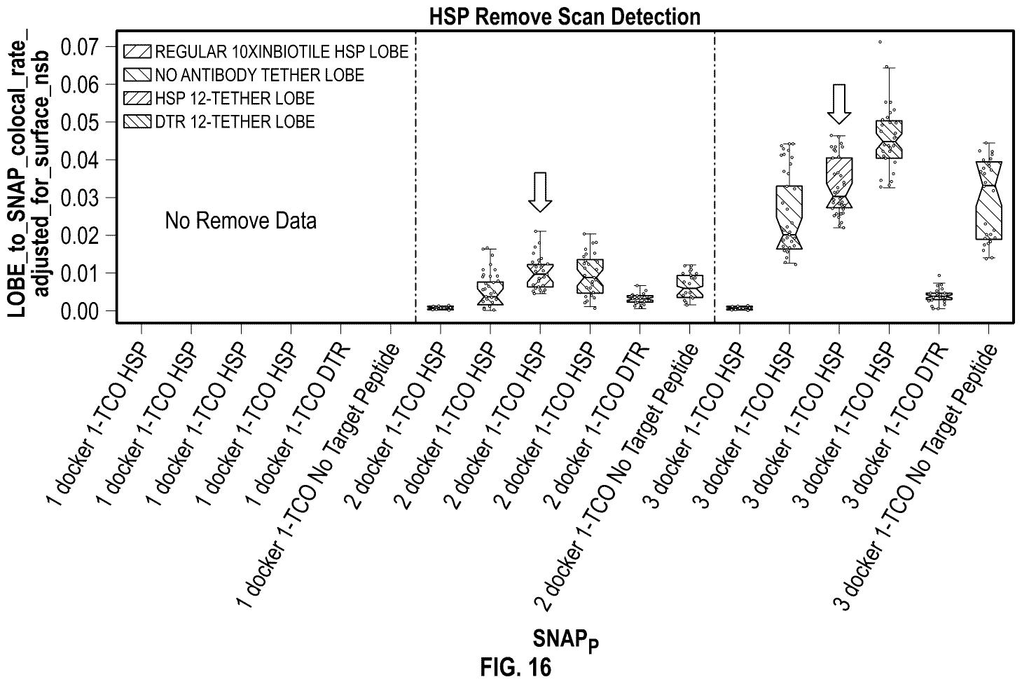

FIG. 15 plots Lobe-SNAP colocalization for various combinations of Lobes and SNAPs following Lobe delivery and removal of unbound Lobes. The SNAPs are identified on the x axis according to the number of dockers present and the epitope present (all epitopes were present as a single copy and connected to the SNAP using TCO-based click chemistry, see U.S. Pat. No. 11,505,796, which is incorporated herein by reference). The Lobes contacted with each SNAP are identified in the legend. For each of the three panels in the plot the following series of Lobes was used: Lobe having anti-HSP antibodies, with no dockers (“Regular 10×inbiotile HSP lobe”), Lobe having no antibody and 12 tethers (“No antibody tether Lobe:), Lobe having anti-HSP antibody and 12 tethers (“HSP 12-tether lobe”), Lobe having anti-DTR antibody and 12 tethers (“DTR 12-tether lobe”), HSP 12-tether lobe and No antibody tether Lobe. The arrows identify Lobe-SNAP combinations having compatible antibodies and epitopes, respectively. FIG. 16 plots Lobe-SNAP colocalization for various combinations of Lobes and SNAPs following Lobe delivery, removal of unbound Lobes and a stripping step to remove bound Lobes. Bound lobes were removed by 10 minute incubation in 100 mM CHAPS in deionized water. The x axis and series of Lobe deliveries in each panel is the same as set forth above for FIG. 15.

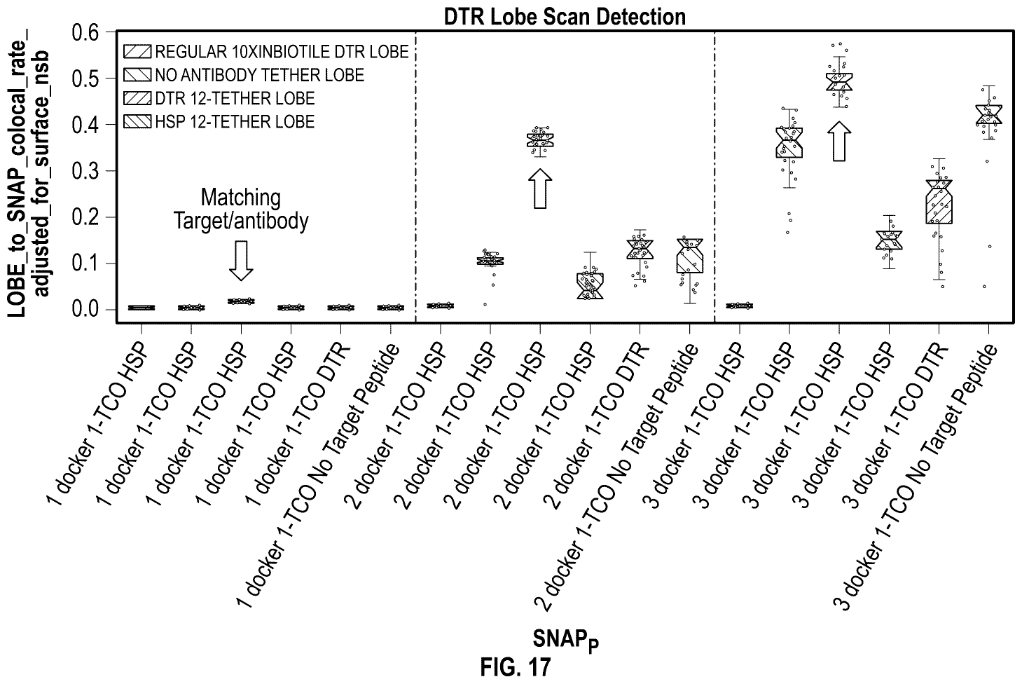

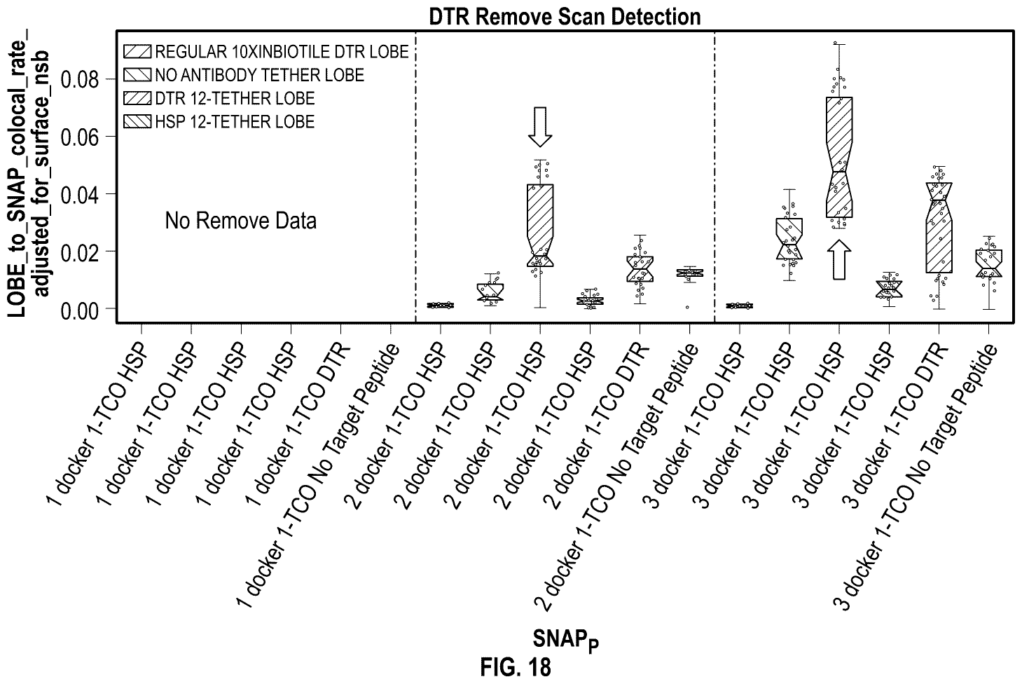

FIG. 17 plots Lobe-SNAP colocalization for various combinations of Lobes and SNAPs following Lobe delivery and removal of unbound Lobes. The SNAPs are identified on the x axis according to the number of dockers present and the epitope present (all epitopes were present as a single copy and attached to the SNAP using TCO-based click chemistry, see U.S. Pat. No. 11,505,796, which is incorporated herein by reference). The Lobes contacted with each SNAP are identified in the legend. For each of the three panels in the plot the following series of Lobes was used: Lobe having anti-DTR antibodies, with no dockers (“Regular 10×inbiotile DTR lobe”), Lobe having no antibody and 12 tethers (“No antibody tether Lobe:), Lobe having anti-DTR antibody and 12 tethers (“DTR 12-tether lobe”), Lobe having anti-HSP antibody and 12 tethers (“HSP 12-tether lobe”), DTR 12-tether lobe and No antibody tether Lobe. The arrows identify Lobe-SNAP combinations having compatible antibodies and epitopes, respectively. FIG. 18 plots Lobe-SNAP colocalization for various combinations of Lobes and SNAPs following Lobe delivery, removal of unbound Lobes and Lobe stripping step. The x axis and series of Lobe deliveries in each panel is the same as set forth above for FIG. 17.

The data shows approximately 20% single molecule detection for on-target HSP with 2 tethers and a background binding (various negative controls) ranging from 5-10% for an effective single molecule binding proportion of better than 10%. The data shows approximately 40% single molecule detection for on-target HSP with 3 tethers and a background binding (various negative controls) ranging from 5-30% for an effective single molecule binding proportion of better than 10%. Removal proportions were a reduction of binding to approximately 1-2%. For DTR, single molecule detection for on-target DTR with 2 tethers was approximately 37%, with background binding around 5-10% for an effective single molecule binding of 25%.

### Example IV

### Evaluation of Lobes Having Aptamers and Tethers

This example describes evaluation of the effect of different tether-docker pairs on binding between Lobes having aptamer-based affinity moieties and SNAPs having target epitopes for the aptamers.

Lobes and SNAPs were designed as set forth in Example I, with the following modifications. Lobes included 12 tethers at locations shown in FIG. 11 and sequences as set forth in Example I. Lobes also had 10 streptavidin linkers located near the perimeter of the origami structure. The streptavidin moieties were loaded with 5′ biotinylated aptamers. Two different aptamers were tested, including the B1 aptamer having affinity for the HHH trimer amino acid epitope and the 7123 aptamer having affinity for the FRRF (SEQ ID NO: 738) tetramer amino acid epitope. Because the two aptamers had been previously determined not to substantially recognize the epitope of the other aptamer, the HHH epitope was used as an off-target control for the 7123 aptamer and the FRRF (SEQ ID NO: 738) epitope was used as an off-target control for the B1 aptamer. SNAPs included various numbers of dockers at locations shown in FIG. 9 and sequences as set forth in Example I. Target epitopes (HHH) or non-target epitopes (FRRF (SEQ ID NO: 738)) were attached to the post of the SNAPs.

The custom fluorescence microscope of Example II was used to measure binding between (a) Lobes having various combinations of epitopes and dockers and (b) SNAPs having various combinations of paratopes and tethers.

Table 6 shows the results for binding of Lobes having the B1aptamer with SNAPs having various numbers of dockers (lx being 1 docker, 2× being 2 dockers, 3× being 3 dockers etc.) and capable of forming an 8 nucleotide (8 nt) or 10 nucleotide duplex with a Lobe tether. The docker configurations are listed in the first column, and the second through fourth columns list the percent of colocalization for the Lobes at array sites having SNAPs with on-target epitopes, off-target epitopes, or no epitopes, respectively.

TABLE 6

| | On-Target | Off-Target | Empty SNAP |
|---|---|---|---|
| 1xdocker-8 nt | 0.5% | 0.1% | 0.1% |
| 2xdocker-8 nt | 1.1% | 0.1% | 0.1% |
| 3xdocker-8 nt | 5.5% | 0.5% | 0.5% |
| 4xdocker-8 nt | 5% | 0% | 0% |
| 5xdocker-8 nt | 20% | 1% | 1% |
| 6xdocker-8 nt | 20% | 1% | 1% |
| 1xdocker-10 nt | 5% | 0% | 0% |
| 2xdocker-10 nt | 20% | 3% | 2% |
| 3xdocker-10 nt | 32% | 11% | 12% |
| 4xdocker-10 nt | 43% | 20% | 10% |
| 5xdocker-10 nt | 50% | 20% | 20% |
| 6xdocker-10 nt | 50% | 22% | 21% |

Table 7 shows the results for binding of Lobes having the 7123 aptamer with SNAPs having various numbers of dockers using the nomenclature of Table 6.

TABLE 7

| | On-Target | Off-Target | Empty SNAP |
|---|---|---|---|
| 1xdocker-8 nt | 0.1% | 0% | 0% |
| 2xdocker-8 nt | 0.2% | 0% | 0% |
| 3xdocker-8 nt | 2.2% | 0.2% | 0.2% |
| 4xdocker-8 nt | 2% | 0% | 0% |
| 5xdocker-8 nt | 10% | 1% | 1% |
| 6xdocker-8 nt | 20% | 2% | 3% |
| 1xdocker-10 nt | 0.5% | 0% | 0% |
| 2xdocker-10 nt | 8.5% | 1% | 1% |
| 3xdocker-10 nt | 26% | 5% | 5% |
| 4xdocker-10 nt | 30% | 5% | 5% |
| 5xdocker-10 nt | 21% | 8% | 5% |
| 6xdocker-10 nt | 24% | 5% | 5% |

The results indicated that varying the number and/or duplex lengths of docker-tether pairs can be effective to tune binding results between affinity reagents and their target epitopes. More specifically, on-target detection increased with the number of dockers and the length of the docker-tether duplex. However, off-target binding also increased. The docker-tether designs with the most favorable results were those having 6 dockers capable of forming 8 nucleotide duplexes with tethers and those having 2 dockers capable of forming 10 nucleotide duplexes with tethers.

### Example V

### Structured Nucleic Acid Particles Having Dockers and Labelled Probes Having Tethers; Peg-Board Origami Particles

This example describes structured nucleic acid particles (SNAPs) having nucleic acid dockers. Labelled probes (Lobes) having nucleic acid tethers are also described.

The origami structure of the SNAP is composed of the p7249 scaffold (Bayou Biolabs, LA) and the oligonucleotides listed in Table 8. The origami SNAP folds to produce a PegBoard structure having a rigid post.

TABLE 8

| | | SEQ ID |
|---|---|---|
| Sequence Name | Sequence | NO |
| 1-026111 | AGGGGGATGTGCATGCAAATC | 300 |
| 2-057140 | GTGTCTGTAGTTTGCAGAGCA | 301 |
| 3-075093 | CGACTTGCGGGAGGTTTTGCCAGCGC | 302 |
| 4-027077 | AACGGTAGTGAGGCCACCGAGTCGGCCT | 303 |
| 5-031077 | GCCAACAAATACGTGGCACAGTAAAACA | 304 |
| 6-043077 | GCTTCTGAATCCTTGAAAACAGTTGGGT | 305 |
| 7-048055 | GAGGGTTGTACCAGGCGGATAAAAGTAT | 306 |
| 8-050118 | GTAGCAAAAAACGAAAGAGGCTTGTGTC | 307 |
| 9-060182 | TTTACCCTGAACAAAGTCAGAGGAGCCTA | 308 |
| 10-060111 | TTATTAGAGAATCAATAGCCGAACAAAGTTACC | 309 |
| 11-004083 | GAACCCTAAAGGGATGTAGCGATGAGGAAGTTTC | 310 |
| | A | |
| 12-034118 | TTCAACGCAGTCAAATCACCAGGCTATCGCGCCAT | 311 |
| 13-034132 | GGAGAAGGAATTAGGATACATATAACTAAAGTAC | 312 |
| | G | |
| 14-036139 | ACCATTACAAAATTAAGCAATGTAATACTGTGTAC | 313 |
| 15-038048 | ACCAGTAACCAATGCGATTGGAAAGCCACCAGTA | 314 |
| | A | |
| 16-039091 | TGCACGTTATATTTCATTAACATCCAATAAAAATT | 315 |
| 17-077133 | TCAGATATAAAACAGGGAAGCGAGCGCTAATATT | 316 |
| | T | |
| 18-026161 | TTTGAAGGGCGATCGGTGCGGGCCTCTTCCGCCAT | 317 |
| | T | |
| 19-062160-RJ8- | AAATAGCCCACAAGAATTGAGTTAAGCCCAATATT | 318 |
| Docker.3p | TATACATCT | |
| 20-054186-RJ8- | TTTATAAACAGCCATATTAATTTGCCAGTTACAAA | 319 |
| Docker.3p | TTTATACATCT | |
| 21-058179-RJ8- | TTTTGAAAATAGCAGCCTTTGTTTAACGTCAAAAA | 320 |
| Docker.3p | TTTATACATCT | |
| 22-032165 | TTTTGCCGGAGAGGGTAGCCTAGCTGTGACCCTAA | 321 |
| | AGCCT | |
| 23-012097 | CCTGAAAAGTATTACGAGAATAGCGGATTGCATCA | 322 |
| | CCAGAAC | |
| 24-020146 | AGTGATGAAGGGTAGGGCGGTTTTTGCGGATATTC | 323 |
| | AACCGTT | |
| 25-022146 | GAAGGGATAGCTCTAGTCCCGGAGAGATATAAGC | 324 |
| | AAATATTT | |
| 26-028083 | CAACCGGAATCATAATTACTATACCAGCTTTCATC | 325 |
| | AACATTA | |
| 27-035119 | TCATAAACACTGTTGCCCTGCCCCACGCAATTATT | 326 |
| | GGGAGGG | |
| 28-035147 | CAGCAAAAGCCAGCTCACGGTGTTCTTCCGCTCAC | 327 |
| | AGTGTAA | |
| 29-036153 | GTAGATTGAAGTTTTGTAGCTGTCATTTAGGTCAG | 328 |
| | TTAATTC | |
| 30-042153 | GAATCGTGGGGTAAGAAAGATTCTACGTAACGTA | 329 |
| | AATCAAGA | |
| 31-057119 | AAGGTAATTCGCAATCGTCGCACGATGCTGATTGC | 330 |
| | GCTCATT | |
| 32-058160 | TACAGAGAGAATAACATAAGATTGCACCCAGAAA | 331 |
| | CGATTTTT | |
| 33-059140 | ATAAGAGCAACATGGTCCAGCGCTGGTCTGGTCAG | 332 |
| | CTCCGGC | |
| 34-061105 | AAAAGATAACAGTTCCAAAATAATGCAGAGAACT | 333 |
| | GACGAGAA | |
| 35-069084 | CGTAATGCTAAAGATGATACCTATCGGTTTATCAG | 334 |
| | ACGATCT | |
| 36-071063 | CCGTGAATTTCTTAAACAGCTCTTTTTCGTCACGCT | 335 |
| | GCGCGT | |
| 37-073063 | TCATAGTTAGCGTACTTGCTTTCGAGATGTACCGT | 336 |
| | ACAGCCC | |
| 38-004090 | TAAATCGAAAGTTTTGTCGTCTTAATTGGATAGTT | 337 |
| | GCGCCGAAGAGGCT | |
| 39-004132 | CACCCAAGTATGGGATTTTGCTCACGTTACCGATA | 338 |
| | TATTCGGAGCAGCG | |
| 40-004146 | ACTACGTGAACCATATTAAAGAAAGACATACACT | 339 |
| | AAAACACTTTTGTAT | |
| 41-016097 | AGGAGCATATCATTTATCACCGTCACCGACTTGAG | 340 |
| | AGTTTGAGTAACAT | |
| 42-018076 | GGAGCGGACCTACCAGCTGAAAAGGTTAATCGGC | 341 |
| | TATGGAAGGGTTAGA | |
| 43-024125 | CACGGGAACGGATAAAGTTGGTTTTTAATCTCCGT | 342 |
| | GGGAACATGAGGGG | |
| 44-026083 | AAGACAAAGAACGCATAAGAATAAACGCCAGCTT | 343 |
| | TCAATCGCCTATATG | |
| 45-027042 | TAAACAGCGAGCACAAGGAGCGGGGAAAGTAGCA | 344 |
| | TTCCACAGAACACTG | |
| 46-028139 | TGCATCTGCCAGTTAACGGCGTTAAATCAGATTGT | 345 |
| | CTACAAATCAATAT | |
| 47-030076 | GCCTTCCTGTAGATTAAAAACTAGCATGTCAATCA | 346 |
| | GCGTCTGTATATAA | |
| 48-033140 | GCCGTTTGGTGCCGGTGCCCCTACACTGGAGTACC | 347 |
| | TTTAAAGAAAGACT | |
| 49-048153 | ACGGAGACATCTTTCACCCTCTCGCTGAGAATAAT | 348 |
| | AATTTTTTAAACAA | |
| 50-051042 | CGAGCCATAAAGTATTCAGCTTAAGTCCACGAGCA | 349 |
| | CTTATCACTCATCG | |
| 51-054132 | GAAGAAAATTCATATGGTTTAAAGCCTTAACGCGA | 350 |
| | GGCGTTTATCATTA | |
| 52-054167 | TTTATCCCAATCCAAATAAGCCGAGCGTTAAAAGA | 351 |
| | ATTAAGACTCCTTA | |
| 53-056132 | ACGGAATATACCCAAAAGAACCGAAGCCTGTAGC | 352 |
| | GCGCAGAGAGATAAC | |
| 54-062153 | AATAGCTATCTTACTGGCATGAACGCAAAGACACC | 353 |
| | CTTACCATTCATCG | |
| 55-071049 | AGCCCAATAGGAACGAACCGCAGGGCGCTATGGT | 354 |
| | TGCTTTGAGAGGCCG | |
| 56-081126 | GTCAGACCTTTTTAAGAAAAGTAACGGAAAGTTTA | 355 |
| | TTCAGAGCCACCAC | |
| 57-047020 | TTTAAATTTAATGGTTTGAAATACCGACCTTTTAGT | 356 |
| | GAGACTACGCTGAG | |
| 58-062182 | TTTATAAGAGCAAGAAACAATGTTACGCAAAGGT | 357 |
| | GGCAACATACTTTCCA | |
| 59-004055 | GCTTGACGGGCGCTCACCCTCTTAGTACCGCCACC | 358 |
| | CCGTCGAATTAAAGGGATTTT | |
| 60-004097 | AAAGCACAACCACCTTGAGGACCACTACGAAGGC | 359 |
| | AGCTCCATGAGCCCTTCACCGC | |
| 61-006076 | TACAGGGAGACAGGGCAGACGGTCAATGCTCCAT | 360 |
| | TAAACGGGTAAAATAACGAGGC | |
| 62-006111 | CGAGATAGGGTTACCCGCCGCATCCTGAGTTACTT | 361 |
| | TTGAAAGAGGACAGACACCAG | |
| 63-006118 | AATAGCCCTGGCCCGAAATCCGTGTACAGACCAG | 362 |
| | GTAAGGCTGTCGGGAAACCTGT | |
| 64-008055 | CATCACGTGAGTAGTAAGAGGCTATTATTCTGAAA | 363 |
| | TCAGTGCAGTAATAAAAGGGA | |
| 65-008097 | TGTTTTTAGAACAAAACGAGTCTTATGCGATTTTA | 364 |
| | ATACATACTGAATTCGTAATC | |
| 66-010076 | AAAAACGCATTCTGCATAGTAAGAGCGGCATTTCA | 365 |
| | ACTTTAATCATTGTTACGAGG | |
| 67-010111 | TAATTGCGTTGCTTACCGCCAAACCCTTACGCCAA | 366 |
| | GACGACGATAAAAACAGAAAA | |
| 68-010118 | CTCACATATGGTCATTCAACTAGCGAGAGGCTTTT | 367 |
| | CCTCAAAGCTGCGGCCAGAAT | |
| 69-012055 | TGGCTATGAACGAATACCGTTGAATGGAAAGCGC | 368 |
| | ATGAGGCAAATCAACAGTTGAA | |
| 70-014076 | GAGAGCCAGGAATTCATTGACAGGAGGTGTACCC | 369 |
| | TGACTATTATAGTCACCGCCAG | |
| 71-014118 | CCAGCGCTTGCTCGCCTCAGAGAACCGCCACCCTC | 370 |
| | TGACGGAAACCAGCTTACGGC | |
| 72-018118 | ATGCCAATTTCGTCATGGTCAACAGGCAAGGCAAA | 371 |
| | CCTTTATTGCCGCCAGCAGTT | |
| 73-020055 | ATGAATATGCTTTGCAATAGAAATGCAGAACGCGC | 372 |
| | GTACCGATATATGTGAGTGAA | |
| 74-020097 | CAGAAATTTCATTTTTTAGAAAAGGGTGAGAAAGG | 373 |
| | GAGTCTGTAACAATCGGCGAA | |
| 75-022076 | TGAAACATAACCTTAACGGTAATCGTAACTTGAGT | 374 |
| | AATGTGTAGGTAAAATCGATG | |
| 76-022111 | ACAGCGGATCAAATTACCTGATCGCTATGAGCAAA | 375 |
| | CCCGGTTGATAATCGAACGCC | |
| 77-022118 | CGCAGAAACGTACAAGGTCATCCCCAAAAACAGG | 376 |
| | AAGCTCATGTAACGCCAGGGTT | |
| 78-024055 | ATTAAGACCTTTTTCGCCATATCAACAGTAGGGCT | 377 |
| | GAAAAAGAAATAAGGCGTTAA | |
| 79-024097 | TAATTTTTAAATGCATCAAAAGCGAGTAACAACCC | 378 |
| | GGCCTCACGCTTCTGGTGCCG | |
| 80-025140 | AACGTGGATCGGCAAAATCCCCAAGCGGCATCGC | 379 |
| | CGCTGGCTGACCTTCCAAAGCT | |
| 81-026118 | TGGCGAAGAAACCAACGACGACAGTATCGTCGGA | 380 |
| | TCCAATAGAGAAAAGTGCCTGA | |
| 82-027119 | TGAGAGAACCAGTGAGACGGGCTTTCCATGCCCTG | 381 |
| | GCTCATTATACCAGTACCACA | |
| 83-027140 | TCCACGCGCCAGGGTGGTTTTCGTGCCAGCTCATT | 382 |
| | GTTGGGAAGAAAAATCATCAG | |
| 84-029056 | AAGAACTAAATACCTACATTTCACGACCCTTGAGT | 383 |
| | TAATAAGTTTTAACCTGAATT | |
| 85-029140 | GCTGCATAGCCTGGGGTGCCTGTGAAATTTGAGAT | 384 |
| | AAGTTTTGCCAGAGCATAAAT | |
| 86-029147 | TAATGAAATTGGGCTGGTTTGTTCCGAAACTCCAA | 385 |
| | ATGGCCCCTTTCAACAGTTTC | |
| 87-031119 | TAGCTGTGTTGAGGATCCCCGCTGTGGTTGCTTTAT | 386 |
| | AAGAGGAAGCCCGCCACCAC | |
| 88-031140 | TGTTATCGCGTCCGTGAGCCTGCGGCGGATTCATT | 387 |
| | TCAAATATCGCGTTGATTAGA | |
| 89-033056 | CCACCAGAATGAAAAATCTAAGTTGGCAGGTCAG | 388 |
| | AAAACCATCGATAGCGAGCCAG | |
| 90-034076 | AAATGCAATGCCGTAAGGCATCAATTCTACTAATA | 389 |
| | ATATTTTGCAGAGGCGAATTA | |
| 91-034090 | CCCTCATGTAGTAGTCATTTGGGGCGCGATATCAA | 390 |
| | TTGCGTAGATTTTCAAATCGC | |
| 92-035042 | CTGGTCAAGCATCAAAATACCTAGTCTTCCAGTCA | 391 |
| | TGACGCTACTTGCCCAAATTA | |
| 93-035098 | CTGTAAAGGTTTCTAGTGTCACTGCGACCGCCTGC | 392 |
| | AAAATATCACCACCAGAGCCG | |
| 94-035140 | GTGTGTTCGGTTGCGGTATGATGGAGGTTTTTAAA | 393 |
| | TATGCATAGTTGCTCCTTTTG | |
| 95-037056 | CCGAACGATCATATTCCTGATCTGAATAGTCTTTCT | 394 |
| | GTAGAAACCAATCTTATCAA | |
| 96-041056 | AATACCAAGAAAACAAAATTAAAATCAACAAAAG | 395 |
| | GGTAATAAGAGAATATGAGAAT | |
| 97-042076 | AATCAGGTCTTTCTGGAACACTATCATAACCCTCG | 396 |
| | AATCAAAGAGGTGAGGCGGTC | |
| 98-042132 | GAATCCCGCAAAAGTTAGGAATCAGGACCAGTGA | 397 |
| | ACGCATAGTGATAAAAAAAGAA | |
| 99-043042 | AGTACATATTACATGCCTGATTACAGTATATACTT | 398 |
| | TATCAGAATTAAATGAGGATT | |
| 100-045056 | AACCTCCCTTTTTCAAATATAGTGTGATCCTGTTTA | 399 |
| | GTATCACCAACGCTTTAACA | |
| 101-046076 | GAGATGGTTTAATGAGTCATAAGGGAACCGAACT | 400 |
| | GTGGGCTTTGCTGGTAATATCC | |
| 102-047091 | CCGGCACGGAAGATCGCACTCAATGTGAATAATTC | 401 |
| | TATGTACCAAGAGAGATTCAA | |
| 103-050132 | GCATCGGACGCATAGAAAATCTCCAAAAATTTTCT | 402 |
| | ATCAAGTTTTTTGGAGTTTGG | |
| 104-053105 | CCGGAGACAAGGATAAATCATATAACCTCGATTG | 403 |
| | ACATTAAAACCCTCAGCCGCCA | |
| 105-061084 | GAAGCAAGACCATATTTACCAAAGGAATGAATTA | 404 |
| | CAGTAAATACCAACTAGCCGGA | |
| 106-063042 | GTACTGGAACAGTGTCGGAACCTGAGACTTGCTCA | 405 |
| | GATATAAAGGAGGTAGAACCG | |
| 107-067105 | ATGAACGGCGACCTCCAACCTCGGCTACCAATGAC | 406 |
| | TCCAAAAGGAGCCTTTTCCAG | |
| 108-073105 | ACGTTAGTAAATGAAAAAGGCAACAACCATCGCC | 407 |
| | CAACGAGGAACAAGAGTCCACT | |
| 109-079105 | AATCACCGGAACTTAGCCCCCCCGCGCCCAATAGC | 408 |
| | TTCTAAGAAATCAAGATTAGT | |
| 110-079140 | ACGCTAATACAATTTTATCCTTGCTATTAGGCTTAT | 409 |
| | CCGGTAAAGCAAAGCATTTT | |
| 111-083105 | AGGAAACGCAATAATAAGCAGAGTTTGCCTTTAGC | 410 |
| | CGGTCATGTCACAATCAATAT | |
| 112-022161 | TTTTTTCTCCGTGGTATCGACAGGCCTTTCATCCTC | 411 |
| | GCGTGGTATCAGCGCCGGGCG | |
| 113-024161 | TTTCGGCCAGTGCCAAGCTTTCAGAGGTGTTCCCA | 412 |
| | GATTTTTGGATTGACCGTAATG | |
| 114-028146- | GTAACCGCAGGCTGCGCAACTGTTGGTTTCGTTTC | 413 |
| DYE40.22.9.3p | TTTGCTTGTGTCTTGTCTTCGTCGTGCTTGTTCC | |
| 115-030146- | GCATTAATCACGACGTTGTAAAACGATTTCGTTTC | 414 |
| DYE40.22.9.3p | TTTGCTTGTGTCTTGTCTTCGTCGTGCTTGTTCC | |
| 116-032146- | TATTTTTGAATTTGTGAGAGATAGACTTTCGTTTCT | 415 |
| DYE40.22.9.3p | TTGCTTGTGTCTTGTCTTCGTCGTGCTTGTTCC | |
| 117-052160- | GAATTGCGGCTTGCAGGGAGTTAAAGTTTCGTTTC | 416 |
| DYE40.22.9.3p | TTTGCTTGTGTCTTGTCTTCGTCGTGCTTGTTCC | |
| 118-071042- | GATAGCAAGTTTCGTCACCAGTACAATTTCGTTTC | 417 |
| DYE40.22.9.3p | TTTGCTTGTGTCTTGTCTTCGTCGTGCTTGTTCC | |
| 119-034175- | TTTGGTTGTACCAAAAACATTAATAAATTAATTTC | 418 |
| DYE40.22.9.3p | GTTTCTTTGCTTGTGTCTTGTCTTCGTCGTGCTTGTT | |
| | CC | |
| 120-073027- | TTTACTACAACGCCTGCCGGCGAACGTGGCGAGAT | 419 |
| DYE40.22.9.3p | TTCGTTTCTTTGCTTGTGTCTTGTCTTCGTCGTGCTT | |
| | GTTCC | |
| 121-004172- | TTTACCGTCTATCAGGGCGCGTCAAAGGGCGAAAA | 420 |
| DYE40.22.9.3p | TTTCGTTTCTTTGCTTGTGTCTTGTCTTCGTCGTGCT | |
| | TGTTCC | |
| 122-006172- | TTTTCCTGTTTGATGGTGGCCCCAGCAGGCGAAAA | 421 |
| DYE40.22.9.3p | TTTCGTTTCTTTGCTTGTGTCTTGTCTTCGTCGTGCT | |
| | TGTTCC | |
| 123-008172- | TTTGAGAGGCGGTTTGCGTTCGGCCAACGCGCGGG | 422 |
| DYE40.22.9.3p | TTTCGTTTCTTTGCTTGTGTCTTGTCTTCGTCGTGCT | |
| | TGTTCC | |
| 124-010172- | TTTCGAGCCGGAAGCATAAAATTCCACACAACATA | 423 |
| DYE40.22.9.3p | TTTCGTTTCTTTGCTTGTGTCTTGTCTTCGTCGTGCT | |
| | TGTTCC | |
| 125-012172- | TTTGCCAGCACGCGTGCCTCATACCGGGGGTTTCT | 424 |
| DYE40.22.9.3p | TTTCGTTTCTTTGCTTGTGTCTTGTCTTCGTCGTGCT | |
| | TGTTCC | |
| 126-014172- | TTTATGCCGGGTTACCTGCTCGTTAACGGCATCAG | 425 |
| DYE40.22.9.3p | TTTCGTTTCTTTGCTTGTGTCTTGTCTTCGTCGTGCT | |
| | TGTTCC | |
| 127-016172- | TTTTTTCGCACTCAATCCGGGGTCATTGCAGGCGC | 426 |
| DYE40.22.9.3p | TTTCGTTTCTTTGCTTGTGTCTTGTCTTCGTCGTGCT | |
| | TGTTCC | |
| 128-018172- | TTTGACTTGTAGAACGTCAATAACGGAACGTGCCG | 427 |
| DYE40.22.9.3p | TTTCGTTTCTTTGCTTGTGTCTTGTCTTCGTCGTGCT | |
| | TGTTCC | |
| 129-020172- | TTTAAAAAGCCGCACAGGCTAAAAAAATCCCGTA | 428 |
| DYE40.22.9.3p | ATTTCGTTTCTTTGCTTGTGTCTTGTCTTCGTCGTGC | |
| | TTGTTCC | |
| 130-028165- | TTTTGTAGATGGGCGCATCGGATAGGTCACGTTGG | 429 |
| DYE40.22.9.3p | TTTCGTTTCTTTGCTTGTGTCTTGTCTTCGTCGTGCT | |
| | TGTTCC | |
| 131-030165- | TTTTATTTTGTTAAAATTCAAATTGTAAACGTTAAT | 430 |
| DYE40.22.9.3p | TTCGTTTCTTTGCTTGTGTCTTGTCTTCGTCGTGCTT | |
| | GTTCC | |
| 132-049023- | TTTTCTTACCAGTATAAAGTATGCGTTATACAAATT | 431 |
| DYE40.22.9.3p | TTCGTTTCTTTGCTTGTGTCTTGTCTTCGTCGTGCTT | |
| | GTTCC | |
| 133-051023- | TTTTAGGCAGAGGCATTTTACGCCAACATGTAATT | 432 |
| DYE40.22.9.3p | TTTCGTTTCTTTGCTTGTGTCTTGTCTTCGTCGTGCT | |
| | TGTTCC | |
| 134-052179- | TTTAAAGGAACAACTAAAGAGCGGAGTGAGAATA | 433 |
| DYE40.22.9.3p | GTTTCGTTTCTTTGCTTGTGTCTTGTCTTCGTCGTGC | |
| | TTGTTCC | |
| 135-053023- | TTTGACAATAAACAACATGATTCTGTCCAGACGAC | 434 |
| DYE40.22.9.3p | TTTCGTTTCTTTGCTTGTGTCTTGTCTTCGTCGTGCT | |
| | TGTTCC | |
| 136-055023- | TTTTATCCCATCCTAATTTTGAACAAGAAAAATAA | 435 |
| DYE40.22.9.3p | TTTCGTTTCTTTGCTTGTGTCTTGTCTTCGTCGTGCT | |
| | TGTTCC | |
| 137-057023- | TTTTAAACCAAGTACCGCATTCCAAGAACGGGTAT | 104 |
| DYE40.22.9.3p | TTTCGTTTCTTTGCTTGTGTCTTGTCTTCGTCGTGCT | |
| | TGTTCC | |
| 138-059023- | TTTCAAGGCCGGAAACGTCGCACCATTACCATTAG | 436 |
| DYE40.22.9.3p | TTTCGTTTCTTTGCTTGTGTCTTGTCTTCGTCGTGCT | |
| | TGTTCC | |
| 139-061023- | TTTCAAATAAATCCTCATTCCTTGATATTCACAAAT | 437 |
| DYE40.22.9.3p | TTCGTTTCTTTGCTTGTGTCTTGTCTTCGTCGTGCTT | |
| | GTTCC | |
| 140-063023- | TTTTTGATGATACAGGAGTGCGTCATACATGGCTT | 438 |
| DYE40.22.9.3p | TTTCGTTTCTTTGCTTGTGTCTTGTCTTCGTCGTGCT | |
| | TGTTCC | |
| 141-065023- | TTTATGCCCCCTGCCTATTCCCGTATAAACAGTTAT | 439 |
| DYE40.22.9.3p | TTCGTTTCTTTGCTTGTGTCTTGTCTTCGTCGTGCTT | |
| | GTTCC | |
| 142-067023- | TTTTAGGATTAGCGGGGTTTCCTCAAGAGAAGGAT | 440 |
| DYE40.22.9.3p | TTTCGTTTCTTTGCTTGTGTCTTGTCTTCGTCGTGCT | |
| | TGTTCC | |
| 143-069023- | TTTGTGTATCACCGTACTCGTATAGCCCGGAATAG | 441 |
| DYE40.22.9.3p | TTTCGTTTCTTTGCTTGTGTCTTGTCTTCGTCGTGCT | |
| | TGTTCC | |
| 144-071023- | TTTCACCCTCATTTTCAGGCCACCCTCAGAGCCACT | 442 |
| DYE40.22.9.3p | TTCGTTTCTTTGCTTGTGTCTTGTCTTCGTCGTGCTT | |
| | GTTCC | |
| 145-025020- | TTTAAGGAAGGGAAGAAAGCGAGTATAACGTGCT | 443 |
| DYE40.22.9.3p | TTCCTCGTTTCGTTTCTTTGCTTGTGTCTTGTCTTCG | |
| | TCGTGCTTGTTCC | |
| 146-027020- | TTTTTAGAATCAGAGCGGGAGCACCGTTGTAGCAA | 444 |
| DYE40.22.9.3p | TACTTCTTTCGTTTCTTTGCTTGTGTCTTGTCTTCGT | |
| | CGTGCTTGTTCC | |
| 147-029020- | TTTTTTGATTAGTAATAACATCCAATCGTCTGAAAT | 445 |
| DYE40.22.9.3p | GGATTTTTCGTTTCTTTGCTTGTGTCTTGTCTTCGTC | |
| | GTGCTTGTTCC | |
| 148-031020- | TTTATTTACATTGGCAGATTCATAATGCGCGAACT | 446 |
| DYE40.22.9.3p | GATAGCTTTCGTTTCTTTGCTTGTGTCTTGTCTTCG | |
| | TCGTGCTTGTTCC | |
| 149-033020- | TTTCCTAAAACATCGCCATTAACCTTGCTGAACCT | 447 |
| DYE40.22.9.3p | CAAATATTTCGTTTCTTTGCTTGTGTCTTGTCTTCG | |
| | TCGTGCTTGTTCC | |
| 150-035020- | TTTTCAAACCCTCAATCAATATTAGAAGTATTAGA | 448 |
| DYE40.22.9.3p | CTTTACTTTCGTTTCTTTGCTTGTGTCTTGTCTTCGT | |
| | CGTGCTTGTTCC | |
| 151-036175- | TTTTTCCCAATTCTGCGAACGACAGAGCATAAAGC | 449 |
| DYE40.22.9.3p | TAAATCTTTCGTTTCTTTGCTTGTGTCTTGTCTTCGT | |
| | CGTGCTTGTTCC | |
| 152-037020- | TTTAAACAATTCGACAACTCGTTGATGGCAATTCA | 450 |
| DYE40.22.9.3p | TCAATATTTCGTTTCTTTGCTTGTGTCTTGTCTTCGT | |
| | CGTGCTTGTTCC | |
| 153-038175- | TTTTAATTGCTGAATATAATGCCATTCCATATAAC | 451 |
| DYE40.22.9.3p | AGTTGATTTCGTTTCTTTGCTTGTGTCTTGTCTTCGT | |
| | CGTGCTTGTTCC | |
| 154-039020- | TTTTAATCCTGATTGTTTGGATACAGTACCTTTTAC | 452 |
| DYE40.22.9.3p | ATCGGTTTCGTTTCTTTGCTTGTGTCTTGTCTTCGTC | |
| | GTGCTTGTTCC | |
| 155-040175- | TTTACCGGAAGCAAACTCCAACTTGCGGATGGCTT | 453 |
| DYE40.22.9.3p | AGAGCTTTTCGTTTCTTTGCTTGTGTCTTGTCTTCG | |
| | TCGTGCTTGTTCC | |
| 156-041020- | TTTGAGAAACAATAACGGATTCTTAACAATTTCAT | 454 |
| DYE40.22.9.3p | TTGAATTTTCGTTTCTTTGCTTGTGTCTTGTCTTCGT | |
| | CGTGCTTGTTCC | |
| 157-042175- | TTTGATAGCGTCCAATACTGCGGAGCTTCAAAGCG | 455 |
| DYE40.22.9.3p | AACCAGTTTCGTTTCTTTGCTTGTGTCTTGTCTTCG | |
| | TCGTGCTTGTTCC | |
| 158-043020- | TTTTACCTTTTTTAATGGAAACAAGAGTCAATAGT | 456 |
| DYE40.22.9.3p | GAATTTTTTCGTTTCTTTGCTTGTGTCTTGTCTTCGT | |
| | CGTGCTTGTTCC | |
| 159-044175- | TTTAACAACATTATTACAGGTATAGTAAAATGTTT | 457 |
| DYE40.22.9.3p | AGACTGTTTCGTTTCTTTGCTTGTGTCTTGTCTTCG | |
| | TCGTGCTTGTTCC | |
| 160-045020- | TTTATCAAAATCATAGGTCTGATAATTTCATCTTCT | 458 |
| DYE40.22.9.3p | GACCTTTTCGTTTCTTTGCTTGTGTCTTGTCTTCGTC | |
| | GTGCTTGTTCC | |
| 161-046175- | TTTGATATTCATTACCCAAATCTAATAAAACGAAC | 459 |
| DYE40.22.9.3p | TAACGGTTTCGTTTCTTTGCTTGTGTCTTGTCTTCG | |
| | TCGTGCTTGTTCC | |
| 162-048175- | TTTAAGCGCGAAACAAAGTACAGTAATCTTGACAA | 460 |
| DYE40.22.9.3p | GAACCGTTTCGTTTCTTTGCTTGTGTCTTGTCTTCG | |
| | TCGTGCTTGTTCC | |
| 163-050175- | TTTGCCGCTTTTGCGGGATCGTGACCCCCAGCGAT | 461 |
| DYE40.22.9.3p | TATACCTTTCGTTTCTTTGCTTGTGTCTTGTCTTCGT | |
| | CGTGCTTGTTCC | |
| 164-006069- | CGCGTACTGGCAAGGCCCCCGATTTAGATTTACAG | 462 |
| T3.3p- | TTATTAGCCCGCATTT | |
| JF20.22.9.3p | | |
| 165-008090- | ATAATCACGCCAGAGCTTAATGCGCCGCTTTACAG | 463 |
| T3.3p- | TTATTAGCCCGCATTT | |
| JF20.22.9.3p | | |
| 166-008132- | TCTTTTCGTTGCAGTTATAAATCAAAAGTTTACAGT | 464 |
| T3.3p- | TATTAGCCCGCATTT | |
| JF20.22.9.3p | | |
| 167-010069- | CTCATGGCAAACTATAAAAGAGTCTGTCTTTACAG | 465 |
| T3.3p- | TTATTAGCCCGCATTT | |
| JF20.22.9.3p | | |
| 168-012090- | GCGTAAGGAGATAGGCCATTGCAACAGGTTTACA | 466 |
| T3.3p- | GTTATTAGCCCGCATTT | |
| JF20.22.9.3p | | |
| 169-012132- | CCTCACATTCCTGTAATGAGTGAGCTAATTTACAG | 467 |
| T3.3p- | TTATTAGCCCGCATTT | |
| JF20.22.9.3p | | |
| 170-014069- | AGCAGCACAGAAGAACAATATTTTTGAATTTACAG | 468 |
| T3.3p- | TTATTAGCCCGCATTT | |
| JF20.22.9.3p | | |
| 171-016090- | CTAACAAGTTATCTAACAGTGCCACGCTTTTACAG | 469 |
| T3.3p- | TTATTAGCCCGCATTT | |
| JF20.22.9.3p | | |
| 172-016132- | GCCGGGTCATCCCTCTGCATCAGACGATTTTACAG | 470 |
| T3.3p- | TTATTAGCCCGCATTT | |
| JF20.22.9.3p | | |
| 173-018069- | AATTATCTTATTAACCGTCAATAGATAATTTACAG | 471 |
| T3.3p- | TTATTAGCCCGCATTT | |
| JF20.22.9.3p | | |
| 174-020090- | AAAGAAAAATTATTAGAAACCACCAGAATTTACA | 472 |
| T3.3p- | GTTATTAGCCCGCATTT | |
| JF20.22.9.3p | | |
| 175-020132- | AAGTTAATGGCAGCCAGCAACCGCAAGATTTACA | 473 |
| T3.3p- | GTTATTAGCCCGCATTT | |
| JF20.22.9.3p | | |
| 176-022069- | AACATCAAGTTACAAGGTTTAACGTCAGTTTACAG | 474 |
| T3.3p- | TTATTAGCCCGCATTT | |
| JF20.22.9.3p | | |
| 177-024090- | CCCTTAGTAAATCGGCAAAAGAAGATGATTTACAG | 475 |
| T3.3p- | TTATTAGCCCGCATTT | |
| JF20.22.9.3p | | |
| 178-024132- | GAGCCGCGTTTACCCACGGAAAAAGAGATTTACA | 476 |
| T3.3p- | GTTATTAGCCCGCATTT | |
| JF20.22.9.3p | | |
| 179-025098- | ACGAGTGTTGTTCCGGTCGAGGTGCCGTTTTACAG | 477 |
| T3.3p- | TTATTAGCCCGCATTT | |
| JF20.22.9.3p | | |
| 180-026069- | GAGAAAAGGCTTAGTAGCGATAGCTTAGTTTACAG | 478 |
| T3.3p- | TTATTAGCCCGCATTT | |
| JF20.22.9.3p | | |
| 181-029098- | TAGCTCACTGCCCGCAACAGCTGATTAGTTTACAG | 479 |
| T3.3p- | TTATTAGCCCGCATTT | |
| JF20.22.9.3p | | |
| 182-033098- | ACCGCCTGTGCACTGGTACCGAGCTCGATTTACAG | 480 |
| T3.3p- | TTATTAGCCCGCATTT | |
| JF20.22.9.3p | | |
| 183-041098- | CAACTTAAATTTCTCGTTCCGGCAAAAATTTACAG | 481 |
| T3.3p- | TTATTAGCCCGCATTT | |
| JF20.22.9.3p | | |
| 184-045098- | TGTGCAAGGCGATTACCTCACCGGAAATTTTACAG | 482 |
| T3.3p- | TTATTAGCCCGCATTT | |
| JF20.22.9.3p | | |
| 185-047119- | GGCAAAGCGCCATTGCTATTACGCCAGCTTTACAG | 483 |
| T3.3p- | TTATTAGCCCGCATTT | |
| JF20.22.9.3p | | |
| 186-016055 | TACATTTCCTTTGCCAAAATCAGAACAAGCAAGCC | 484 |
| | GTTTTTA | |
| 187-059095 | CAGAGCCGCCGGTGAATTTCGGTGGTGCCATGGCT | 485 |
| | GGTAATGGTT | |
| 188-035077 | GAGGAAGCTAATAGATTAGAGTTTTAAACCATTTG | 486 |
| | GGAATTAAGCACCG | |
| 189-057095 | GACATTCAACGTTTAGCAACGCGGTCCGTTTCGGC | 487 |
| | AGCACCGTGCGGAACAA | |
| 190-077095 | CATCGTAGGATAGCGAACCTCC | 488 |
| 191-081095 | CAGTAGCGACCGTTTGCCATCT | 489 |
| 192-079093 | TTTCATAATCAACAAAGACAAAA | 490 |
| 193-083093 | AGAAGGAAACCGCGGAACCGCCT | 491 |
| 196-060160-RJ8- | GGTAATTGCATTAGACGGGAGAATTAACTGAACTT | 492 |
| Docker.3p | TATACATCT | |
| 195-056186- | TTTAGAAAATACATACATAGTATGTTAGCAAACGT | 493 |
| APT40.2.3p | TTTCACTCACCTCCATCTCCACTCCTACCCATCCAA | |
| | CTCCCAC | |

The SNAP has a nucleic acid origami structure that forms a Pegboard. A post protrudes from the planar surface of the Pegboard and is configured to attach analytes such as proteins or short peptide epitopes. The SNAP further includes 48 handles that are capable of being attached to a label (sequences with the word “DYE” in the first column of Table 8). Four dockers also attached to the SNAP. The dockers include the sequences identified as 19-062160-RJ8-Docker.3p, 20-054186-RJ8-Docker.3p, 21-058179-RJ8-Docker.3p, and 196-060160-RJ8-Docker.3p, each of which includes an 8 nucleotide sequence region (ATACATCT) at the 3′ end.

The Lobe includes an origami structure composed of the p8064 scaffold (Tilibit Nanosystems, Munich Germany) and the oligonucleotides listed in Table 9. The origami SNAP folds to produce a PegBoard structure having a rigid post.

TABLE 9

| | | SEQ ID |
|---|---|---|
| Sequence Name | Sequence | NO |
| 1-028062 | TAACCCTATACACTAAAACAC | 494 |
| 2-032062 | TTAAACAAATCTCCAAAAAAA | 495 |
| 3-038083 | GCGGCCATGCCCCCTGCCTAT | 496 |
| 4-044062 | GTAGCATTTGAGCCATTTGGG | 497 |
| 5-020097 | ATTTGTAGCGCATAAAGATAAGAGCCAGTTTCACT | 498 |
| | CACCTCCATCTCCACTCCTACCCATCCAACTCCCAC | |
| | TTTCTAGATGTAT | |
| 6-020118 | ATTGCGTATATTCCTACCGAATCTAAAGTTTCACTC | 499 |
| | ACCTCCATCTCCACTCCTACCCATCCAACTCCCACT | |
| | TTCTAGATGTAT | |
| 7-020160 | TACCATACTGATTGTTAATGCATCAATATTTCACTC | 500 |
| | ACCTCCATCTCCACTCCTACCCATCCAACTCCCACT | |
| | TTCTAGATGTAT | |
| 8-047140 | AGGCAAAGCAAGGCAACAGCCATATTAT | 501 |
| 9-029073 | ATTTACATTGGGTGAGGCGGTGTACAGACCAG | 502 |
| 10-033073 | CGAACGTGGCGTTTTAGACCTCAGCAGCGAAA | 503 |
| 11-002055 | TTGAGGGCACCGACTAACATCTCAATTCTACTA | 504 |
| 12-000090 | ACGCAATGTCAAATCACCATCAGCCCCAGTTAAAA | 505 |
| | TTTCACTCACCTCCATCTCCACTCCTACCCATCCAA | |
| | CTCCCACTTTCTAGATGTAT | |
| 13-000132 | AAGCAGAAAATTAATGCCGGAACTAGCATAACCAA | 506 |
| 14-001098 | AATATGATATTCAACCGTTCTACCCCGGTTGTTAAT | 507 |
| | TTCACTCACCTCCATCTCCACTCCTACCCATCCAAC | |
| | TCCCACTTTCTAGATGTAT | |
| 15-001140 | GAGGGTAGCTATTTTTGAGAGTCGATGAAAAATAA | 508 |
| | TTTCACTCACCTCCATCTCCACTCCTACCCATCCAA | |
| | CTCCCACTTTCTAGATGTAT | |
| 16-016118 | ATCGTCGAAAGAAGAGAGCGGAAAGAGTCTGTCCA | 509 |
| 17-022097 | AATCTTGTGAATTATTTTAAGAACTGGCTCATTAT | 510 |
| 18-022139 | AAGAAACACAAACAACTAACAACTAATAGATTAGA | 511 |
| 19-022160 | ACATTATATTAAATATCTAAAATATCTTACCCTCA | 512 |
| 20-031133 | AATTAACCGTTGTAATCCAGAAGTAACAGTACCTT | 513 |
| 21-033112 | CGGGCGCTAGGGCGTAGAATCATGATGAAACAAAC | 514 |
| 22-035133 | AGTCCACTATTAAAAATCAAGAACATAGCGATAGC | 515 |
| 23-036132 | GAGCCGGAGCCTCCCAGACGAAGGTTTCACGCAAC | 516 |
| 24-037112 | TTAATGAATCGGCCGCGGTCCTAAATGCTGATGCA | 517 |
| 25-039049 | TAAGAGGTCATTTTAGACCGGAGGTGTATCACCGT | 518 |
| 26-039133 | TCACAGTTGAGGATTCCACACCTAGAAAAAGCCTG | 519 |
| 27-041112 | CTGGTAATGGGTAATCCAGCGAGGCAGAGGCATTT | 520 |
| 28-041154 | TTACACTGGTGTGTTTACCTGACCGACAAAAGGTA | 521 |
| 29-043049 | CCATTAGATACATTGAAGTTTTTGAGGCAGGTCAG | 522 |
| 30-043133 | CTCCGGCCAGAGCAGGTGGTGAAACCAATCAATAA | 523 |
| 31-045084 | GATAACCGACGGCCCTCAGGAGTAACCGATATTTT | 524 |
| 32-045112 | ACGTACAGCGCCATTACATCGTATAGAAGGCTTAT | 525 |
| 33-045154 | TAGACTTTCTCCGTTTAAATTAGCGAACCTCCCGA | 526 |
| 34-045168 | GGTGAAGACGCCAGGCGCAACGTAACAACTGGCCT | 527 |
| | TTTCACTCACCTCCATCTCCACTCCTACCCATCCAA | |
| | CTCCCACTTTCTAGATGTAT | |
| 35-037069 | CACTGCCCGCTTTCCGATGGTGAGCGTAACGATCTA | 528 |
| 36-010055bio | /5Biosg/TTTGGGGTTTCCGGAATAAGCAAACGAGCT | 529 |
| | TCAAAGCGAACGCT | |
| 37-000076 | GAATACCCAAAGACGCCAGTTTGAGGAAATATTTA | 530 |
| | AATTGTATTTCACTCACCTCCATCTCCACTCCTACC | |
| | CATCCAACTCCCACTTTCTAGATGTAT | |
| 38-000097 | CGAGGAATTATTTTGCGCATCAGATCGCACTCCAGC | 531 |
| | GACGTT | |
| 39-001056 | TTCAAAAGGGTGAGAAAGGCCGTATAAGCAAATAA | 532 |
| | AAATTTTCACTCACCTCCATCTCCACTCCTACCCAT | |
| | CCAACTCCCACTTTCTAGATGTAT | |
| 40-002097 | TGGTTTACAGTAGCGTAAAACTCACCGGAAACAAT | 533 |
| | CGTAAAA | |
| 41-004076bio | /5Biosg/TTTGATAGCACGTTTGCAGTGATGAAGGGG | 534 |
| | CAAATGGTCAATAAC | |
| 42-004097 | AACGTCACAAAATCAAAGCCGTCCGGCAAACGCGG | 535 |
| | CAGCATC | |
| 43-006097 | ACCGCCTAAACAAAAGCGGGGCGGGTCACTGTTGC | 536 |
| | GCCTGTG | |
| 44-008076bio | /5Biosg/TTTACCGTTCCAGTTAAGAATGCGGCGGGC | 537 |
| | GGATGGCTTAGAGCT | |
| 45-008097 | GAAAGCGTTCGGAACACTCTGTCTGCCAGCACGCG | 538 |
| | GGGTGCC | |
| 46-010097 | ATTAAGACACCCTCTAATGAGAAACCTGTCGTGCC | 539 |
| | CAGCAGG | |
| 47-011042 | GTGCCTTTTTGATGGCATTGACCACCCTGCATTTTG | 540 |
| | AATCAA | |
| 48-012097 | ACCCTCAAAGTTTTCGAAAATTAGCCCGAGATAGG | 541 |
| | GGAACCC | |
| 49-014097 | TGAATTTATTGTATTAAAGGGAAGGGAAGAAAGCG | 542 |
| | ACAGGAG | |
| 50-016097 | TGAATTTGACAGCAGCCGATTAATCAGTGAGGCCA | 543 |
| | GCTCATG | |
| 51-017042 | TTTTTCAGAGTGAGACGCCTGACCCATGGTATAGCT | 544 |
| | GCTCAG | |
| 52-018097 | CAGAGGCTATACCAGAAATACACCAGTCACACGAC | 545 |
| | CCAGCAG | |
| 53-022076 | TTCATTATAATTTCACCAGTCAGGACGTAGCACCGC | 546 |
| | CTGCAATTTCACTCACCTCCATCTCCACTCCTACCC | |
| | ATCCAACTCCCACTTTCTAGATGTAT | |
| 54-025098 | CCTTAACATTTGAGGATTTAGGCCGTCAATAGATAA | 547 |
| | TTGCGA | |
| 55-031081 | GTGTTGACGCTCAATCGTCTGACAGGGCCAGAATC | 548 |
| | CTGAGAA | |
| 56-031084 | TTTTTATAAAGGGAAGAAAGGAGCCCCCAAAAGAA | 549 |
| | CCTGTTT | |
| 57-032083 | GATTTAGAGCTTGACGGGGCTAAGCAAAATCCCTT | 550 |
| | ATAAATC | |
| 58-036083 | ACTCACATTAATTGCGTTGCCTGCCGTTTTCACGGT | 551 |
| | CATACC | |
| 59-037105 | AGCTGCAAAGCCTGTGCCTGTACTGCGCCCTGCGG | 552 |
| | AGGTGTC | |
| 60-040083 | AGGCGCTTTCGCACTCAATTGTCTAAAGTTAAACGA | 553 |
| | TGCTGA | |
| 61-044083 | AGTGCCAAGCTTTCAGAGGTATAGGACGACGACAG | 554 |
| | TATCGGC | |
| 62-045061 | ATAGTGGAGCCGCCACGGGAACGGGCCTTTCATCT | 555 |
| | TTTCATAAT | |
| 63-014055 | TTCAGCGCGTTGAAGTTCAGAGAATCCCCCTCAAAT | 556 |
| | GAAAGCCGG | |
| 64-018055 | CATTAAACAAAAGACGTTTACGTAAGAGCAACACT | 557 |
| | ATAATGGATT | |
| 65-000118 | ACAAAGTCCCTGAAAGGTCACTCCGGCACCGCTTC | 558 |
| | ACGCCAGGGTTTTC | |
| 66-000160 | TCTTACCAGATAACGATTCTCTCGCCATTCAGGCTC | 559 |
| | TGGCGAAAGGGGG | |
| 67-002139 | GAAGCGCCAAAATAGATTAAGAGTCCCGGAATTTG | 560 |
| | GCCAGCAGTTGGGC | |
| 68-004118 | GAGCGTCAATCAGAACATAAATTTCGTCTCGTCGCC | 561 |
| | AGCTTACGGCTGG | |
| 69-004160 | GCACCCAGCGTTTTTCTGCTCATAACGGAACGTGCA | 562 |
| | ATGCCAACGGCAG | |
| 70-006139 | GTAGGAACATGTAGCCATCCCTTTGCTCGTCATAAG | 563 |
| | GTGCCCCCTGCAT | |
| 71-008118 | AAGAAAAGTAATTTCAGTGTCTCTTCGCGTCCGTGA | 564 |
| | AGCATAAAGTGTA | |
| 72-008160 | TGCAGAAATAAAGTCAGCCAGTACCGAGCTCGAAT | 565 |
| | AAATTGTTATCCGC | |
| 73-010139 | TTGAGAAATAATTAAACATACGGGGAGAGGCGGTT | 566 |
| | GCCCTGAGAGAGTT | |
| 74-012118 | TAAGGCGCTATATGACGCTGGGTTGTTCCAGTTTGG | 567 |
| | GTGCCGTAAAGCA | |
| 75-012160 | TGACCTAACGCGAGCCCTTCAGACTCCAACGTCAA | 568 |
| | CACTACGTGAACCA | |
| 76-014139 | TTTTAACCCTTGAATTTTTTGGTGTAGCGGTCACGC | 569 |
| | GTATAACGTGCTT | |
| 77-016160 | ACATAAAACATTTATGCTTTGTTCTTTGATTAGTAA | 570 |
| | CTATCGGCCTTGC | |
| 78-020076 | ATTGTGTGATGAACGGTCAGTATTAAATTTAGGAAT | 571 |
| | ACCACAAGATTCA | |
| 79-022048 | TGCTCATCCGAACTTGTTACTAAAGAGGCGGGTAA | 572 |
| | CAGGGAGAACCATC | |
| 80-022055 | ACAAAGCTAAATTGAAAAATCTACGTTAGGTAGAA | 573 |
| | TTCAACTAGGCATA | |
| 81-028111 | GAAAAACCCGAGTAGAGCTAAAAAGGAGCTAAAT | 574 |
| | CGTTGAGTTTTGCCC | |
| 82-028125 | AGCCATTGCAACAGAAAAGGGACATTCTTTAAAAA | 575 |
| | TGATTATCAGATGA | |
| 83-043105 | TCCGTTTAAAATCCCGGCGAACCAGTCACCAGCTTG | 576 |
| | TTGGTGTAGATGG | |
| 84-043126 | TGGCAGCGGTTGTGGTTTACCTTGGGTATGGTGCCG | 577 |
| | ACCGTACATTTTT | |
| 85-005042 | TCACCGTAGGGAAGATAAAGGGACTCCTTGTGTAG | 578 |
| | GTAAAGATAGAACCATTTCAA | |
| 86-006055 | AGAGCCACAGGAGGCATTCCAACTAAAGTACGGTG | 579 |
| | TCCCGCCGGGCGCGGTTGCGG | |
| 87-012076 | ATTTTCACATAGTTGTTCCGAAATCGAGCGGATTGC | 580 |
| | ATCAAATTATAGTCAGAAGC | |
| 88-016076 | TACCGATTCGTCACCAGGAACGGTACTAATAGTAA | 581 |
| | AATGTTTGTTTTGCCAGAGGG | |
| 89-018139 | GAGGCGAAATATACACAATATAGAGATAGAACCCT | 582 |
| | GATAGCCCTAAAACACCTCAA | |
| 90-026153 | GCGAACTTCTGACCTGGTAATGCAATACACGAGCA | 583 |
| | CTGCGCGTCACCCAGAACGTG | |
| 91-028132 | TACCGCCTCACGCATCCTCGTCTGGCAAGGGTCGA | 584 |
| | GAACAAGGCAGCAAAACGCGC | |
| 92-034048 | AAGATTATTTAATTCTCCAACCTTTTGATAATTGCA | 585 |
| | TATGCATATAACAGTTGATT | |
| 93-034153 | CCGCCTGTGCGTATTCACAATCCCCGGGCGGTGCCA | 586 |
| | CATCCCCACCGTCCATCCTC | |
| 94-037084 | AGTCGGGTGAGCTAGGGGGTTTGGTGCTTATGAGC | 587 |
| | TCATTGCTTGCCGTCACAGGC | |
| 95-042153 | ATTTGCCTGAGAGAATGTGCTGCGCCATCGTGGGA | 588 |
| | GCCATCAACGGTAATCGTAAA | |
| 96-003020 | TTTAAACGTAGAAAAGACCCTGTATTT | 589 |
| 97-005020 | TTTATTCATTAAAGGGGCAAGGCATTT | 590 |
| 98-007020 | TTTCTGTAGCGCGTTTTTCATTTGTTT | 591 |
| 99-009020 | TTTACCACCAGAGCCCCCAATTCTTTT | 592 |
| 100-011020 | TTTATAAGTTTTAACAATGCTGTATTT | 593 |
| 101-013020 | TTTGTCGAGAGGGTTGATTAGAGATTT | 594 |
| 102-015020 | TTTGTCACCAGTACAGCCCGAAAGTTT | 595 |
| 103-017020 | TTTAGGAATTGCGAAATAAATCAATTT | 596 |
| 104-019020 | TTTATTCGGTCGCTGCCAATACTGTTT | 597 |
| 105-021020 | TTTAAGGCACCAACCAACCAAAATTTT | 598 |
| 106-023020 | TTTACGGTCAATCATATACATAACTTT | 599 |
| 107-025020 | TTTCTGACGAGAAACGAACTAACGTTT | 600 |
| 108-040196 | TTTCTGGTCTGGTCAACGGGTATTTTT | 601 |
| 109-042196 | TTTAGAGACGCAGAAGAGGTTTTGTTT | 602 |
| 110-044196 | TTTTGCGGGCCTCTTTTTGTTTAATTT | 603 |
| 111-046196 | TTTCAACATTAAATGCAATAATAATTT | 604 |
| 112-000174 | AATAGCAAAGGCTATCAGGTCATTGCTTT | 605 |
| 113-009035 | GCCGCCAATACAGGAGTGTACTGGTATTT | 606 |
| 114-012182 | TTTTTTAGTTAATTTCGTTATACAAATTTT | 607 |
| 115-016182 | TTTCTTTTTTAATGGTGAGAAGAGTCATTT | 608 |
| 116-020182 | TTTTAATGGAAGGGTACAATAACGGATTTT | 609 |
| 117-043023 | TTTGCGAACGAGTAGATTTAGTTTGACTGTTTA | 610 |
| 118-008193 | TTTACGACGACAATAAACAAAGTAATTCTGTCCAG | 611 |
| | TTT | |
| 119-026186 | TTTTATTTTTGAATGGCTATACGTGGCACAGACAAT | 612 |
| | TT | |
| 120-028186 | TTTGAGTAGAAGAACTCAAATAACATCACTTGCCTT | 613 |
| | TT | |
| 121-030186 | TTTCGCTACAGGGCGCGTAGCCGCGCTTAATGCGCT | 614 |
| | TT | |
| 122-032186 | TTTTATCAGGGCGATGGCCAGGGCGAAAAACCGTC | 615 |
| | TTT | |
| 123-034186 | TTTGTGAGACGGGCAACAGGTTTTTCTTTTCACCAT | 616 |
| | TT | |
| 124-036186 | TTTAGCTGTTTCCTGTGTGTCGTAATCATGGTCATTT | 617 |
| | T | |
| 125-038186 | TTTGGCATCAGATGCCGGGTCAGCAAATCGTTAACT | 618 |
| | TT | |
| 126-001025 | TTTTTATGCCTGAGTAATATTACGCAGTATGTTAGC | 619 |
| | TTT | |
| 127-048191 | TTTTTCTGAGAGTCTGGTCCTGTAGCCAGCTTTCAT | 620 |
| | TTT | |
| 128-024188 | TTTTTGTTGAAAGGAATTGAGAGTTGGCAAATCAA | 621 |
| | CATTT | |
| 129-033023 | TTTCGGAATCGTCATAAATATTCATTAAACGAGCTG | 622 |
| | ACTA | |
| 130-027021 | TTTTTGAACAACATTATTACAATAAAACACCAGAA | 623 |
| | CGAGTAG | |
| 131-029154 | TGAAAGCGTAAGAATTAGTCTTTTGGATTATACTTC | 624 |
| | TGAATTT | |
| 132-033154 | TAACCACCACACCCCTATGGTACAATTTCATTTGAA | 625 |
| | TTACTTT | |
| 133-037154 | TGGGCGCCAGGGTGCTGATTGAAAACTTTTTCAAAT | 626 |
| | ATATTTT | |
| 134-006193 | TTTAAACCAAGTACCGCACTCCAAGAGCAGCAACC | 627 |
| | GCAAGCGGACTTATCAAAC | |
| 135-000193 | TTTGAGCAAGAAACAATGATTAAGCCTGAGCGATG | 628 |
| | TTGGGAAGGGCGATCGGTTT | |
| 136-002193 | TTTCGTCAAAAATGAAAATACGATTTCGCTATTGGA | 629 |
| | TAGCTCTCACGGAAAATTT | |
| 137-004193 | TTTAAGCCTTAAATCAAGACTTGCGGACAGCGGGT | 630 |
| | AGAACGTCAGCGTGGTGTTT | |
| 138-029023 | TTTGCCAAAAGGAATTACGAATGCAGAAGGGAATC | 631 |
| | AGTGAATAAGGCTTGCCTTT | |
| 139-031023 | TTTAGCGAGAGGCTTTTGCCGATAAATAAAACGTA | 632 |
| | GCCGGAACGAGGCGCAGTTT | |
| 140-035023 | TTTAAATCAGGTCTTTACCAATGACCTAATAATGCC | 633 |
| | CACGCATAACCGATATTTT | |
| 141-037023 | TTTACTTCAAATATCGCGTAGAGGAAAACTACAAA | 634 |
| | TAGAAAGGAACAACTAATTT | |
| 142-039023 | TTTGTACCTTTAATTGCTCAGGTCAGGATATAATAC | 635 |
| | CGTAACACTGAGTTTCTTT | |
| 143-041023 | TTTGCTCAACATGTTTTAATGAATATGGGGTCATAC | 636 |
| | CAGGCGGATAAGTGCCTTT | |
| 144-045023 | TTTGGGCGCGAGCTGAAAAGCTATATTTCATCGCA | 637 |
| | GAGCCGCCACCAGAACCTTT | |
| 145-047023 | TTTAAGAATTAGCAAAATTTCATACATGAATTAGTT | 638 |
| | TGCCTTTAGCGTCAGATTT | |
| 146-049023 | TTTATACTTTTGCGGGAGAACATTATTACATACGTA | 639 |
| | AATATTGACGGAAATTTTT | |
| 147-028048 | CAGACGAAAAAGAAAGACTGGATAGCGTAGGCTTG | 640 |
| | AATACGTAATGCCACTACGTTT | |
| 148-045042 | GGTGGCACAATAAAAAGCAATACCAAAAAGCCTTT | 641 |
| | CTCATATATTTTAAATGCATTT | |
| 149-010182- | TTTTCTTACCAGTATAAAGCCATTTCGTTTCTTTGCT | 642 |
| T3.3p- | TGTGTCTTGTCTTCGTCGTGCTTGTTCC | |
| DYE40.22.9.3p | | |
| 150-014182- | TTTATAGTGAATTTATCAAAATTTTCGTTTCTTTGCT | 643 |
| T3.3p- | TGTGTCTTGTCTTCGTCGTGCTTGTTCC | |
| DYE40.22.9.3p | | |
| 151-018182- | TTTTCGCCTGATTGCTTTGAATTTTCGTTTCTTTGCT | 644 |
| T3.3p- | TGTGTCTTGTCTTCGTCGTGCTTGTTCC | |
| DYE40.22.9.3p | | |
| 152-022182- | TTTAATTTTAAAAGTTTGAGTATTTCGTTTCTTTGCT | 645 |
| T3.3p- | TGTGTCTTGTCTTCGTCGTGCTTGTTCC | |
| DYE40.22.9.3p | | |
| 153-002069- | CGACATTAGAAACGCAAAAGAACTGGCATTTCGTT | 646 |
| T3.3p- | TCTTTGCTTGTGTCTTGTCTTCGTCGTGCTTGTTCC | |
| DYE40.22.9.3p | | |
| 154-002153- | AACATAAATCAGAGGAAGCCCTTTTTAATTTCGTTT | 647 |
| T3.3p- | CTTTGCTTGTGTCTTGTCTTCGTCGTGCTTGTTCC | |
| DYE40.22.9.3p | | |
| 155-003098- | GTCACAATCAATCATACCAGAAGGAAACTTTCGTTT | 648 |
| T3.3p- | CTTTGCTTGTGTCTTGTCTTCGTCGTGCTTGTTCC | |
| DYE40.22.9.3p | | |
| 156-004090- | CCAATGAAAATCACCCAGCGCCAAAGACTTTCGTT | 649 |
| T3.3p- | TCTTTGCTTGTGTCTTGTCTTCGTCGTGCTTGTTCC | |
| DYE40.22.9.3p | | |
| 157-004132- | TTACCAACCAGTTAATTAGACGGGAGAATTTCGTTT | 650 |
| T3.3p- | CTTTGCTTGTGTCTTGTCTTCGTCGTGCTTGTTCC | |
| DYE40.22.9.3p | | |
| 158-004174- | TTAGTTGATAAGAAAGCAGCCTTTACAGTTTCGTTT | 651 |
| T3.3p- | CTTTGCTTGTGTCTTGTCTTCGTCGTGCTTGTTCC | |
| DYE40.22.9.3p | | |
| 159-006069- | GAACCGCTTATTAGGCACCGTAATCAGTTTTCGTTT | 652 |
| T3.3p- | CTTTGCTTGTGTCTTGTCTTCGTCGTGCTTGTTCC | |
| DYE40.22.9.3p | | |
| 160-006153- | TTTTTATACGCGAGGCTACAATTTTATCTTTCGTTTC | 653 |
| T3.3p- | TTTGCTTGTGTCTTGTCTTCGTCGTGCTTGTTCC | |
| DYE40.22.9.3p | | |
| 161-007098- | ACCGGAACCAGACATTAGCAAGGCCGGATTTCGTT | 654 |
| T3.3p- | TCTTTGCTTGTGTCTTGTCTTCGTCGTGCTTGTTCC | |
| DYE40.22.9.3p | | |
| 162-008090- | CAGTCTCTATTCACCCCTCAGAGCCGCCTTTCGTTT | 655 |
| T3.3p- | CTTTGCTTGTGTCTTGTCTTCGTCGTGCTTGTTCC | |
| DYE40.22.9.3p | | |
| 163-008132- | GATAAGTTTACGAGTCATTACCGCGCCCTTTCGTTT | 656 |
| T3.3p- | CTTTGCTTGTGTCTTGTCTTCGTCGTGCTTGTTCC | |
| DYE40.22.9.3p | | |
| 164-008174- | ACATGTTTTATCATTCATCGAGAACAAGTTTCGTTT | 657 |
| T3.3p- | CTTTGCTTGTGTCTTGTCTTCGTCGTGCTTGTTCC | |
| DYE40.22.9.3p | | |
| 165-010069- | GGATTAGGTATAAACAGTAAGCGTCATATTTCGTTT | 658 |
| T3.3p- | CTTTGCTTGTGTCTTGTCTTCGTCGTGCTTGTTCC | |
| DYE40.22.9.3p | | |
| 166-010153- | ACAGTAGAGAGAATCGCGCCTGTTTATCTTTCGTTT | 659 |
| T3.3p- | CTTTGCTTGTGTCTTGTCTTCGTCGTGCTTGTTCC | |
| DYE40.22.9.3p | | |
| 167-011098- | CCTATTATTCTGATATAAAGCCAGAATGTTTCGTTT | 660 |
| T3.3p- | CTTTGCTTGTGTCTTGTCTTCGTCGTGCTTGTTCC | |
| DYE40.22.9.3p | | |
| 168-012090- | GAGCCACGTACCGCGGCTGAGACTCCTCTTTCGTTT | 661 |
| T3.3p- | CTTTGCTTGTGTCTTGTCTTCGTCGTGCTTGTTCC | |
| DYE40.22.9.3p | | |
| 169-012132- | GACCGTGCGGAATCTCGCCATATTTAACTTTCGTTT | 662 |
| T3.3p- | CTTTGCTTGTGTCTTGTCTTCGTCGTGCTTGTTCC | |
| DYE40.22.9.3p | | |
| 170-014069- | CAACTTTCAGCCCTGGGATAGCAAGCCCTTTCGTTT | 663 |
| T3.3p- | CTTTGCTTGTGTCTTGTCTTCGTCGTGCTTGTTCC | |
| DYE40.22.9.3p | | |
| 171-014153- | CTGAGAGACAAAGAAATTTAATGGTTTGTTTCGTTT | 664 |
| T3.3p- | CTTTGCTTGTGTCTTGTCTTCGTCGTGCTTGTTCC | |
| DYE40.22.9.3p | | |
| 172-015098- | GTCGTCTTTCCAAATTCTCAGAACCGCCTTTCGTTT | 665 |
| T3.3p- | CTTTGCTTGTGTCTTGTCTTCGTCGTGCTTGTTCC | |
| DYE40.22.9.3p | | |
| 173-016090- | CTTAAACGCCTTTATCTGTATGGGATTTTTTCGTTTC | 666 |
| T3.3p- | TTTGCTTGTGTCTTGTCTTCGTCGTGCTTGTTCC | |
| DYE40.22.9.3p | | |
| 174-016132- | CCTTGCTTTAGAATCTCCGGCTTAGGTTTTTCGTTTC | 667 |
| T3.3p- | TTTGCTTGTGTCTTGTCTTCGTCGTGCTTGTTCC | |
| DYE40.22.9.3p | | |
| 175-018069- | CATGAGGTGCGGGAAGTTGCGCCGACAATTTCGTT | 668 |
| T3.3p- | TCTTTGCTTGTGTCTTGTCTTCGTCGTGCTTGTTCC | |
| DYE40.22.9.3p | | |
| 176-018153- | TACAAAAATTAATTTCAATATATGTGAGTTTCGTTT | 669 |
| T3.3p- | CTTTGCTTGTGTCTTGTCTTCGTCGTGCTTGTTCC | |
| DYE40.22.9.3p | | |
| 177-019098- | TCGGAACGAGGGCACTTTGCTTTCGAGGTTTCGTTT | 670 |
| T3.3p- | CTTTGCTTGTGTCTTGTCTTCGTCGTGCTTGTTCC | |
| DYE40.22.9.3p | | |
| 178-020090- | TCATCGCCAGCGATTTTGAGGACTAAAGTTTCGTTT | 671 |
| T3.3p- | CTTTGCTTGTGTCTTGTCTTCGTCGTGCTTGTTCC | |
| DYE40.22.9.3p | | |
| 179-020132- | ACAGAAATCAGATGATTATTCATTTCAATTTCGTTT | 672 |
| T3.3p- | CTTTGCTTGTGTCTTGTCTTCGTCGTGCTTGTTCC | |
| DYE40.22.9.3p | | |
| 180-022069- | CCCAAATGAGGACACGAAATCCGCGACCTTTCGTT | 673 |
| T3.3p- | TCTTTGCTTGTGTCTTGTCTTCGTCGTGCTTGTTCC | |
| DYE40.22.9.3p | | |
| 181-022153- | CATTTTGTATAATCTCAAAATTATTTGCTTTCGTTTC | 674 |
| T3.3p- | TTTGCTTGTGTCTTGTCTTCGTCGTGCTTGTTCC | |
| DYE40.22.9.3p | | |
| 182-023098- | GGCTGGCTGACCTCAGAGTACAACGGAGTTTCGTTT | 675 |
| T3.3p- | CTTTGCTTGTGTCTTGTCTTCGTCGTGCTTGTTCC | |
| DYE40.22.9.3p | | |
| 183-025077- | AACTTTAATCATTGACAAGAACCGGATATTTCGTTT | 676 |
| T3.3p- | CTTTGCTTGTGTCTTGTCTTCGTCGTGCTTGTTCC | |
| DYE40.22.9.3p | | |
| 184-025119- | AAGTATTAGACTTTCACCAGAAGGAGCGTTTCGTTT | 677 |
| T3.3p- | CTTTGCTTGTGTCTTGTCTTCGTCGTGCTTGTTCC | |
| DYE40.22.9.3p | | |
| 185-002118 | TTAACTGAAAGAAAATTCATA | 678 |
| 186-006118 | AATAGCAAGGCCACCACCGGA | 679 |
| 187-010118 | AACGCCAACAAACATGAAAGT | 680 |
| 188-014118 | GGGTTATATGACGTTAGTAAA | 681 |
| 189-018118 | TTACCTGAGTAGCAACGGCTA | 682 |
| 190-022118 | GAATTATCATTCATCAAGAGT | 683 |
| 191-000055 | TGATTAATGGCAACATATAAACAACCGA | 684 |
| 192-000139 | GAAAAGTAATTGAGCGCTAATAAACAGG | 685 |
| 193-002076 | AAAAGGGAATTAGAGCCAGCAAACCATC | 686 |
| 194-002160 | AGAGAATTTATCCCAATCCAACTATTTT | 687 |
| 195-004055 | AGCGACACGGTCATAGCCCCCCACCCTC | 688 |
| 196-004139 | CTGAATCCCGGTATTCTAAGATTTCATC | 689 |
| 197-006076 | ACCCTCAACGATTGGCCTTGATGAATTT | 690 |
| 198-006160 | CAAGCCGTCGGCTGTCTTTCCCAGCTAA | 691 |
| 199-008055 | CATGGCTGAGTAACAGTGCCCGATTAGC | 692 |
| 200-008139 | AACAATATCGAGCCAGTAATAGGCTTAA | 693 |
| 201-010076 | AAGAGAAACTCAGGAGGTTTACACCCTC | 694 |
| 202-010160 | ACGCTCATTTAGTATCATATGCATCTTC | 695 |
| 203-012055 | AATAGGATAGCATTCCACAGACAACAGT | 696 |
| 204-012139 | AAATACCAATCCAATCGCAAGACTACCT | 697 |
| 205-014076 | TGCTAAAAGGCTCCAAAAGGAAGCTTGA | 698 |
| 206-014160 | CATAGGTTTAGATTAAGACGCAAACAGT | 699 |
| 207-016055 | TGACAACTTAAAGGCCGCTTTAAGTTTC | 700 |
| 208-016139 | TGAATAAATCAAGAAAACAAATCGCGCA | 701 |
| 209-018076 | ACTTTTTTCATCTTTGACCCCCTGATAA | 702 |
| 210-018160 | ACCAAGTTTACATCGGGAGAATAGAACC | 703 |
| 211-020055 | TGCTCCAGACCAACTTTGAAACAACGTA | 704 |
| 212-020139 | ACGTAAATGGCAATTCATCAACGGAACA | 705 |
| 213-048090 | AAAACAGGAAGATTGGAGACAAATAACG | 706 |
| 214-048132 | TGTCAATCATATGTAGCTGATTAGCCGA | 707 |
| 215-048174 | AGCAAACAAGAGAAATCTACAATAGCTA | 708 |
| 216-005098 | ACCATTACCATTTCCAGAGCCTAATTTGCGCTAAC | 709 |
| 217-009098 | TAAATCCTCATTAATATCCCATCCTAATCCTGAAC | 710 |
| 218-013098 | AGAACCGCCACCAAATAAGAATAAACACTGATAAA | 711 |
| 219-017098 | CGGTTTATCAGCATTAATTAATTTTCCCTCTGTAA | 712 |
| 220-021098 | AGCGCGAAACAAATTTTCAGGTTTAACGTAAAGAA | 713 |
| 221-027056- | /5Biosg/TTTTCAGTTGTGGGAAGGGCTTGAGATGGTT | 714 |
| T3.5p-BIO.5p | | |
| 222-027098- | /5Biosg/TTTCAGCAAATGAAAAACGAACCACAGTAA | 715 |
| T3.5p-BIO.5p | T | |
| 223-027140- | /5Biosg/TTTATATCAATAGGAGCATTCGACAACTCGT | 716 |
| T3.5p-BIO.5p | | |
| 224-049098- | /5Biosg/TTTTTCGCATTAAATTTTTGATAATCAGAAA | 717 |
| T3.5p-BIO.5p | | |
| 225-049140- | /5Biosg/TTTTAGGAACACAAACGGCGGATTGGAAAC | 718 |
| T3.5p-BIO.5p | C | |
| 226-049056- | /5Biosg/TTTCGCAAGGGCTAAATCGGTTGTAAAGCCT | 719 |
| T3.5p-BIO.5p | CAGAGCA | |
| 227-027119- | /5Biosg/TTTCATCACCTTGCTGAATCGCCAGGCCAAC | 720 |
| T3.5p-BIO.5p | | |
| 228-049077- | /5Biosg/TTTAACGTTATGCATCTACCACGGAATAAGT | 721 |
| T3.5p-BIO.5p | | |
| 229-049119- | /5Biosg/TTTATCAGCTATGGGATCAAAGTCAGAGGG | 722 |
| T3.5p-BIO.5p | T | |
| 230-049161- | /5Biosg/TTTTTCGCGTCCCGTCGCCACAAGAATTGAG | 723 |
| T3.5p-BIO.5p | | |
| 231-027077- | /5Biosg/TTTCAGTGCCACGCTGAAACAGAGCAGATT | 724 |
| T3.5p-BIO.5p | CCTACATT | |
| 232-027161- | /5Biosg/TTTTCTGGTCGAAGGTTCCTTTGCCCGAACG | 725 |
| T3.5p-BIO.5p | TTATTTTT | |
| 221-027056- | TTTTCAGTTGTGGGAAGGGCTTGAGATGGTT | 726 |
| T3.5p-NULL.5p | | |
| 222-027098- | TTTCAGCAAATGAAAAACGAACCACAGTAAT | 727 |
| T3.5p-NULL.5p | | |
| 223-027140- | TTTATATCAATAGGAGCATTCGACAACTCGT | 728 |
| T3.5p-NULL.5p | | |
| 224-049098- | TTTTTCGCATTAAATTTTTGATAATCAGAAA | 729 |
| T3.5p-NULL.5p | | |
| 225-049140- | TTTTAGGAACACAAACGGCGGATTGGAAACC | 730 |
| T3.5p-NULL.5p | | |
| 226-049056- | TTTCGCAAGGGCTAAATCGGTTGTAAAGCCTCAGA | 731 |
| T3.5p-NULL.5p | GCA | |
| 227-027119- | TTTCATCACCTTGCTGAATCGCCAGGCCAAC | 732 |
| T3.5p-NULL.5p | | |
| 228-049077- | TTTAACGTTATGCATCTACCACGGAATAAGT | 733 |
| T3.5p-NULL.5p | | |
| 229-049119- | TTTATCAGCTATGGGATCAAAGTCAGAGGGT | 734 |
| T3.5p-NULL.5p | | |
| 230-049161- | TTTTTCGCGTCCCGTCGCCACAAGAATTGAG | 735 |
| T3.5p-NULL.5p | | |
| 231-027077- | TTTCAGTGCCACGCTGAAACAGAGCAGATTCCTAC | 736 |
| T3.5p-NULL.5p | ATT | |
| 232-027161- | TTTTCTGGTCGAAGGTTCCTTTGCCCGAACGTTATT | 737 |
| T3.5p-NULL.5p | TTT | |

The Lobe has a nucleic acid origami structure with 10 locations for attachment of tethers. The tethers include sequences having an 8 nucleotide sequence region (AGATGTAT) at the 3′ end. The 8 nucleotide sequence at the 3′ end of the tethers is complementary to the 8 nucleotide sequence at the 3′ end of the dockers. The origami structure of the Lobe is formed using the p8064 scaffold (Tilibit Nanosystems, Munich Germany) and the oligonucleotides listed in Table 9. The Lobe also includes Alexa 647 fluorescent labels attached at 36 locations (sequences with the word “DYE” in the first column of Table 9). The origami Lobe folds to produce a pegboard structure. The Lobe also includes 15 biotinylated oligonucleotides (indicated “/5Biosg/” in Table 9) for attachment of streptavidin modified antibodies or other affinity components.

SNAPS and Lobes were folded and evaluated as set forth in Example I.

Notwithstanding the appended claims, the disclosure set forth herein is also defined by the following clauses:

1. A method of identifying an analyte, comprising
(a) providing an array of analytes, wherein each of the analytes is attached to a unique identifier in the array, wherein a linker is attached to each unique identifier in the array;
(b) providing an affinity reagent, wherein the affinity reagent comprises a plurality of paratopes that independently recognize an epitope of an analyte at a first unique identifier of the array, wherein the paratopes preferentially recognize the analyte compared to other analytes in the array;
(c) attaching a docker to the linker that is attached to each unique identifier in the array, wherein the docker comprises the epitope or analog thereof,
(d) contacting the array with the affinity reagent, whereby the affinity reagent associates with the first unique identifier via binding of at least one of the paratopes to the epitope of the analyte and via binding of at least one of the paratopes to the epitope of the docker; and
(e) detecting association of the affinity reagent with the first unique identifier, thereby identifying the analyte at the first unique identifier.

2. The method of clause 1, wherein the analyte at the first unique identifier further comprises a second epitope that differs from the epitope.

3. The method of clause 2, wherein the paratopes preferentially recognize the epitope compared to the second epitope.

4. The method of clause 2 or 3, further comprising:
(f) removing the affinity reagent and the docker from the array;
(g) attaching a second docker to the linker that is attached to each unique identifier in the array, wherein the second docker comprises the second epitope or analog thereof,
(h) contacting the array with a second affinity reagent comprising a plurality of second paratopes that independently recognize the second epitope, whereby the second affinity reagent associates with the first unique identifier via binding of at least one of the second paratopes to the second epitope of the analyte and via binding of at least one of the second paratopes to the second epitope of the second docker; and
(i) detecting association of the second affinity reagent with the first unique identifier, thereby identifying the analyte at the first unique identifier

5. The method of any one of clauses 1 through 4, wherein the linker comprises a universal linker, each of the unique identifiers in the array being attached to a universal linker that is the same as a universal linker attached to the other unique identifiers in the array.

6. The method of any one of clauses 1 through 5, wherein the analytes comprise proteins and the epitope comprises amino acids.

7. The method of any one of clauses 1 through 5, wherein the linker comprises a first nucleotide sequence and wherein the docker comprises a second nucleotide sequence that is complementary to the first nucleotide sequence.

8. The method of clause 7, wherein the attaching of step (c) comprises hybridizing the first nucleotide sequence to the second nucleotide sequence.

9. The method of any one of clauses 6 through 8, wherein the array comprises at least 1,000 different proteins attached to respective unique identifiers of the array.

10. The method of any one of clauses 1 through 9, wherein a plurality of dockers is attached to each unique identifier in the array during step (d), each of the dockers comprising the epitope.

11. The method of clause 10, wherein the plurality of dockers comprises at least six dockers comprising the epitope.

12. The method of any one of clauses 1 through 11, wherein the array comprises a solid support and wherein the unique identifiers comprise addresses on the solid support.

13. The method of clause 12, wherein the analytes are attached to the addresses via particles.

14. The method of clause 13, wherein the particles comprise structured nucleic acid particles.

15. The method of any one of clauses 1 through 14, wherein the unique identifiers comprise particles in fluid phase.

16. The method of clause 15, wherein the particles comprise structured nucleic acid particles.

17. The method of any one of clauses 1 through 16, wherein individual unique identifiers in the array are each attached to a single analyte.

18. The method of any one of clauses 1 through 17, wherein the affinity reagent further comprises a plurality of labels.

19. The method of clause 18, wherein the affinity reagent further comprises a structured nucleic acid particle attached to the plurality of paratopes and to the plurality of labels.

20. A method of identifying an analyte, comprising
(a) providing an array of analytes, wherein each of the analytes is attached to a unique identifier in the array, wherein a linker is attached to each unique identifier in the array;
(b) providing an affinity reagent, wherein the affinity reagent comprises a paratope that recognizes an epitope of an analyte at a first unique identifier of the array, wherein the paratope preferentially recognizes the analyte compared to other analytes in the array, wherein the affinity reagent further comprises a first reactive moiety;
(c) attaching a second reactive moiety to the linker, wherein the second reactive moiety is reactive toward the first reactive moiety to form a covalently linked product;
(d) contacting the array with the affinity reagent, whereby the affinity reagent associates with the first unique identifier via binding of at least one of the paratopes to the epitope and via the covalently linked product of reaction between the first reactive moiety with the second reactive moiety; and
(e) detecting association of the affinity reagent with the first unique identifier, thereby identifying the analyte at the first unique identifier.

21. The method of clause 20, wherein the linker comprises a universal linker, each of the unique identifiers in the array being attached to a universal linker that is the same as a universal linker attached to the other unique identifiers in the array.

22. The method of clause 20 or 21, wherein the first reactive moiety and the second reactive moiety comprise bioorthogonal reactants or click reactants.

23. The method of any one of clauses 20 through 22, wherein the analytes comprise proteins and the epitope comprises amino acids.

24. The method of clause 23, wherein the array comprises at least 1,000 different proteins attached to respective unique identifiers of the array.

25. The method of any one of clauses 20 through 24, wherein the linker comprises a first nucleotide sequence and wherein the second reactive moiety comprises a second nucleotide sequence that is complementary to the first nucleotide sequence.

26. The method of clause 25, wherein the attaching of step (c) comprises hybridizing the first nucleotide sequence to the second nucleotide sequence.

27. The method of any one of clauses 20 through 26, further comprising:
(f) removing the affinity reagent and the covalently linked product from the array;
(g) attaching a third reactive moiety to the linker, wherein the third reactive moiety is reactive toward a fourth reactive moiety to form a covalently linked product;
(h) contacting the array with a second affinity reagent, wherein the second affinity reagent comprises a paratope that recognizes a second epitope of the analyte at the first unique identifier of the array, wherein the paratope preferentially recognizes the analyte compared to other analytes in the array, wherein the affinity reagent further comprises the fourth reactive moiety, whereby the affinity reagent associates with the first unique identifier via binding of at least one of the paratopes to the epitope and via the covalently linked product of reaction between the third reactive moiety with the fourth reactive moiety; and
(i) detecting association of the second affinity reagent with the first unique identifier, thereby identifying the analyte at the first unique identifier.

28. The method of clause 27, wherein the linker comprises a first nucleotide sequence and wherein the third reactive moiety comprises a third nucleotide sequence that is complementary to the first nucleotide sequence.

29. The method of clause 28, wherein the attaching of step (g) comprises hybridizing the first nucleotide sequence to the third nucleotide sequence.

30. The method of any one of clauses 27 through 29, wherein the first through fourth reactive moieties comprise bioorthogonal reactants or click reactants.

31. The method of any one of clauses 27 through 30, wherein the removing of the covalently linked product from the array comprises denaturing a double stranded nucleic acid attaching the covalently linked product to the first unique identifier.

32. The method of any one of clauses 27 through 30, wherein the removing of the covalently linked product from the array comprises cleaving a covalent bond attaching the covalently linked product to the first unique identifier.

33. The method of any one of clauses 20 through 32, wherein the linker comprises a universal linker, each of the unique identifiers in the array being attached to a universal linker that is the same as a universal linker attached to the other unique identifiers in the array.

34. The method of any one of clauses 20 through 33, wherein the array comprises a solid support and wherein the unique identifiers comprise addresses on the solid support.

35. The method of clause 34, wherein the analytes are attached to the addresses via particles.

36. The method of clause 35, wherein the particles comprise structured nucleic acid particles.

37. The method of any one of clauses 20 through 36, wherein the unique identifiers comprise particles in fluid phase.

38. The method of clause 37, wherein the particles comprise structured nucleic acid particles.

39. The method of any one of clauses 20 through 38, wherein individual unique identifiers in the array are each attached to a single analyte.

40. The method of any one of clauses 20 through 39, wherein the affinity reagent further comprises a plurality of labels.

41. The method of clause 40, wherein the affinity reagent further comprises a structured nucleic acid particle attached to the linker, plurality of paratopes and plurality of labels.

42. An assay composition, comprising:
(a) an analyte; and
(b) an affinity reagent;
wherein the analyte comprises an epitope attached to a docker; and
wherein the affinity reagent comprises a paratope that preferentially recognizes the epitope compared to the docker, and a tether that preferentially recognizes the docker compared to the epitope.

43. The assay composition of clause 42, further comprising:
(a) a plurality of different analytes; and
(b) a plurality of different affinity reagents;
wherein each analyte in the plurality of different analytes comprises:
   (i) a different epitope compared to other analytes in the plurality of analytes, and
   (ii) a universal docker, wherein analytes comprising different epitopes further comprise the same universal docker; and
wherein each of affinity reagent in the plurality of different affinity reagents comprises:
(i) a different paratope that preferentially recognizes a first epitope in the plurality of different analytes compared to a second epitope in the plurality of different analytes, and
(ii) a universal tether that recognizes the universal docker, wherein affinity reagents comprising different paratopes further comprise the same universal tether.

44. The assay composition of clause 43, wherein the plurality of different analytes comprises an array of at least 1,000 different proteins attached to respective unique identifiers of the array.

45. The assay composition of clause 43, wherein a plurality of dockers is attached to each unique identifier in the array.

46. The assay composition of clause 45, wherein the affinity reagent comprises a plurality of tethers that recognize the docker.

47. The assay composition of any one of clauses 43 through 46, wherein the array comprises a solid support and wherein the unique identifiers comprise addresses on the solid support.

48. The assay composition of clause 47, wherein the proteins are attached to the addresses via particles.

49. The assay composition of clause 48, wherein the particles comprise structured nucleic acid particles.

50. The assay composition of any one of clauses 44 through 49, wherein individual unique identifiers in the array are each attached to a single protein.

51. The assay composition of clause 42, wherein the analyte comprises a protein and the epitope comprises amino acids.

52. The assay composition of clause 51, wherein the docker comprises a first nucleotide sequence.

53. The assay composition of clause 52, wherein the tether comprises a second nucleotide sequence that is complementary to the first nucleotide sequence, and wherein the second sequence optionally comprises a palindromic sequence capable of forming a hairpin.

54. The assay composition of clause 52, wherein the tether comprises a protein that recognizes the first nucleotide sequence.

55. The assay composition of clause 42, wherein the docker comprises an antibody, Fab′ fragment, F(ab′)₂ fragment, single-chain variable fragments, di-scFv, tri-scFv, microantibody, epitope, paratope, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag.

56. The assay composition of clause 55, wherein the tether comprises an antibody, Fab′ fragment, F(ab′)₂ fragment, single-chain variable fragments, di-scFv, tri-scFv, microantibody, epitope, paratope, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag.

57. The assay composition of any one of clauses 42 through 56, wherein the affinity reagent comprises a plurality of paratopes, and wherein each of the paratopes recognizes the epitope of the analyte.

58. The assay composition of clause 57, wherein the affinity reagent further comprises a plurality of labels.

59. The assay composition of clause 57 or 58, wherein the affinity reagent further comprises a plurality of tethers.

60. The assay composition of any one of clauses 42 through 59, wherein the affinity reagent further comprises a structured nucleic acid particle attached to the paratope and the tether.

61. The assay composition of any one of clauses 42 through 60, wherein the analyte further comprises a structured nucleic acid particle attached to the epitope and the docker.

62. An affinity reagent for an analyte, comprising:
(a) a paratope having affinity for an epitope of the analyte; and
(b) a tether having affinity for a docker, wherein the docker is attached to the analyte, and wherein the paratope has greater affinity for the epitope compared to the affinity of the tether for the docker.

63. The affinity reagent of clause 62, wherein the affinity reagent has higher avidity for the analyte compared to the avidity of the paratope for the epitope.

64. The affinity reagent of clause 62, wherein the paratope is orthogonal to the tether.

65. The affinity reagent of clause 64, wherein the paratope is a moiety of an antibody and the tether is a nucleotide sequence of a nucleic acid.

66. The affinity reagent of clause 64, wherein the paratope comprises an amino acid sequence and the tether comprises a nucleotide sequence.

67. The affinity reagent of clause 62, further comprising a plurality of paratopes having affinity for the epitope of the analyte.

68. The affinity reagent of clause 67, further comprising a plurality of tethers having affinity for the docker.

69. The affinity reagent of any one of clauses 62 through 68, wherein the affinity reagent is associated with the analyte via binding of the tether to the docker and via binding of the paratope to the epitope.

70. The affinity reagent of clause 69, wherein the affinity reagent comprises a plurality of tethers and the analyte comprises a plurality of dockers.

71. The affinity reagent of clause 70, wherein the plurality of tethers is bound to the plurality of dockers.

72. The affinity reagent of any one of clauses 69 through 71, wherein the affinity reagent comprises a plurality of paratopes.

73. The affinity reagent of any one of clauses 69 through 72, wherein the analyte is attached to an address in an array.

74. The affinity reagent of any one of clauses 69 through 73, wherein the analyte is attached to a structured nucleic acid particle.

75. The affinity reagent of clause 74, wherein the docker is attached to the structured nucleic acid particle.

76. The affinity reagent of any one of clauses 62 through 75, wherein the analyte comprises a protein and the epitope comprises amino acids.

77. The affinity reagent of clause 76, wherein the tether comprises a first nucleotide sequence, and wherein the first nucleotide sequence optionally comprises a palindromic sequence capable of forming a hairpin.

78. The affinity reagent of clause 77, wherein the docker comprises a second nucleotide sequence that is complementary to the first nucleotide sequence.

79. The affinity reagent of any one of clauses 62 through 78, wherein the tether comprises an antibody, Fab′ fragment, F(ab′)₂ fragment, single-chain variable fragments, di-scFv, tri-scFv, microantibody, epitope, paratope, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag.

80. The affinity reagent of any one of clauses 62 through 79, comprising a plurality of paratopes, and wherein each of the paratopes has affinity for the epitope of the analyte.

81. The affinity reagent of clause 80, further comprising a plurality of labels.

82. The affinity reagent of any one of clauses 62 through 81, further comprising a plurality of tethers having affinity for the docker.

83. The affinity reagent of any one of clauses 62 through 82, further comprising a structured nucleic acid particle attached to the paratope and the tether.

84. A composition, comprising:
(a) an analyte attached to a first particle or solid support, wherein the analyte comprises an epitope, and wherein a set of dockers is attached to fixed positions on the first particle or solid support; and
(b) an affinity reagent attached to a second particle, wherein the affinity reagent comprises a paratope, wherein a set of tethers is attached to fixed positions on the second particle, wherein the paratope recognizes the epitope, wherein the tethers recognize the dockers, and
wherein the fixed positions of the set of dockers on the first particle or solid support and the fixed positions of the set of tethers on the second particle prevent at least one of the tethers from contacting the set of dockers while (i) the paratope is bound to the epitope and (ii) a subset of the tethers is simultaneously bound to a subset of the dockers.

85. The composition of clause 84, wherein the analyte is bound to the affinity reagent via (i) the paratope being bound to the epitope and (ii) the subset of the tethers being simultaneously bound to the subset of the dockers.

86. The composition of clause 84 or 85, wherein the set of dockers is in molar excess compared to the set of tethers.

87. The composition of any one of clauses 84 through 86, wherein the set of tethers comprises three tethers.

88. The composition of any one of clauses 84 through 87, wherein the subset of tethers comprises two tethers.

89. The composition of any one of clauses 84 through 88, wherein the set of tethers comprises a first tether, a second tether and a third tether, wherein the fixed position for the second tether is equidistant from the fixed position for the first tether and from the fixed position for the third tether, and wherein the subset of the tethers comprises the second tether.

90. The composition of clause 89, wherein the fixed position for the first tether is separated from the fixed position for the third tether by a distance that is greater than the equidistance.

91. The composition of clause 89 or 90, wherein the fixed positions of the set of dockers on the first particle or solid support and the fixed positions of the set of tethers on the second particle prevent the first tether from contacting the set of dockers while (i) the paratope is bound to the epitope and (ii) the second tether and the third tether are simultaneously bound to the subset of the dockers.

92. The composition of any one of clauses 89 through 91, wherein the fixed positions for individual tethers in the set of tethers are separated from each other by a distance, wherein the individual tethers each have a length, and wherein the sum of the length of any two individual tethers in the set is less than the distance separating the fixed positions for the two individual tethers.

93. The composition of any one of clauses 84 through 92, wherein the set of dockers comprises four dockers.

94. The composition of clause 93, wherein the fixed position for a first docker of the four dockers is equidistant to the fixed positions for a second and third docker of the four dockers.

95. The composition of clause 94, wherein the fixed position for the first docker is separated from the fixed positions for a fourth docker of the four dockers by a distance that is greater than the equidistance.

96. The composition of any one of clauses 84 through 95, wherein the analyte is equidistant to the fixed positions for the set of dockers.

97. The composition of any one of clauses 84 through 96, wherein the paratope is equidistant to the fixed positions for the set of tethers.

98. A flow cell comprising the composition of any one of clauses 84 through 97.

99. The flow cell of clause 98, wherein the first particle is solid-phase immobilized in the flow cell or wherein the solid support is in the flow cell.

100. The flow cell of clause 98 or 99, wherein the second particle is in solution-phase in the flow cell.

101. The flow cell of any one of clauses 98 through 100, wherein the flow cell is mounted to a detection system and observed by a detector of the detection system.

102. The flow cell of clause 101, wherein the flow cell is in fluidic communication with a fluidic component of the detection system.

103. A kit comprising the composition of any one of clauses 84 through 97.

104. The kit of clause 103, wherein the kit is in fluidic communication with a fluidic component of a detection system.

105. A method comprising
(a) providing an analyte attached to a first particle or solid support, wherein the analyte comprises an epitope, and wherein a set of dockers is attached to fixed positions on the first particle or solid support;
(b) contacting the analyte with an affinity reagent, wherein the affinity reagent is attached to a second particle, wherein the affinity reagent comprises a paratope, wherein a set of tethers is attached to fixed positions on the second particle,
wherein the paratope binds to the epitope,
wherein a subset of the tethers binds to a subset of the dockers, and
   wherein the fixed positions of the set of dockers on the first particle or solid support and the fixed positions of the set of tethers on the second particle prevent at least one of the tethers from contacting the set of dockers while (i) the paratope is bound to the epitope and (ii) the subset of the tethers is simultaneously bound to the subset of the dockers, thereby forming a complex comprising the analyte and the affinity reagent.

106. The method of clause 105, further comprising (c) detecting the complex.

107. The method of clause 106, wherein the detecting comprises detecting signal from a label attached to the affinity reagent.

108. The method of clause 105, wherein the method is carried out in a fluid and wherein the method further comprises (c) separating the complex from the fluid.

109. The method of clause 108, further comprising (d) dissociating the analyte from the affinity reagent after step (c).

110. The method of any one of clauses 105 through 109, wherein the first particle or solid support is immobilized in a flow cell and step (b) comprises delivering the affinity reagent to the flow cell.

111. The method of clause 110, further comprising detecting the complex.

112. The method of clause 110 or 111, further comprising dissociating the affinity reagent from the analyte and removing the affinity reagent from the flow cell.

113. The method of any one of clauses 110-112, further comprising contacting the analyte with a second affinity reagent.

114. A method of identifying an analyte, comprising
(a) providing an array of analytes, wherein each of the analytes is attached to a unique identifier in the array, wherein a docker comprising a protein is attached to each unique identifier in the array;
(b) providing an affinity reagent, wherein the affinity reagent comprises a paratope that recognizes an epitope of an analyte at a first unique identifier of the array, wherein the paratope preferentially recognizes the analyte compared to other analytes in the array, wherein the affinity reagent further comprises a tether, wherein the tether comprises a binding ligand of the protein of the docker;
(c) contacting the array with the affinity reagent, whereby the affinity reagent associates with the first unique identifier via binding of the paratope to the epitope and via binding of the binding ligand to the protein; and
(d) detecting association of the affinity reagent with the first unique identifier, thereby identifying the analyte at the first unique identifier.

115. The method of clause 114, wherein the binding ligand comprises an allosteric regulator.

116. The method of clause 115, wherein the protein has a binding specificity for the allosteric regulator.

117. The method of clause 114, wherein the protein comprises a luminescent enzyme.

118. The method of clause 117, wherein the binding ligand comprises a cofactor of the luminescent enzyme.

119. The method of clause 118, wherein detecting association of the affinity reagent with the first unique identifier comprises detecting a luminescent signal from the luminescent enzyme associated with the first unique identifier.

1. A method of identifying an analyte, comprising
(a) providing an array of analytes, wherein each of the analytes is attached to a unique identifier in the array, wherein a docker is attached to each unique identifier in the array;
(b) providing an affinity reagent, wherein the affinity reagent comprises a paratope that recognizes an epitope of an analyte at a first unique identifier of the array, wherein the paratope preferentially recognizes the analyte compared to other analytes in the array, wherein the affinity reagent further comprises a tether that recognizes the docker;
(c) contacting the array with the affinity reagent, whereby the affinity reagent associates with the first unique identifier via binding of the paratope to the epitope and via binding of the tether to the docker; and
(d) detecting association of the affinity reagent with the first unique identifier, thereby identifying the analyte at the first unique identifier.

2. The method of claim 1, wherein the docker comprises a universal docker, each of the unique identifiers in the array being attached to a universal docker that is the same as a universal docker attached to the other unique identifiers in the array.

3.-42. (canceled)

43. The method of claim 1, wherein the docker comprises a first nucleotide sequence, wherein the tether comprises a second nucleotide sequence that is complementary to the first nucleotide sequence, and wherein the second nucleotide sequence comprises a sequence of 3 to 10 nucleotides that is complementary to the first nucleotide sequence.

44. The method of claim 1, wherein a plurality of dockers is attached to each unique identifier in the array.

45. The method of claim 1, wherein the affinity reagent comprises a plurality of tethers that recognize the docker.

46. The method of claim 1, wherein the affinity reagent comprises a plurality of paratopes, and wherein each of the paratopes recognizes the epitope of the analyte at the first unique identifier of the array.

47. The method of claim 46, wherein the affinity reagent further comprises a structured nucleic acid particle attached to: (i) the plurality of paratopes, (ii) a plurality of labels and (iii) a plurality of tethers.

48. The method of claim 1, wherein a first subset of indexed dockers is attached to a first subset of unique identifiers in the array and a subset of indexed dockers is attached to a second subset of unique identifiers in the array.

49. The method of claim 48, wherein step (c) comprises contacting the array with a first set of affinity reagents, wherein the first set of affinity reagents is attached to first tethers that bind selectively to the dockers of the first subset of indexed dockers compared to dockers of the second subset of indexed dockers, whereby the first set of affinity reagents associate with the first subset of unique identifiers via binding of the paratope to the epitope and via binding of the tether to the docker.

50. The method of claim 49, further comprising: (e) contacting the array with a second set of affinity reagents, wherein the second set of affinity reagents is attached to second tethers that bind selectively to dockers of the second subset of indexed dockers compared to dockers of the first subset of indexed dockers, whereby the second set of affinity reagents associate with the second subset of unique identifiers via binding of the paratope to the epitope and via binding of the tether to the docker.

51. The method of claim 1, further comprising:
(e) removing the affinity reagent from the array;
(f) contacting the array with a second affinity reagent, wherein the second affinity reagent comprises a second paratope that recognizes a second epitope of the analyte at the first unique identifier of the array, wherein the second paratope preferentially recognizes the analyte compared to other analytes in the array, wherein the second affinity reagent further comprises a second tether, whereby the second affinity reagent associates with the first unique identifier via binding of the second paratope to the second epitope and via binding of the second tether to the docker; and
(g) detecting association of the second affinity reagent with the first unique identifier, thereby identifying the analyte at the first unique identifier.

52. The method of claim 51, wherein the first unique identifier comprises a plurality of dockers.

53. The method of claim 52, wherein the first unique identifier comprises a greater number of dockers than the number of second tethers of the second affinity reagent.

54. The method of claim 53, wherein the affinity reagent comprises a plurality of tethers.

55. The method of claim 54, wherein the affinity reagent comprises a greater number of tethers than the number of second tethers of the second affinity reagent.

56. The method of claim 51, wherein the docker, tether and second tether comprise nucleic acid strands, wherein the docker hybridizes with the tether to form a duplex having a first length, and the docker hybridizes with the second tether to form a duplex having a second length that differs from the first length.

57. The method of claim 56, wherein the docker comprises a first nucleotide sequence, wherein the tether comprises a second nucleotide sequence that is complementary to the first nucleotide sequence, and wherein the second tether comprises a third nucleotide sequence that is complementary to the first nucleotide sequence.

58. The method of claim 57, wherein the second nucleotide sequence is the same length as the third nucleotide sequence or has a same melting temperature as the third nucleotide sequence.

59. The method of claim 57, wherein the second nucleotide sequence is a different length from the third nucleotide sequence or has a different melting temperature from the third nucleotide sequence.

60. The method of claim 57, wherein the second nucleotide sequence is the same as the third nucleotide sequence.
